(19) United States
(12) Patent Application Publication
(10) Pub. No.: US 2022/0364184 A1
(43) Pub. Date: Nov. 17, 2022

(54) COMPOSITIONS AND METHODS FOR TREATMENT OF A POOR PROGNOSIS SUBTYPE OF COLORECTAL CANCER

(71) Applicant: DANA-FARBER CANCER INSTITUTE, INC., Boston, MA (US)

(72) Inventors: Ruben D. Carrasco, Waban, MA (US); Meng Jiang, Harbin, Heilongjiang (CN); Tomasz Sewastianik, Boston, MA (US)

(73) Assignee: DANA-FARBER CANCER INSTITUTE, INC., Boston, MA (US)

(21) Appl. No.: 17/761,040

(22) PCT Filed: Sep. 24, 2020
(86) PCT No.: PCT/US2020/052500

§ 371 Date: Mar. 16, 2022

Related U.S. Application Data
(60) Provisional application No. 62/907,369, filed on Sep. 27, 2019.

(51) Int. Cl. C12Q 1/6886

(52) U.S. Cl. CPC C12Q 1/6886; C12Q 2600/112

(57) ABSTRACT

The present invention relates to compositions and methods for diagnosing and treating colorectal cancer.

### RELATED APPLICATIONS

This application claims the benefit of priority under 35 U.S.C. § 119(e) to U.S. Provisional Application No. 62/907,369, filed Sep. 27, 2019, which is incorporated herein by reference in its entirety.

### GOVERNMENT LICENSE RIGHTS

This invention was made with government support under grant number R01 CA151391 awarded by the National Institutes of Health. The Government has certain rights in the invention.

### SEQUENCE LISTING

The instant application contains a Sequence Listing which has been submitted electronically in ASCHII format and is hereby incorporated by reference in its entirety. Said ASCII copy, created on Sep. 24, 2020, is named 52095-5870001WO_ST25.txt and is 157 kilobytes in size.

### BACKGROUND OF THE INVENTION

Colorectal cancer (CRC), also referred to as bowel cancer, colon cancer, or rectal cancer, includes any cancer that affects the colon and the rectum. The American Cancer Society estimates that about one in 21 men and one in 23 women in the United States will develop colorectal cancer during their lifetime. The B-cell lymphoma 9 (BCL9) oncogene functions as a transcriptional co-activator of B-catenin in the canonical Wnt pathway, which plays critical roles in the pathogenesis of CRC. However, prior to the invention described herein, due to tumor heterogeneity and cell-type specific functions of BCL9, its role had not been well-defined across various CRC subtypes. Thus, prior to the invention described herein, there was a pressing need to determine the role of BCL9 in the pathogenesis CRC, thereby identifying new treatment modalities.

### SUMMARY OF THE INVENTION

The present invention is based upon the surprising discovery that the interaction of BCL9 with paraspeckle proteins provide neural-like, multi-cellular communication properties among tumor cells, thereby remodeling the tumor microenvironment and promoting tumor progression in a poor prognosis molecular subtype of colorectal cancer.

Methods of determining whether a subject (e.g., a human subject) has a C1 subtype of CRC are carried out by obtaining a test sample from a subject having or at risk of having CRC; determining the expression level of at least one C1 subtype-associated gene in the test sample; comparing the expression level of the C1 subtype-associate gene in the test sample with the expression level of the C1 subtype-associated gene in a reference sample; and identifying an elevated expression level of at least one C1 subtype-associated gene in the test sample as compared to the expression level of the C1 subtype-associated gene in a reference sample, wherein the C1 subtype-associated gene comprises a gene associated with wound healing, tissue remodeling, or neuron projection, thereby determining that the subject has a C1 subtype of colorectal cancer (CRC). In some cases, the methods include identifying an elevated expression level of at least two, at least three, at least four, at least five, or more C1 subtype-associated genes in the test sample as compared to the expression level of the C1 subtype-associated gene in a reference sample.

For example, the C1 subtype-associated gene comprises fibroblast activating protein (FAP), platelet derived growth factor subunit B (PDGFB), complement C3 (C3), calcium voltage-gated channel auxiliary subunit alpha2delta 1 (CACNA2D1), or regulator of G protein signaling 4 (RGS4).

In some cases, the methods further comprise identifying an elevated level of stromal cells in the test sample as compared to a reference sample. Exemplary stromal cells include fibroblasts, pericytes, and macrophages. Alternatively, the methods further comprise identifying an elevated level of stromal cells in the test sample as compared to the level of immune cells in the test sample. In another aspect, the methods further comprise identifying an elevated level of neural cells (i.e., ganglion cells) in the test sample as compared to a reference sample.

In some cases, the methods also include identifying an elevated level of nuclear BCL9 expression in tumor cells as compared to stromal cells from the test sample. For example, the BCL9 expression is localized adjacent to one or more paraspeckles within the nucleus. In some cases, the nuclear BCL9 expression in tumor cells exhibits a punctate (i.e., dotted) pattern. Optionally, the BCL9 expression or activity is independent of B-catenin expression or activity.

In some cases, the BCL9 co-localizes adjacent to one or more paraspeckle proteins selected from the group consisting of valosin containing protein (VCP), non-POU domain octamer binding protein (NONO), splicing factor proline and glutamine rich protein (SFPQ), and interleukin enhancer binding factor 2 protein (ILF2).

In one aspect, the test sample is obtained from a CRC tissue, a tumor microenvironment, a plasma sample, or a blood sample. For example, the sample comprises a deoxyribonucleic acid (DNA), a ribonucleic acid (RNA), or an amino acid.

In some cases, the reference sample is obtained from healthy normal tissue or CRC tissue. In another case, the reference sample is obtained from healthy normal tissue from the same individual as the test sample or one or more healthy normal tissues from different individuals.

For example, the expression level of the C1 subtype-associated gene is detected via an Affymetrix Gene Array hybridization, next generation sequencing, ribonucleic acid sequencing (RNA-seq), a real time reverse transcriptase polymerase chain reaction (real time RT-PCR) assay, immunohistochemistry (IHC), or immunofluorescence (IF).

In some cases, the subject is treated after being diagnosed with C1 subtype CRC. Methods of treating a subject with a C1 subtype of CRC are carried out by determining whether a subject has a C1 subtype of CRC according to the methods described herein, and administering a therapeutically effective amount of one or more BCL9 inhibitors to the subject, thereby treating a subject with a C1 subtype of CRC.

For example, the one or more BCL9 inhibitors comprise a small molecule inhibitor, RNA interference (RNAi), microRNA (miRNA), an antibody, an antibody fragment, an antibody drug conjugate, an aptamer, a chimeric antigen receptor (CAR), a T cell receptor, or any combination thereof.

In some cases, the antibody (e.g., anti-BCL9 antibody) or antibody fragment is partially humanized, fully humanized, or chimeric.

In another example, the BCL9 inhibitor comprises a stabilized alpha helix (SAH), hydrocarbon-stapled, BCL9.

An exemplary SAH BCL9 hydrocarbon-stapled polypeptide sequence is set forth below with the location of the hydrocarbon shown (SEQ ID NO: 1):

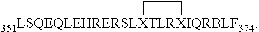

Another exemplary SAH BCL9 hydrocarbon-stapled polypeptide sequence is set forth below with the location of the hydrocarbon shown (SEQ ID NO: 2):

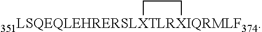

Another exemplary SAH BCL9 hydrocarbon-stapled polypeptide sequence is set forth below with the location of the hydrocarbon shown (SEQ ID NO: 3):

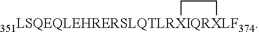

Another exemplary SAH BCL9 hydrocarbon-stapled polypeptide sequence is set forth below with the location of the hydrocarbon shown (SEQ ID NO: 4):

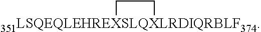

In yet another example, the BCL9 inhibitor comprises a miR-30 polynucleotide. For example, the miR-30 polynucleotide comprises a polynucleotide comprising one or more sequences selected from the group consisting of SEQ ID NOs: 9-13.

In some cases, the BCL9 inhibitor comprises a nanoparticle (e.g., a lipid nanoparticle) comprising at least one BCL9 inhibitor. For example, the BCL9 inhibitor comprises a small interfering ribonucleic acid (siRNA). Exemplary BCL9 siRNA sequences include:

(SEQ ID NO: 5)
5-AAUAACACUGUGUAUUGCAGC-3,
and
(SEQ ID NO: 6)
5-UUCCAGGGAUAUUCACAGAGG-3.

In some cases, the BCL9 inhibitor reduces the interaction between BCL9 and one or more paraspeckles. Preferably, BCL9 inhibition reduces tumor cell proliferation, tumor metastases, stromal cell infiltration, and response to cellular stress by e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

Preferably, the BCL9 inhibition reduces expression or activity of one or more genes associated with calcium signaling or neural differentiation including regulator of G protein signaling 4 (RGS4), calcium voltage-gated channel auxiliary subunit alpha 2 delta 1 (CACNA2D1), calcium channel, voltage-dependent, L type, alpha 1D subunit (CACNA1D), and adrenoceptor beta 1 (ADRB1). For example, expression or activity of these genes is reduced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

In one aspect, the methods also include administering a calcium channel receptor inhibitor or a beta-adrenergic antagonist (i.e., a beta blocker).

Exemplary calcium channel receptor inhibitors include verapamil, fendiline, gallopamil, amlodipine, aranidipine, azelnidipine, barnidipine, benidipine, cilidipine, clevidipine, efonidipine, felodipine, isradipine, lacidipine, lercanidipine, manidipine, nicardipine, nifedipine, nilvadipine, nimodipine, nisoldipine, nitrendipine, and pranidipine.

Suitable beta-adrenergic antagonists include propranolol, bucindolol, carteolol, carvedilol, labetalol, nadolol, oxprenolol, penbutolol, pindolol, sotalol, timolol, acebutolol, atenolol, betaxolol, bisoprolol, celiprolol, metoprolol, nebivolol, esmolol, butaxamine, and nebivolol.

In some cases, the methods also include treating the subject with a chemotherapeutic agent, radiation therapy, cryotherapy, hormone therapy, or immunotherapy. For example, the chemotherapeutic agent comprises fluorouracil, capecitabine, oxaliplatin, irinotecan, or tegafur/uracil.

Exemplary CRCs include adenocarcinoma, gastrointestinal stromal tumors (GIST), lymphoma, a carcinoid tumor, familial colorectal cancer (FCC), and juvenile polyposis coli.

### Definitions

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term “about.”

The phrase “aberrant expression” is used to refer to an expression level that deviates from (i.e., an increased or decreased expression level) the normal reference expression level of the gene.

The term “antineoplastic agent” is used herein to refer to agents that have the functional property of inhibiting a development or progression of a neoplasm in a human, e.g., a colorectal cancer. Inhibition of metastasis is frequently a property of antineoplastic agents.

By “agent” is meant any small compound, antibody, nucleic acid molecule, or polypeptide, or fragments thereof.

By “alteration” is meant a change (increase or decrease) in the expression levels or activity of a gene or polypeptide as detected by standard art-known methods such as those described herein. As used herein, an alteration includes at least a 1% change in expression levels, e.g., at least a 2%, 3%, 4%, 5%, 6%, 7%, 8%, 9%, 10%, 20%, 30%, 40%, 50%, 60%, 70%, 80%, 90%, or 100% change in expression levels. For example, an alteration includes at least a 5%-10% change in expression levels, preferably a 25% change, more preferably a 40% change, and most preferably a 50% or greater change in expression levels.

The term “antibody” (Ab) as used herein includes monoclonal antibodies, polyclonal antibodies, multispecific antibodies (e.g., bispecific antibodies), and antibody fragments, so long as they exhibit the desired biological activity. The term “immunoglobulin” (Ig) is used interchangeably with “antibody” herein.

An “isolated antibody” is one that has been separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In preferred embodiments, the antibody is purified: (1) to greater than 95% by weight of antibody as determined by the Lowry method, and most preferably more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

By “binding to” a molecule is meant having a physicochemical affinity for that molecule.

By “control” or “reference” is meant a standard of comparison. In one aspect, as used herein, “changed as compared to a control” sample or subject is understood as having a level that is statistically different than a sample from a normal, untreated, or control sample. Control samples include, for example, cells in culture, one or more laboratory test animals, or one or more human subjects. Methods to select and test control samples are within the ability of those in the art. An analyte can be a naturally occurring substance that is characteristically expressed or produced by the cell or organism (e.g., an antibody, a protein) or a substance produced by a reporter construct (e.g, β-galactosidase or luciferase). Depending on the method used for detection, the amount and measurement of the change can vary. Determination of statistical significance is within the ability of those skilled in the art, e.g., the number of standard deviations from the mean that constitute a positive result.

“Detect” refers to identifying the presence, absence, or amount of the agent (e.g., a nucleic acid molecule, for example deoxyribonucleic acid (DNA) or ribonucleic acid (RNA)) to be detected.

A “detection step” may use any of a variety of known methods to detect the presence of nucleic acid (e.g., DNA) or polypeptide. The types of detection methods in which probes can be used include Western blots, Southern blots, dot or slot blots, and Northern blots.

As used herein, the term “diagnosing” refers to classifying pathology or a symptom, determining a severity of the pathology (e.g., grade or stage), monitoring pathology progression, forecasting an outcome of pathology, and/or determining prospects of recovery.

By the terms “effective amount” and “therapeutically effective amount” of a formulation or formulation component is meant a sufficient amount of the formulation or component, alone or in a combination, to provide the desired effect. For example, by “an effective amount” is meant an amount of a compound, alone or in a combination, required to ameliorate the symptoms of a disease, e.g., CRC, relative to an untreated patient. The effective amount of active compound(s) used to practice the present invention for therapeutic treatment of a disease varies depending upon the manner of administration, the age, body weight, and general health of the subject Ultimately, the attending physician or veterinarian will decide the appropriate amount and dosage regimen. Such amount is referred to as an “effective” amount.

The term “expression profile” is used broadly to include a genomic expression profile. Profiles may be generated by any convenient means for determining a level of a nucleic acid sequence, e.g., quantitative hybridization of microRNA, labeled microRNA, amplified microRNA, complementary/synthetic DNA (cDNA), etc., quantitative polymerase chain reaction (PCR), and ELISA for quantitation, and allow the analysis of differential gene expression between two samples. A subject or patient tumor sample is assayed. Samples are collected by any convenient method, as known in the art. According to some embodiments, the term “expression profile” means measuring the relative abundance of the nucleic acid sequences in the measured samples.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. For example, a fragment may contain 10, 20, 30, 40, 50, 60, 70, 80, 90, or 100, 200, 300, 400, 500, 600, 700, 800, 900, or 1000 nucleotides or amino acids. However, the invention also comprises polypeptides and nucleic acid fragments, so long as they exhibit the desired biological activity of the full length polypeptides and nucleic acid, respectively. A nucleic acid fragment of almost any length is employed. For example, illustrative polynucleotide segments with total lengths of about 10,000, about 5000, about 3000, about 2,000, about 1,000, about 500, about 200, about 100, about 50 base pairs in length (including all intermediate lengths) are included in many implementations of this invention. Similarly, a polypeptide fragment of almost any length is employed. For example, illustrative polypeptide segments with total lengths of about 10,000, about 5,000, about 3,000, about 2,000, about 1,000, about 5,000, about 1,000, about 500, about 200, about 100, or about 50 amino acids in length (including all intermediate lengths) are included in many implementations of this invention.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation.

A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

By “isolated nucleic acid” is meant a nucleic acid that is free of the genes which flank it in the naturally-occurring genome of the organism from which the nucleic acid is derived. The term covers, for example: (a) a DNA which is part of a naturally occurring genomic DNA molecule, but is not flanked by both of the nucleic acid sequences that flank that part of the molecule in the genome of the organism in which it naturally occurs; (b) a nucleic acid incorporated into a vector or into the genomic DNA of a prokaryote or eukaryote in a manner, such that the resulting molecule is not identical to any naturally occurring vector or genomic DNA; (c) a separate molecule such as a synthetic cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Isolated nucleic acid molecules according to the present invention further include molecules produced synthetically, as well as any nucleic acids that have been altered chemically and/or that have modified backbones. For example, the isolated nucleic acid is a purified cDNA or RNA polynucleotide. Isolated nucleic acid molecules also include messenger ribonucleic acid (mRNA) molecules.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by high performance liquid chromatography (HPLC) analysis.

The term, “normal amount” refers to a normal amount of a complex in an individual known not to be diagnosed with CRC. The amount of the molecule can be measured in a test sample and compared to the “normal control level,” utilizing techniques such as reference limits, discrimination limits, or risk defining thresholds to define cutoff points and abnormal values (e.g., for CRC). The “normal control level” means the level of one or more proteins (or nucleic acids) or combined protein indices (or combined nucleic acid indices) typically found in a subject known not to be suffering from CRC. Such normal control levels and cutoff points may vary based on whether a molecule is used alone or in a formula combining other proteins into an index. Alternatively, the normal control level can be a database of protein patterns from previously tested subjects who did not convert to CRC over a clinically relevant time horizon. In another aspect, the normal control level can be a level relative to a housekeeping gene.

The level that is determined may be the same as a control level or a cut off level or a threshold level, or may be increased or decreased relative to a control level or a cut off level or a threshold level. In some aspects, the control subject is a matched control of the same species, gender, ethnicity, age group, smoking status, body mass index (BMI), current therapeutic regimen status, medical history, or a combination thereof, but differs from the subject being diagnosed in that the control does not suffer from the disease in question or is not at risk for the disease.

Relative to a control level, the level that is determined may be an increased level. As used herein, the term “increased” with respect to level (e.g., expression level, biological activity level, etc.) refers to any % increase above a control level. The increased level may be at least or about a 1% increase, at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, or at least or about a 95% increase, relative to a control level.

Relative to a control level, the level that is determined may be a decreased level. As used herein, the term “decreased” with respect to level (e.g., expression level, biological activity level, etc.) refers to any % decrease below a control level. The decreased level may be at least or about a 1% decrease, at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, or at least or about a 95% decrease, relative to a control level.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity, e.g., at least 80%, at least 85%, at least 90%, at least 95%, or at least 99% identity. Polynucleotides having “substantial identity” to an endogenous sequence are typically capable of hybridizing with at least one strand of a double-stranded nucleic acid molecule.

As used herein, in one aspect, “next-generation sequencing” (NGS), also known as high-throughput sequencing, is the catch-all term used to describe a number of different sequencing methodologies including, but not limited to, Illumina® sequencing, Roche 454 Sequencing™, Ion Torrent™: Proton/personal genome machine (PGM) sequencing, and SOLiD sequencing. These recent technologies allow for sequencing DNA and RNA much more quickly and cheaply than the previously used Sanger sequencing. See, LeBlanc et al., 2015 Cancers, 7: 1925-1958, incorporated herein by reference; and Goodwin et al., 2016 Nature Reviews Genetics, 17: 333-351, incorporated herein by reference.

As used herein, “obtaining” as in “obtaining an agent” includes synthesizing, purchasing, or otherwise acquiring the agent.

Unless specifically stated or obvious from context, as used herein, the term “or” is understood to be inclusive. Unless specifically stated or obvious from context, as used herein, the terms “a”, “an”, and “the” are understood to be singular or plural.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier should be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

By “protein” or “polypeptide” or “peptide” is meant any chain of more than two natural or unnatural amino acids, regardless of post-translational modification (e.g., glycosylation or phosphorylation), constituting all or part of a naturally-occurring or non-naturally occurring polypeptide or peptide, as is described herein.

The terms “preventing” and “prevention” refer to the administration of an agent or composition to a clinically asymptomatic individual who is at risk of developing, susceptible, or predisposed to a particular adverse condition, disorder, or disease, and thus relates to the prevention of the occurrence of symptoms and/or their underlying cause.

The term “prognosis,” “staging,” and “determination of aggressiveness” are defined herein as the prediction of the degree of severity of the neoplasia, e.g., CRC, and of its evolution as well as the prospect of recovery as anticipated from usual course of the disease. Once the aggressiveness has been determined, appropriate methods of treatments are chosen.

The terms “microRNA” or “miRNA” or “miR” are used interchangeably herein refer to endogenous RNA molecules, which act as gene silencers to regulate the expression of protein-coding genes at the post-transcriptional level. Endogenous microRNA are small RNAs naturally present in the genome which are capable of modulating the productive utilization of mRNA. The term artificial microRNA includes any type of RNA sequence, other than endogenous microRNA, which is capable of modulating the productive utilization of mRNA. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

By “reduces” is meant a negative alteration of at least 10%, 25%, 50%, 75%, or 100%.

The term “sample” as used herein refers to a biological sample obtained for the purpose of evaluation in vitro. Exemplary tissue samples for the methods described herein include tissue samples from CRC tumors or the surrounding microenvironment (i.e., the stroma). With regard to the methods disclosed herein, the sample or patient sample preferably may comprise any body fluid or tissue. In some embodiments, the bodily fluid includes, but is not limited to, blood, plasma, serum, lymph, breast milk, saliva, mucous, semen, vaginal secretions, cellular extracts, inflammatory fluids, cerebrospinal fluid, feces, vitreous humor, or urine obtained from the subject. In some aspects, the sample is a composite panel of at least two of a blood sample, a plasma sample, a serum sample, and a urine sample. In exemplary aspects, the sample comprises blood or a fraction thereof (e.g., plasma or serum). Preferred samples are whole blood, serum, plasma, or urine. A sample can also be a partially purified fraction of a tissue or bodily fluid.

A reference sample can be a “normal” sample, from a donor not having the disease or condition fluid, or from a normal tissue in a subject having the disease or condition. A reference sample can also be from an untreated donor or cell culture not treated with an active agent (e.g., no treatment or administration of vehicle only). A reference sample can also be taken at a “zero time point” prior to contacting the cell or subject with the agent or therapeutic intervention to be tested or at the start of a prospective study.

A “solid support” describes a strip, a polymer, a bead, or a nanoparticle. The strip may be a nucleic acid-probe (or protein) coated porous or non-porous solid support strip comprising linking a nucleic acid probe to a carrier to prepare a conjugate and immobilizing the conjugate on a porous solid support. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to a binding agent (e.g., an antibody or nucleic acid molecule). Thus, the support configuration may be spherical, as in a bead, or cylindrical, as in the inside surface of a test tube, or the external surface of a rod. Alternatively, the surface may be flat such as a sheet, or test strip, etc. For example, the supports include polystyrene beads. Those skilled in the art will know many other suitable carriers for binding antibody or antigen, or will be able to ascertain the same by use of routine experimentation. In other aspects, the solid support comprises a polymer, to which an agent is chemically bound, immobilized, dispersed, or associated. A polymer support may be a network of polymers, and may be prepared in bead form (e.g., by suspension polymerization). The location of active sites introduced into a polymer support depends on the type of polymer support. For example, in a swollen-gel-bead polymer support the active sites are distributed uniformly throughout the beads, whereas in a macroporous-bead polymer support they are predominantly on the internal surfaces of the macropores. The solid support, e.g., a device contains a binding agent alone or together with a binding agent for at least one, two, three or more other molecules.

By “specifically binds” is meant a compound or antibody that recognizes and binds a polypeptide of the invention, but which does not substantially recognize and bind other molecules in a sample, for example, a biological sample, which naturally includes a polypeptide of the invention.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, at least 70%, at least 80%, at least 85%, at least 90%, at least 95%, or at least 99% identical at the amino acid level or nucleic acid to the sequence used for comparison.

The term “subject” as used herein includes all members of the animal kingdom prone to suffering from the indicated disorder. In some aspects, the subject is a mammal, and in some aspects, the subject is a human. The methods are also applicable to companion animals such as dogs and cats as well as livestock such as cows, horses, sheep, goats, pigs, and other domesticated and wild animals.

A subject “suffering from or suspected of suffering from” a specific disease, condition, or syndrome has a sufficient number of risk factors or presents with a sufficient number or combination of signs or symptoms of the disease, condition, or syndrome such that a competent individual would diagnose or suspect that the subject was suffering from the disease, condition, or syndrome. Methods for identification of subjects suffering from or suspected of suffering from conditions associated with cancer (e.g., CRC) is within the ability of those in the art. Subjects suffering from, and suspected of suffering from, a specific disease, condition, or syndrome are not necessarily two distinct groups.

As used herein, “susceptible to” or “prone to” or “predisposed to” or “at risk of developing” a specific disease or condition refers to an individual who based on genetic, environmental, health, and/or other risk factors is more likely to develop a disease or condition than the general population. An increase in likelihood of developing a disease may be an increase of about 10%, 20%, 50%, 100%, 150%, 200%, or more.

The terms “treating” and “treatment” as used herein refer to the administration of an agent or formulation to a clinically symptomatic individual afflicted with an adverse condition, disorder, or disease, so as to effect a reduction in severity and/or frequency of symptoms, eliminate the symptoms and/or their underlying cause, and/or facilitate improvement or remediation of damage. It will be appreciated that, although not precluded, treating a disorder or condition does not require that the disorder, condition or symptoms associated therewith be completely eliminated.

As used herein, in one aspect, the “tumor microenvironment” (TME) is the cellular environment in which a tumor exists, including surrounding blood vessels, immune cells, fibroblasts, bone marrow-derived inflammatory cells, lymphocytes, signaling molecules and the extracellular matrix (ECM). The tumor and the surrounding microenvironment are closely related and interact constantly. Tumors can influence the microenvironment by releasing extracellular signals, promoting tumor angiogenesis and inducing peripheral immune tolerance, while the immune cells in the microenvironment can affect the growth and evolution of cancerous cells, such as in immuno-editing.

In some cases, a composition of the invention is administered orally or systemically. Other modes of administration include rectal, topical, intraocular, buccal, intravaginal, intracistemal, intracerebroventricular, intratracheal, nasal, transdermal, within/on implants, or parenteral routes. The term “parenteral” includes subcutaneous, intrathecal, intravenous, intramuscular, intraperitoneal, or infusion. Intravenous or intramuscular routes are not particularly suitable for long-term therapy and prophylaxis. They could, however, be preferred in emergency situations. Compositions comprising a composition of the invention can be added to a physiological fluid, such as blood. Oral administration can be preferred for prophylactic treatment because of the convenience to the patient as well as the dosing schedule. Parenteral modalities (subcutaneous or intravenous) may be preferable for more acute illness, or for therapy in patients that are unable to tolerate enteral administration due to gastrointestinal intolerance, ileus, or other concomitants of critical illness. Inhaled therapy may be most appropriate for pulmonary vascular diseases (e.g., pulmonary hypertension).

Pharmaceutical compositions may be assembled into kits or pharmaceutical systems for use in arresting cell cycle in rapidly dividing cells, e.g., cancer cells. Kits or pharmaceutical systems according to this aspect of the invention comprise a carrier means, such as a box, carton, and tube, having in close confinement therein one or more container means, such as vials, tubes, ampoules, bottles, syringes, or bags. The kits or pharmaceutical systems of the invention may also comprise associated instructions for using the kit.

Any compositions or methods provided herein can be combined with one or more of any of the other compositions and methods provided herein.

Where applicable or not specifically disclaimed, any one of the embodiments described herein are contemplated to be able to combine with any other one or more embodiments, even though the embodiments are described under different aspects of the invention.

These and other embodiments are disclosed and/or encompassed by, the following Detailed Description.

### BRIEF DESCRIPTION OF THE DRAWINGS

FIG. 1A-FIG. 1H is a series of graphs, schematics, and immunostains illustrating BCL9 expression within specific molecular subtypes of CRC. FIG. 1A is a diagram containing CRC clusters and the corresponding number, histology, and stage of samples (top). Adenocarcinoma (AC); mucinous adenocarcinoma (MAC); tumor, node, metastasis (TNM). A heat map of unsupervised clustering of log 2 gene expression levels for CRC clusters (middle) and the type of gene mutation (bottom). FIG. 1B is a series of Cox proportional hazard plots of CRC survival probability according to cluster and BCL9 expression levels (high: top 25%; low: bottom 25%). FIG. 1C is a schematic depicting GSEA (gene set enrichment analysis) of CRC clusters. Color-code response to log 10 FDR level (red and blue: high and low confidence, respectively). FIG. 1D is a group of photomicrographs that are representative immunostains of BCL9 and Fibroblast Activating Protein (FAP) (top) and immunofluorescence of BCL9 (bottom) according to FAP levels. Tu: tumor; St: stroma. Scale bars: black, 50 μm (top, left) and 10 μm (inset); white, 20 μm (top, left) and 2 μm (inset). FIG. 1E is a bar graph of the distribution of BCL9 punctate staining according to FAP levels. ***: P<0.001. FIG. 1F is a 3D scatter plot of immunostain H-scores for the indicated proteins. FIG. 1G is a series dot plots depicting immunostain H-scores of indicated proteins in FAP low and high groups. *: P<0.05; ***: P<0.001; ns: not significant. FIG. 1H is a series of Pearson correlation plots of immunostain H-scores of indicated proteins in FAP low and high groups.

FIG. 2A-FIG. 2D is a series of diagrams, schematics, and heat maps depicting transcriptional correlation network of BCL9-assisted biological processes in CRC subtypes and BCL9-dependent regulation of calcium signaling related genes. FIG. 2A is a GSEA Chow-Ruskey diagram of BCL9-interacting proteins as determined by immunoprecipitation (IP) coupled with mass spectrometry (MS) analysis. Size of colored areas indicates the number of functional gene sets in each module eigengene (ME). Upper heat map depicts normalized RNA-seq data of indicated genes and CRC clusters. Colors represent normalized ribonucleic acid (RNA)-seq expression levels in CRC clusters. Each row represents the mean value of three independent biological repeats. Lower heat map shows IP coupled MS data. Colors represent total peptide number of indicated proteins. FIG. 2B is a diagram with GSEA in pathway gene set for down regulated genes in normalized RNA-seq data of indicated genes and CRC clusters. The left heat map depicts normalized RNA-seq data of indicated genes and CRC clusters. The right heat map shows normalized expression value of RNA-seq results of control and BCL9 knockout RKO cells. FIG. 2C is a heat map of the adjacencies in the eigengene network including patient survival for CRC Cluster 1. In the outer boxed heat map, each colored square in the column or row corresponds to one ME or survival time (red arrowhead). In the inner boxed heat map, blue and red colors represent low adjacency (negative correlation), and high adjacency (positive correlation), respectively. Red squares along the diagonal are the meta-modules. Black dots along this diagonal highlight ME displaying a negative correlation with survival time. Colored interconnecting lines indicate group affiliation between ME and BCL9-interacting proteins (top) and genes down regulated in BCL9 knockout RKO cells (left). FIG. 2D shows the GSEA of ME-Black, ME-Brown, and ME-Blue groups.

FIG. 3A-FIG. 3J is a series of images, schematics, graphs, heat maps, and immunoblots showing that BCL9 regulates mRNA levels of calcium wave-associated genes through paraspeckle proteins. FIG. 3A is an illustration of the network of BCL9-interacting proteins identified by Co-IP MS. Each node represents a group of BCL9-interacting proteins with functional relationships. Lines between different nodes represent the “weight” of the protein-protein interaction according to String database (colored) or total peptides in Co-IP assay (black). Groups were clustered by k-means unsupervised classification according to the interacting “weight” among different nodes. FIG. 3B is a group of images of immunofluorescence (IF) showing co-localization of BCL9, Non-POU Domain Containing Octamer Binding (NONO), and Interleukin Enhancer Binding Factor 2 (ILF2) in CRC but not in normal colon epithelial cells. Scale bars: 20 μm (top, left), and 2 μm (inset). FIG. 3C is a graph showing high fold change: genes whose mRNA levels decreased more than 1.5-fold in BCL9 knockout cells and low fold change: others. **, P<0.001. FIG. 3D is a depiction of the Splicing Factor Proline and Glutamine Rich (SFPQ) binding motif in the 3′UTR region of indicated messenger ribonucleic acid (mRNA)-encoding genes down-regulated in BCL9-knockout RKO cells. FIG. 3E is a series of bar graphs for RNA immunoprecipitation couple with polymerase chain reaction (RIP-PCR) verification of BCL9, NONO, and SFPQ interaction with RGS4 mRNA in the indicated cell lines. GAPDH was used as a negative control. FIG. 3F is an IF time course of BCL9 localization (left) and an immunoblot showing the expression in Poly I:C treatment RKO cells. Scale bar: 1 μm. FIG. 3G is an IF co-localization of BCL9 and NONO after 6 hrs of Poly I:C treatment in the indicated cells. White triangles indicate co-localized pixels of NONO and BCL9. Dotted circles represent a group of pixels which have high intensity of both BCL9 and indicated protein. Scale bar: 2 μm. FIG. 3H is a Co-IP of NONO and ILF2 interaction in two different BCL9 knockout RKO clones. FIG. 3I is a bar graph for RGS4 mRNA expression in actinomycin D treated wild-type and BCL9 knockout RKO cells. FIG. 3J is a heat map of RNA-seq data of wild-type and BCL9 knockout RKO cells treated with vehicle or Poly I:C (P<0.05) and a graph that highlights the changes of RGS4 transcript isoforms. Data are displayed as mean±SD for three independent experiments and repeated twice.

FIG. 4A-FIG. 4I is a series of photomicrographs, plots, graphs, heat maps, and spectra supporting that a lack of BCL9 inhibits the occurring and spreading of calcium transients. FIG. 4A is a time-lapse of images demonstrating the spread of calcium transient among cells (left) and average calcium transient curve (ΔF/F0) of all cells with calcium transients in one microscope field (right). Initiating cells (white arrows), direction of propagation (yellow arrows). Synchronicity of calcium transients is highlighted by red triangle. Scale bar: 10 μm. FIG. 4B is a group of dot-plots of synchronized calcium transients in the indicated wild-type and BCL9 knockout cells (left). Each dot represents one cell, the colored dots indicate the number of calcium wave events during 30 mins. Edged-linked dots represent synchronized calcium transients, colored edge indicates the timing of synchronized calcium transients. Synchronicity of global calcium transients in the indicated cells was calculated in wild-type and BCL9 knockout by FluoroSNNAP, n.d. non-detectable. ****: P<0.0001 (right). FIG. 4C is a bar graph depicting the frequency of calcium waves occurring in the indicated wild-type and BCL9 knockout RKO cells in the absence or presence of Verapamil or ethylene diamine tetra-acetic acid (EDTA). *: P<0.05; ns: not significant. FIG. 4D is a bar graph showing peak height of calcium transients in wild-type and BCL9 knockout RKO cells. FIG. 4E is a group of graphs portraying the network of calcium transients spreading (left) and ΔF/F0 of synchronized “secondary waves” (right) after wound scratching in wild-type and BCL9 knockout RKO cells. Each circle represents one cell, colors represent times of simultaneous calcium transients. Edged-linked dots represent synchronized calcium transients, colored edge indicates times of synchronized calcium transients. Red triangle: “secondary waves”; Black line: wound edge. Scale bar: 5 μm. FIG. 4F is a group of graphs showing ΔF/F0 and heat map showing display of “secondary waves” in Colo320 but not in DLD-1 cells. FIG. 4G is the frequency spectrum of calcium waves of indicated region of interest (ROI) in FIGS. 4E and 4F. FIG. 4H is a Co-IP of the indicated proteins in RKO cells treated for 2 hrs with vehicle (−) or verapamil (+). FIG. 4I is a time-course IF depicting changes in BCL9 staining area in cells located adjacent or distant from scratched edge in vehicle (left) or Verapamil (right) treated RKO cells. Data is displayed as mean±SD for three independent experiments and repeated twice. Scale bar: 1 μm.

FIG. 5A-FIG. 5H is a series of graphs, schematics, photomicrographs, and structures illustrating the identification of calcium transient inducers by MS analysis. FIG. 5A is a dot plot showing RGS4 mRNA expression in five different BCL9 knockout RKO clones mock treated or treated with conditioned medium (CM) from wild-type RKO cells, with or without proteinase K pre-treatment. ***, P<0.001; **, P<0.005; ns, not significant. FIG. 5B is a diagram of strategy used to identify “extracellular factor(s)” inducers of calcium waves spreading. FIG. 5C is a series of graphs depicting retention time and molecular size (M/Z) of small molecules identified by MS in CM from 3 different BCL9 knockout RKO cell clones and compared with CM from wild-type RKO cells. FIG. 5D molecular structures of small-molecules identified by MS in CM from 3 different BCL9 knockout RKO cell clones and compared with CM from wild-type RKO cells. FIG. 5E is a heat map of calcium transient (ΔF/F0) in the indicated ROI (red dots) in wild-type and BCL9 knockout RKO cells. White dotted line indicates the time of adding Terbutaline to the cell culture medium. The frequency spectrum shows calcium waves of indicated ROI (bottom). Red and yellow arrows indicate the direction of primary and secondary way propagation, respectively. Scale bar: 20 μm. FIG. 5F contains a dot-plot showing a network of synchronous calcium transients of calcium wave spreading. Time-lapse imaging (right) after scratching the monolayer surface in control and propranolol treated RKO cells. The heat map shows cell response time after scratching. Yellow: fast; Blue: slow. Scale bar: 20 μm. FIG. 5G contains time-lapse imaging of calcium waves in THP-1 cells co-cultured with wild-type or BCL9 knockout RKO cells showing direct communication of macrophages and colorectal cancer cells. White arrows: RKO cells; Red arrows: THP-1 cells. THP-1 cells were transfected with GCAMP5 and ds Red, RKO cells were transfected with GCAMP5 only. A dot-plot displaying the synchronous calcium transient network of wild type RKO and THP-1 cells co-culture system and the frequency spectrum of calcium waves of wild-type and BCL9 knockout RKO and THP-1 co-culture system. Characteristic spectral lines are marked by red triangle. Scale bar: 20 μm. FIG. 5H is a bar graph showing the RT-qPCR analysis of TGFB2 and IL10 mRNA expression in THP-1 cells treated with PMA and protein free CM from wild-type or BCL9 knockout RKO cells.

FIG. 6A-FIG. 6I is a series of plots, schematics, and photomicrographs demonstrating knockout expression of BCL9 and propranolol treatment reduces tumor CRC progression and stromal cell infiltration in vivo. FIG. 6A is a plot depicting tumor burden assessed by body live imaging over time. Each dot represents mean; error bars, standard error of the mean. P values were calculated using Student's t test. N=7 for both groups. Day 0 indicates first measurement. FIG. 6B is a series of body live images taken on day 34 (top) and a representative histology and BCL9 immunostain of xenograft tumors (bottom). Arrowheads indicate accumulation of BCL9 around paraspeckles. Scale bars: Black 50 μm; White 20 μm. FIG. 6C is a collection of photomicrographs that are representative immunohistochemical analysis of xenografn tumors. Scale bars: white, 20 μm; black, 50 μm. FIG. 6D is a series of dot-plots of the percentage of 0-catenin (left) and Ki-67 (right) positive cells per ROI. N represents number of ROIs analyzed. Tumor nodules dissected from three mice implanted with wild-type or BCL9 knockout RKO cells were used for analysis. Crosses represent mean and standard deviation. P values were calculated using Welch's t test. ***, P<0.001. FIG. 6E is a series of dot-plots of the percentage of the area positive for CD163, CD31, and αSMA staining per ROI. ****: P<0.0001. n.s.: not significant. FIG. 6F is a plot depicting tumor burden assessed by body live imaging over time. Each dot represents mean; error bars, standard error of the mean fold change. P values were calculated using Student's t test n=7 for both groups. Day 1 indicates first measurement. FIG. 6G is a series of body live images taken on day 1, 7, 14, and 21. FIG. 6H is a series of graphs depicting the percentage of indicated cells of ROI. N: number of ROIs analyzed. **: P<0.01. ***: P<0.001. n.s.: not significant. FIG. 6I is a series of representative immunohistochemical analysis of xenograft tumors. Scale bars: 50 μm.

FIG. 7 is a schematic model for BCL9-dependent upregulation of calcium wave propagation among CRC cells and with the tumor microenvironment. In wild-type C1 CRC cells (left) (e.g. RKO), the generation of spontaneous calcium transients through voltage-gated calcium channel opening, leads to neurotransmitter release and activation of neighboring cells bearing its receptor (e.g. GPCR (G-protein-coupled receptors)) to promote calcium release and wave propagation. Simultaneously, CRC cell stimulation and calcium influx promote BCL9 accumulation around and interaction with paraspeckles, generating a positive feedback loop to stabilize mRNA of calcium associated genes (e.g. CACNA2D1 (voltage-dependent calcium channel subunit alpha-2/delta-1)) and ensuring the occurrence of the subsequent calcium transients. Thus, BCL9 translocation into paraspeckles provides C1 CRC cells with neuronal-like properties (e.g. cytoplasmic projections, calcium waves) to enhance communication among tumor cells and cells from the tumor microenvironment; this subsequently promotes tumor progression by enhancing tumor/stromal cell migration, invasion, proliferation, and survival, as well as tissue remodeling. In C1 CRC cells lacking BCL9 (right), the neuronal-like properties are lost; this is due to the lack of expression of genes associated with the generation of calcium waves, the disruption of the positive feed-back loop, and the fact that calcium waves are not strong enough to induce neurotransmitter release. This consequently terminates the propagation of calcium waves and communication among tumor cells and with the tumor microenvironment. I.R.C. represents interchromosomal region.

FIG. 8A-FIG. 8E is a series of matrixes, plots, and graphs depicting the CRC clusters and the corresponding number, histology, and stage of samples. FIG. 8A is a group of consensus clustering matrixes of unsupervised classification for k=3, 4, 8, and 10. In each consensus matrix, both the rows and the columns were indexed with the same sample order and samples belonging to the same cluster are adjacent to each other. The consensus index for each pair of samples is shown in color from white (0%) to blue (100%). Colored boxes on the top of heat map indicates a different cluster. FIG. 8B is a tracking plot of k values in unsupervised classification. Bottom back rows: response to patient samples. Columns: response to k value, colors indicate different clusters. FIG. 8C is a graph showing relative change area of the Cumulative Distribution Function (CDF) curve for k=2 to k=10. At the black arrowhead, the gradient of the curve becomes less steep. Note that the curve tends to plateau after k=4, suggesting that the clustering is overfitting. FIG. 8D is a group of bar graphs showing the frequency of the indicated gene mutations in each cluster. FIG. 8E is a series of bar graphs depicting the frequency of patient's TNM tumor stage for each cluster.

FIG. 9A-FIG. 9E is a series of graphs, plots, and tables illustrating analysis of CRC clusters. FIG. 9A is a series of gene set enrichment analysis for the indicated CRC clusters and gene function. For each indicated cluster, signature genes were compared with the average expression of the other three clusters FIG. 9B is a series of graphs for the expression analysis of BCL9 and the indicated genes in different clusters. Red dots: normal colon epithelium, Green dots: CRC samples in cluster 1. FIG. 9C is a series of graphs for the analysis of tumor cell signaling purity across clusters. Cluster 1 displays the lower purity score and highest stromal cell score among all four clusters. FIG. 9D is a series of matrixes depicting GSEA of CRC clusters generated by GSE39582. Color-code response to log 10 FDR level (red and blue: high and low confidence, respectively). FIG. 9E is a series of Kaplan-Meier plots of CRC survival probability according to GSE39582 cluster and BCL9 expression levels (high: top 25%; low: bottom 25%).

FIG. 10A-FIG. 10F is a series of graphs, tables, and photomicrographs depicting BCL9 with high and low levels of FAP. FIG. 10A is a series of immunostains showing high expression in stromal and neuronal, but low expression of BCL9 in epithelial cells of normal colon mucosa. Scale bar: 10 μm. FIG. 10B is a series of representative immunostains of the indicated proteins in serial consecutive sections from a tissue microarray containing 89 CRC and 30 normal colon mucosal samples in FAP low and high groups. Scale bar: 20 μm. FIG. 10C is a group of representative IF stains (top) and fluorescent colocalization threshold (bottom) of BCL9 and β-catenin in a tissue biopsy containing areas with inactivate (left) and active (right) β-catenin CRC patient samples. Correlation of pixels intensity was calculated by Image J, note colocalization of BCL9 and β-catenin was shown in case #8 but not case #7. FIG. 10D is a table showing the number of cases with BCL9 and/or β-catenin nuclear staining in FAP low and high groups. FIG. 10E is a set of representative IF of the indicated cancer types. FIG. 10F is a set of heat maps of BCL9 and β-catenin staining in tissue microarray (TMA) containing the indicated cancer types. Numbers and color intensities indicate the intensity of nuclear staining; Red, 2: strong; Pink, 1: weak; White, 0: no staining. Scale bar: 20 μm, 5 μm.

FIG. 11A-FIG. 11G is a series of photomicrographs, graphs, and dot-plots showing BCL9-associated biological processes in CRC. FIG. 11A is a heat map depicting gene expression profiling of CRC cell lines which represented different CRC clusters. FIG. 11B is a series of 3D dot-plots showing PCA analysis in two different perspective of CRC clustered cell lines. FIG. 11C is a series of photomicrographs of IF of BCL9 and β-catenin in the indicated cell lines shown at low (top), high (middle) magnification, and higher 2% intensity of BCL9 staining (bottom). Scale bar: low magnification, 20 μm; high magnification, 1 μm. FIG. 11D is a schematic representation of BCL9 high frequency signaling removal. FFT: Fast Fourier Transform. FIG. 11E is a bar graph and dot-plot showing frequency (left) and size area (right) of BCL9 punctate staining in the indicated cell lines. ****: P<le-6. FIG. 11F is an immunoblot of BCL9 in the indicated cell lines after lentiviral transduction with or shBCL9 or shLacZ used as controls. FIG. 11G is a bar graph depicting cell viability in the indicated cell lines after lentiviral transduction with or shBCL9 or shLacZ used as controls. shBCL9-1 and shBCL9-2 indicate two different hairpins. *: P<0.01; **: P<0.005; ns: not significant.

FIG. 12A-FIG. 12E is a series of immunoblots, microphotographs, and tables depicting BCL9 activity. FIG. 12A is a group of BCL9 immunoblots (left) and silver stained gels (right) of immunoprecipitated proteins in colo320 cells. *, **, and *** indicate bands present in anti-BCL9 but not in anti-IgG groups. FIG. 12B is a table containing protein names and the average of total peptide number for each of the bands indicated and excised from gels above and analyzed by MS. FIG. 12C is a group of IP coupled immunoblots of the indicated proteins and cell lines. BCL9-1 and BCL9-2 correspond to two different anti-BCL9 antibodies. FIG. 12D is a series of microphotographs showing representative IF of NONO and SFPQ in Hela cells. Colocalization threshold was calculated by ImageJ, dotted areas represent a group of pixels that have high intensity for both NONO and SFPQ. Scale bar: 5 μm. FIG. 12E is a series of microphotographs of representative IF of BCL9 colocalization with NONO (left), SFPQ (middle), and ILF2 (right) in RKO (top) and Colo320 (bottom) cells. Colocalization pixels and colocalization threshold are shown. Dotted areas represent a group of pixels which have high intensity for both BCL9 and indicated protein. Arrow heads indicate dotted areas with highest pixels for colocalization of both BCL9 and indicated proteins. Scale bar: 2 μm.

FIG. 13A-FIG. 13F is a group of graphs, immunoblots, and heat maps showing down regulated genes in normalized RNA-seq data of indicated genes. FIG. 13A is an immunoblot depicting the expression of the indicated proteins and cell lines in wild-type and BCL9 knockout clones. WT: wild-type; Numbers next to BCL9 indicate different knockout clones. FIG. 13B is a bar graph showing real-time quantitative polymerase chain reaction (RT qPCR verified the results of RNA-seq in five knockout clones. Data are displayed as mean fSD for three independent experiments and repeated twice. FIG. 13C is a bar graph showing RT qPCR verified the results of RNA-seq in one rescued clone. Data are displayed as mean SD for three independent experiments and repeated twice. FIG. 13D is a group of graphs showing gene set enrichment analysis of down-regulated genes in BCL9 deficient RKO (top) and Colo320 (bottom) cells. FIG. 13E is a 3D-plot depicting PCA analysis of differentially expressed gene between wild-type and BCL9 knock out cells. FIG. 13F is a heat map of indicated gene expression (top) and pathway activation (bottom) in the indicated CRC cell lines.

FIG. 14A-FIG. 14E is a series of graphs, dot-plots, gene denrograms, and microphotographs illustrating down regulated genes in normalized RNA-seq data of indicated genes and CRC clusters. FIG. 14A is a gene dendrogram obtained by average linkage hierarchical clustering of CRC Cluster 1. The colored row underneath the dendrogram shows the module assignment determined by Dynamic Tree Cut (upper) and merged dynamic (lower). FIG. 14B is a distribution plots of downregulated genes in BCL9 knockout cells (left) and genes encoding BCL9-interacting proteins as per IP studies (right) in the transcriptional network matrix. FIG. 14C is a group of dot-plots for immunohistochemistry (IHC) H-scores of the indicated proteins in FAP low and high groups from a CRC TMA. The H-scores and FAP low and high groups were defined in material and methods. *: P<0.05; ***: P<0.001; ns: not significant. FIG. 14D is a correlation plot of IHC H-score of BCL9 and RGS4 in FAP low and high groups. FIG. 14E is a group of microphotographs portraying representative IHC BCL9, RGS4 and FAP in FAP low and high groups; the staining intensity of the region of interest (ROI) was displayed by 3D surface plot. Scale bar: 10 μm.

FIG. 15A-FIG. 15G is a series of images, immunoblots, and bar graphs showing BCL9 regulation. FIG. 15A is a series of Co-IP with the indicated specific antibodies in the indicated CRC cells. FIG. 15B is a series of combined BCL9 IF with NEAT1 FISH in RKO cells. Dotted areas represent interchromosomal regions. FIG. 15C is a series of IF of NONO and BCL9 in RKO cells. Dotted areas indicated the position of BCL9 dotted staining. White arrows heads indicate interchromosomal region. ROI: region of interest. Scale bar: 5 μm. FIG. 15D are bar graphs for RIP-PCR detected BCL9, NONO interaction with NEAT1 non-coding RNA in RKO cells. FIG. 15E are a series of immunoblots depicting the knockdown of NONO and ILF2 with two different short hairpin ribonucleic acids (shRNAs) in RKO and Colo320 cell lines. FIG. 15F is a bar graph showing cell viability of control and RKO cells overexpressing BCL9 after lentiviral transduction with shRNAs against LacZ, NONO, or ILF2. ****: P<0.0001; ns: not significant. shNONO-1, shNONO-2, shILF2-1 and shILF-2-2 indicate two different hairpins. FIG. 15G is an immunoblot showing Wnt/β-catenin downstream target genes in RKO cells overexpressing BCL9. Data are displayed as mean±SD for three independent experiments and repeated twice.

FIG. 16A-FIG. 16L is a series of graphs, charts, immunoblots, and microphotographs demonstrating BCL9 regulation. FIG. 16A is a co-IP with BCL9 specific antibody in RKO cell lysates with or without RNase treatment. FIG. 16B is a representative IF staining (top) and colocalization threshold (bottom) of BCL9 and NONO in control or RNase treated RKO cells. Scale bar: 10 μm. FIG. 16C is a series of IF of BCL9 in RKO cells untreated or treated with Poly I:C, CpG deoxyribonucleic acid (DNA) and LPS for 6 hrs (top). BCL9 staining area as calculated by ImageJ2 (bottom). Scale bar: 2 μm. FIG. 16D is a time course immunoblot of the indicated protein in RKO and Colo320 cells treated in the absence or presence of CpG DNA. FIG. 16E is an immunoblot of the indicated proteins in cytoplasmic and nuclear protein fractions of RKO cells untreated or treated with Poly I:C for 6 hrs. FIG. 16F is a bar graph showing cell viability analysis in wild-type and BCL9 knockout RKO cells after treatment with Poly I: C. ****: P<0.0001. Numbers after BCL9−/− represent individual clones. FIG. 16G and FIG. 16H are co-IP with the indicated antibodies in untreated or Poly I:C treated indicated cells. FIG. 16I is a series of combined BCL9 IF with NEAT1 FISH in RKO cells treated or untreated with Poly I:C. White arrowhead indicates the NEAT1 FISH signal, which is adjacent to and partially overlaps with BCL9 IF signal. Scale bar: 2 μm. FIG. 16J is a co-IP with anti-β-catenin antibody in untreated and poly I:C treated Colo320 cells. FIG. 16K is a co-IP with anti-BCL9 antibody in untreated and Wnt3A treated RKO cells. FIG. 16L is a schematic model for BCL9 interaction with paraspeckles. I.R.C represents interchromosomal regions.

FIG. 17A-FIG. 17D is a series of graphs depicting the inhibition of calcium transients. FIG. 17A is a bar graph showing qRT-PCR analysis of RGS4 and CACNA2D1 mRNA expression in untreated or dopamine treated wild-type or BCL9 knockout RKO cells. ****: P<0.0001. 12 and 21 represent different BCL9 knockout clones. FIG. 17B is a bar graph showing NFATC2-dependent luciferase reporter activity in the indicated cells lentivirally transduced with either shLacZ, shBCL9, or shILF2. Numbers 1 and 2 indicate two different hairpins. FIG. 17C is a bar graph showing qRT-PCR analysis of CACNA2D1 mRNA expression in indicated cell lines that have been treated with individual siRNAs. FIG. 17D is a bar graph showing NFATC2-dependent luciferase reporter activity in the indicated cells transfected with siRNA of control or CACNA2D1. Data are displayed as means±SD for triplicate experiments and repeated twice.

FIG. 18A-FIG. 18G is a series images, heat maps, and graphs showing the inhibition of calcium transients. FIG. 18A is time-lapse imaging of calcium waves (left) and ΔF/F0 heat map (right) of indicated region of interest (ROI, red dots) in indicated CRC cells. On the left, red color indicates waves pike, blue color represents baseline. Scale bar: 10 μm. FIG. 18B is time-lapse imaging of calcium wave in indicated ROIs (top), ΔF/F0 (bottom) revealed that the extension of cell cytoplasm (white arrows) occurred after strenuous calcium transient (black line). Scale bar: 10 μm. FIG. 18C is a group of contrast phase images of wild-type and BCL9 knockout RKO cells (left). The length of cytoplasmic extension was calculated by imageJ2 (right). ***: P<0.001. Data are displayed as mean±SD for three independent experiments and repeated twice. Scale bar: 10 μm. FIG. 18D is a series graphs of wavespike library of calcium waves in wild-type and BCL9 knockout RKO cells in the absence or presence of Poly I: C. FIG. 18E is a group of bar graphs showing mRNA expression fold changes in the RKO cells transfected with indicated siRNAs. ****: P<0.0001. FIG. 18F is a series of dot-plots depicting the network of synchronized calcium transients in control and RGS4 or CACNA2D1 knockdown RKO cells. FIG. 18G is a series of dot-plots depicting a network of synchronized calcium transients in DMSO or CCG50014/Amlodipine treated RKO cells. Each dot represents one cell, the colored dots indicate the number of calcium wave events during 30 mins. Edged-linked dots represent synchronized calcium transients, colored edge indicates the timing of synchronized calcium transients. Data are displayed as mean fSD for three independent experiments and repeated twice.

FIG. 19A-FIG. 19F is a series of time-lapse images and plots illustrating the inhibition of calcium transients. FIG. 19A is time-lapse imaging of calcium wave spreading (top) and wound healing (bottom) at the indicated times after scratching of the monolayer surface in wild-type and BCL9 knockout RKO cells. Top: Red dotted lines indicate the wound edge; numbered white dotted lines indicate two coordinates that are close (1) or distant (2) to the wound edge. Bottom: white dotted line indicates edges of the wound. Wound healing in BCL9 knockout Colo320 cells is delayed after 12 hs. Scale bar: 20 μm. FIG. 19B is a plot of ΔF/F0 in cells at position 1 and 2 in wild-type or BCL9 knockout Colo320 cells. FIG. 19C is time-lapse imaging of calcium wave spreading (top) and wound healing (bottom) at the indicated times after scratching of monolayer surface in wild-type and BCL9 knockout Colo320 cells. Top: Red dotted lines indicate the wound edge; numbered white dotted lines indicate two coordinates that are close (1) or distant (2) to the wound edge. Bottom: white dotted line indicates edges of the wound. Scale bar: 20 μm. FIG. 19D is a plot of ΔF/F0 in cells at position 1 and 2 in wild-type or BCL9 knockout RKO cells. FIG. 19E is time-lapse imaging revealed that the calcium wave disappeared after scratching monolayer surface in EDTA or verapamil treated RKO cells. Scale bar: 20 μm. FIG. 19F is a plot of ΔF/F0 in cells at position 1 and 2 in EDTA and verapamil treatment RKO cells.

FIG. 20A-FIG. 20D is a series of chromatograms, dot-plots, graphs, and models of calcium transient inducers. FIG. 20A is a total ion chromatogram of CM from wild type and BCL9 knockout RKO cells (n=3). FIG. 20B is a group of plots showing the network of calcium transients spreading and ΔF/F0 (top) of synchronized calcium waves in control and propranolol treated RKO cells (bottom). FIG. 20C is a chart showing cell viability of indicated CRC cell lines treated in the absence or increased concentrations of propranolol (top) or verapamil (bottom). FIG. 20D is model of calcium wave spreading in Colo320, RKO and DLD-1 cells. Data are displayed as mean fSD for three independent experiments and repeated twice.

### DETAILED DESCRIPTION OF THE INVENTION

The present invention is based upon the surprising discovery that the interaction of BCL9 with paraspeckle proteins provide neural-like, multi-cellular communication properties in a poor prognosis molecular subtype of colorectal cancer.

### B-Cell Lymphoma 9 (BCL9)

BCL9 is a protein that in humans is encoded by the BCL9 gene and functions as a transcriptional co-activator of β-catenin in the canonical Wnt pathway, which plays critical roles in CRC pathogenesis. However, prior to the invention described herein, CRC subtype-specific functions of BCL9 had not been well defined. Herein, a new β-catenin-independent function of BCL9 in a poor-prognosis subtype of CRC tumors characterized by the expression of stromal and neural associated genes is described. In response to spontaneous calcium transients or cellular stress, BCL9 is recruited adjacent to the interchromosomal regions, where it stabilizes the mRNA of calcium signaling and neural associated genes by interacting paraspeckle proteins. BCL9 subsequently promotes tumor progression and microenvironment remodeling by sustaining the calcium transients and neurotransmitter-dependent communication among CRC cells. This unique role of BCL9 in tumor pathogenesis can be harnessed for new avenues of therapeutic intervention.

An exemplary BCL9 polypeptide sequence is provided at NCBI Accession No. NM_004317, version NM_004317.2, incorporated herein by reference and set forth below (SEQ ID NO: 7):

| | |
|---|---|
| 1 | mhssnpkvrs spsgntqssp kskqevmvrp ptvmspsgnp qldskfsnqg kqggsasqsq |
| 61 | pspcdsksgg htpkalpgpg gsmglkngag ngakgkgkre rsisadsfdq rdpgtpndds |
| 121 | dikecnsadh iksqdsqhtp hsmtpsnata prsstpshgq ttateptpaq ktpakvvyvf |
| 181 | stemankaae avlkgqveti vsfhiqnisn nkterstapl ntqisalrnd pkplpqqppa |
| 241 | panqdqnssq ntrlqptppi papapkpaap prpldrespg venklipsvg spasstplpp |
| 301 | dgtgpnstpn nravtpvsqg snsssadpka pppppvssge pptlgenpdg isqeqlehre |
| 361 | rslqtlrdiq rmlfpdekef tgaqsggpqq npgvldgpqk kpegpiqamm aqsqslgkgp |
| 421 | gprtdvgapf gpqghrdvpf spdemvppsm nsqsgtigpd hldhmtpeqi awlklqqefy |
| 481 | eekrrkqeqv vvqqcslqdm mvhqhgprgv vrgppppyqm tpsegwapgg tepfsdginm |
| 541 | phslpprgma phpnmpgsqm rlpgfagmin semegpnvpn pasrpglsgv swpddvpkip |
| 601 | dgrnfppgqg ifsgpgrger fpnpqglsee mfqqqlaekq lglppgmame girpsmemnr |
| 661 | mipgsqrhme pgnnpifpri pvegplspsr gdfpkgippq mgpgrelefg mvpsgmkgdv |
| 721 | nlnvnmgsns qmipqkmrea gagpeemlkl rpggsdmlpa qqkmvplpfg ehpqqeygmg |
| 781 | prpflpmsqg pgsnsglrnl repigpdqrt nsrlshmppl plnpssnpts lntappvqrg |
| 841 | lgrkpldisv agsqvhspgi nplksptmhq vqspmlgsps gnlkspqtps qlagmlagpa |
| 901 | aaasiksppv lgsaaaspvh lkspslpaps pgwtsspkpp lqspgippnh kapltmaspa |
| 961 | mlgnvesggp ppptasqpas vnipgslpss tpytmppept lsqnplsimm srmskfamps |
| 1021 | stplyhdaik tvassdddsp parspnlpsm nnmpgmgint qnprisgpnp vvpmptlspm |
| 1081 | gmtqplshsn qmpspnavgp nipphgvpmg pglmshnpim ghgsqeppmv pqgrmgfpqg |
| 1141 | fppvqsppqq vpfphngpsg gqgsfpggmg fpgegplgrp snlpqssada alckpggpgg |
| 1201 | pdsftvlgns mpsvftdpdl qevirpgatg ipefdlsrii psekpsqtlq yfprgevpgr |
| 1261 | kqpqgpgpgf shmqgmmgeq aprmglalpg mggpgpvgtp diplgtapsm pghnpmrppa |
| 1321 | flqqgmmgph hrmmspaqst mpgqptlmsn paaavgmipg kdrgpaglyt hpgpvgspgm |
| 1381 | mmsmqgmmgp qqnimippqm rprgmaadvg mggfsqgpgn pgnmmf |

An exemplary BCL9 nucleic acid sequence is provided at NCBI Accession No. NM_004326, version NM_004326.3, incorporated herein by reference and set forth below (SEQ ID NO: 8):

| | |
|---|---|
| 1 | agaccagagc agacaatagg cccctaaagt gttcccccta agttgctttg atgttgtcct |
| 61 | ggtgtcttga taccaggagg ccagggattg cgggaaaagg gtcttttttg tcttcattca |
| 121 | ctttcccccc tcagtttctg aaatgattct ccagaatttc tcctcataaa aaaggactga |
| 181 | atgtgggccc agttggcgtc attctgcttt gacctaaaca ttcccatctg attgggtggc |
| 241 | agagatcatt tttggaaagt tcttccgtgt cccgatgtag aagaaatagc aaattggaca |
| 301 | tattgaaaga caagggtcat ctttgagaag ggggttcctg gactcctcac ctccaggatg |
| 361 | agcactgcag tgtcgtgacc cttggggttt tgtatgccct ggagatgcga gattttcctc |
| 421 | tggcagcagg aggcacgcac ccagagaatg ctggagctgc aaggggaaag gacccacttc |
| 481 | cacagcagag aaaaacaaag aggaaaaagg catacaggca gcgagcgcta agggacgcac |
| 541 | ccagcaagca gtgggccagt gccactgccc ccagcagctg tttctgctgc aacccgagag |
| 601 | gaactcggtg agcctgtccc gtttgtgact gcaagctcag gatttcaatc aatgcattcc |
| 661 | agtaacccta aagtgaggag ctctccatca ggaaacacac agagtagccc taagtcaaag |
| 721 | caggaggtga tggtccgtcc ccctacagtg atgtccccat ctggaaaccc ccagctggat |
| 781 | tccaaattct ccaatcaggg taaacagggg ggctcagcca gccaatccca gccatccccc |
| 841 | tgtgactcca agagtggggg ccatacccct aaagcactcc ctggcccagg tgggagcatg |
| 901 | gggctgaaga atggggctgg aaatggtgcc aagggcaagg ggaaaaggga gcgaagtatt |
| 961 | tccgccgact cctttgatca gagagatcct gggactccaa acgatgactc tgacattaaa |
| 1021 | gaatgtaatt ctgctgacca cataaagtcc caggattccc agcacacacc acactcgatg |
| 1081 | accccatcaa atgctacagc ccccaggtct tctaccccct cccatggcca aactactgcc |
| 1141 | acagagccca cacctgctca gaagactcca gccaaagtgg tgtacgtgtt ttctactgag |
| 1201 | atggccaata aagctgcaga agctgttttg aagggccagg ttgaaactat cgtctctttc |
| 1261 | cacatccaga acatttctaa caacaagaca gagagaagca cagcgcctct gaacacacag |
| 1321 | atatctgccc ttcggaatga tccgaaacct ctcccacaac agcccccagc tccggccaac |
| 1381 | caggaccaga attcttccca gaataccaga ctgcagccaa ctccacccat tccggcacca |
| 1441 | gcacccaagc ctgccgcacc cccacgtccc ctggaccggg agagtcctgg ggtagaaaac |
| 1501 | aaactgattc cttctgtagg aagtcctgcc agctccactc cactgccccc agatggtact |
| 1561 | gggcccaact caactcccaa caatagggca gtgacccctg tctcccaggg gagcaatagc |
| 1621 | tcttcagcag atcccaaagc ccctccgcct ccaccagtgt ccagtggcga gccccccaca |
| 1681 | ctgggagaga atcccgatgg cctatctcag gagcagctgg agcaccggga gcgctcctta |
| 1741 | caaactctca gagatatcca gcgcatgctt tttcctgatg agaaagaatt cacaggagca |
| 1801 | caaagtgggg gaccgcagca gaatcctggg gtattagatg ggcctcagaa aaaaccagaa |
| 1861 | gggccaatac aggccatgat ggcccaatcc caaagcctag gtaagggacc tgggccccgg |
| 1921 | acagacgtgg gagctccatt tggccctcaa ggacatagag atgtaccctt ttctccagat |
| 1981 | gaaatggttc caccttctat gaactcccag tctgggacca taggacccga ccaccttgac |
| 2041 | catatgactc ccgagcagat agcgtggctg aaactgcagc aggagtttta tgaagagaag |
| 2101 | aggaggaagc aggaacaagt ggttgtccag cagtgttccc tccaggacat gatggtccat |
| 2161 | cagcacgggc ctcggggagt ggteegagga cccccccctc cataccagat gacccctagt |
| 2221 | gaaggctggg cacctggggg tacagagcca ttttctgatg gtatcaacat gccacattct |
| 2281 | ctgcccccga ggggcatggc tccccacccc aacatgccag ggagccagat gcgcctccct |
| 2341 | ggatttgcag gcatgataaa ctctgaaatg gaagggccga atgtccccaa ccctgcatct |
| 2401 | agaccaggtc tttctggagt cagttggcca gatgatgtgc caaaaatccc agatggtcga |
| 2461 | aattttcctc ctggccaggg cattttcagc ggtcctggcc gaggggaacg cttcccaaac |
| 2521 | ccccaaggat tgtctgaaga gatgtttcag cagcagctgg cagagaaaca gctgggtctc |
| 2581 | cccccaggga tggccatgga aggcatcagg cccagcatgg agatgaacag gatgattcca |
| 2641 | ggctcccagc gccacatgga gcctgggaat aaccccattt tccctcgaat accagttgag |
| 2701 | ggccctctga gtccttctag gggtgacttt ccaaaaggaa ttcccccaca gatgggccct |
| 2761 | ggtcgggaac ttgagtttgg gatggttcct agtgggatga agggagatgt caatctaaat |
| 2821 | gtcaacatgg gatccaactc tcagatgata cctcagaaga tgagagaggc tggggcgggc |
| 2881 | cctgaggaga tgctgaaatt acgcccaggt ggctcagaca tgctgcctgc tcagcagaag |
| 2941 | atggtgccac tgccatttgg tgagcacccc cagcaggagt atggcatggg ccccagacca |
| 3001 | ttccttccca tgtctcaggg tccaggcagc aacagtggct tgcggaatct cagagaacca |
| 3061 | attgggcccg accagaggac taacagccgg ctcagtcata tgccaccact acctctcaac |
| 3121 | ccttccagta accccaccag cctcaacaca gctcctccag ttcagcgcgg cctggggcgg |
| 3181 | aagcccttgg atatatctgt ggcaggcagc caggtgcatt ccccaggcat taaccctctg |
| 3241 | aagtctccca cgatgcacca agtccagtca ccaatgctgg gctcgccctc ggggaacctc |
| 3301 | aagtcccccc agactccatc gcagctggca ggcatgctgg cgggcccagc tgctgctgct |
| 3361 | tccattaagt ccccccctgt tttggggtct gctgctgctt cacctgtcca cctcaagtct |
| 3421 | ccatcacttc ctgccccgtc acctggatgg acctcttctc caaaacctcc ccttcagagt |
| 3481 | cctgggatcc ctccaaacca taaagcaccc ctcaccatgg cctccccagc catgctggga |
| 3541 | aatgtagagt caggtggccc cccacctcct acagccagcc agcctgcctc tgtgaatatc |
| 3601 | cctggaagtc ttccctctag tacaccttat accatgcctc cagagccaac cctttcccag |
| 3661 | aacccactct ctattatgat gtctcgaatg tccaagtttg caatgcccag ttccaccccg |
| 3721 | ttataccatg atgctatcaa gactgtggcc agctcagatg acgactcccc tccagctcgt |
| 3781 | tctcccaact tgccatcaat gaataatatg ccaggaatgg gcattaatac acagaatcct |
| 3841 | cgaatttcag gtccaaaccc cgtggttccg atgccaaccc tcagcccaat gggaatgacc |
| 3901 | cagccacttt ctcactccaa tcagatgccc tctccaaatg ccgtgggacc caacatacct |
| 3961 | cctcatgggg tcccaatggg gcctggcttg atgtcacaca atcctatcat ggggcatggg |
| 4021 | tcccaggagc caccgatggt acctcaagga cggatgggct tcccccaggg cttccctcca |
| 4081 | gtacagtctc ccccacagca ggttccattc cctcacaatg gccccagtgg ggggcagggc |
| 4141 | agcttcccag gagggatggg tttcccagga gaaggccccc ttggccgccc cagcaacctg |
| 4201 | ccccaaagtt cagcagatgc agcactttgc aagcctggag gccccggggg tcctgactcc |
| 4261 | ttcactgtcc tggggaacag catgccttcg gtgtttacag acccagatct gcaggaggtc |
| 4321 | atccgacctg gagccaccgg aatacctgag tttgatctat cccgcattat tccatctgag |
| 4381 | aagcccagcc agacgctgca atatttccct cgaggggaag ttccaggccg taaacagccc |
| 4441 | cagggtcctg gacctgggtt ttcacacatg caggggatga tgggcgaaca agcccccaga |
| 4501 | atgggactag cattacctgg catgggaggt ccagggccag tgggaactcc ggacatccct |
| 4561 | cttggtacag ctccatccat gccaggccac aaccccatga gaccaccagc ctttctccaa |
| 4621 | caaggcatga tgggacctca ccatcggatg atgtcaccag cacaatctac aatgcccggc |
| 4681 | cagcccaccc tgatgagcaa tccagctgct gccgtgggca tgattcctgg caaggatcgg |
| 4741 | gggcctgccg ggctctacac ccaccctggg cctgtgggct ctccaggcat gatgatgtcc |
| 4801 | atgcagggca tgatgggacc ccaacagaac atcatgatcc ccccacagat gaggccccgg |
| 4861 | ggcatggctg ctgacgtggg catgggtgga tttagccaag gacctggcaa cccaggaaac |
| 4921 | atgatgtttt aagctgctaa gatgggatgt gccgatcctt gtcaagatga gattccaggt |
| 4981 | cctgagagct gctttgaggg agttccagga gtacttacta ttggtcatgc aataggagaa |
| 5041 | cagagacccg agggctgctt tgggggaggg gggaactcga gaatgtatgg atttacctga |
| 5101 | aaacaaatta ttcatttaat caacaggtgt gtttttttta agatttattt tttaaaaatt |
| 5161 | atttttgtgg acttgggtat caatgatggc acctactttt gggaatctgt agctgtgctt |
| 5221 | tgagaattgc catcggtcat gtgttgcacc gttctctgta tgtttacgtc ctttggactg |
| 5281 | gcttctccca ggattctttt ctgtttttgt ttttttgatt tgggctttat ttttttctgt |
| 5341 | gtactgtact atattgtaaa agggatttta gcagagactt tagtctttgg ggcaagagga |
| 5401 | gaacaggaat gctgggctgt ttactttagg tggagaatcc atcttcagac ctttggacta |
| 5461 | ttttctttca actgcagtgt atagaaaaac caaactacga cctcagagca gagtattaat |
| 5521 | gaaaagcaca aaaaaaggaa ctaagttcag cgaggggtgg ggggaggggg gagatttttc |
| 5581 | ttttgaaaaa taatgactct taggacattt gtttttcagt tcaagtgctc ttcagcactg |
| 5641 | tcttgtctcc caatatacca acccactggc acatttttct cttgttttct ctctccgatt |
| 5701 | ttgctctgtc tcctcagtta agtgtttcct tcctttgtgc cccccgctgg tgaccctctg |
| 5761 | cttccctctc tctttccctt tggcagctgc aatacacagt gttattttgg ggaaataaat |
| 5821 | ctagcaaagc ctcgccttcc atgccgagcg tcctcttggc tctgagaggg aaaggtctgt |
| 5881 | ccttgggatg ctctctggtc ttttttcccc ctaagtcttt ctctttccca tcataccctt |
| 5941 | ccctgcccac cttgttttct gttctccttt tattaggaat tcccaagtga attttattaa |
| 6001 | tgtgggagtg gaacagatgc taaaagctat ccaggatttt gtttctgttt gttttaaatt |
| 6061 | ttgtggttcc ttccctttcc tccccctccc atgcgtaaga cgttctgtgt aacctccatt |
| 6121 | aaatttggta caaaaccact cgccagagct gtggtgtcag aaaaataaaa tatattgttt |
| 6181 | cttacaaaat tg |

Exemplary inhibitors of BCL9 include those that disrupt the interaction between BCL9 and β-catenin. For example, a stabilized alpha helix (SAH), hydrocarbon-stapled, BCL9 disrupts the BCL9/β-catenin complex (Takada et al., 2012 Sci Trans Med, 4(148): 148ra117, incorporated herein by reference). An exemplary SAH-BCL9 hydrocarbon-stapled polypeptide sequence is:

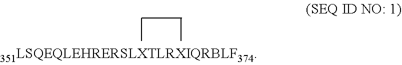

Another exemplary SAH-BCL9 hydrocarbon-stapled polypeptide sequence is:

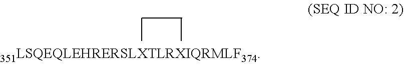

An additional exemplary SAH-BCL9 hydrocarbon-stapled polypeptide sequence is:

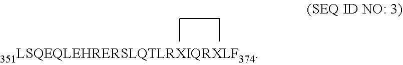

A further exemplary SAH-BCL9 hydrocarbon-stapled polypeptide sequence is:

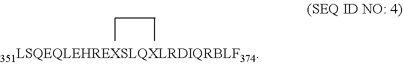

In some cases, the BCL9 inhibitors comprise a small molecule inhibitor, RNA interference (RNAi), microRNA (miRNA), an antibody, an antibody fragment, an antibody drug conjugate, an aptamer, a chimeric antigen receptor (CAR), a T cell receptor, or any combination thereof.

Exemplary anti-BCL9 antibodies may include polyclonal anti-BCL9 antibody produced in rabbit (Catalogue No. 37305; Abcam®; Cambridge, Mass.), monoclonal 1A2 anti-BCL9 antibody (Catalogue No. WH0000607M1; MilliporeSigma®; Burlington, Mass.), or monoclonal 2D4 anti-BCL9 antibody (Catalogue No. SAB1403599; MilliporeSigma®; Burlington, Mass.).

Exemplary small molecule inhibitors of BCL9 include 4′-fluoro-N-phenyl-[1,1′-biphenyl]-3-carboxamide and analogs thereof (Hoggard et al., 2015 J. Am. Chem. Soc. 137:12249-12260, incorporated herein by reference), camosic acid (de la Roche et al., 2012 Nat. Commun., 3:680, incorporated herein by reference), and 1,4-dibenzoylpiperazine compounds (Wisniewski et al., 2016 ACS Med. Chem. Lett., 7:508-513, incorporated herein by reference).

It has also been identified that miR-30-5p functions as a tumor suppressor by targeting the β-catenin/BCL9 pathway (Zhao et al., 2014 Cancer Res, 74(6): 1801-1813, incorporated herein by reference). Suitable miR-30 polynucleotides include the following:

has-miR-30a
(UGUAAACAUCCUCGACUGGAAG (SEQ ID NO: 9)),
has-miR-30b
(UGUAAACAUCCUACACUCAGCU (SEQ ID NO: 10)),
has-miR-30c
(UGUAAACAUCCUACACUCUCAGC (SEQ ID NO: 11)),
hasmiR-30d
(UGUAAACAUCCCCGACUGGAAG (SEQ ID NO: 12)),
and
has-miR-30e,
(UGUAAACAUCCUUGACUGAAG (SEQ ID NO: 13)).

### Colorectal Cancer

CRC is the third most commonly diagnosed cancer, which despite recent advances in treatment, prior to the invention described herein, remains essentially incurable due to a complex array of tumor-specific molecular features. Colorectal cancer, also known as bowel cancer, colon cancer, or rectal cancer, is any cancer that affects the colon and the rectum. It is the second leading cause of cancer death in women, and the third for men. Colorectal cancer may be benign, or non-cancerous, or malignant. A malignant cancer can spread to other parts of the body via metastasizing. Symptoms of colorectal cancer include changes in bowel habits, diarrhea or constipation, a feeling that the bowel does not empty properly after a bowel movement, blood in the stool, bleeding from the rectum, pain and bloating in the abdomen, a feeling of fullness in the abdomen, even after not eating for a while, fatigue or tiredness, unexplained weight loss, or an unexplained iron deficiency. Most of these symptoms may also indicate other possible conditions. It is important to see a doctor if symptoms persist for 4 weeks or more. It is not clear why colorectal cancer develops in some people and not in others. Greater than 75-95% of colorectal cancer occurs in people with little or no genetic risk. Risk factors include older age, male sex, high intake of fat, alcohol, red meat, processed meats, obesity, smoking, and a lack of physical exercise. People with inflammatory bowel disease, such as ulcerative colitis and Crohn's disease, are at increased risk of colon cancer.

Screening can detect polyps before they become cancerous, as well as detecting colon cancer during its early stages when the chances of a cure are much higher. There are a number of common screening and diagnostic procedures for colorectal cancer. Procedures include blood stool test and stool DNA test where the stool is analyzed for several DNA markers that colon cancers or precancerous polyps cells shed in the stool. An individual can undergo a sigmoidoscopy which involves the doctor using a sigmoidoscope to examine the patient's rectum and sigmoid for polyps or colon cancer. A sigmoidoscopy will only detect polyps or cancer in the end third of the colon and the rectum. Another option is a colonoscopy in which the doctor uses a colonoscope. The colonoscope is longer than a sigmoidoscope and attached to a video camera and monitor. In this manner, the doctor can see the whole of the colon and rectum. Any polyps discovered during this exam can be removed during the procedure, and sometimes tissue samples, or biopsies, are taken instead. An individual can also undergo CT colonography where a CT machine takes images of the colon, after clearing the colon. If anything abnormal is detected, a conventional colonoscopy may be necessary.

The treatment of CRC depends on several factors, including the size, location, and stage of the cancer, whether or not it is recurrent, and the current overall state of health of the patient. Treatment options include chemotherapy, radiotherapy, and surgery. Surgery is the most common treatment. The affected malignant tumors and any nearby lymph nodes will be removed, to reduce the risk of the cancer spreading. The bowel is usually sewn back together, but sometimes the rectum is removed completely and a colostomy bag is attached for drainage. The colostomy bag collects stools. This is usually a temporary measure, but it may be permanent if it is not possible to join up the ends of the bowel. Chemotherapy involves using a medicine or chemical to destroy the cancerous cells. It can be used before surgery as it may help shrink the tumor. Targeted therapy is a kind of chemotherapy that specifically targets the proteins that encourage the development of some cancers. They may have fewer side effects than other types of chemotherapy. Drugs that are used for CRC include Avastin and Cyramza. Radiation therapy uses high energy radiation beams to destroy the cancer cells and to prevent them from multiplying. This is more commonly used for rectal cancer treatment. It also can be used before surgery in an attempt to shrink the tumor.

The outcome of the treatment can vary widely due to a complex array of tumor-specific molecular features. In this study, a β-catenin-independent function of BCL9 is described in the pathogenesis and progression of a subtype of CRC that is characterized by stromal cell infiltration. Evidence is provided herein for a role of BCL9 in affording neural-like and multi-cellular communication properties of CRC cells through sustaining calcium transient and neurotransmitter release. Therefore, described herein is a heretofore unrecognized role for BCL9 in CRC progression, which has important avenues for therapeutic intervention via inhibition of BCL9 function or neurotransmitter receptors blockade.

The human BCL9 gene, a homologue of the Drosophila segment polarity gene, Legless, was first identified by cloning the t(1;14)(q21;q32) translocation from a patient with precursor B-cell acute lymphoblastic leukemia (B-ALL) (Willis et al., (1998), Blood 91, 1873-1881). BCL9/Legless function as transcriptional co-activators of the canonical Wnt pathway, by binding to β-catenin through a highly conserved HD2 domain (BCL9-HD2) (de la Roche et al., (2008), BMC Cancer 8, 199; Cantu et al., (2017), Sci. Signal 10; Deka et al., (2010), Cancer Res. 70, 6619-6628; Miller et al., (2010), J. Mol. Biol. 401, 969-984). The oncogenic potential of BCL9 in human cancer, is further highlighted by studies showing that: i) chromosome 1q21 amplifications harboring the BCL9 locus are observed in a broad range of cancers and are associated with lack of therapeutic response and poor clinical outcome (Banck et al. (2013), J. Clin. Invest 123, 2502-2508; Jia et al., (2011), Mol. Cancer Res. 9, 1732-1745); ii) BCL9 is upregulated in various malignancies as a consequence of downregulation of micro-RNAs (Jia et al. (2011), Mol. Cancer Res. 9, 1732-1745); Liu et al., (2017), Sci. Rep. 7, 7113; Yang et al., (2017), Cell Death Dis. 8, e2999; Ling et al., (2016), Oncol. Lett. 11, 2001-2008; Zhao et al., (2014), Cancer Res. 74, 1801-1813; Luna et al., (2017), Mol. Cell 67, 400-410 e407; Zhao et al., (2014), Cancer Res. 74, 5351-5358) that function as endogenous tumor suppressors of BCL9; iii) LATS2 (Li et al., (2013), Cell Rep. 5, 1650-1663) and SOX7 (Fan, et al., (2017), DNA Cell Biol. 37, 126-132) proteins, which suppress oncogenic Wnt signaling by disrupting β-catenin/BCL9 interaction, are downregulated in tumor tissues; and iv) pharmacologic disruption of β-catenin/BCL9 interaction (Takada et al., (2012), Sci. Transl. Med. 4, 148ra117; de la Roche et al., (2012), Nat. Commun. 3, 680) is associated with antitumor activity.

Thus far, the oncogenic activity of BCL9 has only been ascribed to its selective binding to β-catenin, and thus to its role as a Wnt transcriptional co-activator (Deka et al., (2010), Cancer Res. 70, 6619-6628; Valenta et al., (2011), Genes Dev. 25, 2631-2643). However, induction of Wnt target gene transcription by BCL9 is cell type-specific and dependent on the cellular context (Sustmann et al., (2008), Mol. Cell Biol. 28, 3526-3537). Moreover, there is growing evidence that BCL9 interacts with proteins other than β-catenin and that its oncogenic activity may be, in part, independent of Wnt/β-catenin. For instance: i) lens development is unaffected in mice with targeted deletion of the BCL9-HD2 domain (Cantu et al., (2014), Genes Dev. 28, 1879-1884); ii) BCL9 acts independently of β-catenin transcription during dental enamel formation (Cantu et al., (2017), Sci. Signal 10); iii) BCL9 binds to proteins that transmit signals from estrogen and androgen receptors (van Tienen et al., (2017), Elife 6); and iv) the BCL9/MEF2D fusion protein found in patients with poor-prognosis B-ALL lacks the BCL9-HD2 domain (Gu et al., (2016), Nat. Commun. 7, 13331; Suzuki et al., (2016), J. Clin. Oncol. 34, 3451-3459). These findings indicate that BCL9 is potentially a multifunctional protein, and that in addition to its critical role as a Wnt/β-catenin co-activator, it may have other unrelated activities. Therefore, the therapeutic effect of targeting the BCL9/β-catenin interaction may result in unpredictable outcomes without knowing the molecular and functional heterogeneity of the tumor.

As described herein, using a series of machine learning-based analytical tools, a unique oncogenic function of BCL9 has been identified. By interacting with paraspeckles proteins (e.g., SFPQ/NONO), which are in post transcriptional regulation, BCL9 stabilizes the mRNA of calcium signaling and neural associated genes to confer neuron-like, multicellular communication properties to a poor prognosis molecular subtype of CRC.

As described in detail below, BCL9 regulates expression of neural-associated genes and promotes tumor progression of a molecular subtype of CRC characterized by stromal cell infiltration. During this regulation process, BCL9 cooperates with NONO, SFPQ, ILF2, and other paraspeckle proteins in stabilizing mRNAs of neural and voltage-dependent calcium-associated genes. As described herein, BCL9 recruitment to paraspeckles occurs in response to cellular stress and calcium transient. When there is a BCL9 deficiency the interaction between NONO and ILF2 is inhibited, which impairs cell proliferation, metastasis, and response to cellular stress.

Like in neural cells, transmission of information among cells in a poor prognosis subtype of CRC tumors occurs through various neurotransmitter-specific projections and the formation of a complex communication network. As described in the examples below, BCL9 deficiency reduced expression of voltage-dependent calcium channels, inhibiting release of neurotransmitters and cell to cell communication. As described herein, human CRC tumors with high stromal cell infiltration and BCL9 localization adjacent to paraspeckles are associated with high expression of neural-associated genes and predict treatment response to neurotransmitter receptor inhibition.

Herein a distinct function of BCL9 has been characterized that is independent of its binding to β-catenin in a poor prognosis molecular subtype of CRC characterized by high expression of stromal and neural associated genes that is designated as C1. Through its interaction with paraspeckle proteins, BCL9 enhances the mRNA stability of neural associated genes and the release of neurotransmitter-like molecules, therefore sustaining communication among tumor cells and the tumor microenvironment and regulating stromal cell infiltration.

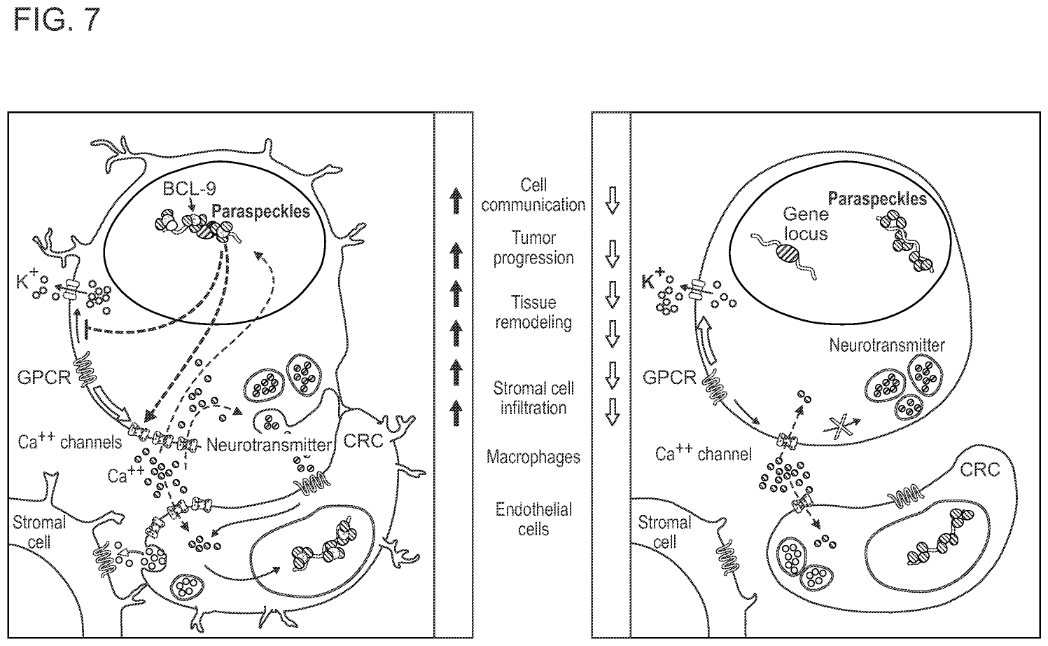

Unsupervised clustering of gene expression profiling studies has uncovered intertumoral cell heterogeneity and allowed the identification of various molecular subtypes in breast, pancreas, and prostate cancers (Bailey et al., (2016) Nature 531:47-52; Neve et al., (2006) Cancer Cell 10:515-527; Lapointe et al., (2004) Proc. Natl. Acad. Sci USA 101:811-816). Similarly, four molecular subtypes of colorectal cancer have been previously identified (CMS1-4) (Dienstmann et al., (2017) Nat. Rev. Cancer 17:79-92). Among them, the CMS4 subgroup is characterized by overexpression of stromal cell infiltration and extracellular remodeling signatures, resembling the C1 cluster identified in the present invention. Using this clustering methodology, primary tumor samples were identified as well as cell lines that share similar biological behavior, overcoming the problem posed by intertumoral heterogeneity, and eliminating bias from a single biomarker selection (e.g. BCL9 alone) (Yuryev A., (2015) Expert Opin. Drug Discov. 10:91-99; Chibon, F., (2013) Eur. J. Cancer 49:2000-2009). Using the same clustering approach and the presence of dotted nuclear staining of BCL9, C1 cell lines were identified for in vivo functional studies. These studies are consistent with a model in which BCL9 promotes neural-like behavior of C1 cells, including the induction of propagating calcium transients and secretion of neurotransmitters. By enhancing communication among tumor cells and cells from the tumor microenvironment, BCL9 promotes tumor progression by increasing tumor growth, tissue remodeling, and infiltration by stromal cells (FIG. 7).

In this model, Toll-like receptor-induced cellular stress promotes calcium overload, enhancing accumulation of BCL9 adjacent to paraspeckles. After binding to paraspeckles by a currently undetermined mechanism, BCL9 stabilizes the mRNAs of neuronal-associated functional genes. Consistent with the model, RNA-seq, live cell imaging, and small MS analyses revealed that lack of BCL9 decreased the expression of voltage dependent calcium channel and synapse-organizing associated genes, inhibiting calcium transient and neurotransmitter release. Notably, among the mRNAs stabilized by BCL9 was RGS4, a regulator of alpha units of heterotrimeric G proteins (Srinivasa et al., (1998), J. Biol. Chem. 273, 1529-1533) which is broadly expressed in excitable tissues such as brain cortex (Gu et al., (2007), Mol Pharmacol 71, 1030-1039) and smooth muscle (Damera et al., (2012), PLoS One 7, e28504). Other mRNAs stabilized by BCL9 include the calcium channel encoding genes CACNA2D1; they are also highly expressed in neuronal cells and their opening is triggered by cell membrane depolarization and G-protein signaling activation (Berger et al., (2014), Cell Tissue Res. 357,463-476; Neef et al., (2009), J. Neurosci. 29, 10730-10740). The role of BCL9 in neuronal cells was supported by high BCL9 expression in ganglion cells but not in normal epithelial cells. In addition, an association between abnormal expression of BCL9 in the brain cortex and negative symptoms in patients with schizophrenia, which is attributed to abnormal activation of calcium signaling and dopamine secretion (Gamock-Jones, et al., (2017) CNS Drugs 31:513-525) has been observed previously (Luo et al., (2014), Schizophr. Bull 40, 1285-1299; Xu et al., (2013), PLoS One 8, e51674; Li et al., (2013), Cell Rep. 5, 1650-1663). Therefore, as postulated in other tumors (Grigore et al., (2015) Front Oncol. 5:37), it seems feasible that C1 CRC cells have hijacked BCL9 function to resemble neuronal cells, and allow them to communicate.

### Paraspeckles

Paraspeckles are unevenly distributed subnuclear bodies that localize within the interchromatin space, adjacent to nuclear speckles, and play a critical role in the control of gene expression during many cellular processes including differentiation, viral infection, and stress responses (Fox et al., (2010), Cold Spring Harb. Perspect. Biol. 2, a000687). Paraspeckles are RNA-protein structures formed by the interaction between a long non-protein-coding RNA species, NET1 and members of the Drosophila Behavior Human Splicing (DBHS) protein family (NONO and SFPQ). They are about 0.5-1.0 μm in size, and their numbers vary both within cell populations and depending on cell type. The formation of paraspeckles is a dynamic process involving the recruitment of DBHS proteins (NONO and SFPQ) and NEAT1 from the nucleoplasm, to the gene locus that is undergoing transcription (Naganuma et al., (2012), EMBO J. 31, 4020-4034). Similarly, multiple copies of BCL9 are recruited adjacent to paraspeckles from a pre-existing pool in the nucleoplasm through a dynamic process that is independent of Wnt activity. Importantly, BCL9 is not needed for paraspeckle formation. Contrary to the formation of paraspeckles, the interaction between BCL9 and paraspeckles is CRC cell type specific. This seems to explain why expression levels of NEAT1 do not show differences among different CRC clusters (data not shown). Likewise to BCL9, other non-DBHS proteins including BCL6 (Liu et al., (2006), Mol. Cancer 5, 18) and SOX9 (Hata et al., (2008), J. Clin. Invest. 118, 3098-3108) transcriptional factors have also been shown to interact with paraspeckle proteins, suggesting the existence of a growing number of non-DBHS proteins at paraspeckles. In addition to cell stress, cell membrane depolarization also induces paraspeckle formation (Adriaens et al., (2016). Nat. Med. 22, 861-868; Lipovich et al., (2012), Genetics 192, 1133-1148). Accordingly, the accumulation of BCL9 in paraspeckles was shown to have occurred after calcium influx. This process could be considered as a positive regulatory mechanism, which ensures that the calcium signaling associated system works normally during cellular stress. By interacting with paraspeckle proteins, BCL9 regulates the spontaneity of calcium transient waves, and enhances cell communication in C1 cell but not in other CRC clusters.

An exemplary NONO polypeptide sequence is provided at NCBI Accession No. CAG33042, version CAG33042.1, incorporated herein by reference and set forth below (SEQ ID NO: 50):

| | |
|---|---|
| 1 | mqsnktfnle kqnhtprkhh qhhhqqqhhq qqqqqppppp ipangqqass qnegltidlk |
| 61 | nfrkpgektf tqrsrlfvgn lppditeeem rklfekygka gevfihkdkg fgfirletrt |
| 121 | laeiakveld nmplrgkqlr vrfachsasl tvrnlpqyvs nelleeafsv fgqveravvi |
| 181 | vddrgrpsgk givefsgkpa arkaldrcse gsfllttfpr pvtvepmdql ddeeglpekl |
| 241 | viknqqfhke reqpprfaqp gsfeyeyamr wkaliemekq qqdqvdrnik eareklemem |
| 301 | eaarhehqvm lmrqdlmrrq eelrrmeelh nqevqkrkql elrqeeerrr reeemrrqqe |
| 361 | emmrrqqegf kgtfpdareq eirmgqmamg gamginnrga mppapvpagt pappgpatmm |
| 421 | pdgtlgltpp tterfgqaat megigaiggt ppafnraapg aefapnkrrr y |

An exemplary NONO nucleic acid sequence is provided at NCBI Accession No. CR456761, version CR456761.1, incorporated herein by reference and set forth below (SEQ ID NO: 14):

| | |
|---|---|
| 1 | atgcagagta ataaaacttt taacttggag aagcaaaacc atactccaag aaagcatcat |
| 61 | caacatcacc accagcagca gcaccaccag cagcaacagc agcagccgcc accaccgcca |
| 121 | atacctgcaa atgggcaaca ggccagcagc caaaatgaag gcttgactat tgacctgaag |
| 181 | aattttagaa aaccaggaga gaagaccttc acccaacgaa gccgtctttt tgtgggaaat |
| 241 | cttcctcccg acatcactga ggaagaaatg aggaaactat ttgagaaata tggaaaggca |
| 301 | ggcgaagtct tcattcataa ggataaagga tttggcttta tccgcttgga aacccgaacc |
| 361 | ctagcggaga ttgccaaagt ggagctggac aatatgccac tccgtggaaa gcagctgcgt |
| 421 | gtgcgctttg cctgccatag tgcatccctt acagttcgaa accttcctca gtatgtgtcc |
| 481 | aacgaactgc tggaagaagc cttttctgtg tttggccagg tagagagggc tgtagtcatt |
| 541 | gtggatgatc gaggaaggcc ctcaggaaaa ggcattgttg agttctcagg gaagccagct |
| 601 | gctcggaaag ctctggacag atgcagtgaa ggctccttcc tgctaaccac atttcctcgt |
| 661 | cctgtgactg tggagcccat ggaccagtta gatgatgaag agggacttcc agagaagctg |
| 721 | gttataaaaa accagcaatt tcacaaggaa cgagagcagc cacccagatt tgcacagcct |
| 781 | ggctcctttg agtatgaata tgccatgcgc tggaaggcac tcattgagat ggagaagcag |
| 841 | cagcaggacc aagtggaccg caacatcaag gaggctcgtg agaagctgga gatggagatg |
| 901 | gaagctgcac gccatgagca ccaggtcatg ctaatgagac aggatttgat gaggcgccaa |
| 961 | gaagaacttc ggaggatgga agagctgcac aaccaagagg tgcaaaaacg aaagcaactg |
| 1021 | gagctcaggc aggaggaaga gcgcaggcgc cgtgaagaag agatgcggcg gcagcaagaa |
| 1081 | gaaatgatgc ggcgacagca ggaaggattc aagggaacct tccctgatgc gagagagcag |
| 1141 | gagattcgga tgggtcagat ggctatggga ggtgctatgg gcataaacaa cagaggtgcc |
| 1201 | atgccccctg ctcctgtgcc agctggtacc ccagctcctc caggacccgc cactatgatg |
| 1261 | ccggatggaa ctttgggatt gaccccacca acaactgaac gctttggtca ggctgctaca |
| 1321 | atggaaggaa ttggggcaat tggtggaact cctcctgcat tcaaccgtgc agctcctgga |
| 1381 | gctgaatttg ccccaaacaa acgtcgccga tattaa |

An exemplary ILF2 polypeptide sequence is provided at NCBI Accession No. NP_004506, version NP_004506.2, incorporated herein by reference and set forth below (SEQ ID NO: 15):

| | |
|---|---|
| 1 | mrgdrgrgrg grfgsrggpg ggfrpfvphi pfdfylcema fprvkpapde tsfseallkr |
| 61 | nqdlapnsae qasilslvtk innvidnliv apgtfevqie evrqvgsykk gtmttghnva |
| 121 | dlvvilkilp tleavaalgn kvveslraqd psevltmltn etgfeisssd atvkilittv |
| 181 | ppnlrkldpe lhldikvlqs alaairharw feenasqstv kvlirllkdl rirfpgfepl |
| 241 | tpwildllgh yavmnnptrq plalnvayrr clqilaaglf lpgsvgitdp cesgnfrvht |
| 301 | vmtleqqdmv cytaqtlvri lshggfrkil gqegdasyla seistwdgvi vtpsekayek |
| 361 | ppekkegeee eenteeppqg eeeesmetqe |

An exemplary ILF2 nucleic acid sequence is provided at NCBI Accession No. NM_004515, version NM_004515.4, incorporated herein by reference and set forth below (SEQ ID NO: 16):

| | |
|---|---|
| 1 | ctcttcagtt gtctgctact cagaggaagg ggcggttggt gcggcctcca ttgttcgtgt |
| 61 | tttaaggcgc catgaggggt gacagaggcc gtggtcgtgg tgggcgcttt ggttccagag |
| 121 | gaggcccagg aggagggttc aggccctttg taccacatat cccatttgac ttctatttgt |
| 181 | gtgaaatggc ctttccccgg gtcaagccag cacctgatga aacttccttc agtgaggcct |
| 241 | tgctgaagag gaatcaggac ctggctccca attctgctga acaggcatct atcctttctc |
| 301 | tggtgacaaa aataaacaat gtgattgata atctgattgt ggctccaggg acatttgaag |
| 361 | tgcaaattga agaagttcga caggtgggat cctataaaaa ggggacaatg actacaggac |
| 421 | acaatgtggc tgacctggtg gtgatactca agattctgcc aacgttggaa gctgttgctg |
| 481 | ccctggggaa caaagtcgtg gaaagcctaa gagcacagga tccttctgaa gttttaacca |
| 541 | tgctgaccaa cgaaactggc tttgaaatca gttcttctga tgctacagtg aagattctca |
| 601 | ttacaacagt gccacccaat cttcgaaaac tggatccaga actccatttg gatatcaaag |
| 661 | tattgcagag tgccttagca gccatccgac atgcccgctg gttcgaggaa aatgcttctc |
| 721 | agtccacagt taaagttctc atcagactac tgaaggactt gaggattcgt tttcctggct |
| 781 | ttgagcccct cacaccctgg atccttgacc tactaggcca ttatgctgtg atgaacaacc |
| 841 | ccaccagaca gcctttggcc ctaaacgttg catacaggcg ctgcttgcag attctggctg |
| 901 | caggactgtt cctgccaggt tcagtgggta tcactgaccc ctgtgagagt ggcaacttta |
| 961 | gagtacacac agtcatgacc ctagaacagc aggacatggt ctgctataca gctcagactc |
| 1021 | tcgtccgaat cctctcacat ggtggcttta ggaagatcct tggccaggag ggtgatgcca |
| 1081 | gctatcttgc ttctgaaata tctacctggg atggagtgat agtaacacct tcagaaaagg |
| 1141 | cttatgagaa gccaccagag aagaaggaag gagaggaaga agaggagaat acagaagaac |
| 1201 | cacctcaagg agaggaagaa gaaagcatgg aaactcagga gtgacattcc cttcactcct |
| 1261 | tttcctaccc aagggggaag actggagcct aagctgcctg ctactgggct ttacatggtg |
| 1321 | acagacattt ccgtgggata gggaagatag caggaagaaa agtaaactcc atagaagtgt |
| 1381 | cattccactg ggttttgata ttggcttagc tgccagtctc ccatttgtga cctatgccat |
| 1441 | ccatctataa tggaggatac caacatttct tcctaatatt ctataatctc caactcctga |
| 1501 | aaacccctct ctcaactaat actttgctgt tgaaatgttg tgaaatgtta agtgtctgga |
| 1561 | aatttttttt tctaagaaaa actattaaag tacttcctag tagggctctt ggtctttctg |
| 1621 | gttggtgttt tttgtttgtt tgtttttttg catgtggcaa ccttttttgc ttttggcttt |
| 1681 | gtgtactctt ttgttgaaaa tgataattga gtcatttaat tataaatact cagatcacaa |
| 1741 | tttgaagctg tctttgaatt aaaggacaaa ctcctggaac ctttccatat accagagtaa |
| 1801 | actagaatat cattttaagt atttattgaa taaaatgatt ctatcaacac tg |

An exemplary SFPQ polypeptide sequence is provided at NCBI Accession No. NP_005057, version NP_005057.1, incorporated herein by reference and set forth below (SEQ ID NO: 17):

| | |
|---|---|
| 1 | msrdrfrsrg gggggfhrrg ggggrgglhd frspppgmgl nqnrgpmgpg pgqsgpkppi |
| 61 | ppppphqqqq qpppqqpppq qppphqppph pqphqqqqpp pppqdsskpv vaqgpgpapg |
| 121 | vgsappasss appatpptsg appgsgpgpt ptpppavtsa ppgappptpp ssgvpttppq |
| 181 | aggpppppaa vpgpgpgpkq gpgpggpkgg kmpggpkpgg gpglstpggh pkpphrggge |
| 241 | prggrqhhpp yhqqhhqgpp pggpggrsee kisdsegfka nlsllrrpge ktytqrcrlf |
| 301 | vgnlpadite defkrlfaky gepgevfink gkgfgfikle sralaeiaka elddtpmrgr |
| 361 | qlrvrfatha aalsvrnlsp yvsnelleea fsqfgpiera vvivddrgrs tgkgivefas |
| 421 | kpaarkafer csegvflltt tprpvivepl eqlddedglp eklaqknpmy qkeretpprf |
| 481 | aqhgtfeyey sqrwksldem ekqqreqvek nmkdakdkle semedayheh qanllrqdlm |
| 541 | rrqeelrrme elhnqemqkr kemqlrqeee rrrreeemmi rqremeeqmr rqreesysrm |
| 601 | gymdprerdm rmggggamnm gdpygsggqk fpplgggggi gyeanpgvpp atmsgsmmgs |
| 661 | dmrterfgqg gagpvggqgp rgmgpgtpag ygrgreeyeg pnkkprf |

An exemplary SFPQ nucleic acid sequence is provided at NCBI Accession No. NM_005066, version NM_005066.3, incorporated herein by reference and set forth below (SEQ ID NO: 18):

| | |
|---|---|
| 1 | gcctgtgtca tccgccattt tgtgagaagc aaggtggcct ccacgtttcc tgagcgtctt |
| 61 | cttcgctttt gcctcgaccg ccccttgacc acagacatgt ctcgggatcg gttccggagt |
| 121 | cgtggcggtg gcggtggtgg cttccacagg cgtggaggag gcggcggccg cggcggcctc |
| 181 | cacgacttcc gttctccgcc gcccggcatg ggcctcaatc agaatcgcgg ccccatgggt |
| 241 | cctggcccgg gccagagcgg ccctaagcct ccgatcccgc caccgcctcc acaccaacag |
| 301 | cagcaacagc caccaccgca gcagccaccg ccgcagcagc cgccaccgca tcagccgccg |
| 361 | ccgcatccac agccgcatca gcagcagcag ccgccgccac cgccgcagga ctcttccaag |
| 421 | cccgtcgttg ctcagggacc cggccccgct cccggagtag gcagcgcacc accagcctcc |
| 481 | agctcggccc cgcccgccac tccaccaacc tcgggggccc cgccagggtc cgggccaggc |
| 541 | ccgactccga ccccgccgcc tgcagtcacc tcggcccctc ccggggcgcc gccacccacc |
| 601 | ccgccaagca gcggggtccc taccacacct cctcaggccg gaggcccgcc gcctccgccc |
| 661 | gcggcagtcc cgggcccggg tccagggcct aagcagggcc caggtccggg tggtcccaaa |
| 721 | ggcggcaaaa tgcctggcgg gccgaagcca ggtggcggcc cgggcctaag tacgcctggc |
| 781 | ggccacccca agccgccgca tcgaggcggc ggggagcccc gcgggggccg ccagcaccac |
| 841 | ccgccctacc accagcagca tcaccagggg cccccgcccg gcgggcccgg cggccgcagc |
| 901 | gaggagaaga tctcggactc ggaggggttt aaagccaatt tgtctctctt gaggaggcct |
| 961 | ggagagaaaa cttacacaca gcgatgtcgg ttgtttgttg ggaatctacc tgctgatatc |
| 1021 | acggaggatg aattcaaaag actatttgct aaatatggag aaccaggaga agtttttatc |
| 1081 | aacaaaggca aaggattcgg atttattaag cttgaatcta gagctttggc tgaaattgcc |
| 1141 | aaagccgaac tggatgatac acccatgaga ggtagacagc ttcgagttcg ctttgccaca |
| 1201 | catgctgctg ccctttctgt tcgtaatctt tcaccttatg tttccaatga actgttggaa |
| 1261 | gaagccttta gccaatttgg tcctattgaa agggctgttg taatagtgga tgatcgtgga |
| 1321 | agatctacag ggaaaggcat tgttgaattt gcttctaagc cagcagcaag aaaggcattt |
| 1381 | gaacgatgca gtgaaggtgt tttcttactg acgacaactc ctcgtccagt cattgtggaa |
| 1441 | ccacttgaac aactagatga tgaagatggt cttcctgaaa aacttgccca gaagaatcca |
| 1501 | atgtatcaaa aggagagaga aacccctcct cgttttgccc agcatggcac gtttgagtac |
| 1561 | gaatattctc agcgatggaa gtctttggat gaaatggaaa aacagcaaag ggaacaagtt |
| 1621 | gaaaaaaaca tgaaagatgc aaaagacaaa ttggaaagtg aaatggaaga tgcctatcat |
| 1681 | gaacatcagg caaatctttt gcgccaagat ctgatgagac gacaggaaga attaagacgc |
| 1741 | atggaagaac ttcacaatca agaaatgcag aaacgtaaag aaatgcaatt gaggcaagag |
| 1801 | gaggaacgac gtagaagaga ggaagagatg atgattcgtc aacgtgagat ggaagaacaa |
| 1861 | atgaggcgcc aaagagagga aagttacagc cgaatgggct acatggatcc acgggaaaga |
| 1921 | gacatgcgaa tgggtggcgg aggagcaatg aacatgggag atccctatgg ttcaggaggc |
| 1981 | cagaaatttc cacctctagg aggtggtggt ggcataggtt atgaagctaa tcctggcgtt |
| 2041 | ccaccagcaa ccatgagtgg ttccatgatg ggaagtgaca tgcgtactga gcgctttggg |
| 2101 | cagggaggtg cggggcctgt gggtggacag ggtcctagag gaatggggcc tggaactcca |
| 2161 | gcaggatatg gtagagggag agaagagtac gaaggcccaa acaaaaaacc ccgattttag |
| 2221 | atgtgatatt taggctttca ttccagtttg ttttgttttt ttgtttagat accaatcttt |
| 2281 | taaattcttg cattttagta agaaagctat ctttttatgg atgttagcag tttattgacc |
| 2341 | taatatttgt aaatggtctg tttgggcagg taaaattatg taatgcagtg tttggaacag |
| 2401 | gagaattttt ttttcctttt tatttcttta ttttttcttt tttactgtat aatgtccctc |
| 2461 | aagtttatgg cagtgtacct tgtgccactg aatttccaaa gtgtaccaat tttttttttt |
| 2521 | ttactgtgct tcaaataaat agaaaaatag ttataatatt gatcttcaac tttgccattc |
| 2581 | atgcttctat gcatattagg ctacgtattc cacattgaaa gcatgagagt gtctaggcct |
| 2641 | ttgaatggca tatgccattt ctgggaaatg catctggagg ctaagtattg ctttctacaa |
| 2701 | ataattgccc cctttgtttt aaaaagaaga aatgcatatt gaagtagttt gatgatttgt |
| 2761 | ttggcatata ggaagcacgc tggtgctaag tattttttaa atggttatgt aagcaaagct |
| 2821 | gaactgtaaa tcttcaggaa tatgtattaa gattgtggaa tgggtgtaag acaattggta |
| 2881 | gggggtgaaa gtgggtttga ttaaatggat cttttatggc cctatgatct atcctttact |
| 2941 | tgaaagcttt tgaaaagtgg aaaggtcatt ttgttgcatt tccccatttc ttgtttttaa |
| 3001 | aagaccaaca aatctcaagc cctataaatg gcttgtattg aacttttaca tttgaattaa |
| 3061 | agatgttaaa catgaagtct tttcatgtat tcaagtgatt tataaagatg gggtgtagtc |
| 3121 | taaaaaacaa aacttgaaag taagaaggtt tagtaactgg gccgggcgcg gtggctcacg |
| 3181 | cctgtaatcc cagcactttg ggaggctgag gtgggcaaat catgagatca ggagttcaag |
| 3241 | accagcctgg ccaacatggt gaaaccccgt ctctactaaa aatacaaaaa attagctggg |
| 3301 | cgtggtggca ggcgcctgta atcccagcta ctcgggaggc tgagccagga gaattgcttg |
| 3361 | aacctggaga tggaggttgc agtgagctga gactgccact gcactccagc ctggacaaca |
| 3421 | gagcgagact ctgtctcaaa aaaaaaaaaa ggtttagtac tgaagtaggt ttagttatat |
| 3481 | atacatttgt actctgtaac ttttacatct ccttgtgcta cttctgttgg gcaaaatcta |
| 3541 | taggactata aaaatctatt ggttctcttc taagagctag tggtgattgt taacaaatag |
| 3601 | ggttgcagat aatcattctt attgtaaatt taacttgttt tcacatagtt tcgttttcat |
| 3661 | gtagcactaa atctggagaa agttgctcat gctacacagt acaaatatcc cttcctccca |
| 3721 | ccataaaggc ttggaatgtt |

An exemplary Valosin Containing Protein (VCP) polypeptide sequence is provided at NCBI Accession No. P55072, version P55072.4, incorporated herein by reference and set forth below (SEQ ID NO: 19):

| | |
|---|---|
| 1 | masgadskgd dlstailkqk nrpnrlivde ainednsvvs lsqpkmdelq lfrgdtvllk |
| 61 | gkkrreavci vlsddtcsde kirmnrvvrn nlrvrlgdvi siqpcpdvky gkrihvlpid |
| 121 | dtvegitgnl fevylkpyfl eayrpirkgd iflvrggmra vefkvvetdp spycivapdt |
| 181 | vihcegepik redeeeslne vgyddiggcr kqlaqikemv elplrhpalf kaigvkpprg |
| 241 | illygppgtg ktliaravan etgaffflin gpeimsklag esesnlrkaf eeaeknapai |
| 301 | ifideldaia pkrekthgev errivsqllt lmdglkqrah vivmaatnrp nsidpalrrf |
| 361 | grfdrevdig ipdatgrlei lqihtknmkl addvdleqva nethghvgad laalcseaal |
| 421 | qairkkmdli dledetidae vmnslavtmd dfrwalsqsn psalretvve vpqvtwedig |
| 481 | gledvkrelq elvqypvehp dkflkfgmtp skgvlfygpp gcgktllaka ianecqanfi |
| 541 | sikgpelltm wfgeseanvr eifdkarqaa pcvlffdeld siakarggni gdgggaadrv |
| 601 | inqiltemdg mstkknvfii gatnrpdiid pailrpgrld qliyiplpde ksrvailkan |
| 661 | lrkspvakdv dleflakmtn gfsgadltei cqracklair esieseirre rerqtnpsam |
| 721 | eveeddpvpe irrdhfeeam rfarrsvsdn dirkyemfaq tlqqsrgfgs frfpsgnqgg |
| 781 | agpsqgsggg tggsvytedn dddlyg |

An exemplary VCP nucleic acid sequence is provided at NCBI Accession No. NM_007126, version NM_007126.5, incorporated herein by reference and set forth below (SEQ ID NO: 20):

| | |
|---|---|
| 1 | agtctcagcg aagcgtctgc gaccgtcgtt tgagtcgtcg ctgccgctgc cgctgccact |
| 61 | gccactgcca cctcgcggat caggagccag cgttgttcgc ccgacgcctc gctgccggtg |
| 121 | ggaggaagcg agagggaagc cgcttgcggg tttgtcgccg ctgctcgccc accgcctgga |
| 181 | agagccgagc cccggcccag tcggtcgctt gccaccgctc gtagccgtta cccgcgggcc |
| 241 | gccacagccg ccggccggga gaggcgcgcg ccatggcttc tggagccgat tcaaaaggtg |
| 301 | atgacctatc aacagccatt ctcaaacaga agaaccgtcc caatcggtta attgttgatg |
| 361 | aagccatcaa tgaggacaac agtgtggtgt ccttgtccca gcccaagatg gatgaattgc |
| 421 | agttgttccg aggtgacaca gtgttgctga aaggaaagaa gagacgagaa gctgtttgca |
| 481 | tcgtcctttc tgatgatact tgttctgatg agaagattcg gatgaataga gttgttcgga |
| 541 | ataaccttcg tgtacgccta ggggatgtca tcagcatcca gccatgccct gatgtgaagt |
| 601 | acggcaaacg tatccatgtg ctgcccattg atgacacagt ggaaggcatt actggtaatc |
| 661 | tcttcgaggt ataccttaag ccgtacttcc tggaagcgta tcgacccatc cggaaaggag |
| 721 | acatttttct tgtccgtggt gggatgcgtg ctgtggagtt caaagtggtg gaaacagatc |
| 781 | ctagccctta ttgcattgtt gctccagaca cagtgatcca ctgcgaaggg gagcctatca |
| 841 | aacgagagga tgaggaagag tccttgaatg aagtagggta tgatgacatt ggtggctgca |
| 901 | ggaagcagct agctcagata aaggagatgg tggaactgcc cctgagacat cctgccctct |
| 961 | ttaaggcaat tggtgtgaag cctcctagag gaatcctgct ttacggacct cctggaacag |
| 1021 | gaaagaccct gattgctcga gctgtagcaa atgagactgg agccttcttc ttcttgatca |
| 1081 | atggtcctga gatcatgagc aaattggctg gtgagtctga gagcaacctt cgtaaagcct |
| 1141 | ttgaggaggc tgagaagaat gctcctgcca tcatcttcat tgatgagcta gatgccatcg |
| 1201 | ctcccaaaag agagaaaact catggcgagg tggagcggcg cattgtatca cagttgttga |
| 1261 | ccctcatgga tggcctaaag cagagggcac atgtgattgt tatggcagca accaacagac |
| 1321 | ccaacagcat tgacccagct ctacggcgat ttggtcgctt tgacagggag gtagatattg |
| 1381 | gaattcctga tgctacagga cgcttagaga ttcttcagat ccataccaag aacatgaagc |
| 1441 | tggcagatga tgtggacctg gaacaggtag ccaatgagac tcacgggcat gtgggtgctg |
| 1501 | acttagcagc cctgtgctca gaggctgctc tgcaagccat ccgcaagaag atggatctca |
| 1561 | ttgacctaga ggatgagacc attgatgccg aggtcatgaa ctctctagca gttactatgg |
| 1621 | atgacttccg gtgggccttg agccagagta acccatcagc actgcgggaa accgtggtag |
| 1681 | aggtgccaca ggtaacctgg gaagacatcg ggggcctaga ggatgtcaaa cgtgagctac |
| 1741 | aggagctggt ccagtatcct gtggagcacc cagacaaatt cctgaagttt ggcatgacac |
| 1801 | cttccaaggg agttctgttc tatggacctc ctggctgtgg gaaaactttg ttggccaaag |
| 1861 | ccattgctaa tgaatgccag gccaacttca tctccatcaa gggtcctgag ctgctcacca |
| 1921 | tgtggtttgg ggagtctgag gccaatgtca gagaaatctt tgacaaggcc cgccaagctg |
| 1981 | ccccctgtgt gctattcttt gatgagctgg attcgattgc caaggctcgt ggaggtaaca |
| 2041 | ttggagatgg tggtggggct gctgaccgag tcatcaacca gatcctgaca gaaatggatg |
| 2101 | gcatgtccac aaaaaaaaat gtgttcatca ttggcgctac caaccggcct gacatcattg |
| 2161 | atcctgccat cctcagacct ggccgtcttg atcagctcat ctacatccca cttcctgatg |
| 2221 | agaagtcccg tgttgccatc ctcaaggcta acctgcgcaa gtccccagtt gccaaggatg |
| 2281 | tggacttgga gttcctggct aaaatgacta atggcttctc tggagctgac ctgacagaga |
| 2341 | tttgccagcg tgcttgcaag ctggccatcc gtgaatccat cgagagtgag attaggcgag |
| 2401 | aacgagagag gcagacaaac ccatcagcca tggaggtaga agaggatgat ccagtgcctg |
| 2461 | agatccgtcg agatcacttt gaagaagcca tgcgctttgc gcgccgttct gtcagtgaca |
| 2521 | atgacattcg gaagtatgag atgtttgccc agacccttca gcagagtcgg ggctttggca |
| 2581 | gcttcagatt cccttcaggg aaccagggtg gagctggccc cagtcagggc agtggaggcg |
| 2641 | gcacaggtgg cagtgtatac acagaagaca atgatgatga cctgtatggc taagtggtgg |
| 2701 | tggccagcgt gcagtgagct ggcctgcctg gaccttgttc cctgggggtg ggggcgcttg |
| 2761 | cccaggagag ggaccagggg tgcgcccaca gcctgctcca ttctccagtc tgaacagttc |
| 2821 | agctacagtc tgactctgga cagggggttt ctgttgcaaa aatacaaaac aaaagcgata |
| 2881 | aaataaaagc gattttcatt tggtaggcgg agagtgaatt accaacaggg aattgggcct |
| 2941 | tgggcctatg ccatttctgt tgtagtttgg ggcagtgcag gggacctgtg tggggtgtga |
| 3001 | accaaggcac tactgccacc tgccacagta aagcatctgc acttgactca atgctgcccg |
| 3061 | agccctccct tccccctatc caacctgggt aggtgggtag gggccacagt tgctggatgt |
| 3121 | ttatatagag agtaggttga tttattttac atgcttttga gttaatgttg gaaaactaat |
| 3181 | cacaagcagt ttctaaacca aaaaatgaca tgttgtaaaa ggacaataaa cgttgggtca |
| 3241 | aaatggagcc tgagtcctgg gccctgtgcc tgcttctttt cctgggaaca gccttgggct |
| 3301 | acccaccact cccaaggcat tcttccaaat gtgaaatcct ggaagtaaga ttgcaccttc |
| 3361 | ttcctctcct gatcaacatc ggtatgatgt ctcctgttgc ctcacccttt gtctgcagta |
| 3421 | tcactggata ggactggtgg aaagggagca gcctgacaga gctccaaatg tggagaatat |
| 3481 | ggcatccctc cacctatatt tgatgtggac ggtaaggcta ggcctgcagg atcccttatc |
| 3541 | ctgaccaaag actgtgttgg ggtgccattt gaaaatcgca gggttgcaaa agaatacaat |
| 3601 | cttacttgca ggtggatatt ctctatactc tcttttaatg catctaaaaa tcccaaacat |
| 3661 | cccctggttg gtgatcactt acagttgtgt ccacctttat tttatgtact ttgattaaaa |
| 3721 | aaaaact ttttgttaat ataaaa |

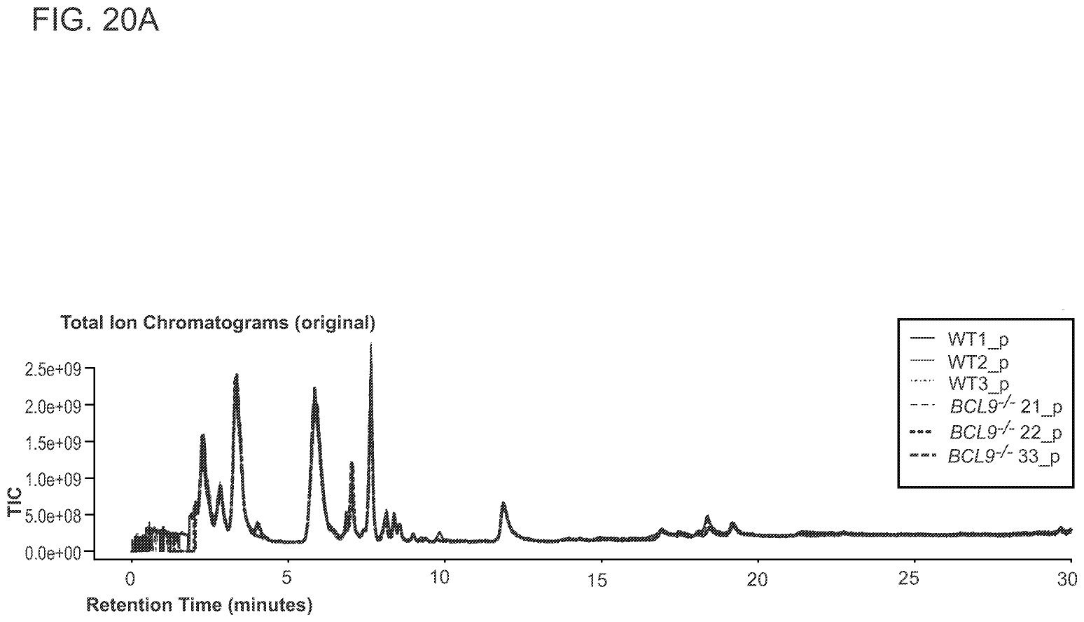

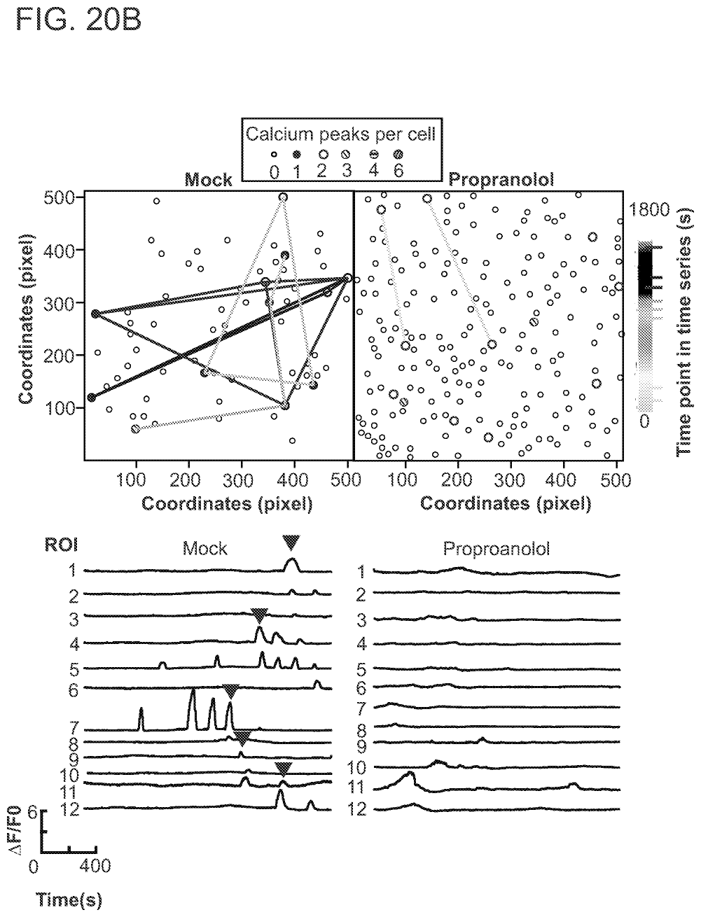

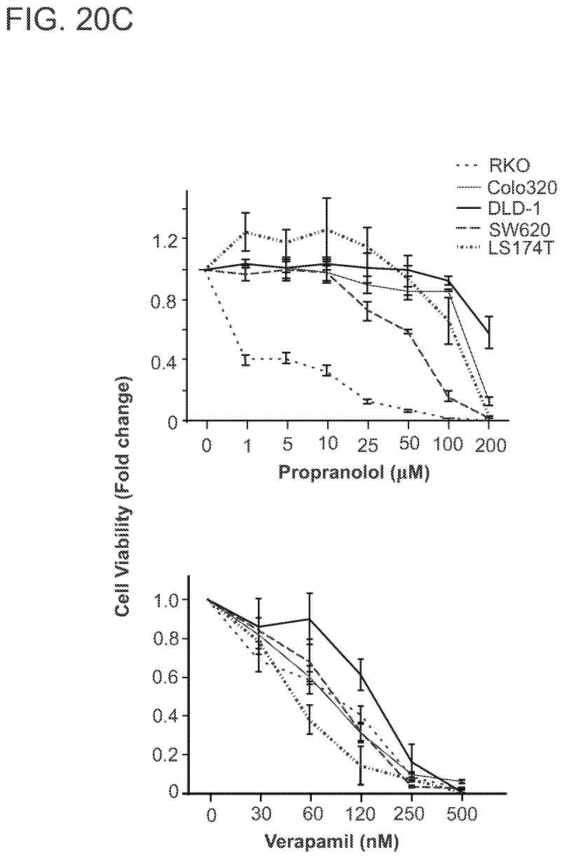

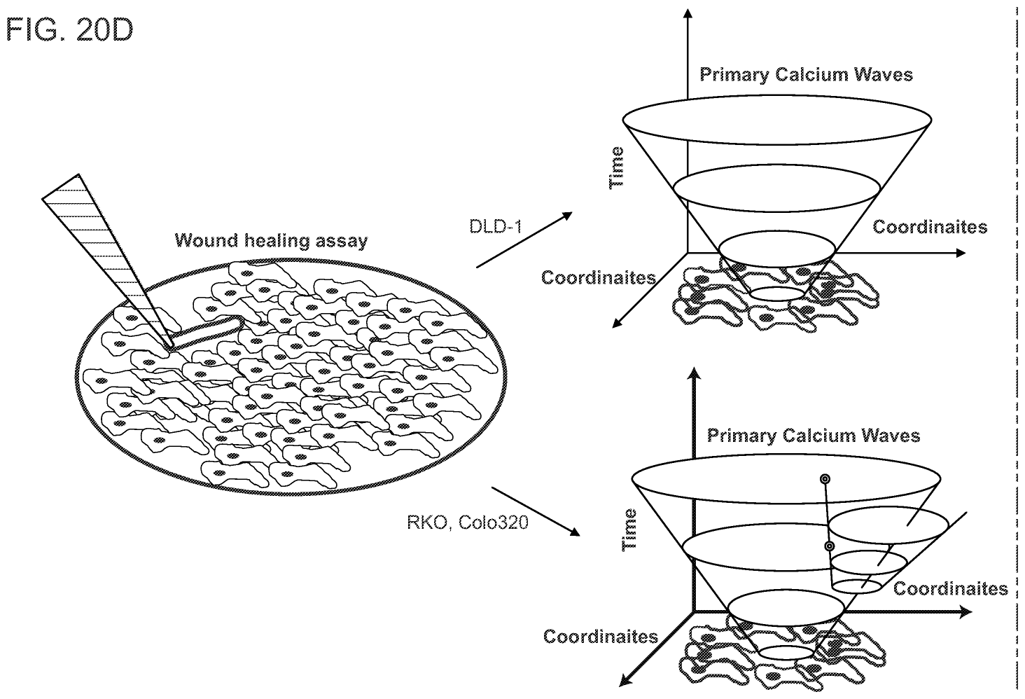

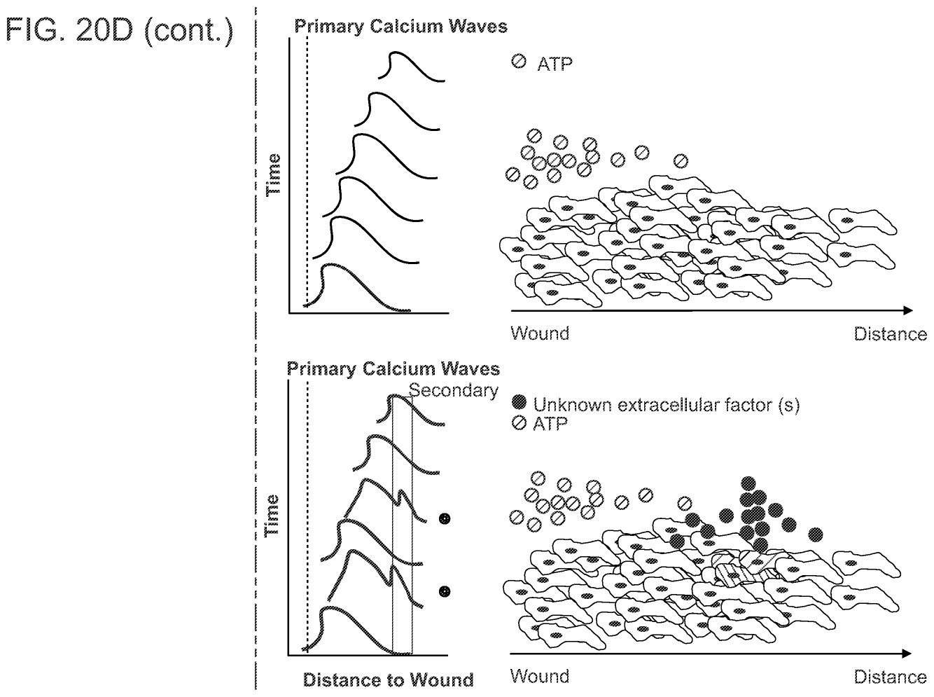

Spontaneous calcium transients among C1 CRC cells have been identified, in which spreading was dependent on specific projection of neurotransmitters and the induction of calcium channel opening in distant cells. Similar phenomena have also been described in other cancer types (Zhu et al., (2014) Oncotarget 5:3455-3471; Zhou et al., (2019) FASEB J. 33:4675-4687), including breast cancer, in which calcium influx was found to be driven by the TRP or ORAI family of calcium channels (McAndrew et al. (2011) Mol. Cancer Ther. 10:448-460; Prevarskaya et al., (2011) Nat. Rev. Cancer 11:609-618). Expression of these calcium channels is also increased in C1 compared with other CRC clusters, and like L-type calcium channel genes (e.g. CACNA2D1), many of the TRP or ORAI genes also belong to the “black” group in the correlation coefficient matrix, indicating a synergy between these proteins in C1 tumor. In wound healing assays, the spread of calcium transients in BCL9 knockout cells were observed to be blocked in most of the cells adjacent to the wound edge. In contrast, propranolol treatment inhibited calcium transients only in a subset of cells, indicating that several types of neurotransmitters might be involved in the spread of calcium transients in BCL9 wild-type cells. The identification of several molecular structures including terbutaline-like compounds in the MS analysis was consistent with this scenario. The spreading of calcium transients among CRC cells was not promiscuous and followed a cell to cell specific pattern, allowing CRC cells to establish a complex communication network (FIG. 20D). This communication system can reduce the response time to stimulation and can rapidly transmit the information to distant cells. Like normal neural cells, which have been shown to regulate the behavior of M2 resident macrophage in normal colon mucosa and participate in the formation of neural-immune network (Gabanyi et al., (2016), Cell 164, 378-391), C1 cells may also regulate M2 macrophages differentiation in vitro and tumor infiltration in vivo. This is also reflected by the strong correlation of the innate immune and neural gene signatures in the correlation coefficient matrix.

### Calcium Channel Blockers

Calcium channel blockers relax blood vessels and increase the supply of blood and oxygen to the heart while also reducing the heart's workload. This is done by slowing the movement of calcium into the cells of the heart and blood vessel walls. Benzeneacetonitrile, α-[3-[[2-(3,4-dimethoxyphenyl)ethyl] methylamino]propyl]-3,4-dimethoxy-α-(1-methylethyl), also known as Verapamil, is an example of a calcium channel blocker. Verapamil should be given as a

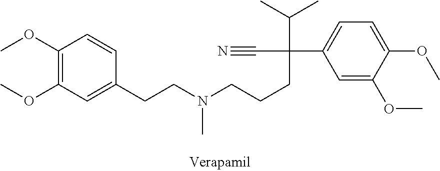

slow intravenous injection over at least a two-minute period of time under continuous ECG and blood pressure monitoring. The recommended intravenous doses for an adult for an initial dose is 5-10 mg (0.075-0.15 mg/kg body weight) given as an intravenous bolus. The repeat dose is 10 mg (0.15 mg/kg body weight) 30 minutes after the first dose if the initial response is not adequate. The recommended initial intravenous doses for a pediatric is as follows: 0-1 year) 0.1-0.2 mg/kg body weight (usual single dose range: 0.75-2 mg) should be administered as an intravenous bolus. 1-15 years) 0.1-0.3 mg/kg body weight (usual single dose range: 2-5 mg) should be administered as an intravenous bolus. The repeat dose for 0-1 year is 0.1-0.2 mg/kg body weight (usual single dose range: 0.75-2 mg) 30 minutes after the first dose if the initial response is not adequate. The repeat dose for 1-15 years is 0.1-0.3 mg/kg body weight (usual single dose range: 2-5 mg) 30 minutes after the first dose if the initial response is not adequate without exceeding 10 mg as a single dose.

### Adrenergic Receptor Inhibitors

Adrenergic receptor inhibitors are used to treat high blood pressure and irregular heart beats by blocking the action of certain natural chemicals in the body, such as epinephrine, that affect the heart and blood vessels. 2-propanol, 1-[(1-methylethyl)amino]-3-(1-naphthalenyloxy), also known as Propranolol, is an example of an adrenergic receptor inhibitor. The usual initial

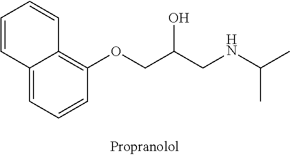 [SMILES: CC(C)NCC(O)COc1cccc2ccccc12]

dosage is 40 mg of Propranolol twice daily. Dosage may be increased gradually until adequate blood pressure control is achieved. The usual maintenance dosage is 120 mg to 240 mg per day. In some instances a dosage of 640 mg a day may be required. The time needed for full antihypertensive response to a given dosage is variable and may range from a few days to several weeks.

### Tissue Remodeling and Wound Healing

Tissue remodeling is the reorganization or renovation of existing tissues and it is critical during development and wound healing. The process can either change the characteristic of a tissue such as in blood vessel remodeling, or result in the dynamic equilibrium of a tissue such as in bone remodeling. Wound healing is comprised of a continuous sequence of inflammation and repair, in which epithelial, endothelial, inflammatory cells, platelets and fibroblasts briefly come together and interact to restore a semblance of their usual discipline and normal function. A number of proteins play crucial roles in activating processes, such as chemotaxis and proliferation, which are necessary in order for wound healing to commence. Some examples of these proteins include Fibroblast Activation Protein (FAP), Platelet Derived Growth Factor Subunit B (PDGFB), Complement C3 (C3), calcium voltage-gated channel auxiliary subunit alpha2delta 1 (CACNA2D1), or regulator of G protein signaling 4 (RGS4). Another example includes synaptophysin (SYP).

An exemplary FAP polypeptide sequence is provided at NCBI Accession No. Q12884, version Q12884.5, incorporated herein by reference and set forth below (SEQ ID NO: 21):

| | |
|---|---|
| 1 | mktwvkivfg vatsavlall vmcivlrpsr vhnseentmr altlkdilng tfsyktffpn |
| 61 | wisgqeylhq sadnnivlyn ietgqsytil snrtmksvna snyglspdrq fvylesdysk |
| 121 | lwrysytaty yiydlsngef vrgnelprpi qylcwspvgs klayvyqnni ylkqrpgdpp |
| 181 | fqitfngren kifngipdwv yeeemlatky alwwspngkf layaefndtd ipviaysyyg |
| 241 | deqyprtini pypkagaknp vvrifiidtt ypayvgpqev pvpamiassd yyfswltwvt |
| 301 | dervclqwlk rvqnvsvlsi cdfredwqtw dcpktqehie esrtgwaggf fvstpvfsyd |
| 361 | aisyykifsd kdgykhihyi kdtvenaiqi tsgkweaini frvtqdslfy ssnefeeypg |
| 421 | rrniyrisig syppskkcvt chlrkercqy ytasfsdyak yyalvcygpg ipistlhdgr |
| 481 | tdqeikilee nkelenalkn iqlpkeeikk levdeitlwy kmilppqfdr skkyplliqv |
| 541 | yggpcsqsvr svfavnwisy laskegmvia lvdgrgtafq gdkllyavyr klgvyevedq |
| 601 | itavrkfiem gfidekriai wgwsyggyvs slalasgtgl fkcgiavapv ssweyyasvy |
| 661 | terfmglptk ddnlehykns tvmaraeyfr nvdyllihgt addnvhfqns aqiakalvna |
| 721 | qvdfqamwys dqnhglsgls tnhlythmth flkqcfslsd |

An exemplary FAP nucleic acid sequence is provided at NCBI Accession No. NM_004460, version NM_004460.5, incorporated herein by reference and set forth below (SEQ ID NO: 22):

| | |
|---|---|
| 1 | gtcctggctt cagcttccaa ctacaaagac agacttggtc cttttcaacg gttttcacag |
| 61 | atccagtgac ccacgctctg aagacagaat tagctaactt tcaaaaacat ctggaaaaat |
| 121 | gaagacttgg gtaaaaatcg tatttggagt tgccacctct gctgtgcttg ccttattggt |
| 181 | gatgtgcatt gtcttacgcc cttcaagagt tcataactct gaagaaaata caatgagagc |
| 241 | actcacactg aaggatattt taaatggaac attttcttat aaaacatttt ttccaaactg |
| 301 | gatttcagga caagaatatc ttcatcaatc tgcagataac aatatagtac tttataatat |
| 361 | tgaaacagga caatcatata ccattttgag taatagaacc atgaaaagtg tgaatgcttc |
| 421 | aaattacggc ttatcacctg atcggcaatt tgtatatcta gaaagtgatt attcaaagct |
| 481 | ttggagatac tcttacacag caacatatta catctatgac cttagcaatg gagaatttgt |
| 541 | aagaggaaat gagcttcctc gtccaattca gtatttatgc tggtcgcctg ttgggagtaa |
| 601 | attagcatat gtctatcaaa acaatatcta tttgaaacaa agaccaggag atccaccttt |
| 661 | tcaaataaca tttaatggaa gagaaaataa aatatttaat ggaatcccag actgggttta |
| 721 | tgaagaggaa atgcttgcta caaaatatgc tctctggtgg tctcctaatg gaaaattttt |
| 781 | ggcatatgcg gaatttaatg atacggatat accagttatt gcctattcct attatggcga |
| 841 | tgaacaatat cctagaacaa taaatattcc atacccaaag gctggagcta agaatcccgt |
| 901 | tgttcggata tttattatcg ataccactta ccctgcgtat gtaggtcccc aggaagtgcc |
| 961 | tgttccagca atgatagcct caagtgatta ttatttcagt tggctcacgt gggttactga |
| 1021 | tgaacgagta tgtttgcagt ggctaaaaag agtccagaat gtttcggtcc tgtctatatg |
| 1081 | tgacttcagg gaagactggc agacatggga ttgtccaaag acccaggagc atatagaaga |
| 1141 | aagcagaact ggatgggctg gtggattctt tgtttcaaca ccagttttca gctatgatgc |
| 1201 | catttcgtac tacaaaatat ttagtgacaa ggatggctac aaacatattc actatatcaa |
| 1261 | agacactgtg gaaaatgcta ttcaaattac aagtggcaag tgggaggcca taaatatatt |
| 1321 | cagagtaaca caggattcac tgttttattc tagcaatgaa tttgaagaat accctggaag |
| 1381 | aagaaacatc tacagaatta gcattggaag ctatcctcca agcaagaagt gtgttacttg |
| 1441 | ccatctaagg aaagaaaggt gccaatatta cacagcaagt ttcagcgact acgccaagta |
| 1501 | ctatgcactt gtctgctacg gcccaggcat ccccatttcc acccttcatg atggacgcac |
| 1561 | tgatcaagaa attaaaatcc tggaagaaaa caaggaattg gaaaatgctt tgaaaaatat |
| 1621 | ccagctgcct aaagaggaaa ttaagaaact tgaagtagat gaaattactt tatggtacaa |
| 1681 | gatgattctt cctcctcaat ttgacagatc aaagaagtat cccttgctaa ttcaagtgta |
| 1741 | tggtggtccc tgcagtcaga gtgtaaggtc tgtatttgct gttaattgga tatcttatct |
| 1801 | tgcaagtaag gaagggatgg tcattgcctt ggtggatggt cgaggaacag ctttccaagg |
| 1861 | tgacaaactc ctctatgcag tgtatcgaaa gctgggtgtt tatgaagttg aagaccagat |
| 1921 | tacagctgtc agaaaattca tagaaatggg tttcattgat gaaaaaagaa tagccatatg |
| 1981 | gggctggtcc tatggaggat acgtttcatc actggccctt gcatctggaa ctggtctttt |
| 2041 | caaatgtggt atagcagtgg ctccagtctc cagctgggaa tattacgcgt ctgtctacac |
| 2101 | agagagattc atgggtctcc caacaaagga tgataatctt gagcactata agaattcaac |
| 2161 | tgtgatggca agagcagaat atttcagaaa tgtagactat cttctcatcc acggaacagc |
| 2221 | agatgataat gtgcactttc aaaactcagc acagattgct aaagctctgg ttaatgcaca |
| 2281 | agtggatttc caggcaatgt ggtactctga ccagaaccac ggcttatccg gcctgtccac |
| 2341 | gaaccactta tacacccaca tgacccactt cctaaagcag tgtttctctt tgtcagacta |
| 2401 | aaaacgatgc agatgcaagc ctgtatcaga atctgaaaac cttatataaa cccctcagac |
| 2461 | agtttgctta ttttattttt tatgttgtaa aatgctagta taaacaaaca aattaatgtt |
| 2521 | gttctaaagg ctgttaaaaa aaagatgagg actcagaagt tcaagctaaa tattgtttac |
| 2581 | attttctggt actctgtgaa agaagagaaa agggagtcat gcattttgct ttggacacag |
| 2641 | tgttttatca cctgttcatt tgaagaaaaa taataaagtc agaagttcaa gtgcta |

An exemplary PDGFB polypeptide sequence is provided at NCBI Accession No. P01127, version P01127.1, incorporated herein by reference and set forth below (SEQ ID NO: 23):

| | |
|---|---|
| 1 | mnrcwalfls lccylrlvsa egdpipeely emlsdhsirs fddlqrllhg dpgeedgael |
| 61 | dlnmtrshsg geleslargr rslgsltiae pamiaecktr tevfeisrrl idrtnanflv |
| 121 | wppcvevqrc sgccnnrnvq crptqvqlrp vqvrkieivr kkpifkkatv tledhlackc |
| 181 | etvaaarpvt rspggsqeqr aktpqtrvti rtvrvrrppk gkhrkfkhth dktalketlg |
| 241 | a |

An exemplary PDGFB nucleic acid sequence is provided at NCBI Accession No. NM_002608, version NM_002608.3, incorporated herein by reference and set forth below (SEQ ID NO: 24):

| | |
|---|---|
| 1 | gaggaaaggc tgtctccacc cacctctcgc actctccctt ctcctttata aaggccggaa |
| 61 | cagctgaaag ggtggcaact tctcctcctg cagccgggag cggcctgcct gcctccctgc |
| 121 | gcacccgcag cctcccccgc tgcctcccta gggctcccct ccggccgcca gcgcccattt |
| 181 | ttcattccct agatagagat actttgcgcg cacacacata catacgcgcg caaaaaggaa |
| 241 | aaaaaaaaaa aaaagcccac cctccagcct cgctgcaaag agaaaaccgg agcagccgca |
| 301 | gctcgcagct cgcagctcgc agcccgcagc ccgcagagga cgcccagagc ggcgagcggg |
| 361 | cgggcagacg gaccgacgga ctcgcgccgc gtccacctgt cggccgggcc cagccgagcg |
| 421 | cgcagcgggc acgccgcgcg cgcggagcag ccgtgcccgc cgcccgggcc ccgcgccagg |
| 481 | gcgcacacgc tcccgccccc ctacccggcc cgggcgggag tttgcacctc tccctgcccg |
| 541 | ggtgctcgag ctgccgttgc aaagccaact ttggaaaaag ttttttgggg gagacttggg |
| 601 | ccttgaggtg cccagctccg cgctttccga ttttgggggc ctttccagaa aatgttgcaa |
| 661 | aaaagctaag ccggcgggca gaggaaaacg cctgtagccg gcgagtgaag acgaaccatc |
| 721 | gactgccgtg ttccttttcc tcttggaggt tggagtcccc tgggcgcccc cacacggcta |
| 781 | gacgcctcgg ctggttcgcg acgcagcccc ccggccgtgg atgctcactc gggctcggga |
| 841 | tccgcccagg tagcggcctc ggacccaggt cctgcgccca ggtcctcccc tgccccccag |
| 901 | cgacggagcc ggggccgggg gcggcggcgc ccgggggcca tgcgggtgag ccgcggctgc |
| 961 | agaggcctga gcgcctgatc gccgcggacc cgagccgagc ccacccccct ccccagcccc |
| 1021 | ccaccctggc cgcgggggcg gcgcgctcga tctacgcgtc cggggccccg cggggccggg |
| 1081 | cccggagtcg gcatgaatcg ctgctgggcg ctcttcctgt ctctctgctg ctacctgcgt |
| 1141 | ctggtcagcg ccgaggggga ccccattccc gaggagcttt atgagatgct gagtgaccac |
| 1201 | tcgatccgct cctttgatga tctccaacgc ctgctgcacg gagaccccgg agaggaagat |
| 1261 | ggggccgagt tggacctgaa catgacccgc tcccactctg gaggcgagct ggagagcttg |
| 1321 | gctcgtggaa gaaggagcct gggttccctg accattgctg agccggccat gatcgccgag |
| 1381 | tgcaagacgc gcaccgaggt gttcgagatc tcccggcgcc tcatagaccg caccaacgcc |
| 1441 | aacttcctgg tgtggccgcc ctgtgtggag gtgcagcgct gctccggctg ctgcaacaac |
| 1501 | cgcaacgtgc agtgccgccc cacccaggtg cagctgcgac ctgtccaggt gagaaagatc |
| 1561 | gagattgtgc ggaagaagcc aatctttaag aaggccacgg tgacgctgga agaccacctg |
| 1621 | gcatgcaagt gtgagacagt ggcagctgca cggcctgtga cccgaagccc ggggggttcc |
| 1681 | caggagcagc gagccaaaac gccccaaact cgggtgacca ttcggacggt gcgagtccgc |
| 1741 | cggcccccca agggcaagca ccggaaattc aagcacacgc atgacaagac ggcactgaag |
| 1801 | gagacccttg gagcctaggg gcatcggcag gagagtgtgt gggcagggtt atttaatatg |
| 1861 | gtatttgctg tattgccccc atggggtcct tggagtgata atattgtttc cctcgtccgt |
| 1921 | ctgtctcgat gcctgattcg gacggccaat ggtgcttccc ccacccctcc acgtgtccgt |
| 1981 | ccacccttcc atcagcgggt ctcctcccag cggcctccgg cgtcttgccc agcagctcaa |
| 2041 | gaagaaaaag aaggactgaa ctccatcgcc atcttcttcc cttaactcca agaacttggg |
| 2101 | ataagagtgt gagagagact gatggggtcg ctctttgggg gaaacgggct ccttcccctg |
| 2161 | cacctggcct gggccacacc tgagcgctgt ggactgtcct gaggagccct gaggacctct |
| 2221 | cagcatagcc tgcctgatcc ctgaacccct ggccagctct gaggggaggc acctccaggc |
| 2281 | aggccaggct gcctcggact ccatggctaa gaccacagac gggcacacag actggagaaa |
| 2341 | acccctccca cggtgcccaa acaccagtca cctcgtctcc ctggtgcctc tgtgcacagt |
| 2401 | ggcttctttt cgttttcgtt ttgaagacgt ggactcctct tggtgggtgt ggccagcaca |
| 2461 | ccaagtggct gggtgccctc tcaggtgggt tagagatgga gtttgctgtt gaggtggctg |
| 2521 | tagatggtga cctgggtatc ccctgcctcc tgccacccct tcctccccac actccactct |
| 2581 | gattcacctc ttcctctggt tcctttcatc tctctacctc caccctgcat tttcctcttg |
| 2641 | tcctggccct tcagtctgct ccaccaaggg gctcttgaac cccttattaa ggccccagat |
| 2701 | gatcccagtc actcctctct agggcagaag actagaggcc agggcagcaa gggacctgct |
| 2761 | catcatattc caacccagcc acgactgcca tgtaaggttg tgcagggtgt gtactgcaca |
| 2821 | aggacattgt atgcagggag cactgttcac atcatagata aagctgattt gtatatttat |
| 2881 | tatgacaatt tctggcagat gtaggtaaag aggaaaagga tccttttcct aattcacaca |
| 2941 | aagactcctt gtggactggc tgtgcccctg atgcagcctg tggcttggag tggccaaata |
| 3001 | ggagggagac tgtggtaggg gcagggaggc aacactgctg tccacatgac ctccatttcc |
| 3061 | caaagtcctc tgctccagca actgcccttc caggtgggtg tgggacacct gggagaaggt |
| 3121 | ctccaaggga gggtgcagcc ctcttgcccg cacccctccc tgcttgcaca cttccccatc |
| 3181 | tttgatcctt ctgagctcca cctctggtgg ctcctcctag gaaaccagct cgtgggctgg |
| 3241 | gaatggggga gagaagggaa aagatcccca agaccccctg gggtgggatc tgagctccca |
| 3301 | cctcccttcc cacctactgc actttccccc ttcccgcctt ccaaaacctg cttccttcag |
| 3361 | tttgtaaagt cggtgattat atttttgggg gctttccttt tattttttaa atgtaaaatt |
| 3421 | tatttatatt ccgtatttaa agttgtaaaa aaaaataacc acaaaacaaa accaaatgaa |
| 3481 | tccgccggag gtctgtctgt tggcatcgtg cgtgacaatt aacctttctg ccttggcagg |
| 3541 | atgtgccgac agcttgcggc gtgttcctct cactctggga gcctcaggcg tgatctcaca |
| 3601 | cactggcgtg cacatacaca cacacacaca tacatgctca cacatgcgtg cacatacacg |
| 3661 | caggcctgca acttggggga ggcctctgtc tggcgggaag aagagacaca caggctactc |
| 3721 | tgttggtctt ggtcctggca cagctcctga cacgtggact tgtgcgtgtc tctggcagtg |
| 3781 | acgagagatg ggtttctgca g |

An exemplary Complement C3 polypeptide sequence is provided at NCBI Accession No. NP_000055, version NP_000055.2, incorporated herein by reference and set forth below (SEQ ID NO: 25):

| | |
|---|---|
| 1 | mgptsgpsll llllthlpla igspmysiit pnilrlesee |
| | tmvleahdaq gdvpvtvtvh |
| 61 | dfpgkklvls sektvitpat nhmgnvtfti panrefksek |
| | grnkfvtvqa tfgtqvvekv |
| 121 | vlvslqsgyl fiqtdktiyt pgstvlyrif tvnhkllpvg |
| | rtvmvnienp egipvkqdsl |
| 181 | ssqnqlgvlp iswdipelvn mgqwkirayy enspqqvfst |
| | efevkeyvlp sfevivepte |
| 241 | kfyyiynekg levtitarfl ygkkvegtaf vifgiqdgeq |
| | rislpeslkr ipiedgsgev |
| 301 | vlsrkvlldg vqnpraedlv gkslyvsatv ilhsgsdmvq |
| | aersgipivt spyqihftkt |
| 361 | pkyfkpgmpf dimvfvtnpd gspayrvpva vqgedtvqsl |
| | tqgdgvakls inthpsqkpl |
| 421 | sitvrtkkqe Iseaeqatrt mqalpystvg nsnnylhlsv |
| | irtelrpget Invnfllrmd |
| 481 | raheakiryy tylimnkgrl lkagrqvrep gqdlvvlpls |
| | ittdfipsfr ivayytliga |
| 541 | sgqrevvads vwvdvkdscv gslvvksgqs edrqpvpgqq |
| | mtikiegdhg arvvivavdk |
| 601 | gvfvlnkknk ltqskiwdvv ekadigctpg sgkdyagvfs |
| | dagltftsss gqqtaqrael |
| 661 | qcpqpaarrr rsvqltekrm dkvgkypkel rkccedgmre |
| | npmrfscqrr trfislgeac |
| 721 | kkvfldccny itelrrqhar ashlglarsn idediiaeen |
| | ivsrsefpes wlwnvedlke |
| 781 | ppkngistkl mniflkdsit tweilavsms dkkgicvadp |
| | fevtvmqdff idlripysvv |
| 841 | rneqveirav lynyrqnqel kvrvellhnp afcslattkr |
| | rhqqtvtipp ksslsvpyvi |
| 901 | vplktglqev evkaavyhhf isdgvrkslk vvpegirmnk |
| | tvavrtldpe rlgregvqke |
| 961 | dippadlsdq vpdtesetri llqgtpvaqm tedavdaerl |
| | khlivtpsgc geqnmigmtp |
| 1021 | tviavhylde teqwekfgle krqgalelik kgytqqlafr |
| | qpssafaafv krapstwita |
| 1081 | yvvkvfslav nliaidsqvl cgavkwlile kqkpdgvfqe |
| | dapvihqemi gglrnnnekd |
| 1141 | maltafvlis iqeakdicee qvnslpgsit kagdfleany |
| | mnlqrsytva iagyalaqmg |
| 1201 | rlkgpllnkf lttakdknrw edpgkqlynv eatsyallal |
| | iqlkdfdfvp pvvrwlneqr |
| 1261 | yygggygstq atfmvfqala qyqkdapdhq elnldvslql |
| | psrsskithr ihwesaslir |
| 1321 | seetkenegf tvtaegkgqg tisvvtmyha kakdqltcnk |
| | fdlkvtikpa petekrpqda |
| 1381 | kntmileict ryrgdqdatm sildismmtg fapdtddlkq |
| | langvdryis kyeldkafsd |
| 1441 | rntliiyldk vshseddcla fkvhqyfnve liqpgavkvy |
| | ayynleesct rfyhpekedg |
| 1501 | klnklcrdel crcaeencfi qksddkvtle erldkacepg |
| | vdyvyktrlv kvqlsndfde |
| 1561 | yimaieqtik sgsdevqvgq qrtfispikc realkleekk |
| | hylmwglssd fwgekpnlsy |
| 1621 | iigkdtwveh wpeedecqde enqkqcqdlg aftesmvvfg |
| | cpn |

An exemplary Complement C3 nucleic acid sequence is provided at NCBI Accession No. NM_000064, version NM_000064.3, incorporated herein by reference and set forth below (SEQ ID NO: 26):

| | |
|---|---|
| 1 | agataaaaag ccagctccag caggcgctgc tcactcctcc |
| | ccatcctctc cctctgtccc |
| 61 | tctgtccctc tgaccctgca ctgtcccagc accatgggac |
| | ccacctcagg tcccagcctg |
| 121 | ctgctcctgc tactaaccca cctccccctg gctctgggga |
| | gtcccatgta ctctatcatc |
| 181 | acccccaaca tcttgcggct ggagagcgag gagaccatgg |
| | tgctggaggc ccacgacgcg |
| 241 | caaggggatg ttccagtcac tgttactgtc cacgacttcc |
| | caggcaaaaa actagtgctg |
| 301 | tccagtgaga agactgtgct gacccctgcc accaaccaca |
| | tgggcaacgt caccttcacg |
| 361 | atcccagcca acagggagtt caagtcagaa aaggggcgca |
| | acaagttcgt gaccgtgcag |
| 421 | gccaccttcg ggacccaagt ggtggagaag gtggtgctgg |
| | tcagcctgca gagcgggtac |
| 481 | ctcttcatcc agacagacaa gaccatctac acccctggct |
| | ccacagttct ctatcggatc |
| 541 | ttcaccgtca accacaagct gctacccgtg ggccggacgg |
| | tcatggtcaa cattgagaac |
| 601 | ccggaaggca tcccggtcaa gcaggactcc ttgtcttctc |
| | agaaccagct tggcgtcttg |
| 661 | cccttgtctt gggacattcc ggaactcgtc aacatgggcc |
| | agtggaagat ccgagcctac |
| 721 | tatgaaaact caccacagca ggtcttctcc actgagtttg |
| | aggtgaagga gtacgtgctg |
| 781 | cccagtttcg aggtcatagt ggagcctaca gagaaattct |
| | actacatcta taacgagaag |
| 841 | ggcctggagg tcaccatcac cgccaggttc ctctacggga |
| | agaaagtgga gggaactgcc |
| 901 | tttgtcatct tcgggatcca ggatggcgaa cagaggattt |
| | ccctgcctga atccctcaag |
| 961 | cgcattccga ttgaggatgg ctcgggggag gttgtgctga |
| | gccggaaggt actgctggac |
| 1021 | ggggtgcaga acccccgagc agaagacctg gtggggaagt |
| | ctttgtacgt gtctgccacc |
| 1081 | gtcatcttgc actcaggcag tgacatggtg caggcagagc |
| | gcagcgggat ccccatcgtg |
| 1141 | acctctccct accagatcca cttcaccaag acacccaagt |
| | acttcaaacc aggaatgccc |
| 1201 | tttgacctca tggtgttcgt gacgaaccct gatggctctc |
| | cagcctaccg agtccccgtg |
| 1261 | gcagtccagg gcgaggacac tgtgcagtct ctaacccagg |
| | gagatggcgt ggccaaactc |
| 1321 | agcatcaaca cacaccccag ccagaagccc ttgagcatca |
| | cggtgcgcac gaagaagcag |
| 1381 | gagctctcgg aggcagagca ggctaccagg accatgcagg |
| | ctctgcccta cagcaccgtg |
| 1441 | ggcaactcca acaattacct gcatctctca gtgctacgta |
| | cagagctcag acccggggag |
| 1501 | accctcaacg tcaacttcct cctgcgaatg gaccgcgccc |
| | acgaggccaa gatccgctac |
| 1561 | tacacctacc tgatcatgaa caagggcagg ctgttgaagg |
| | cgggacgcca ggtgcgagag |
| 1621 | cccggccagg acctggtggt gctgcccctg tccatcacca |
| | ccgacttcat cccttccttc |
| 1681 | cgcctggtgg cgtactacac gctgatcggt gccagcggcc |
| | agagggaggt ggtggccgac |
| 1741 | tccgtgtggg tggacgtcaa ggactcctgc gtgggctcgc |
| | tggtggtaaa aagcggccag |
| 1801 | tcagaagacc ggcagcctgt acctgggcag cagatgaccc |
| | tgaagataga gggtgaccac |
| 1861 | ggggcccggg tggtactggt ggccgtggac aagggcgtgt |
| | tcgtgctgaa taagaagaac |
| 1921 | aaactgacgc agagtaagat ctgggacgtg gtggagaagg |
| | cagacatcgg ctgcaccccg |
| 1981 | ggcagtggga aggattacgc cggtgtcttc tccgacgcag |
| | ggctgacctt cacgagcagc |
| 2041 | agtggccagc agaccgccca gagggcagaa cttcagtgcc |
| | cgcagccagc cgcccgccga |
| 2101 | cgccgttccg tgcagctcac ggagaagcga atggacaaag |
| | tcggcaagta ccccaaggag |
| 2161 | ctgcgcaagt gctgcgagga cggcatgcgg gagaacccca |
| | tgaggttctc gtgccagcgc |
| 2221 | cggacccgtt tcatctccct gggcgaggcg tgcaagaagg |
| | tcttcctgga ctgctgcaac |
| 2281 | tacatcacag agctgcggcg gcagcacgcg cgggccagcc |
| | acctgggcct ggccaggagt |
| 2341 | aacctggatg aggacatcat tgcagaagag aacatcgttt |
| | cccgaagtga gttcccagag |
| 2401 | agctggctgt ggaacgttga ggacttgaaa gagccaccga |
| | aaaatggaat ctctacgaag |
| 2461 | ctcatgaata tatttttgaa agactccatc accacgtggg |
| | agattctggc tgtgagcatg |
| 2521 | tcggacaaga aagggatctg tgtggcagac cccttcgagg |
| | tcacagtaat gcaggacttc |
| 2581 | ttcatcgacc tgcggctacc ctactctgtt gttcgaaacg |
| | agcaggtgga aatccgagcc |
| 2641 | gttctctaca attaccggca gaaccaagag ctcaaggtga |
| | gggtggaact actccacaat |
| 2701 | ccagccttct gcagcctggc caccaccaag aggcgtcacc |
| | agcagaccgt aaccatcccc |
| 2761 | cccaagtcct cgttgtccgt tccatatgtc atcgtgccgc |
| | taaagaccgg cctgcaggaa |
| 2821 | gtggaagtca aggctgctgt ctaccatcat ttcatcagtg |
| | acggtgtcag gaagtccctg |
| 2881 | aaggtcgtgc cggaaggaat cagaatgaac aaaactgtgg |
| | ctgttcgcac cctggatcca |
| 2941 | gaacgcctgg gccgtgaagg agtgcagaaa gaggacatcc |
| | cacctgcaga cctcagtgac |
| 3001 | caagtcccgg acaccgagtc tgagaccaga attctcctgc |
| | aagggacccc agtggcccag |
| 3061 | atgacagagg atgccgtcga cgcggaacgg ctgaagcacc |
| | tcattgtgac cccctcgggc |
| 3121 | tgcggggaac agaacatgat cggcatgacg cccacggtca |
| | tcgctgtgca ttacctggat |
| 3181 | gaaacggagc agtgggagaa gttcggccta gagaagcggc |
| | agggggcctt ggagctcatc |
| 3241 | aagaaggggt acacccagca gctggccttc agacaaccca |
| | gctctgcctt tgcggccttc |
| 3301 | gtgaaacggg cacccagcac ctggctgacc gcctacgtgg |
| | tcaaggtctt ctctctggct |
| 3361 | gtcaacctca tcgccatcga ctcccaagtc ctctgcgggg |
| | ctgttaaatg gctgatcctg |
| 3421 | gagaagcaga agcccgacgg ggtcttccag gaggatgcgc |
| | ccgtgataca ccaagaaatg |
| 3481 | attggtggat tacggaacaa caacgagaaa gacatggccc |
| | tcacggcctt tgttctcatc |
| 3541 | tcgctgcagg aggctaaaga tatttgcgag gagcaggtca |
| | acagcctgcc aggcagcatc |
| 3601 | actaaagcag gagacttcct tgaagccaac tacatgaacc |
| | tacagagatc ctacactgtg |
| 3661 | gccattgctg gctatgctct ggcccagatg ggcaggctga |
| | aggggcctct tcttaacaaa |
| 3721 | tttctgacca cagccaaaga taagaaccgc tgggaggacc |
| | ctggtaagca gctctacaac |
| 3781 | gtggaggcca catcctatgc cctcttggcc ctactgcagc |
| | taaaagactt tgactttgtg |
| 3841 | cctcccgtcg tgcgttggct caatgaacag agatactacg |
| | gtggtggcta tggctctacc |
| 3901 | caggccacct tcatggtgtt ccaagccttg gctcaatacc |
| | aaaaggacgc ccctgaccac |
| 3961 | caggaactga accttgatgt gtccctccaa ctgcccagcc |
| | gcagctccaa gatcacccac |
| 4021 | cgtatccact gggaatctgc cagcctcctg cgatcagaag |
| | agaccaagga aaatgagggt |
| 4081 | ttcacagtca cagctgaagg aaaaggccaa ggcaccttgt |
| | cggtggtgac aatgtaccat |
| 4141 | gctaaggcca aagatcaact cacctgtaat aaattcgacc |
| | tcaaggtcac cataaaacca |
| 4201 | gcaccggaaa cagaaaagag gcctcaggat gccaagaaca |
| | ctatgatcct tgagatctgt |
| 4261 | accaggtacc ggggagacca ggatgccact atgtctatat |
| | tggacatatc catgatgact |
| 4321 | ggctttgctc cagacacaga tgacctgaag cagctggcca |
| | atggtgttga cagatacatc |
| 4381 | tccaagtatg agctggacaa agccttctcc gataggaaca |
| | ccctcatcat ctacctggac |
| 4441 | aaggtctcac actctgagga tgactgtcta gctttcaaag |
| | ttcaccaata ctttaatgta |
| 4501 | gagcttatcc agcctggagc agtcaaggtc tacgcctatt |
| | acaacctgga ggaaagctgt |
| 4561 | acccggttct accatccgga aaaggaggat ggaaagctga |
| | acaagctctg ccgtgatgaa |
| 4621 | ctgtgccgct gtgctgagga gaattgcttc atacaaaagt |
| | cggatgacaa ggtcaccctg |
| 4681 | gaagaacggc tggacaaggc ctgtgagcca ggagtggact |
| | atgtgtacaa gacccgactg |
| 4741 | gtcaaggttc agctgtccaa tgactttgac gagtacatca |
| | tggccattga gcagaccatc |
| 4801 | aagtcaggct cggatgaggt gcaggttgga cagcagcgca |
| | cgttcatcag ccccatcaag |
| 4861 | tgcagagaag ccctgaagct ggaggagaag aaacactacc |
| | tcatgtgggg tctctcctcc |
| 4921 | gatttctggg gagagaagcc caacctcagc tacatcatcg |
| | ggaaggacac ttgggtggag |
| 4981 | cactggcccg aggaggacga atgccaagac gaagagaacc |
| | agaaacaatg ccaggacctc |
| 5041 | ggcgccttca ccgagagcat ggttgtcttt gggtgcccca |
| | actgaccaca cccccattcc |
| 5101 | cccactccag ataaagcttc agttatatct caaaaaaaaa |
| | aaaaaaaa |

An exemplary CACNA2D1 polypeptide sequence is provided at NCBI Accession No. NP_000713, version NP000713.2, incorporated herein by reference and set forth below (SEQ ID NO: 27):

| | |
|---|---|
| 1 | maagcllalt ltlfqsllig psseepfpsa vtikswvdkm |
| | qedlvtlakt asgvnqlvdi |
| 61 | yekyqdlytv epnnarqlve iaardiekll snrskalvrl |
| | aleaekvqaa hqwredfasn |
| 121 | evvyynakdd ldpekndsep gsqrikpvfi edanfgrqis |
| | yqhaavhipt diyegstivl |
| 181 | nelnwtsald evfkknreed psllwqvfgs atglaryypa |
| | spwvdnsrtp nkidlydvrr |
| 241 | rpwyiqgaas pkdmlilvdv sgsvsgltlk lirtsvseml |
| | etlsdddfvn vasfnsnaqd |
| 301 | vscfqhlvqa nvrnkkvlkd avnnitakgi tdykkgfsfa |
| | feqllnynvs rancnkiiml |
| 361 | ftdggeeraq eifnkynkdk kvrvftfsvg qhnydrgpiq |
| | wmacenkgyy yeipsigair |
| 421 | intqeyldvl grpmvlagdk akqvqwtnvy idalelglvi |
| | tgtlpvfnit gqfenktnlk |
| 481 | nqlilgvmgv dvsledikrl tprftlcpng yyfaidpngy |
| | vllhpnlqpk npksqepvtl |
| 541 | dfidaelend ikveirnkmi dgesgektfr tlvksqdery |
| | idkgnrtytw tpvngtdysl |
| 601 | alvlptysfy yikakleeti tqarskkgkm kdsetikpdn |
| | feesgytfia prdycndlki |
| 661 | sdnnteflln fnefidrktp nnpscnadli nrvlldagft |
| | nelvqnywsk qknikgvkar |
| 721 | fvvtdggitr vypkeagenw qenpetyeds fykrsldndn |
| | yvftapyfnk sgpgayesgi |
| 781 | mvskaveiyi qgkllkpavv gikidvnswi enftktsird |
| | pcagpvcdck rnsdvmdcvi |
| 841 | iddggfllma nhddytnqig rffgeidpsl mrhivnisvy |
| | afnksydyqs vcepgaapkq |
| 901 | gaghrsayvp svadilqigw wataaawsil qqfllsltfp |
| | rlleavemed ddftaslskq |
| 961 | sciteqtqyf fdndsksfsg vldcgncsri fhgeklmntn |
| | lifimveskg tcpcdtrlli |
| 1021 | qaeqtsdgpn pcdmvkqpry rkgpdvcfdn nvledytdcg |
| | gvsginpslw yiigiqflll |
| 1081 | wlvsgsthrl l |

An exemplary CACNA2D1 nucleic acid sequence is provided at NCBI Accession No. NM_000722, version NM_000722.4, incorporated herein by reference and set forth below (SEQ ID NO: 28):

| | |
|---|---|
| 1 | agcgagagcg cgcgagcgcc ggcgggctcg ccgaggtctg |
| | tttccaaagt cgcccttgat |
| 61 | ggcggcggag gcaaggcggc cgcggcgcgg agcagccgac |
| | gcacgctagt gggtccgccc |
| 121 | gccaccgccc cttcctcggc gtccgctccc gcccttgccg |
| | tcccccgcgc ggctccgcgc |
| 181 | ctcgggcccc gggcgcagcc agccctccag acgcccgcgg |
| | tcccggcggc gtgtgctgct |
| 241 | cttcctccgc ccgcggtttc cagcgccgct ccttcccccg |
| | cttgggcagg gagggggcat |
| 301 | tgatcttcga tcgcgaagat ggctgctggc tgcctgctgg |
| | ccttgactct gacacttttc |
| 361 | caatctttgc tcatcggccc ctcgtcggag gagccgttcc |
| | cttcggccgt cactatcaaa |
| 421 | tcatgggtgg ataagatgca agaagacctt gtcacactgg |
| | caaaaacagc aagtggagtc |
| 481 | aatcagcttg ttgatattta tgagaaatat caagatttgt |
| | atactgtgga accaaataat |
| 541 | gcacgccagc tggtagaaat tgcagccagg gatattgaga |
| | aacttctgag caacagatct |
| 601 | aaagccctgg tgcgcctggc attggaagcg gagaaagttc |
| | aagcagctca ccagtggaga |
| 661 | gaagattttg caagcaatga agttgtctac tacaatgcaa |
| | aggatgatct cgatcctgag |
| 721 | aaaaatgaca gtgagccagg cagccagagg ataaaacctg |
| | ttttcattga agatgctaat |
| 781 | tttggacgac aaatatctta tcagcacgca gcagtccata |
| | ttcctactga catctatgag |
| 841 | ggctcaacaa ttgtgttaaa tgaactcaac tggacaagtg |
| | ccttagatga agttttcaaa |
| 901 | aagaatcgcg aggaagaccc ttcattattg tggcaggttt |
| | ttggcagtgc cactggccta |
| 961 | gctcgatatt atccagcttc accatgggtt gataatagta |
| | gaactccaaa taagattgac |
| 1021 | ctttatgatg tacgcagaag accatggtac atccaaggag |
| | ctgcatctcc taaagacatg |
| 1081 | cttattctgg tggatgtgag tggaagtgtt agtggattga |
| | cacttaaact gatccgaaca |
| 1141 | tctgtctccg aaatgttaga aaccctctca gatgatgatt |
| | tcgtgaatgt agcttcattt |
| 1201 | aacagcaatg ctcaggatgt aagctgtttt cagcaccttg |
| | tccaagcaaa tgtaagaaat |
| 1261 | aaaaaagtgt tgaaagacgc ggtgaataat atcacagcca |
| | aaggaattac agattataag |
| 1321 | aagggcttta gttttgcttt tgaacagctg cttaattata |
| | atgtttccag agcaaactgc |
| 1381 | aataagatta ttatgctatt cacggatgga ggagaagaga |
| | gagcccagga gatatttaac |
| 1441 | aaatacaata aagataaaaa agtacgtgta ttcacgtttt |
| | cagttggtca acacaattat |
| 1501 | gacagaggac ctattcagtg gatggcctgt gaaaacaaag |
| | gttattatta tgaaattcct |
| 1561 | tccattggtg caataagaat caatactcag gaatatttgg |
| | atgttttggg aagaccaatg |
| 1621 | gttttagcag gagacaaagc taagcaagtc caatggacaa |
| | atgtgtacct ggatgcattg |
| 1681 | gaactgggac ttgtcattac tggaactctt ccggtcttca |
| | acataaccgg ccaatttgaa |
| 1741 | aataagacaa acttaaagaa ccagctgatt cttggtgtga |
| | tgggagtaga tgtgtctttg |
| 1801 | gaagatatta aaagactgac accacgtttt acactgtgcc |
| | ccaatgggta ttactttgca |
| 1861 | atcgatccta atggttatgt tttattacat ccaaatcttc |
| | agccaaagaa ccccaaatct |
| 1921 | caggagccag taacattgga tttccttgat gcagagttag |
| | agaatgatat taaagtggag |
| 1981 | attcgaaata agatgattga tggggaaagt ggagaaaaaa |
| | cattcagaac tctggttaaa |
| 2041 | tctcaagatg agagatatat tgacaaagga aacaggacat |
| | acacatggac acctgtcaat |
| 2101 | ggcacagatt acagtttggc cttggtatta ccaacctaca |
| | gtttttacta tataaaagcc |
| 2161 | aaactagaag agacaataac tcaggccaga tcaaaaaagg |
| | gcaaaatgaa ggattcggaa |
| 2221 | accctgaagc cagataattt tgaagaatct ggctatacat |
| | tcatagcacc aagagattac |
| 2281 | tgcaatgacc tgaaaatatc ggataataac actgaatttc |
| | ttttaaattt caacgagttt |
| 2341 | attgatagaa aaactccaaa caacccatca tgtaacgcgg |
| | atttgattaa tagagtcttg |
| 2401 | cttgatgcag gctttacaaa tgaacttgtc caaaattact |
| | ggagtaagca gaaaaatatc |
| 2461 | aagggagtga aagcacgatt tgttgtgact gatggtggga |
| | ttaccagagt ttatcccaaa |
| 2521 | gaggctggag aaaattggca agaaaaccca gagacatatg |
| | aggacagctt ctataaaagg |
| 2581 | agcctagata atgataacta tgttttcact gctccctact |
| | ttaacaaaag tggacctggt |
| 2641 | gcctatgaat cgggcattat ggtaagcaaa gctgtagaaa |
| | tatatattca agggaaactt |
| 2701 | cttaaacctg cagttgttgg aattaaaatt gatgtaaatt |
| | cctggataga gaatttcacc |
| 2761 | aaaacctcaa tcagagatcc gtgtgctggt ccagtttgtg |
| | actgcaaaag aaacagtgac |
| 2821 | gtaatggatt gtgtgattct ggatgatggt gggtttcttc |
| | tgatggcaaa tcatgatgat |
| 2881 | tatactaatc agattggaag attttttgga gagattgatc |
| | ccagcttgat gagacacctg |
| 2941 | gttaatatat cagtttatgc ttttaacaaa tcttatgatt |
| | atcagtcagt atgtgagccc |
| 3001 | ggtgctgcac caaaacaagg agcaggacat cgctcagcat |
| | atgtgccatc agtagcagac |
| 3061 | atattacaaa ttggctggtg ggccactgct gctgcctggt |
| | ctattctaca gcagtttctc |
| 3121 | ttgagtttga cctttccacg actccttgag gcagttgaga |
| | tggaggatga tgacttcacg |
| 3181 | gcctccctgt ccaagcagag ctgcattact gaacaaaccc |
| | agtatttctt cgataacgac |
| 3241 | agtaaatcat tcagtggtgt attagactgt ggaaactgtt |
| | ccagaatctt tcatggagaa |
| 3301 | aagcttatga acaccaactt aatattcata atggttgaga |
| | gcaaagggac atgtccatgt |
| 3361 | gacacacgac tgctcataca agcggagcag acttctgacg |
| | gtccaaatcc ttgtgacatg |
| 3421 | gttaagcaac ccagataccg aaaagggcct gatgtctgct |
| | ttgataacaa tgtcttggag |
| 3481 | gattatactg actgtggtgg tgtttctgga ttaaatccct |
| | ccctgtggta tatcattgga |
| 3541 | atccagtttc tactactttg gctggtatct ggcagcacac |
| | accgcctgtt atgaccttct |
| 3601 | aaaaaccaaa tctgcatagt taaactccag accctgccaa |
| | aacatgagcc ctgccctcaa |
| 3661 | ttacagtaac gtagggtcag ctataaaatc agacaaacat |
| | tagctgggcc tgttccatgg |
| 3721 | cataacacta aggcgcagac tcctaaggca cccactggct |
| | gcatgtcagg gtgtcagatc |
| 3781 | cttaaacgtg tgtgaatgct gcatcatcta tgtgtaacat |
| | caaagcaaaa tcctatacgt |
| 3841 | gtcctctatt ggaaaatttg ggagtttgtt gttgcattgt |
| | tggtgattac atgtgaaagg |
| 3901 | gttccccata caattgttaa tgaaccataa gaaatgtctt |
| | gatattgacc tggaattttg |
| 3961 | acttgctgca attttactaa gaaaatctct aaggggaaag |
| | aaattatttt tgccttcact |
| 4021 | ttttcttctg tgaaaagtta attcctgctt taagtagcaa |
| | ttattgaaat atatagagaa |
| 4081 | agagagaatt aacattggtc taaattgttg aaatataaat |
| | aatggctaat tttctatgaa |
| 4141 | aaatttgcca tgaataaaat gccttatgaa gaacggcttc |
| | tttgccaaaa ataaaacaca |
| 4201 | attgatgacc aattcaattt tgatggggta atgaattgat |
| | ggtttgaaaa tagtaactaa |
| 4261 | gaaattatta aactaaaggt gcttgaggga gaaattagaa |
| | tatgtgacat atttcctcat |
| 4321 | aaatgtaagc aagcccttaa taaatgcttt gatgtaatgt |
| | tccttttgtt cacagtacag |
| 4381 | ttatgtagtg ctagccagat atttaaaaat gctctaagaa |
| | aagcttgtca ggggggaggg |
| 4441 | ggaaatgttt ttcttgtgat aaatgaactt tggtttacca |
| | agggtatttt gcatgatgct |
| 4501 | gctgctattt taacttttca gttgtttttt ctcttactgt |
| | agagtgtagt cattgaaaag |
| 4561 | ttaactgagt gatattgtta tgtaagaatc tgaaccttgc |
| | agtgtaaatg cacttaagtc |
| 4621 | atctttaaaa tgttgttaaa ctactctgaa tatactgtgc |
| | aatgtaaatg ctgaatgatt |
| 4681 | ggggttagat aaatggtgaa atgagaatta atgaatttta |
| | tacagattca tgctttcgcc |
| 4741 | tgataaccta tgtacagata ttttaagtac ataaatcaga |
| | tttgacacat ttattttttg |
| 4801 | aaaagtacat atatgttctc aatgaagatt catcctaggt |
| | ttcctctttg ttcgtttcaa |
| 4861 | tagtagcaca tacaatccaa aagttcctgc tactgctgct |
| | tttgcccttc cattttaaat |
| 4921 | ggtatgatcg tttggccaaa atttccatgc aagttgataa |
| | gggttttaca ataatctgaa |
| 4981 | caaataggaa accttgaatc tctatgtatg cacatgaata |
| | cttgattgtg aataataaaa |
| 5041 | gttgtttttt atacagcctc attggtgaaa atggttagtg |
| | tagtaaatca gatatttcat |
| 5101 | tatagactaa tatggtagca gattatttat gcttttaaaa |
| | attattttct ggagcaaaca |
| 5161 | gtatttgaag aaaaggtaaa aactatgaat gtgtaattct |
| | aagaatagtt aataatttta |
| 5221 | ctgaaggctg aacgtatgta gatatttttt atgataaaat |
| | gcagtttttc tttttttgga |
| 5281 | acatgccaat tactggtatg tgtatatgct ggaaattttc |
| | ttacatatcc tttttaagtc |
| 5341 | aaagttatta acattatcca gcatgcttta atatgctcca |
| | catatcaaag atataaccaa |
| 5401 | taatttaacg tagcttaaaa gataacattt gttttgtttt |
| | tagctttgca tacatttgta |
| 5461 | attccctccc accaccaata taccttaggt accatttgtg |
| | tacatttcaa gatttttttt |
| 5521 | ttcaataaaa aggtgggggg aaatggaatc actttgccaa |
| | ctattgttct aagagttgac |
| 5581 | atcagttacc atttctaatg cattgttgtg attttgtagt |
| | gtcattcata actggtattg |
| 5641 | acattttaga aaacaccaaa gtgatttaaa atatatgtgg |
| | ggaaaaaaac ttctcactct |
| 5701 | agtaacaatg ttttaatcat gttgtttgtt aaatatttaa |
| | aagttattgt tcaatttttg |
| 5761 | ctagactatc ttaaatcatg atagtgttgg gaagggggta |
| | tcaaaatata aaattgttag |
| 5821 | tttgtgtata taaacaatgc acattaaaac aattgaattg |
| | cttccagcat acaacaaact |
| 5881 | accttaaaat tatgttcaaa atctattaaa tatattttca |
| | tggctaatgt cgagtatatg |
| 5941 | ttttcatcaa ttacattgtt attgtattaa tcgcggttaa |
| | acttggcatc tcttggcagg |
| 6001 | atcctattta atttctgttg ccatcaaaat atatttatat |
| | gcatgagttt tgctataaac |
| 6061 | ctttattttc tgctataaag ttatatatta tgacaaccat |
| | aaatgcaatt acaaattggg |
| 6121 | taagaacaag ctgaataaaa gttgttccca tgactaatat |
| | tagtcttcat gcttttagca |
| 6181 | tattttttaa gacagtaaca ctcatcccat caaattatta |
| | cattacattt agtatttttc |
| 6241 | tgagttacat tgacaagaac ttaaattata gcctgttatt |
| | aatattgatt tccaacgact |
| 6301 | gaacagtaca acagatcaat aaaaaagttt atacactgct |
| | ttaactaatt tttgttatac |
| 6361 | tcatgtggct tagtgtagtt agtcactaaa acagagttca |
| | agtgagcttg gcaatggtgt |
| 6421 | tgagtaacaa gaaagaattt tgcattaatg atttcttttt |
| | attttcctca tttaaacaac |
| 6481 | cagcattact gccaaacacc aaccgtggtc catatttgta |
| | acaggttttt aacatgacat |
| 6541 | agttagcaag tttgattgaa gagattttta ggtcctgggc |
| | ctataagatt ttagtatgat |
| 6601 | cttttttcat gtttctaatg cttgaggtgt tactgcttaa |
| | cttgggaaaa aaaaaaacaa |
| 6661 | aaaacagtgt tcctgccatt tgtaagtttt cctataatat |
| | tccttttatt aactttaaaa |
| 6721 | ctggacttat cgggttcaaa taggcagtat cccttttaag |
| | tgacctttgc aacccaagtg |
| 6781 | ttacttgctt atttgatgct ctcttgtatt ttaaatctca |
| | tttaaatctt tgtctgtgac |
| 6841 | caacaaagct taagattttg tcttcattta aaactctgaa |
| | tctattccat tgatcatgtt |
| 6901 | agacgttcaa atgtttaatg gtatagtctg acctagtgaa |
| | acaaaatgga tgttgttact |
| 6961 | ttttttaaag aatagaatga aataactatt tcacttgccc |
| | aaaattttaa atattttttc |
| 7021 | atctaaactt gtattgtttc ccatagcaga atgtcaatat |
| | tcacagtaca tttctgtaaa |
| 7081 | gagcaaacca atataatgtt ttgagtgttg aaaaaaattc |
| | cagatttttg aagaattaga |
| 7141 | caactcttca tctaccttat ttctagttca cacagttatc |
| | tcaaattcca ctgaaactaa |
| 7201 | tgggatactg tcttgtgtag atgccagttg agtttataat |
| | gtgacctagt aaagctgtct |
| 7261 | tttttgttgt gttgtatgag tgtcggatca tgcttttagg |
| | aatactttta ttaaaatggt |
| 7321 | gtgcattcat gcaaaaggcc aactggcttt tgtgaacaat |
| | agatcttttc tcccctttat |
| 7381 | tttgttctct tgacactttt gtgaaaatta cctagcctga |
| | taaaaacatt ttgtaaaaat |
| 7441 | ttgtataata ctgttataaa atatttataa tcccagaaaa |
| | tgttgtgtta aatcttgtgc |
| 7501 | aaaaacagta tattcagaat aaaagtttat catttaaagt |
| | ca |

An exemplary CACNA1D polypeptide sequence is provided at NCBI Accession No. NP_000711, version NP000711.1, incorporated herein by reference and set forth below (SEQ ID NO: 29):

| | |
|---|---|
| 1 | mmmmmmmkkm qhqrqqqadh aneanyargt rlplsgegpt |
| | sqpnsskqtv lswqaaidaa |
| 61 | rqakaaqtms tsapppvgsl sqrkrqqyak skkqgnssns |
| | rparalfels lnnpirraci |
| 121 | sivewkpfdi fillaifanc valaiyipfp eddsnstnhn |
| | lekveyafli iftvetflki |
| 181 | iayglllhpn ayvrngwnll dfvivivglf svileqltke |
| | teggnhssgk sggfdvkalr |
| 241 | afrvlrplrl vsgvpslqvv lnsiikamvp llhiallvlf |
| | viiiyaiigl elfigkmhkt |
| 301 | cffadsdiva eedpapcafs gngrqctang tecrsgwvgp |
| | nggitnfdnf afamltvfqc |
| 361 | itmegwtdvl ywvndaigwe wpwvyfvsli ilgsffvlnl |
| | vlgvlsgefs kerekakarg |
| 421 | dfqklrekqq leedlkgyld witqaedidp eneeeggeeg |
| | krntsmptse tesvntenvs |
| 481 | gegenrgccg slwcwwrrrg aakagpsger rwgqaisksk |
| | isrrwrrwnr fnrrrcraav |
| 541 | ksvtfywlvi vlvflntlti ssehynqpdw ltqiqdiank |
| | vllalfteem ivkmyslglq |
| 601 | ayfvslfnrf dcfvvcggit etilveleim splgisvfrc |
| | vrllrifkvt rhwtslsnlv |
| 661 | asllnsmksi aslllllflf iiifsllgmq lfggkfnfde |
| | tqtkrstfdn fpqalltvfq |
| 721 | iltgedwnav mydgimaygg psssgmivci yfiilficgn |
| | yillnvflai avdnladaes |
| 781 | lntaqkeeae ekerkkiark eslenkknnk pevnqiansd |
| | nkvtiddyre ededkdpypp |
| 841 | cdvpvgeeee eeeedepevp agprprrise inmkekiapi |
| | pegsaffils ktnpirvgch |
| 901 | klinhhiftn lilvfimlss aalaaedpir shsfrntilg |
| | yfdyaftaif tveillkmtt |
| 961 | fgafihkgaf crnyfnlldm lvvgvslvsf giqssaisw |
| | kilrvlrvlr plrainrakg |
| 1021 | lkhvvqcvfv airtignimi vttllqfmfa cigvqlfkgk |
| | fyrctdeaks npeecrglfi |
| 1081 | lykdgdvdsp vvreriwqns dfnfdnvisa mmalftvstf |
| | egwpallyka idsngenigp |
| 1141 | iynhrveisi ffiiyiiiva ffmmnifvgf vivtfqeqge |
| | keyknceldk nqrqcveyal |
| 1201 | karplrryip knpyqykfwy vvnsspfeym mfvlimlntl |
| | clamqhyeqs kmfndamdil |
| 1261 | nmvftgvftv emvlkviafk pkgyfsdawn tfdslivigs |
| | iidvalsead ptesenvpvp |
| 1321 | tatpgnsees nrisitffri frvmrivkll srgegirtll |
| | wtfiksfqal pyvalliaml |
| 1381 | ffiyavigmq mfgkvamrdn nqinrnnnfq tfpqavlllf |
| | rcatgeawqe imlacipgkl |
| 1441 | cdpesdynpg eeytcgsnfa ivyfisfyml cafliinlfv |
| | avimdnfdyl trdwsilgph |
| 1501 | hldefkriws eydpeakgri khldvvtllr riqpplgfgk |
| | icphrvackr lvamnmplns |
| 1561 | dgtvmfnati falvrtalki ktegnleqan eelravikki |
| | wkktsmklld qvvppagdde |
| 1621 | vtvgkfyatf liqdyfrkfk krkeqglvgk ypaknttial |
| | qaglrtlhdi gpeirraisc |
| 1681 | dlqddepeet kreeeddvfk rngallgnhv nhvnsdrrds |
| | Iqqtntthrp ihvqrpsipp |
| 1741 | asdtekplfp pagnsvchnh hnhnsigkqv ptstnanlnn |
| | anmskaahgk rpsignlehv |
| 1801 | senghhsshk hdrepqrrss vkrtryyety irsdsgdeql |
| | pticredpei hgyfrdphcl |
| 1861 | geqeyfssee cyeddssptw srqnygyysr ypgrnidser |
| | prgyhhpqgf ledddspvcy |
| 1921 | dsrrsprrrl ipptpashrr ssfnfecirr qssqeevpss |
| | pifphrtalp Ihlmqqqima |
| 1981 | vagldsskaq kyspshstrs watppatppy rdwtpcytpl |
| | iqveqseald qvngslpslh |
| 2041 | rsswytdepd isyrtftpas itvpssfrnk nsdkqrsads |
| | iveavliseg igryardpkf |
| 2101 | vsatkheiad acditideme saastllngn vrprangdvg |
| | plshrqdyel qdfgpgysde |
| 2161 | epdpgrdeed lademicitt l |

An exemplary CACNAID nucleic acid sequence is provided at NCBI Accession No. NM_000720, version NM_000720.3, incorporated herein by reference and set forth below (SEQ ID NO: 30):

| | |
|---|---|
| 1 | agaataaggg cagggaccgc ggctcctacc tcttggtgat |
| | ccccttcccc attccgcccc |
| 61 | cgcctcaacg cccagcacag tgccctgcac acagtagtcg |
| | ctcaataaat gttcgtggat |
| 121 | gatgatgatg atgatgatga aaaaaatgca gcatcaacgg |
| | cagcagcaag cggaccacgc |
| 181 | gaacgaggca aactatgcaa gaggcaccag acttcctctt |
| | tctggtgaag gaccaacttc |
| 241 | tcagccgaat agctccaagc aaactgtcct gtcttggcaa |
| | gctgcaatcg atgctgctag |
| 301 | acaggccaag gctgcccaaa ctatgagcac ctctgcaccc |
| | ccacctgtag gatctctctc |
| 361 | ccaaagaaaa cgtcagcaat acgccaagag caaaaaacag |
| | ggtaactcgt ccaacagccg |
| 421 | acctgcccgc gcccttttct gtttatcact caataacccc |
| | atccgaagag cctgcattag |
| 481 | tatagtggaa tggaaaccat ttgacatatt tatattattg |
| | gctatttttg ccaattgtgt |
| 541 | ggccttagct atttacatcc cattccctga agatgattct |
| | aattcaacaa atcataactt |
| 601 | ggaaaaagta gaatatgcct tcctgattat ttttacagtc |
| | gagacatttt tgaagattat |
| 661 | agcgtatgga ttattgctac atcctaatgc ttatgttagg |
| | aatggatgga atttactgga |
| 721 | ttttgttata gtaatagtag gattgtttag tgtaattttg |
| | gaacaattaa ccaaagaaac |
| 781 | agaaggcggg aaccactcaa gcggcaaatc tggaggcttt |
| | gatgtcaaag ccctccgtgc |
| 841 | ctttcgagtg ttgcgaccac ttcgactagt gtcaggagtg |
| | cccagtttac aagttgtcct |
| 901 | gaactccatt ataaaagcca tggttcccct ccttcacata |
| | gcccttttgg tattatttgt |
| 961 | aatcataatc tatgctatta taggattgga actttttatt |
| | ggaaaaatgc acaaaacatg |
| 1021 | tttttttgct gactcagata tcgtagctga agaggaccca |
| | gctccatgtg cgttctcagg |
| 1081 | gaatggacgc cagtgtactg ccaatggcac ggaatgtagg |
| | agtggctggg ttggcccgaa |
| 1141 | cggaggcatc accaactttg ataactttgc ctttgccatg |
| | cttactgtgt ttcagtgcat |
| 1201 | caccatggag ggctggacag atgtgctcta ctgggtaaat |
| | gatgcgatag gatgggaatg |
| 1261 | gccatgggtg tattttgtta gtctgatcat ccttggctca |
| | tttttcgtcc ttaacctggt |
| 1321 | tcttggtgtc cttagtggag aattctcaaa ggaaagagag |
| | aaggcaaaag cacggggaga |
| 1381 | tttccagaag ctccgggaga agcagcagct ggaggaggat |
| | ctaaagggct acttggattg |
| 1441 | gatcacccaa gctgaggaca tcgatccgga gaatgaggaa |
| | gaaggaggag aggaaggcaa |
| 1501 | acgaaatact agcatgccca ccagcgagac tgagtctgtg |
| | aacacagaga acgtcagcgg |
| 1561 | tgaaggcgag aaccgaggct gctgtggaag tctctggtgc |
| | tggtggagac ggagaggcgc |
| 1621 | ggccaaggcg gggccctctg ggtgtcggcg gtggggtcaa |
| | gccatctcaa aatccaaact |
| 1681 | cagccgacgc tggcgtcgct ggaaccgatt caatcgcaga |
| | agatgtaggg ccgccgtgaa |
| 1741 | gtctgtcacg ttttactggc tggttatcgt cctggtgttt |
| | ctgaacacct taaccatttc |
| 1801 | ctctgagcac tacaatcagc cagattggtt gacacagatt |
| | caagatattg ccaacaaagt |
| 1861 | cctcttggct ctgttcacct gcgagatgct ggtaaaaatg |
| | tacagcttgg gcctccaagc |
| 1921 | atatttcgtc tctcttttca accggtttga ttgcttcgtg |
| | gtgtgtggtg gaatcactga |
| 1981 | gacgatcttg gtggaactgg aaatcatgtc tcccctgggg |
| | atctctgtgt ttcggtgtgt |
| 2041 | gcgcctctta agaatcttca aagtgaccag gcactggact |
| | tccctgagca acttagtggc |
| 2101 | atccttatta aactccatga agtccatcgc ttcgctgttg |
| | cttctgcttt ttctcttcat |
| 2161 | tatcatcttt tccttgcttg ggatgcagct gtttggcggc |
| | aagtttaatt ttgatgaaac |
| 2221 | gcaaaccaag cggagcacct ttgacaattt ccctcaagca |
| | cttctcacag tgttccagat |
| 2281 | cctgacaggc gaagactgga atgctgtgat gtacgatggc |
| | atcatggctt acgggggccc |
| 2341 | atcctcttca ggaatgatcg tctgcatcta cttcatcatc |
| | ctcttcattt gtggtaacta |
| 2401 | tattctactg aatgtcttct tggccatcgc tgtagacaat |
| | ttggctgatg ctgaaagtct |
| 2461 | gaacactgct cagaaagaag aagcggaaga aaaggagagg |
| | aaaaagattg ccagaaaaga |
| 2521 | gagcctagaa aataaaaaga acaacaaacc agaagtcaac |
| | cagatagcca acagtgacaa |
| 2581 | caaggttaca attgatgact atagagaaga ggatgaagac |
| | aaggacccct atccgccttg |
| 2641 | cgatgtgcca gtaggggaag aggaagagga agaggaggag |
| | gatgaacctg aggttcctgc |
| 2701 | cggaccccgt cctcgaagga tctcggagtt gaacatgaag |
| | gaaaaaattg cccccatccc |
| 2761 | tgaagggagc gctttcttca ttcttagcaa gaccaacccg |
| | atccgcgtag gctgccacaa |
| 2821 | gctcatcaac caccacatct tcaccaacct catccttgtc |
| | ttcatcatgc tgagcagcgc |
| 2881 | tgccctggcc gcagaggacc ccatccgcag ccactccttc |
| | cggaacacga tactgggtta |
| 2941 | ctttgactat gccttcacag ccatctttac tgttgagatc |
| | ctgttgaaga tgacaacttt |
| 3001 | tggagctttc ctccacaaag gggccttctg caggaactac |
| | ttcaatttgc tggatatgct |
| 3061 | ggtggttggg gtgtctctgg tgtcatttgg gattcaatcc |
| | agtgccatct ccgttgtgaa |
| 3121 | gattctgagg gtcttaaggg tcctgcgtcc cctcagggcc |
| | atcaacagag caaaaggact |
| 3181 | taagcacgtg gtccagtgcg tcttcgtggc catccggacc |
| | atcggcaaca tcatgatcgt |
| 3241 | caccaccctc ctgcagttca tgtttgcctg tatcggggtc |
| | cagttgttca aggggaagtt |
| 3301 | ctatcgctgt acggatgaag ccaaaagtaa ccctgaagaa |
| | tgcaggggac ttttcatcct |
| 3361 | ctacaaggat ggggatgttg acagtcctgt ggtccgtgaa |
| | cggatctggc aaaacagtga |
| 3421 | tttcaacttc gacaacgtcc tctctgctat gatggcgctc |
| | ttcacagtct ccacgtttga |
| 3481 | gggctggcct gcgttgctgt ataaagccat cgactcgaat |
| | ggagagaaca tcggcccaat |
| 3541 | ctacaaccac cgcgtggaga tctccatctt cttcatcatc |
| | tacatcatca ttgtagcttt |
| 3601 | cttcatgatg aacatctttg tgggctttgt catcgttaca |
| | tttcaggaac aaggagaaaa |
| 3661 | agagtataag aactgtgagc tggacaaaaa tcagcgtcag |
| | tgtgttgaat acgccttgaa |
| 3721 | agcacgtccc ttgcggagat acatccccaa aaacccctac |
| | cagtacaagt tctggtacgt |
| 3781 | ggtgaactct tcgcctttcg aatacatgat gtttgtcctc |
| | atcatgctca acacactctg |
| 3841 | cttggccatg cagcactacg agcagtccaa gatgttcaat |
| | gatgccatgg acattctgaa |
| 3901 | catggtcttc accggggtgt tcaccgtcga gatggttttg |
| | aaagtcatcg catttaagcc |
| 3961 | taaggggtat tttagtgacg cctggaacac gtttgactcc |
| | ctcatcgtaa tcggcagcat |
| 4021 | tatagacgtg gccctcagcg aagcagaccc aactgaaagt |
| | gaaaatgtcc ctgtcccaac |
| 4081 | tgctacacct gggaactctg aagagagcaa tagaatctcc |
| | atcacctttt tccgtctttt |
| 4141 | ccgagtgatg cgattggtga agcttctcag caggggggaa |
| | ggcatccgga cattgctgtg |
| 4201 | gacttttatt aagtcctttc aggcgctccc gtatgtggcc |
| | ctcctcatag ccatgctgtt |
| 4261 | cttcatctat gcggtcattg gcatgcagat gtttgggaaa |
| | gttgccatga gagataacaa |
| 4321 | ccagatcaat aggaacaata acttccagac gtttccccag |
| | gcggtgctgc tgctcttcag |
| 4381 | gtgtgcaaca ggtgaggcct ggcaggagat catgctggcc |
| | tgtctcccag ggaagctctg |
| 4441 | tgaccctgag tcagattaca accccgggga ggagtataca |
| | tgtgggagca actttgccat |
| 4501 | tgtctatttc atcagttttt acatgctctg tgcatttctg |
| | atcatcaatc tgtttgtggc |
| 4561 | tgtcatcatg gataatttcg actatctgac ccgggactgg |
| | tctattttgg ggcctcacca |
| 4621 | tttagatgaa ttcaaaagaa tatggtcaga atatgaccct |
| | gaggcaaagg gaaggataaa |
| 4681 | acaccttgat gtggtcactc tgcttcgacg catccagcct |
| | cccctggggt ttgggaagtt |
| 4741 | atgtccacac agggtagcgt gcaagagatt agttgccatg |
| | aacatgcctc tcaacagtga |
| 4801 | cgggacagtc atgtttaatg caaccctgtt tgctttggtt |
| | cgaacggctc ttaagatcaa |
| 4861 | gaccgaaggg aacctggagc aagctaatga agaacttcgg |
| | gctgtgataa agaaaatttg |
| 4921 | gaagaaaacc agcatgaaat tacttgacca agttgtccct |
| | ccagctggtg atgatgaggt |
| 4981 | aaccgtgggg aagttctatg ccactttcct gatacaggac |
| | tactttagga aattcaagaa |
| 5041 | acggaaagaa caaggactgg tgggaaagta ccctgcgaag |
| | aacaccacaa ttgccctaca |
| 5101 | ggcgggatta aggacactgc atgacattgg gccagaaatc |
| | cggcgtgcta tatcgtgtga |
| 5161 | tttgcaagat gacgagcctg aggaaacaaa acgagaagaa |
| | gaagatgatg tgttcaaaag |
| 5221 | aaatggtgcc ctgcttggaa accatgtcaa tcatgttaat |
| | agtgatagga gagattccct |
| 5281 | tcagcagacc aataccaccc accgtcccct gcatgtccaa |
| | aggccttcaa ttccacctgc |
| 5341 | aagtgatact gagaaaccgc tgtttcctcc agcaggaaat |
| | tcggtgtgtc ataaccatca |
| 5401 | taaccataat tccataggaa agcaagttcc cacctcaaca |
| | aatgccaatc tcaataatgc |
| 5461 | caatatgtcc aaagctgccc atggaaagcg gcccagcatt |
| | gggaaccttg agcatgtgtc |
| 5521 | tgaaaatggg catcattctt cccacaagca tgaccgggag |
| | cctcagagaa ggtccagtgt |
| 5581 | gaaaagaacc cgctattatg aaacttacat taggtccgac |
| | tcaggagatg aacagctccc |
| 5641 | aactatttgc cgggaagacc cagagataca tggctatttc |
| | agggaccccc actgcttggg |
| 5701 | ggagcaggag tatttcagta gtgaggaatg ctacgaggat |
| | gacagctcgc ccacctggag |
| 5761 | caggcaaaac tatggctact acagcagata cccaggcaga |
| | aacatcgact ctgagaggcc |
| 5821 | ccgaggctac catcatcccc aaggattett ggaggacgat |
| | gactcgcccg tttgctatga |
| 5881 | ttcacggaga tctccaagga gacgcctact acctcccacc |
| | ccagcatccc accggagatc |
| 5941 | ctccttcaac tttgagtgcc tgcgccggca gagcagccag |
| | gaagaggtcc cgtcgtctcc |
| 6001 | catcttcccc catcgcacgg ccctgcctct gcatctaatg |
| | cagcaacaga tcatggcagt |
| 6061 | tgccggccta gattcaagta aagcccagaa gtactcaccg |
| | agtcactcga cccggtcgtg |
| 6121 | ggccacccct ccagcaaccc ctccctaccg ggactggaca |
| | ccgtgctaca cccccctgat |
| 6181 | ccaagtggag cagtcagagg ccctggacca ggtgaacggc |
| | agcctgccgt ccctgcaccg |
| 6241 | cagctcctgg tacacagacg agcccgacat ctcctaccgg |
| | actttcacac cagccagcct |
| 6301 | gactgtcccc agcagcttcc ggaacaaaaa cagcgacaag |
| | cagaggagtg cggacagctt |
| 6361 | ggtggaggca gtcctgatat ccgaaggctt gggacgctat |
| | gcaagggacc caaaatttgt |
| 6421 | gtcagcaaca aaacacgaaa tcgctgatgc ctgtgacctc |
| | accatcgacg agatggagag |
| 6481 | tgcagccagc accctgctta atgggaacgt gcgtccccga |
| | gccaacgggg atgtgggccc |
| 6541 | cctctcacac cggcaggact atgagctaca ggactttggt |
| | cctggctaca gcgacgaaga |
| 6601 | gccagaccct gggagggatg aggaggacct ggcggatgaa |
| | atgatatgca tcaccacctt |
| 6661 | gtagccccca gcgaggggca gactggctct ggcctcaggt |
| | ggggcgcagg agagccaggg |
| 6721 | gaaaagtgcc tcatagttag gaaagtttag gcactagttg |
| | ggagtaatat tcaattaatt |
| 6781 | agacttttgt ataagagatg tcatgcctca agaaagccat |
| | aaacctggta ggaacaggtc |
| 6841 | ccaagcggtt gagcctggca gagtaccatg cgctcggccc |
| | cagctgcagg aaacagcagg |
| 6901 | ccccgccctc tcacagagga tgggtgagga ggccagacct |
| | gccctgcccc attgtccaga |
| 6961 | tgggcactgc tgtggagtct gcttctccca tgtaccaggg |
| | caccaggccc acccaactga |
| 7021 | aggcatggcg gcggggtgca ggggaaagtt aaaggtgatg |
| | acgatcatca cacctgtgtc |
| 7081 | gttacctcag ccatcggtct agcatatcag tcactgggcc |
| | caacatatcc atttttaaac |
| 7141 | cctttccccc aaatacactg cgtcctggtt cctgtttagc |
| | tgttctgaaa tacggtgtgt |
| 7201 | aagtaagtca gaacccagct accagtgatt attgcgaggg |
| | caatgggacc tcataaataa |
| 7261 | ggttttctgt gatgtgacgc cagtttacat aagagaatat |
| | cactccgatg gtcggtttct |
| 7321 | gactgtcacg ctaagggcaa ctgtaaactg gaataataat |
| | gcactcgcaa ccaggtaaac |
| 7381 | ttagatacac tagtttgttt aaaattatag atttactgta |
| | catgacttgt aatatactat |
| 7441 | aatttgtatt tgtaaagaga tggtctatat tttgtaatta |
| | ctgtattgta tttgaactgc |
| 7501 | agcaatatcc atgggtccta ataattgtag ttccccacta |
| | aaatctagaa attattagta |
| 7561 | tttttactcg ggctatccag aagtagaaga aatagagcca |
| | attctcattt attcagcgaa |
| 7621 | aatcctctgg ggttaaaatt ttaagtttga aagaacttga |
| | cactacagaa atttttctaa |
| 7681 | aatattttga gtcactataa acctatcatc tttccacaag |
| | ataaaa |

An exemplary RGS4 polypeptide sequence is provided at NCBI Accession No. AAH00737, version AAH00737.1, incorporated herein by reference and set forth below (SEQ ID NO: 31):

| | |
|---|---|
| 1 | mckglaglpa sclrsakdmk hrlgfllqks dscehnsshn |
| | kkdkvvicqr vsqeevkkwa |
| 61 | eslenlishe cglaafkafl kseyseenid fwisceeykk |
| | ikspsklspk akkiynefis |
| 121 | vqatkevnld sctreetsrn mleptitcfd eaqkkifnlm |
| | ekdsyrrf1k srfyldlvnp |
| 181 | sscgaekqkg akssadcasl vpqca |

An exemplary RGS4 nucleic acid sequence is provided at NCBI Accession No. NM_001113380, version NM_001113380.1, incorporated herein by reference and set forth below (SEQ ID NO: 32):

| | |
|---|---|
| 1 | actttcccga ggtgcttcta cagttccctc tgccagcagg |
| | ggaacagatg gaaatagcaa |
| 61 | tcacctgcca gaaggtggcg tgcagcaagg atgtgcatct |
| | tttgccgcta ctgctttctg |
| 121 | attcctaaaa attactcaga gatcactcat gtgttcagtg |
| | attcaggttc tgttgaagat |
| 181 | accaaagata ttcggttggt caaaatgacg ggcatataaa |
| | ggcttctcag gtttctgagt |
| 241 | gcaaaagata tgaaacatcg gctaggtttc ctgctgcaaa |
| | aatctgattc ctgtgaacac |
| 301 | aattcttccc acaacaagaa ggacaaagtg gttatttgcc |
| | agagagtgag ccaagaggaa |
| 361 | gtcaagaaat gggctgaatc actggaaaac ctgattagtc |
| | atgaatgtgg gctggcagct |
| 421 | ttcaaagctt tcttgaagtc tgaatatagt gaggagaata |
| | ttgacttctg gatcagctgt |
| 481 | gaagagtaca agaaaatcaa atcaccatct aaactaagtc |
| | ccaaggccaa aaagatctat |
| 541 | aatgaattca tctcagtcca ggcaaccaaa gaggtgaacc |
| | tggattcttg caccagggaa |
| 601 | gagacaagcc ggaacatgct agagcctaca ataacctgct |
| | ttgatgaggc ccagaagaag |
| 661 | attttcaacc tgatggagaa ggattcctac cgccgcttcc |
| | tcaagtctcg attctatctt |
| 721 | gatttggtca acccgtccag ctgtggggca gaaaagcaga |
| | aaggagccaa gagttcagca |
| 781 | gactgtgctt ccctggtccc tcagtgtgcc taattctcac |
| | ctgaaggcag agggatgaaa |
| 841 | tgccaagact ctatgctctg gaaaacctga ggccaaatat |
| | tgatctgtat taagctccag |
| 901 | tgctttatcc acattgtagc ctaatattca tgctgcctgc |
| | catgtgtgag tcacttctac |
| 961 | gcataaacta gatatagctt ttggtgtttg agtgttcatc |
| | agggtgggac cccattccag |
| 1021 | tccaattttc ctaagtttct ttgagggttc catgggagca |
| | aatatctaaa taatggcctg |
| 1081 | gtaggtctgg attttcaaag attgttggca gtttcctcct |
| | cccaacagtt ttacctcggg |
| 1141 | atggttggtt agtgcatgtc acatgacatc cacatgcaca |
| | tgtattctgt tggccagcac |
| 1201 | gttctccaga ctctagatgt ttagatgagg ttgagctatg |
| | atatgtgctt gtgtgtatgt |
| 1261 | ctatgtgtat atattatata tacattagac acacatatac |
| | attatttctg tatatagatg |
| 1321 | tctgtgtata catatgtatg tgtgagtgta tgtatacaca |
| | cacacacaca cacacacaca |
| 1381 | cacttttgca agagtgatgg gaaagaccct aggtgctcat |
| | aactagagta tgtgtatgta |
| 1441 | cttacatggg tgttttgatc tctgttcttt catactacat |
| | ttgaacaggg caaaatgaac |
| 1501 | taactgccat gtaggctaag aaagaaatgc taacctgtgg |
| | aaagttggtt ttgtaaaatt |
| 1561 | ccatggatct tgctggagaa gcatccaagg aacttcatgc |
| | ttgatttgac cactgacagc |
| 1621 | ctccaccttg agcactattc taaggagcaa ataccttagc |
| | tcccttgagc tggttttctc |
| 1681 | tgatggcact tttgagctcc taagctgcca gccttccctt |
| | cttttcctgg gtgctcaggg |
| 1741 | catgcttatt agcagctggg ttggtatgga gttggcagac |
| | aggatgttca acttaatgaa |
| 1801 | gaaatacagc taaggccttg ccagcaacac ctgccgtaag |
| | ttactggctg agtgagggca |
| 1861 | tagaagttaa aggttactgt ttttatcctc tatccttttt |
| | tcctttcctg atcaaggtgc |
| 1921 | tcttctcatt ttttcctgag aaccttagcc atcagatgag |
| | gctccttagt ttattgtggt |
| 1981 | tggttgtttt ttctttataa tggctctggg ctatatgcct |
| | atatttataa accagcagca |
| 2041 | ggggaaagat tatattttat aagagggaac aaattttcac |
| | aatttgaaaa gcccacataa |
| 2101 | gttttctctt ttaaggtaga atcttgttaa tttcattcca |
| | aacatcgggg ctaacagaga |
| 2161 | ctggaggcat ttctttttag gctctgagac taaatgagag |
| | gaaaagaaaa gaaaaaaaaa |
| 2221 | atgattgtct aaccaattgt gagaattact gtttgaaact |
| | tttcaaggca cattgaaata |
| 2281 | cttgaaaact tctcatttat gttatttatg atgttatttt |
| | gtacgtgtta ttattattat |
| 2341 | attgttttat aaatggaggt acaggatatc acctgaatta |
| | ttaatgaatg cccaggaagt |
| 2401 | aattttcttc tcattcttct aaaactactg cctttcaaag |
| | tgcacacaca cgcgtccaca |
| 2461 | tacactgcat tcgttgctcc agtataaatt acatgcatga |
| | gcacctttct ggcttttaag |
| 2521 | ccaatataat gggctgcaaa atgaagacac cagagtgtat |
| | gcatacaaat ctcactgtat |
| 2581 | taaagatgca ggttttctaa ttgtaccctt cttgtctctc |
| | tggcaatctt gcccttaata |
| 2641 | tccctggagt tcctcatcag tgtcattttc tgttatacac |
| | agttccacaa ttttgtctct |
| 2701 | agttgacttc aaatgtgtaa ctttattggt cttgccctat |
| | tataattgtc atgactttca |
| 2761 | gattgtatct gaactcacag actgctgtct tactaatagg |
| | tctggaaggt cacgctgaat |
| 2821 | gagaagtaaa ttattttatg taatacattt ttgagtgtgt |
| | ttttcagttg tatttccctg |
| 2881 | ttatttcatc actatttcca atggtgagct tgcctgctca |
| | tgctccctgg acagaatact |
| 2941 | ccttcctttt gcatgcctgt ttctatcatg tgcttgatag |
| | gcctcaaagc taatgcttcc |
| 3001 | agtgaaacac acgcatctta ataataaggg taaataaacg |
| | ctccatatga aacta |

An exemplary SYP polypeptide sequence is provided at NCBI Accession No. NP_003170, version NP_003170.1, incorporated herein by reference and set forth below (SEQ ID NO: 33):

| | |
|---|---|
| 1 | mllladmdvv nqlvaggqfr vvkeplgfvk vlqwvfaifa |
| | fatcgsysge lqlsvdcank |
| 61 | tesdlsieve feypfrlhqv yfdaptcrgg ttkvflvgdy |
| | sssaeffvtv avfaflysmg |
| 121 | alatyiflqn kyrennkgpm idflatavfa fmwlvsssaw |
| | akglsdvkma tdpeniikem |
| 181 | pvcrqtgntc kelrdpvtsg lntsvvfgfl nlvlwvgnlw |
| | fvfketgwaa pflrappgap |
| 241 | ekqpapgday gdagygqgpg gygpqdsygp qggyqpdygq |
| | pagsggsgyg pqgdygqqgy |
| 301 | gpqgaptsfs nqm |

An exemplary SYP nucleic acid sequence is provided at NCBI Accession No. NM_003179, version NM_003179.2, incorporated herein by reference and set forth below (SEQ ID NO: 34):

| | |
|---|---|
| 1 | gccccctgca ttgctgatgc tgctgctggc ggacatggac |
| | gtggtgaatc agctggtggc |
| 61 | tgggggtcag ttccgggtgg tcaaggagcc cctcggcttt |
| | gtgaaggtgc tgcaatgggt |
| 121 | cttcgccatc ttcgcctttg ccacatgcgg cagctacagt |
| | ggggagctcc agctgagcgt |
| 181 | ggattgtgcc aacaagaccg agagtgacct cagcatcgag |
| | gtcgagttcg agtacccctt |
| 241 | caggctgcac caagtgtact ttgatgcacc cacctgccga |
| | gggggcacca ccaaggtctt |
| 301 | cttagttggg gactactcct cgtcagccga attctttgtc |
| | accgtggccg tgtttgcctt |
| 361 | cctctactcc atgggggctc tggccaccta catcttcctg |
| | cagaacaagt accgagagaa |
| 421 | taacaaaggg cccatgctgg actttctggc cacggctgtg |
| | ttcgccttca tgtggctagt |
| 481 | tagctcatcg gcatgggcca aggggctgtc agatgtgaag |
| | atggccacag acccagagaa |
| 541 | cattatcaag gagatgcctg tctgccgcca gacagggaac |
| | acatgcaagg agctgagaga |
| 601 | ccctgtgacc tcgggactca acacctcggt ggtgttcggc |
| | ttcctgaacc tggtgctctg |
| 661 | ggtcggcaac ctgtggttcg tgtttaagga gacaggctgg |
| | gccgccccgt tcctgcgcgc |
| 721 | gcctcccggc gcccccgaga aacaaccggc acccggggac |
| | gcctacggcg atgcaggcta |
| 781 | cgggcagggc cccggcgggt acgggcccca ggattcctac |
| | gggcctcagg gcggctacca |
| 841 | gcctgactat ggtcaaccag ccggcagcgg tggcagtggc |
| | tacgggcctc agggcgacta |
| 901 | tgggcagcaa ggctacggcc cgcagggtgc acccacctcc |
| | ttctccaatc agatgtagtc |
| 961 | tggtcagtga agcccaggag gacctggggg gggcaagagc |
| | tcaggagaag gcctgccccc |
| 1021 | cttcccaccc ctatacccta ggtctccacc cctcaagcca |
| | ggagaccctg tctttgctgt |
| 1081 | ttatatatat atatattata tataaatatc tatttatctg |
| | tctgagccct gccctcactc |
| 1141 | cactcccctc atccactagg tgcccagtct tgagtgggcc |
| | ccctctctta ccccgtccct |
| 1201 | ttccctgcat cccttggccc ctctctgttt accctccctg |
| | tcccctgagg ttaaggggat |
| 1261 | ctaaaaggag gacagggagg gaacagacct cggctgtgtg |
| | gggagggtgg gcgtgacttc |
| 1321 | agactctctc ctctctctcc ctccactcct cccaactctg |
| | gccttggttc ctccagcaat |
| 1381 | gcctgcctga acaaaggccg ttagggaaat ccaactccag |
| | ggttaaagaa aggcagagat |
| 1441 | tgggggggct tggggtagag aggacagttt aggacccaag |
| | gtggtcttgg agaggaggtg |
| 1501 | tggagtggag gggtcagcag gggggttggg ttccagacag |
| | agtggatctg gagtctgaag |
| 1561 | gagaggagtg cgctagagca ttctggggtg gggcttggaa |
| | gggcgctgag ggcagggttc |
| 1621 | tagaaggggc gaggctttaa gcgaggcaga atggtgggct |
| | ccagagtagg tgggtcttgg |
| 1681 | attggtacca gagcctatgg aaagggtgtg gcttggaaca |
| | tttgggagac tgagcttgat |
| 1741 | tctaaagggg acagatcttg agcaaggcaa gaagtgggat |
| | tcaggaatgg gccaagccag |
| 1801 | ggttccagac agggtggggc ttagaatggg gcttccatgg |
| | tggtttcaga aagggcagcc |
| 1861 | cctccccatg gtgcagtgaa gaaaatgttt tacaatggct |
| | gggtttgggc agtggagagg |
| 1921 | ggacttggat aggagcttcc agatgggttt tgttaggggt |
| | gggggagaat ggctctggct |
| 1981 | acgacttggg acggaagtgg cctgagaaga gtcgagtgat |
| | atggcttgta gggtgaggcg |
| 2041 | tgggatccag agagaagcac cccaccacac acacccttcc |
| | ccactcccgt gatgaaacag |
| 2101 | ctaggttaat aggaggacag aaccaacggg tctgtgggac |
| | tggcccaccc ctcttccccc |
| 2161 | ttcccctgcg ccctccctcc ctccacacct ccacccgtcc |
| | tggggtggtt ggaggcctgg |
| 2221 | tctggagccc ctatcctgca ccctctgcta tgtctgtgat |
| | gtcagtagtg cctgtgatcg |
| 2281 | tgtgttgcca ttttgtctgg ctgtggcccc tccttctccc |
| | ctccagaccc ctaccctttc |
| 2341 | ccaaaccctt cggtattgtt caaagaaccc ccctccccaa |
| | ggaagaacaa atatgattct |
| 2401 | cctctcccaa ataaactcct taaccaccta gtcaaaaaaa |
| | aaaaaaaaa |

It is determined which types of stromal cells from the tumor microenvironment are recruited by C1 cells and what are the functional consequences on tumor behavior and progression as well as the implications for therapy.

In summary, BCL9 was found to promote neural features through its interaction with paraspeckles in a specific subtype of CRC and enhances tumor progression by promoting neurotransmitter-dependent communication between tumor cells and cells of the tumor-microenvironment. Therefore, the examples described herein provide distinct insights into the role of BCL9 in tumor progression as well as innovative avenues for therapeutic intervention by targeting BCL9 itself or blockade of neurotransmitter receptors or calcium channels with FDA approved drugs such as propranolol or verapamil, which are commonly used to treat hypertension and heart disorders (Frishman et al., (1984) N. Engl. J. Med. 310:830-837; Lundstrom et al., (1990) J. Am. Coll. Cardiol. 16:86-90).

### World Health Organization (WHO) Criteria

The WHO Criteria for evaluating the effectiveness of anti-cancer agents on tumor shrinkage, developed in the 1970s by the International Union Against Cancer and the World Health Organization, represented the first generally agreed specific criteria for the codification of tumor response evaluation. These criteria were first published in 1981 (Miller et al., 1981 Clin Cancer Res., 47(1): 207-14, incorporated herein by reference). WHO Criteria proposed >50% tumor shrinkage for a Partial Response and >25% tumor increase for Progressive Disease.

### Response Evaluation Criteria in Solid Tumors (RECIST)

RECIST is a set of published rules that define when tumors in cancer patients improve (“respond”), stay the same (“stabilize”), or worsen (“progress”) during treatment (Eisenhauer et al., 2009 European Journal of Cancer, 45: 228-247, incorporated herein by reference). Only patients with measureable disease at baseline should be included in protocols where objective tumor response is the primary endpoint.

The response criteria for evaluation of target lesions are as follows:
Complete Response (CR): Disappearance of all target lesions.
Partial Response (PR): At least a 30% decrease in the sum of the longest diameter (LD) of target lesions, taking as reference the baseline sum LD.
Stable Disease (SD): Neither sufficient shrinkage to qualify for PR nor sufficient increase to qualify for PD, taking as reference the smallest sum LD since the treatment started.
Progressive Disease (PD): At least a 20% increase in the sum of the LD of target lesions, taking as reference the smallest sum LD recorded since the treatment started or the appearance of one or more new lesions.

The response criteria for evaluation of non-target lesions are as follows:
Complete Response (CR): Disappearance of all non-target lesions and normalization of tumor marker level.
Incomplete Response/Stable Disease (SD): Persistence of one or more non-target lesion(s) or/and maintenance of tumor marker level above the normal limits.
Progressive Disease (PD): Appearance of one or more new lesions and/or unequivocal progression of existing non-target lesions.

The response criteria for evaluation of best overall response are as follows. The best overall response is the best response recorded from the start of the treatment until disease progression/recurrence (taking as reference for PD the smallest measurements recorded since the treatment started). In general, the patient's best response assignment will depend on the achievement of both measurement and confirmation criteria.
Patients with a global deterioration of health status requiring discontinuation of treatment without objective evidence of disease progression at that time should be classified as having “symptomatic deterioration”. Every effort should be made to document the objective progression even after discontinuation of treatment.
In some circumstances, it may be difficult to distinguish residual disease from normal tissue. When the evaluation of complete response depends on this determination, it is recommended that the residual lesion be investigated (fine needle aspirate/biopsy) to confirm the complete response status.

### Immune-Related Response Criteria

The immune-related response criteria (irRC) is a set of published rules that define when tumors in cancer patients improve (“respond”), stay the same (“stabilize”), or worsen (“progress”) during treatment, where the compound being evaluated is an immuno-oncology drug. The Immune-Related Response Criteria, first published in 2009 (Wolchok et al., 2009 Clin Cancer Res, 15(23):7412, incorporated herein by reference), arose out of observations that immuno-oncology drugs would fail in clinical trials that measured responses using the WHO or RECIST Criteria, because these criteria could not account for the time gap in many patients between initial treatment and the apparent action of the immune system to reduce the tumor burden. The key driver in the development of the irRC was the observation that, in studies of various cancer therapies derived from the immune system such as cytokines and monoclonal antibodies, the looked-for Complete and Partial Responses as well as Stable Disease only occurred after an increase in tumor burden that the conventional RECIST Criteria would have dubbed ‘Progressive Disease.’ RECIST failed to take account of the delay between dosing and an observed anti-tumor T cell response, so that otherwise ‘successful’ drugs—that is, drugs which ultimately prolonged life—failed in clinical trials.

The irRC are based on the WHO Criteria; however, the measurement of tumor burden and the assessment of immune-related response have been modified as set forth below.

### Measurement of Tumor Burden

In the irRC, tumor burden is measured by combining ‘index’ lesions with new lesions. Ordinarily, tumor burden would be measured with a limited number of ‘index’ lesions (that is, the largest identifiable lesions) at baseline, with new lesions identified at subsequent time points counting as ‘Progressive Disease’. In the irRC, by contrast, new lesions are a change in tumor burden. The irRC retained the bidirectional measurement of lesions that had originally been laid down in the WHO Criteria.

### Assessment of Immune-Related Response

In the irRC, an immune-related Complete Response (irCR) is the disappearance of all lesions, measured or unmeasured, and no new lesions; an immune-related Partial Response (irPR) is a 50% drop in tumor burden from baseline as defined by the irRC; and immune-related Progressive Disease (irPD) is a 25% increase in tumor burden from the lowest level recorded. Everything else is considered immune-related Stable Disease (irSD). Even if tumor burden is rising, the immune system is likely to “kick in” some months after first dosing and lead to an eventual decline in tumor burden for many patients. The 25% threshold accounts for this apparent delay.

### Gene Expression Profiling

In general, methods of gene expression profiling may be divided into two large groups: methods based on hybridization analysis of polynucleotides and methods based on sequencing of polynucleotides. Methods known in the art for the quantification of mRNA expression in a sample include northern blotting and in situ hybridization, RNAse protection assays, RNA-seq, and reverse transcription polymerase chain reaction (RT-PCR). Alternatively, antibodies are employed that recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS). For example, RT-PCR is used to compare mRNA levels in different sample populations, in normal and tumor tissues, with or without drug treatment, to characterize patterns of gene expression, to discriminate between closely related mRNAs, and/or to analyze RNA structure.

In some cases, a first step in gene expression profiling by RT-PCR is the reverse transcription of the RNA template into cDNA, followed by amplification in a PCR reaction. For example, extracted RNA is reverse-transcribed using a GeneAmp RNA PCR kit (Perkin Elmer, Calif., USA), following the manufacturer's instructions. The cDNA is then used as template in a subsequent PCR amplification and quantitative analysis using, for example, a TaqMan® (Life Technologies, Inc., Grand Island, N.Y.) assay.

### Microarrays

Differential gene expression can also be identified, or confirmed using a microarray technique. In these methods, polynucleotide sequences of interest (including cDNAs and oligonucleotides) are plated, or arrayed, on a microchip substrate. The arrayed sequences are then hybridized with specific DNA probes from cells or tissues of interest. Just as in the RT-PCR method, the source of mRNA typically is total RNA isolated from human tumors or tumor cell lines and corresponding normal tissues or cell lines. Thus, RNA is isolated from a variety of primary tumors or tumor cell lines. If the source of mRNA is a primary tumor, mRNA is extracted from frozen or archived tissue samples.

In the microarray technique, PCR-amplified inserts of cDNA clones are applied to a substrate in a dense array. The microarrayed genes, immobilized on the microchip, are suitable for hybridization under stringent conditions.

In some cases, fluorescently labeled cDNA probes are generated through incorporation of fluorescent nucleotides by reverse transcription of RNA extracted from tissues of interest (e.g., leukemia tissue). Labeled cDNA probes applied to the chip hybridize with specificity to loci of DNA on the array. After washing to remove non-specifically bound probes, the chip is scanned by confocal laser microscopy or by another detection method, such as a charge-coupled device (CCD) camera. Quantification of hybridization of each arrayed element allows for assessment of corresponding mRNA abundance.

In some configurations, dual color fluorescence is used. With dual color fluorescence, separately labeled cDNA probes generated from two sources of RNA are hybridized pairwise to the array. The relative abundance of the transcripts from the two sources corresponding to each specified gene is thus determined simultaneously. In various configurations, the miniaturized scale of the hybridization can afford a convenient and rapid evaluation of the expression pattern for large numbers of genes. In various configurations, such methods can have sensitivity required to detect rare transcripts, which are expressed at fewer than 1000, fewer than 100, or fewer than 10 copies per cell. In various configurations, such methods can detect at least approximately two-fold differences in expression levels (Schena et al., Proc. Natl. Acad. Sci. USA 93(2): 106-149 (1996)). In various configurations, microarray analysis is performed by commercially available equipment, following manufacturer's protocols, such as by using the Aflymetrix GenChip technology, or Incyte's microarray technology.

### RNA-Seq

RNA sequencing (RNA-seq), also called whole transcriptome shotgun sequencing (WTSS), uses next-generation sequencing (NGS) to reveal the presence and quantity of RNA in a biological sample at a given moment in time.

RNA-Seq is used to analyze the continually changing cellular transcriptome. See, e.g., Wang et al., 2009 Nat Rev Genet, 10(1): 57-63, incorporated herein by reference. Specifically, RNA-Seq facilitates the ability to look at alternative gene spliced transcripts, post-transcriptional modifications, gene fusion, mutations/SNPs and changes in gene expression. In addition to mRNA transcripts, RNA-Seq can look at different populations of RNA to include total RNA, small RNA, such as miRNA, tRNA, and ribosomal profiling. RNA-Seq can also be used to determine exon/intron boundaries and verify or amend previously annotated 5′ and 3′ gene boundaries.

Prior to RNA-Seq, gene expression studies were done with hybridization-based microarrays. Issues with microarrays include cross-hybridization artifacts, poor quantification of lowly and highly expressed genes, and needing to know the sequence of interest. Because of these technical issues, transcriptomics transitioned to sequencing-based methods. These progressed from Sanger sequencing of Expressed Sequence Tag libraries, to chemical tag-based methods (e.g., serial analysis of gene expression), and finally to the current technology, NGS of cDNA (notably RNA-Seq).

Pharmaceutical Therapeutics

For therapeutic uses, the agents (e.g., a BCL9 inhibitor, a calcium channel receptor inhibitor, and/or a beta-adrenergic antagonist) described herein may be administered systemically, for example, formulated in a pharmaceutically-acceptable buffer such as physiological saline. Preferable routes of administration include, for example, subcutaneous, intravenous, intraperitoneal, intramuscular, or intradermal injections that provide continuous, sustained levels of the drug in the patient. Treatment of human patients or other animals will be carried out using a therapeutically effective amount of a therapeutic identified herein in a physiologically-acceptable carrier. Suitable carriers and their formulation are described, for example, in Remington's Pharmaceutical Sciences by E. W. Martin. The amount of the agents to be administered varies depending upon the manner of administration, the age and body weight of the patient, and with the clinical symptoms of the CRC. Generally, amounts will be in the range of those used for other agents used in the treatment of other diseases associated with CRC, although in certain instances lower amounts will be needed because of the increased specificity of the agents. For example, an agent is administered at a dosage that is cytotoxic to a neoplastic cell.

### Formulation of Pharmaceutical Compositions

Human dosage amounts can initially be determined by extrapolating from the amount of the agent used in animal models, as a skilled artisan recognizes it is routine in the art to modify the dosage for humans compared to animal models. In certain embodiments it is envisioned that the dosage may vary from between about 1 μg compound/Kg body weight to about 5000 mg compound/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other cases, this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, or 5000 mg/Kg body weight. In other aspects, it is envisaged that doses may be in the range of about 5 mg compound/Kg body to about 20 mg compound/Kg body. In other embodiments, the doses may be about 8, 10, 12, 14, 16 or 18 mg/Kg body weight. Of course, this dosage amount may be adjusted upward or downward, as is routinely done in such treatment protocols, depending on the results of the initial clinical trials and the needs of a particular patient.

In some cases, the agent of the invention is administered at a dose that is lower than the human equivalent dosage (HED) of the no observed adverse effect level (NOAEL) over a period of three months, four months, six months, nine months, 1 year, 2 years, 3 years, 4 years or more. The NOAEL, as determined in animal studies, is useful in determining the maximum recommended starting dose for human clinical trials. For instance, the NOAELs can be extrapolated to determine human equivalent dosages. Typically, such extrapolations between species are conducted based on the doses that are normalized to body surface area (i.e., mg/m²). In specific embodiments, the NOAELs are determined in mice, hamsters, rats, ferrets, guinea pigs, rabbits, dogs, primates, primates (monkeys, marmosets, squirrel monkeys, and baboons), micropigs or minipigs. For a discussion on the use of NOAELs and their extrapolation to determine human equivalent doses, see Guidance for Industry Estimating the Maximum Safe Starting Dose in Initial Clinical Trials for Therapeutics in Adult Healthy Volunteers, U.S. Department of Health and Human Services Food and Drug Administration Center for Drug Evaluation and Research (CDER), Pharmacology and Toxicology, July 2005, incorporated herein by reference.

The amount of an agent of the invention used in the prophylactic and/or therapeutic regimens which will be effective in the treatment of CRC can be based on the currently prescribed dosage of the agent as well as assessed by methods disclosed herein and known in the art. The frequency and dosage will vary also according to factors specific for each patient depending on the specific agent administered, the severity of the cancerous condition, the route of administration, as well as age, body, weight, response, and the past medical history of the patient. For example, the dosage of an agent of the invention which will be effective in the treatment of cancer can be determined by administering the agent to an animal model such as, e.g., the animal models disclosed herein or known to those skilled in the art. In addition, in vitro assays may optionally be employed to help identify optimal dosage ranges.

In some aspects, the prophylactic and/or therapeutic regimens comprise titrating the dosages administered to the patient so as to achieve a specified measure of therapeutic efficacy. Such measures include a reduction in the cancer cell population in the patient.

In certain cases, the dosage of the agent of the invention in the prophylactic and/or therapeutic regimen is adjusted so as to achieve a reduction in the number or amount of cancer cells found in a test specimen extracted from a patient after undergoing the prophylactic and/or therapeutic regimen, as compared with a reference sample. Here, the reference sample is a specimen extracted from the patient undergoing therapy, wherein the specimen is extracted from the patient at an earlier time point. In one aspect, the reference sample is a specimen extracted from the same patient, prior to receiving the prophylactic and/or therapeutic regimen. For example, the number or amount of cancer cells in the test specimen is at least 2%, 5%, 10%, 15%, 20%, 30%, 40%, 50%, 60%, 70%, 80%, 90%, 95% or 99% lower than in the reference sample.

In some cases, the dosage of the agent of the invention in the prophylactic and/or therapeutic regimen is adjusted so as to achieve a number or amount of cancer cells that falls within a predetermined reference range. In these embodiments, the number or amount of cancer cells in a test specimen is compared with a predetermined reference range.

In other embodiments, the dosage of the agent of the invention in prophylactic and/or therapeutic regimen is adjusted so as to achieve a reduction in the number or amount of cancer cells found in a test specimen extracted from a patient after undergoing the prophylactic and/or therapeutic regimen, as compared with a reference sample, wherein the reference sample is a specimen is extracted from a healthy, noncancer-afflicted patient. For example, the number or amount of cancer cells in the test specimen is at least within 60%, 50%, 40%, 30%, 20%, 15%, 10%, 5%, or 2% of the number or amount of cancer cells in the reference sample.

In treating certain human patients having solid tumors, extracting multiple tissue specimens from a suspected tumor site may prove impracticable. In these cases, the dosage of the agent of the invention in the prophylactic and/or therapeutic regimen for a human patient is extrapolated from doses in animal models that are effective to reduce the cancer population in those animal models. In the animal models, the prophylactic and/or therapeutic regimens are adjusted so as to achieve a reduction in the number or amount of cancer cells found in a test specimen extracted from an animal after undergoing the prophylactic and/or therapeutic regimen, as compared with a reference sample. The reference sample can be a specimen extracted from the same animal, prior to receiving the prophylactic and/or therapeutic regimen. In specific embodiments, the number or amount of cancer cells in the test specimen is at least 2%, 5%, 10%, 15%, 20%, 30%, 40%, 50% or 60% lower than in the reference sample. The doses effective in reducing the number or amount of cancer cells in the animals can be normalized to body surface area (e.g., mg/m²) to provide an equivalent human dose.

The prophylactic and/or therapeutic regimens disclosed herein comprise administration of an agent of the invention or pharmaceutical compositions thereof to the patient in a single dose or in multiple doses (e.g., 1, 2, 3, 4, 5, 6, 7, 8, 10, 15, 20, or more doses).

In one aspect, the prophylactic and/or therapeutic regimens comprise administration of the agent of the invention or pharmaceutical compositions thereof in multiple doses. When administered in multiple doses, the agent or pharmaceutical compositions are administered with a frequency and in an amount sufficient to treat the condition. For example, the frequency of administration ranges from once a day up to about once every eight weeks. In another example, the frequency of administration ranges from about once a week up to about once every six weeks. In another example, the frequency of administration ranges from about once every three weeks up to about once every four weeks.

Generally, the dosage of an agent of the invention administered to a subject to treat cancer is in the range of 0.01 to 500 mg/kg, e.g., in the range of 0.1 mg/kg to 100 mg/kg, of the subject's body weight. For example, the dosage administered to a subject is in the range of 0.1 mg/kg to 50 mg/kg, or 1 mg/kg to 50 mg/kg, of the subject's body weight, more preferably in the range of 0.1 mg/kg to 25 mg/kg, or 1 mg/kg to 25 mg/kg, of the patient's body weight. In another example, the dosage of an agent of the invention administered to a subject to treat cancer in a patient is 500 mg/kg or less, preferably 250 mg/kg or less, 100 mg/kg or less, 95 mg/kg or less, 90 mg/kg or less, 85 mg/kg or less, 80 mg/kg or less, 75 mg/kg or less, 70 mg/kg or less, 65 mg/kg or less, 60 mg/kg or less, 55 mg/kg or less, 50 mg/kg or less, 45 mg/kg or less, 40 mg/kg or less, 35 mg/kg or less, 30 mg/kg or less, 25 mg/kg or less, 20 mg/kg or less, 15 mg/kg or less, 10 mg/kg or less, 5 mg/kg or less, 2.5 mg/kg or less, 2 mg/kg or less, 1.5 mg/kg or less, or 1 mg/kg or less of a patient's body weight.

In another example, the dosage of an agent of the invention administered to a subject to treat cancer in a patient is a unit dose of 0.1 to 50 mg, 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7 mg, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg.

In another example, the dosage of an agent of the invention administered to a subject to treat cancer in a patient is in the range of 0.01 to 10 g/m², and more typically, in the range of 0.1 g/m² to 7.5 g/m², of the subject's body weight. For example, the dosage administered to a subject is in the range of 0.5 g/m² to 5 g/m², or 1 g/m² to 5 g/m² of the subject's body's surface area.

In another example, the prophylactic and/or therapeutic regimen comprises administering to a patient one or more doses of an effective amount of an agent of the invention, wherein the dose of an effective amount achieves a plasma level of at least 0.1 μg/mL, at least 0.5 μg/mL, at least 1 μg/mL, at least 2 μg/mL, at least 5 μg/mL, at least 6 μg/mL, at least 10 μg/mL, at least 15 μg/mL, at least 20 μg/mL, at least 25 μg/mL, at least 50 μg/mL, at least 100 μg/mL, at least 125 μg/mL, at least 150 μg/mL, at least 175 μg/mL, at least 200 μg/mL, at least 225 μg/mL, at least 250 μg/mL, at least 275 μg/mL, at least 300 μg/mL, at least 325 μg/mL, at least 350 μg/mL, at least 375 μg/mL, or at least 400 μg/mL of the agent of the invention.

In another example, the prophylactic and/or therapeutic regimen comprises administering to a patient a plurality of doses of an effective amount of an agent of the invention, wherein the plurality of doses maintains a plasma level of at least 0.1 μg/mL, at least 0.5 μg/mL, at least 1 μg/mL, at least 2 μg/mL, at least 5 μg/mL, at least 6 μg/mL, at least 10 μg/mL, at least 15 μg/mL, at least 20 μg/mL, at least 25 μg/mL, at least 50 μg/mL, at least 100 μg/mL, at least 125 μg/mL, at least 150 μg/mL, at least 175 μg/mL, at least 200 μg/mL, at least 225 μg/mL, at least 250 μg/mL, at least 275 μg/mL, at least 300 μg/mL, at least 325 μg/mL, at least 350 μg/mL, at least 375 μg/mL, or at least 400 μg/mL of the agent of the invention for at least 1 day, 1 month, 2 months, 3 months, 4 months, 5 months, 6 months, 7 months, 8 months, 9 months, 10 months, 11 months, 12 months, 15 months, 18 months, 24 months or 36 months.

In other embodiments, the prophylactic and/or therapeutic regimen comprises administering to a patient a plurality of doses of an effective amount of an agent of the invention, wherein the plurality of doses maintains a plasma level of at least 0.1 μg/mL, at least 0.5 μg/mL, at least 1 μg/mL, at least 2 μg/mL, at least 5 μg/mL, at least 6 μg/mL, at least 10 μg/mL, at least 15 μg/mL, at least 20 μg/mL, at least 25 μg/mL, at least 50 μg/mL, at least 100 μg/mL, at least 125 μg/mL, at least 150 μg/mL, at least 175 μg/mL, at least 200 μg/mL, at least 225 μg/mL, at least 250 μg/mL, at least 275 μg/mL, at least 300 μg/mL, at least 325 μg/mL, at least 350 μg/mL, at least 375 μg/mL, or at least 400 μg/mL of the agent of the invention for at least 1 day, 1 month, 2 months, 3 months, 4 months, 5 months, 6 months, 7 months, 8 months, 9 months, 10 months, 11 months, 12 months, 15 months, 18 months, 24 months or 36 months.

### Combination Therapy

In one example, the agents are administered in combination therapy, i.e., combined with other agents, e.g., therapeutic agents, that are useful for treating pathological conditions or disorders, such as various forms of cancer. The term “in combination” in this context means that the agents are given substantially contemporaneously, either simultaneously or sequentially. If given sequentially, at the onset of administration of the second compound, the first of the two compounds is in some cases still detectable at effective concentrations at the site of treatment.

The administration of a compound or a combination of compounds for the treatment of a neoplasia may be by any suitable means that results in a concentration of the therapeutic that, combined with other components, is effective in ameliorating, reducing, or stabilizing a neoplasia. The agent may be contained in any appropriate amount in any suitable carrier substance, and is generally present in an amount of 1-95% by weight of the total weight of the composition. The agent may be provided in a dosage form that is suitable for parenteral (e.g., subcutaneously, intravenously, intramuscularly, or intraperitoneally) administration route. The agent may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Accordingly, in some examples, the prophylactic and/or therapeutic regimen comprises administration of an agent of the invention in combination with one or more additional anticancer therapeutics. In one example, the dosages of the one or more additional anticancer therapeutics used in the combination therapy is lower than those which have been or are currently being used to treat cancer. The recommended dosages of the one or more additional anticancer therapeutics currently used for the treatment of cancer can be obtained from any reference in the art including, but not limited to, Hardman et al., eds., Goodman & Gilman's The Pharmacological Basis Of Basis Of Terapeutics, 10th ed., McGraw-Hill, New York, 2001; Physician's Desk Reference (60.sup.th ed., 2006), which is incorporated herein by reference in its entirety.

The agent of the invention and the one or more additional anticancer therapeutics can be administered separately, simultaneously, or sequentially. In various aspects, the agent of the invention and the additional anticancer therapeutic are administered less than 5 minutes apart, less than 30 minutes apart, less than 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, 96 hours apart, 120 hours part, or 168 hours apart. In another example, two or more anticancer therapeutics are administered within the same patient visit.

In certain aspects, the agent of the invention and the additional anticancer therapeutic are cyclically administered. Cycling therapy involves the administration of one anticancer therapeutic for a period of time, followed by the administration of a second anticancer therapeutic for a period of time and repeating this sequential administration, i.e., the cycle, in order to reduce the development of resistance to one or both of the agents, to avoid or reduce the side effects of one or both of the agents, and/or to improve the efficacy of the therapies. In one example, cycling therapy involves the administration of a first anticancer therapeutic for a period of time, followed by the administration of a second anticancer therapeutic for a period of time, optionally, followed by the administration of a third anticancer therapeutic for a period of time and so forth, and repeating this sequential administration, i.e., the cycle in order to reduce the development of resistance to the agent, to avoid or reduce the side effects of one of the agent, and/or to improve the efficacy of the agent.

In another example, the agents are administered concurrently to a subject in separate compositions. The combination the agents of the invention may be administered to a subject by the same or different routes of administration.

When an agent of the invention and the additional anticancer therapeutic are administered to a subject concurrently, the term “concurrently” is not limited to the administration of the agent at exactly the same time, but rather, it is meant that they are administered to a subject in a sequence and within a time interval such that they can act together (e.g., synergistically to provide an increased benefit than if they were administered otherwise). For example, the agents may be administered at the same time or sequentially in any order at different points in time; however, if not administered at the same time, they should be administered sufficiently close in time so as to provide the desired therapeutic effect, preferably in a synergistic fashion. The combination of the agents can be administered separately, in any appropriate form and by any suitable route. When the components of the combination the agents are not administered in the same pharmaceutical composition, it is understood that they can be administered in any order to a subject in need thereof. For example, an agent of the invention can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the additional anticancer therapeutic, to a subject in need thereof. In various aspects, the agents are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one example, the agents are administered within the same office visit. In another example, the combination the agents of the invention are administered at 1 minute to 24 hours apart.

### Release of Pharmaceutical Compositions

Pharmaceutical compositions according to the invention may be formulated to release the agents substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with the thymus; (v) formulations that allow for convenient dosing, such that doses are administered, for example, once every one or two weeks; and (vi) formulations that target a neoplasia by using carriers or chemical derivatives to deliver the agent to a particular cell type (e.g., neoplastic cell). For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the agent. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

### Parenteral Compositions

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the agent that reduces or ameliorates a neoplasia, the composition may include suitable parenterally acceptable carriers and/or excipients. The agent may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to the invention may be in the form suitable for sterile injection. To prepare such a composition, the suitable active antineoplastic therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol.

### Controlled Release Parenteral Compositions

Controlled release parenteral compositions may be in form of aqueous suspensions, microspheres, microcapsules, magnetic microspheres, oil solutions, oil suspensions, or emulsions. Alternatively, the active drug may be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutam-nine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

### Kits or Pharmaceutical Systems

The present compositions may be assembled into kits or pharmaceutical systems for use in ameliorating a neoplasia. Kits or pharmaceutical systems according to this aspect of the invention comprise a carrier means, such as a box, carton, tube or the like, having in close confinement therein one or more container means, such as vials, tubes, ampoules, or bottles. The kits or pharmaceutical systems of the invention may also comprise associated instructions for using the agent of the invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Materials and Methods

The following materials and methods were utilized to generate the results described herein.

### Nanoparticle Synthesis

Polyethylenimines (PEIs) have been extensively used as reagents for transfection. The high amine content of PEIs causes significant positive charge density on the surface of the polymer which interacts with the negatively charged components of the cell membrane, resulting in translocation into the cell (Nouri et al., (2017) Int. J. Nanomedicine 12:5557-5569). A major disadvantage of PEI-based nanoparticles is their toxicity. Several factors, such as high molecular weight and increased branching, affect the toxicity of PEIs (Fischer et al., (1999) Pharm. Res. 16:1273-1279). The application of low molecular weight PEIs have been shown to increase the efficiency of transfection.

The nanoparticles used in the present invention were made of low-molecular weight polyamines and lipids (Dahlman et al., (2014) Nat. Nanotechnol. 9:648-655) to package the mixture of two BCL9 siRNAs (SEQ ID NOs: 5 and 6). Small polyamines were conjugated to alkyl tails via an epoxide opening reaction. A variety of amines were used along with different lipid lengths and different molar ratios of lipids:amines to generate structural diversity amongst the nanoparticles.

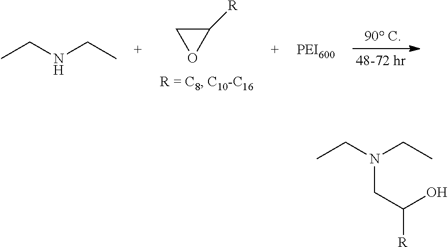

### Generate BCL9 Knockout Cell Line by CRISPR-Cas9 System

To generate a BCL9 knockout cell line, lentiCrisprV2 plasmid (#52%1) containing guide ribonucleic acid (gRNA) targeting either BCL9 (CAGTAGTITGGCCATGGGA (SEQ ID NO: 35)) or AAVS1 (Xu et al., 2015 Genome Res., 25:1147-1157, incorporated herein by reference) was delivered to RKO or Colo320 cells using lipofectamine 2000 (Life Technologies™). After 48 hrs transfection, cells were cultured in 96 well plates according to a serial dilution protocol (Cell Cloning by Serial Dilution in % well Plates, Corning Incorporated Life Sciences). Single clones from the % well plates were harvested once the cell counts reached 1×10², and were transferred to 12-well plates to continue growing. The expression level of BCL9 was analyzed by immunoblotting. Genomic DNA was extracted, the gRNA targeting sequencing was amplified by PCR, and the PCR product was sequenced to identify the frameshift mutation of BCL9. Puromycin selection was not used in order to limit off-target effects caused by continued expression of gRNA and Cas9. The gRNA was specifically designed to target the 5′UTR portion of the BCL9 ORF which codes for the amino acid sequence between the HD1 and HD2 domain; this creates a frameshift mutation which induces loss-of-function of BCL9 but simultaneously preserves the HD1 domain. Mutated BCL9 is therefore still able to occupy the Pygo2 binding site, which further eliminates the possibility that other co-factors will compensate for BCL9 function.

### Gene Expression Profiling and Statistical Analysis

RNA was extracted from wild type and BCL9 knockout RKO cells with or without Poly I:C treatment (1 μg/ml) and subsequently purified using the TURBO DNA-free™ Kit (AM1907, invitrogen) to remove any residual DNA. An RNA library was prepared using the ribosome RNA removing method and sequenced with a 150 bp paired-end protocol in the Center for Cancer Computational Biology at the Dana Farber Cancer Institute (PRJNA554110). After quality control (QC) analysis was performed using fastQC, the first 10 bases were trimmed for each read. STAR software was used to map the readings to the human genome (hg19) and duplicates were removed using Picard. HTSeq was used to evaluate the gene expression levels by the count number for each gene and were subsequently annotated using the Ensemble database. Gene differential analysis was then applied to the expression profiling table using R package edgeR. For further analysis, the difference in exon usage between different conditions was calculated and R package DEXSeq was used to find differences between them.

### Coimmunoprecipitation Assay

To extract nuclear protein, cells were incubated with cytoplasmic lysis buffer (50 mM Tris-HCl pH=8.0, 20 mM NaCl, 2 mM EDTA, 0.5% Tween-20 containing protease/phosphatase Inhibitor, #5872, Cell Signaling®) on ice for 10 mins, centrifuged at 6000×g, and the precipitate collected. This step was repeated 3 times to remove at much cytoplasmic protein as possible. Subsequently, nuclear lysis buffer (50 mM Tris-HCl pH=7.4, 150 mM NaCl, 2 mM EDTA, 1% Triton X-100(v/v) containing protease/phosphatase inhibitor) was added to the precipitate, and sonication was used to lyse the sample before centrifugation at 16000×g for 15 minutes at 4 degrees. 1 mg of the nuclear lysate was blocked with 5% BSA for 1 hr at 4 degrees, before overnight incubation with anti-BCL9 (ab37305 Abcam®, 6109 generated in New England Biolabs), NONO (ab70335, Abcam®), SFPQ (ab38148), ILF2 (H00003608-D01, abnova) or β-catenin (9562L, Cell Signaling®) antibodies. Normal rabbit IgG (sc-3888, Santa Cruz®) was used as a negative control. The following day, Protein G and A DynaBeads (10003D, 10002D, ThermoFisher®) were added to the nuclear lysate at a ratio of 1:1 and rotated for an additional 4 hrs. The beads were washed 3 times with washing buffer (50 mM Tris-HCl pH=7.4, 200 mM NaCl, 2 mM EDTA, 1% Triton X-100(v/v)). The beads were then re-suspended with 2×LDS sample buffer and boiled for 10 mins. For Immunoblotting, the sample was electrophoresed using SDS-PAGE, transferred to nitrocellulose membrane and blocked using non-fat 5% milk. The membrane was subsequently probed using anti-BCL9 (H00000607-M01, abnova), NONO (TA504777, Origene®), SFPQ (MA1-25325, ThermoFisher®), ILF2(PA5-18718, ThermoFisher®) or β-catenin (610154, BD Transduction Laboratories) antibodies. For total protein MS analysis, IP protein samples from anti-IgG and anti-BCL9 groups were recovered by Trichloroacetic acid (TCA 47658-U, Sigma®) precipitation; samples were incubated for 10 minutes at 4 degrees, before centrifugation at 16000×g for 5 minutes. In addition, pulled down protein samples were analyzed by silver staining; bands which existed in anti-BCL9 samples, but not in the anti-normal IgG groups, were cut and used for further MS analysis to validate previous results. All mass spectrometry was performed in the Taplin Mass Spectrometry Facility at Harvard Medical School. To limit any off-target effects of anti-BCL9 antibody, the MS results were verified with two independent anti-BCL9 antibodies (37305 Abcam® and 6109, see KEY RESOURCES table) which target different amino acid sequences of BCL9.

### Consensus Clustering Analysis

This study used RNA sequencing data from 459 colorectal cancer patient samples (The Cancer Genome Atlas (TCGA); cancergenome.nih.gov/) and unsupervised clustering was performed using R Package Consensus Cluster Plus. Gene expression data was normalized by the data size factor of the R package DESeq. The top 2000 genes with the biggest variation in expression were used to generate sample clusters. According to the efficiency of the different number of clusters (K), the patient samples were clustered into 4 groups. The heat map demonstrates the expression of all genes in the 4 clusters. Cox proportional hazards survival analysis was then used to determine whether there is a correlation between survival and the expression level of BCL9 in different clusters. To investigate differential gene expression in each of the 4 clusters, the ANOVA statistical test (followed by post hoc testing) was used, and a P value of <0.01 was considered significant. After identification of the gene expression sets, the enrichment score of each gene was calculated using MSigDB C2 pathway gene sets. GO analysis was applied to summarize the function of specific genes by using SP_PIR_KEYWORDS annotation categories in DAVID. The genes that appeared in both the BCL9 correlated gene set and cluster specific gene set were used to calculate the enrichment score by MSigDB C2 pathway gene sets. The correlation coefficient network was generated with gene expression data from C1 tumor samples using the R package WGCNA (labs.genetics.ucla.edu/horvath/htdocs/CoexpressionNetwork/Rpackages/WGCN). Patients in the same cluster shared a similar gene expression profile but displayed different survival times; the patients with a shorter survival time were observed to be sampled at a later stage of the disease. Therefore survival time was used to evaluate tumor progression. Due to WCGNA, RNA-seq and MS analysis were carried out independently from each other. This purpose of this was to describe the synergistic effect of BCL9 downstream genes or BCL9 partners, and to ensure the BCL9-regulated bio-events existed and were observed during tumor progression.

### Protein-Protein Interaction Network Building and Function go Analysis

The differential nuclear location of BCL9 implied that it may interact with multiple types of protein complexes. As a result, a protein interaction network was established to evaluate the intensity of these interactions. To normalize the samples, any proteins in which the peptide number in anti-lgG was higher than the matched anti-BCL9 group (i.e. the protein didn't exist in at least two independent experiments) were removed. The enrichment score was calculated by subtracting the total lgG peptide value from anti-BCL9. After normalization, 276 proteins demonstrated a score above 2 and were chosen for further analysis. GO analysis in DAVID was used to define the functional groups of the proteins, and the result was presented in Chow-Ruskey diagrams. The candidate proteins were used to generate the protein-protein interacting network; in the map, proteins are represented by a colored dot and the interactions among individual proteins are represented by connected colored lines. Their weight corresponds to the combined score which was collected from the String database (String database, string-db.org/). Then, using the weight score for all the protein interactions as the distance between two proteins, and using a K-mean algorithm to cluster them, the proteins were found to cluster into 7 groups. The number of groups was determined by the first K-value greater than 2 which has a near stable SS (Sum of squared error). The number of SFPQ binding motif in 3′UTR region of BCL9 downstream genes are identified by using RBP map website (rbpmap.technion.ac.il).

### Cell Viability Assay

Cells that were transfected with shRNA and had undergone puromycin selection were plated into 96-well plates (in triplicate) at 4×10³ cells/well. The cell Titer glow viability assay (Promega® G7570) was used according to the manufacturer's instructions; briefly the cells were cultured for 48 hours, incubated with cell titer glow substrate and analyzed using a luciferase reader. The background luminescence of a blank well was subtracted from the sample readings.

### Wound Healing Assay

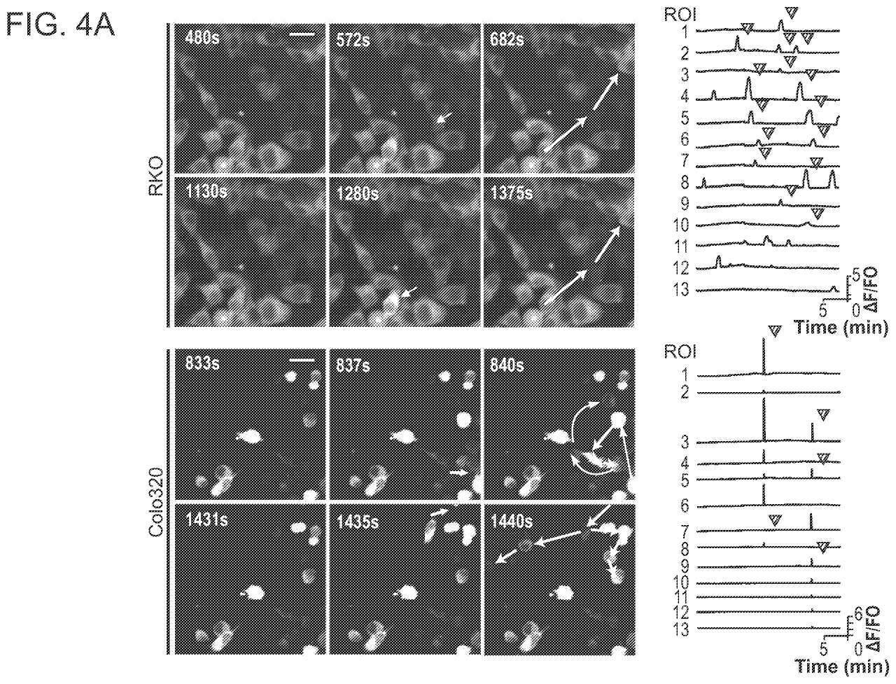

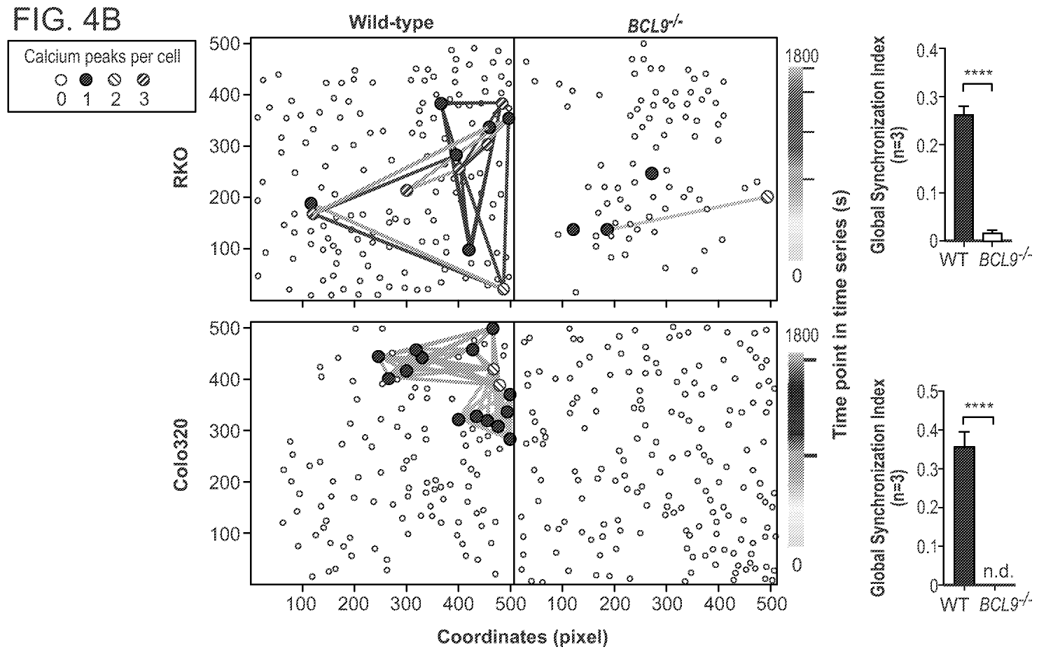

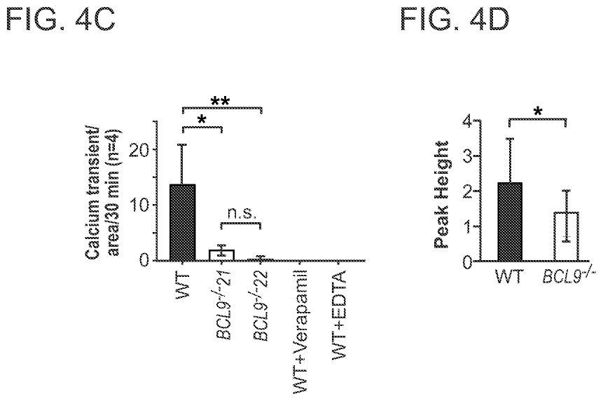

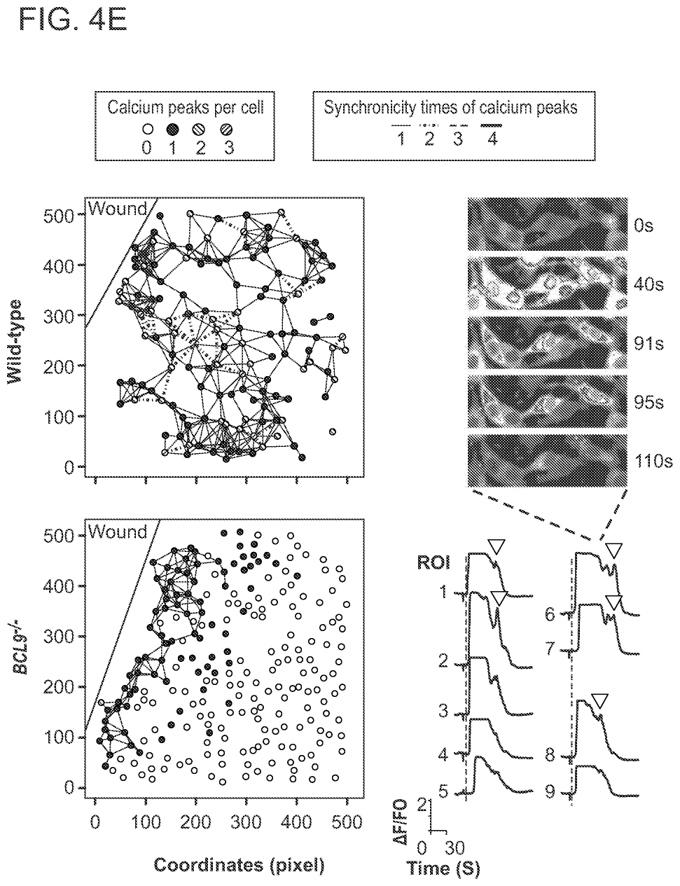

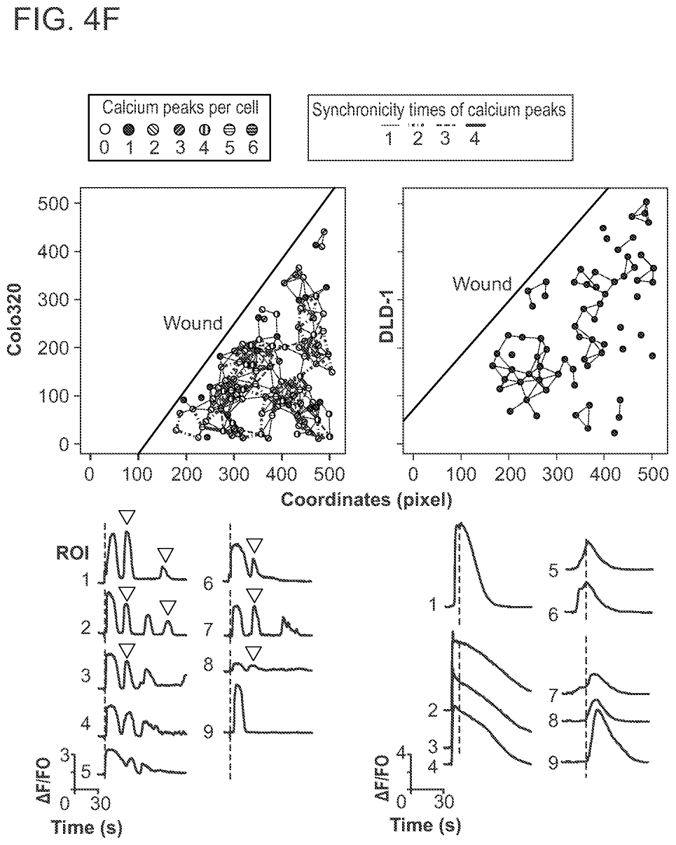

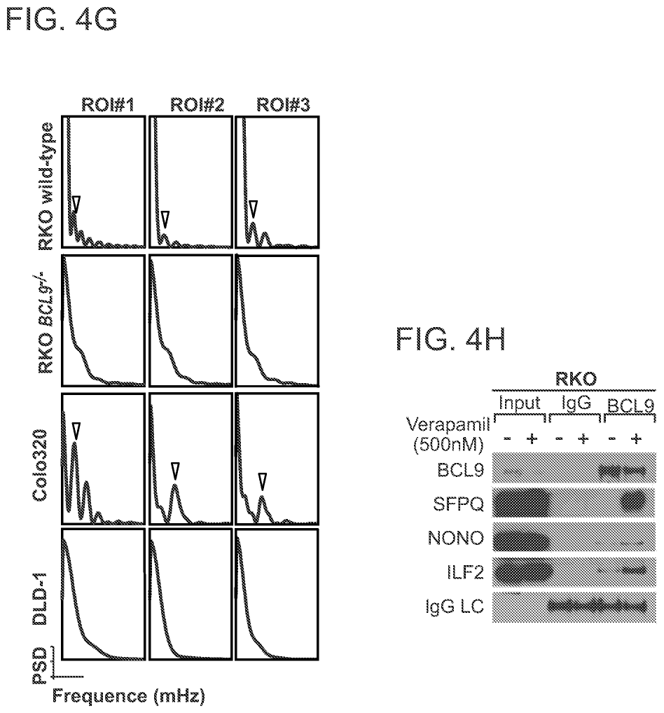

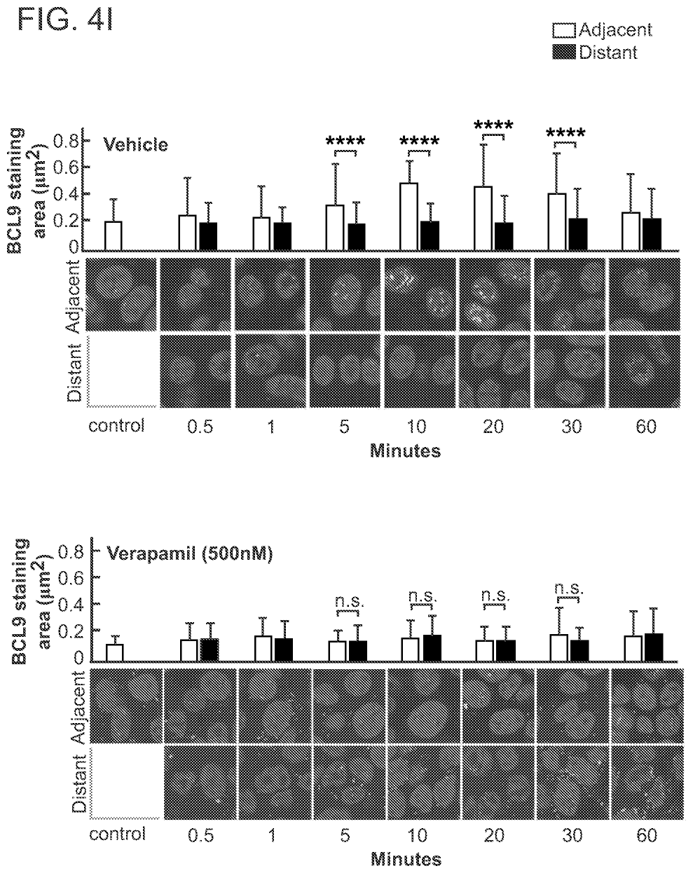

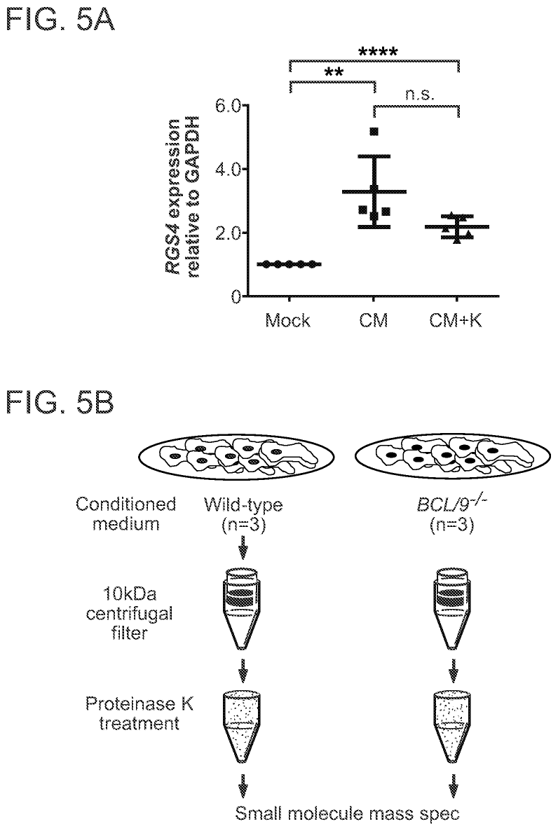

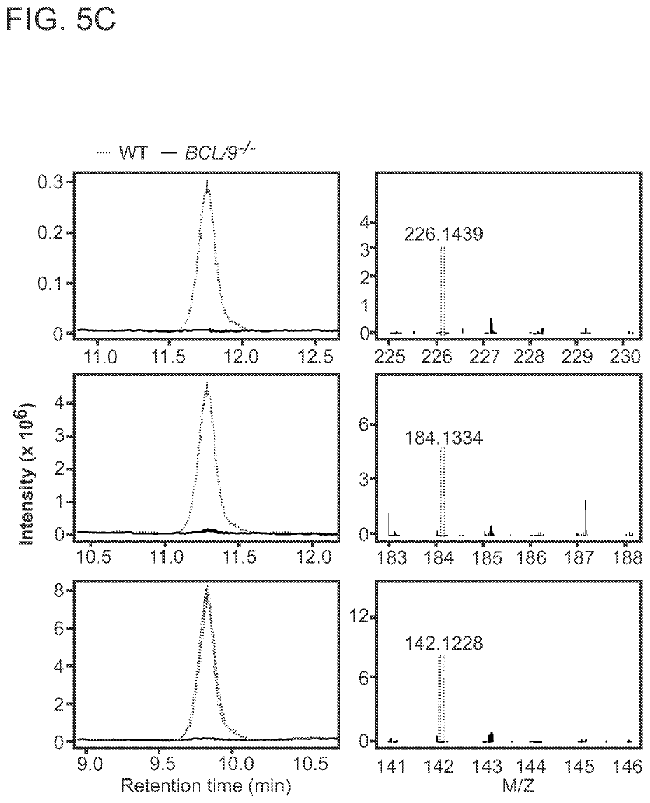

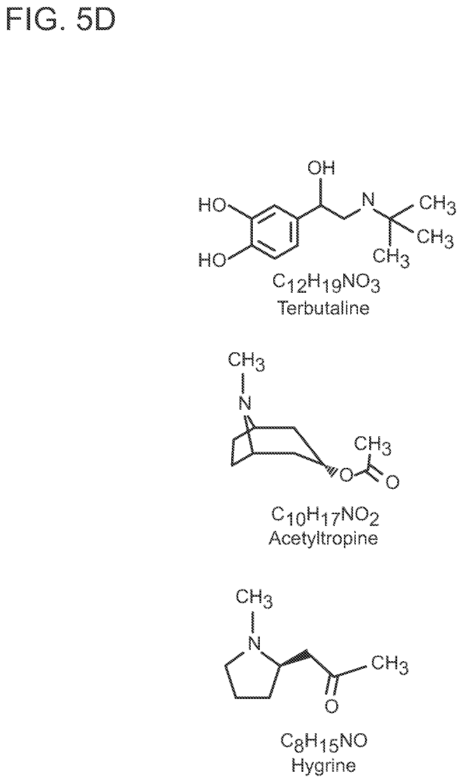

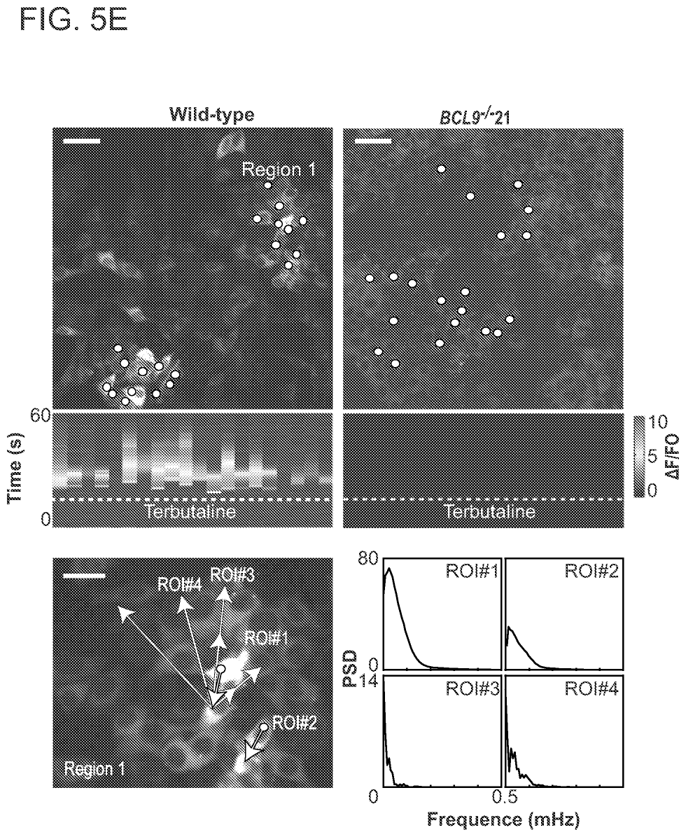

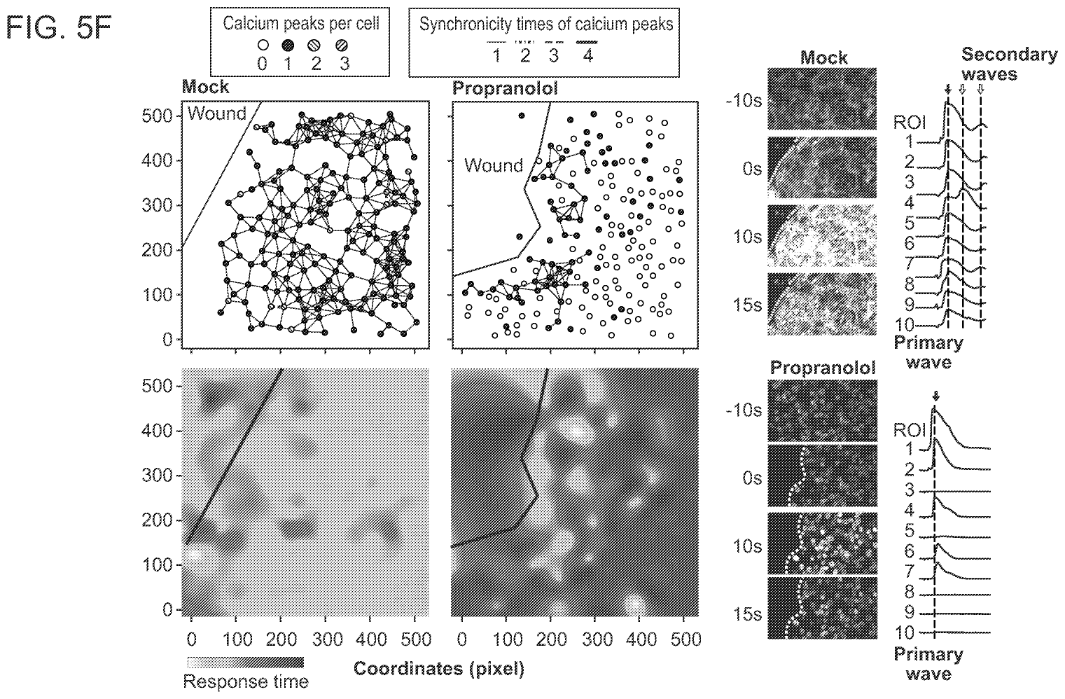

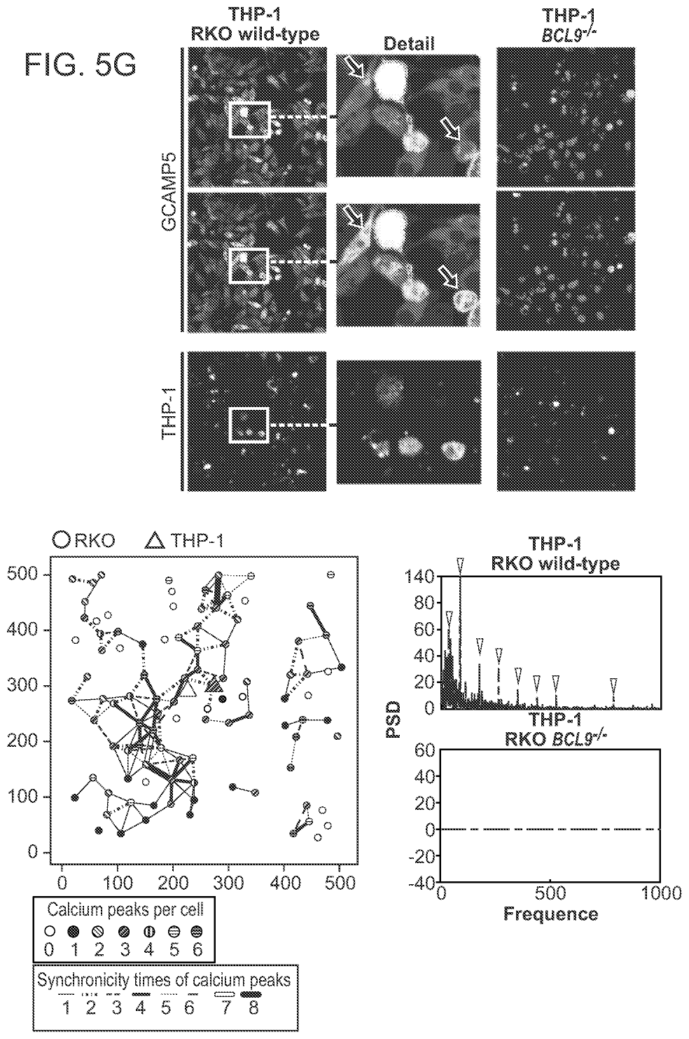

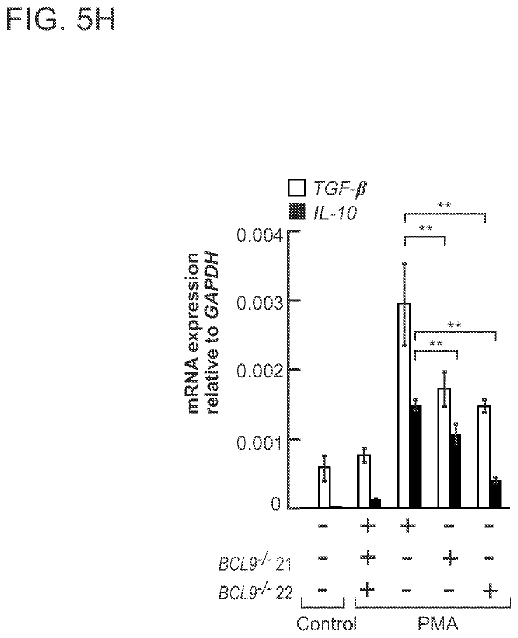

Wild-type or BCL9 knockout cells were grown in 6-well plates or Nunc™ Glass Bottom Dishes (150680, ThermoFisher®) for 24 hrs until cells reached 90% confluency. A 1 ml pipette tip was used to scratch the monolayer of cells across the center of each well. The cells were imaged at 0 and 24 hours after scratching. To investigate the effect of BCL9 on calcium waves (FIG. 5H), cells were cultured in glass-bottomed 96-well plates for 24 hrs until they reached 90% confluency. Verapamil (500 nM, V4629-1G Sigma®) or DMSO was added the cell culture medium 1 hr before scratching. A 200 μl pipette tip was used to scratch the cell monolayer from the top left to the bottom right corner of the well. At various times after the scratch was made, cells were fixed with 4% paraformaldehyde for 15 minutes at room temperature. Immunofluorescence was subsequently used to identify the location of BCL9 (see immunofluorescence method section for more detail). The wound edge in the center of the well was defined as “adjacent” and the area in the top right corner was defined as “distance” (FIG. 4H).

### In Vivo Mouse Xenograft Model

A total of 4×10⁶ wild-type or BCL9 knockout RKO cells stably transduced with a reporter expressing Luciferase were injected intraperitoneally into CB17.Cg-PrkdcscidLystbg-J/Crl (Beige) mice (n=7 per group), and tumor burden was monitored by whole-body imaging using Xenogen system every week for 4 weeks, starting one day after injection of cells. After last imaging, all mice were euthanized and all tumor nodules identified in the peritoneal cavity were dissected and processed for histological and immunohistochemical analysis. To quantify β-catenin and Ki-67 stains serial consecutive sections were scanned using Vectra 2 Intelligent Slide Analysis system; percentage of positive cells (Ki-67, CD163, CD31, or αSMA) or areas (β-catenin) were assessed using inform Cell Analysis software (PerkinElmer®). P-values were calculated using unpaired Student's t-test. Propranolol hydrochloride (Millapore-Sigma®) was added to the drinking water at a concentration of 0.5 g/L on the injection day. Drug solution was renewed every 2 days.

### Immunohistochemistry

TMA sections (from Oncology Pathology, Dana Farber Cancer Institute) were pre-heated at 65 degrees for 20 mins and then deparaffined by performing the following washing steps: xylene 3×5 mins, 100% ethanol 5 mins, 95% ethanol 5 mins, 75% ethanol 5 mins, 50% ethanol 5 mins, 25% ethanol 5 mins, and rinsed by cold water. Sections were subsequently heated in a microwave with antigen retrieval buffer (10 mM Tris Base, 1 mM EDTA Solution, 0.05% Tween 20, pH 9.0). Sections were washed twice with TBS plus 0.5% Triton X-100 (v/v) and blocked with 5% BSA for 1 hr at room temperature. Sections were incubated overnight with the following antibodies at 4 degrees: BCL9 (ab37305, Abcam®), FAP (AF3715, R&D), β-catenin (610154, BD Transduction Laboratories), SYP (PA0299, Lecia), PDGFB (ab23914, Abcam®), C3 (HPA003563, Millapore-Sigma®), RGS4 (sc-398348, Santa Cruz®), CD163 (182422, Abcam®), CD31 (77699, Cell Signaling®), αSMA (19245, Cell Signaling®), or Axin2 (#2151, Cell Signaling®). The sections were washed three times before incubation with secondary antibody for 2 hours at room temperature. The sections were then washed three times before signal amplification with HRP polymer or fluorescence. ImageJ2 software was used to carry out semi-quantitative analysis of the staining intensities, which ranged from 0 (negative) to 3 (strong staining). Subsequently the H-score was calculated using the following formula;

H-Score=(% at 0)×0+(% at 1+)×1+(% at 2+)×2+(% at 3+)×3

The intensity of staining and cell numbers were detected by imageJ2. The top 50% score of FAP was identified as “FAP high”, and the lower 50% score was identified as “FAP low”.

### Immunofluorescence and Fluorescence In Situ Hybridization

Cells were cultured on coming Glass Coverslips (354085, BioCoat) contained within the wells of a 24-well plate for 24 hrs, and then fixed using 4% paraformaldehyde. Cells were washed three times with PBS, permeabilized with 0.5% Triton X-100 (Tris-HCl pH=7.6, 150 mM NaCl, 0.5% Triton X-100 (v/v), washing buffer) for 15 minutes at room temperature, and blocked with 5% BSA for 1 hour at room temperature. The cells were incubated overnight at 4 degrees with the following antibodies; anti-BCL9 (ab37305, Abcam®), NONO (TA504777, Origene®), ILF2 (PA5-18718, ThermoFisher®) or β-catenin (610154, BD Transduction Laboratories). The following day, cells were washed 3 times with PBS and incubated with fluorescently tagged anti-mouse (A11029, ThermoFisher®), rabbit (A11035, ThermoFisher®) or goat (A21447, ThermoFisher®) secondary antibodies for 1 hour at room temperature. Cells were washed 3 times with PBS and stained with DAPI (D9542, Millipore-Sigma®) for two mins. Cells were subsequently washed three times with PBS to remove any residual DAPI stain, and then imaged with an SD confocal microscope. The colocalization threshold is calculated by ImageJ2. Dotted staining area of BCL9 was calculated by the following step: confocal images were processed by High Frequency Signaling Removal to filter out the rapidly changing signal. This process removes the noise and non-dotted staining of BCL9. Then, the top 2% signaling areas were selected. High Frequency Signaling Removal and area size was calculated by ImageJ2. NEAT1 and fluorescence probe was purchased from Biosearch Technology (NEAT1, SMF-2036-1). IF combined FISH of BCL9/NEAT1 was performed as recommended by the supplier.

### Quantitative Real-Time Polymerase Chain Reaction

RNA was extracted from cells or DynaBeads by using Trizol solution and converted to complementary DNA using a High Capacity cDNA Reverse Transcription Kit (4368814, ThermoFisher®). Quantitative PCR was carried out using SYBR Green Master Mix (4309155, ThermoFisher®) and the results were normalized to GAPDH expression. A list of PCR primers are shown in Table 1.

TABLE 1
List of PCR Primers.

| | |
|---|---|
| CACNA2D1 | Forward: CTGACGGTCCAAATCCTTGT |
| | (SEQ ID NO: 36) |
| | Reverse: TGCCAGATACCAGCCAAAGT |
| | (SEQ ID NO: 37) |
| RGS4 | Forward: CCAGAGAGTGAGCCAAGAGG |
| | (SEQ ID NO: 38) |
| | Reverse: ATCTTTTTGGCCTTGGGACT |
| | (SEQ ID NO: 39) |
| HGF | Forward: CATGTCCTCCTGCATCTCCT |
| | (SEQ ID NO: 40) |
| | Reverse: AGCCTTGCAAGTGAATGGAA |
| | (SEQ ID NO: 41) |
| SEMA3D | Forward: GCATCGAGAGGAGTTGAAGC |
| | (SEQ ID NO: 42) |
| | Reverse: GCCCTCTGGGTATTTTCCAT |
| | (SEQ ID NO: 43) |
| TGF-β2 | Forward: ATTGCTGCCTACGTCCACTT |
| | (SEQ ID NO: 44) |
| | Reverse: TTGGGTGTTTTGCCAATGTA |
| | (SEQ ID NO: 45) |
| IL-10 | Forward: TCCCTGTGAAAACAAGAGCA |
| | (SEQ ID NO: 46) |
| | Reverse: ATAGAGTCGCCACCCTGATG |
| | (SEQ ID NO: 47) |
| NEAT1 | Forward: GATCTTTTCCACCCCAAGAG |
| | TACATAA (SEQ ID NO: 48) |
| | Reverse: CTCACACAAACACAGATTCC |
| | ACAAC (SEQ ID NO: 49) |

### Immunoblotting

Cells were lysed using RIPA Buffer (50 mM Tris-HCl pH 7.5, 150 mM NaCl, 1 mM EDTA, 1% NP-40, 0.1% SDS, 0.5% sodium deoxycholate) with addition of protease and phosphatase inhibitor (#5872, Cell Signaling®). Following lysis, cells were incubated on ice for 10 minutes and centrifuged for 10 minutes at 4 degrees at 16000×g. 40 μg of each protein lysate was electrophoresed using SDS-PAGE, transferred to nitrocellulose membrane, blocked with 5% nonfat milk, and incubated overnight with following antibodies; anti-BCL9 (ab37305, Abcam®), NONO (TA504777, Origene®), SFPQ (MA1-25325, ThermoFisher®), ILF2(PA5-18718, ThermoFisher®), TLR3 (ab62566, Abcam®), β-catenin (610154, BD Transduction Laboratories), CD44 (#3570, Cell Signaling®), Flag tag (A8592, Millipore-Sigma®), Axin2 (#2151, Cell Signaling®), p65 (#8242, Cell Signaling®), LaminB1 (sc-6216, Santa Cruz®) and GAPDH (ab9485, Abcam®). Secondary antibodies conjugated to horseradish peroxidase were purchased from Santa Cruz Biotechnology (sc2020, Santa Cruz®) and Cell Signaling (#7074, #7076, Cell Signaling®).

### Reporter Assay

Cells were co-transfected with shLacZ, shBCL9 and shILF2 plasmids, and subsequently transfected with NFAT luciferase reporter (Plasmid #10959, Addgene®) and SV40 drive renilla luciferase reporter (E2231, Promega®). 24 hrs post-transfection, 1×10⁵ cells were plated per well in 96-well plates. 24 hours after seeding, the Dual-Luciferase Reporter Assay System (Promega®) was used to measure luminescence according to the manufacturer's instructions. The luciferase signal from the NFAT reporter was normalized to the luciferase signal from the renilla reporter.

shRNA, siRNA, and ORF Expression

The shNONO-1, shNONO-2, shILF2-1 and shILF2-2 plasmids were purchased from Sigma®. shBCL9, shLacZ and the BCL9 expression ORF were obtained from Ruben Carrasco's lab, and the shRNA sequences are noted in Key resource table. A plasmid was used to express BCL9 ORF. RGS4 and CACNA2D1 siRNAs were purchased from Sigma® (siRGS4 #1 SAS1_Hs02_00323157, siRGS4 #2 SAS1_Hs02_00323155, siCACNA2D1 #1 SAS1_Hs02_00303191, siCACNA2D1 #2 SAS1_Hs01_00135469), and siCACNA2D1 #2 SAS1_Hs02_00303142). Lentivirus was produced in 293T cells using the three-vector system. Virus was diluted 1:1 to culture medium and added to cells in a T25 flask containing 1:1000 (v/v) polybrene (sc134220, Santa Cruz®). 10 μg/ml puromycin was used after 48 hrs infection to screen infected cells. siRNA was transfected by lipofectamine 2000 (ThermoFisher®, 11668030).

### Small Molecular Mass Spec Analysis

Cells were cultured in 6-well plates for 24 hrs in antibiotic free medium with 10% FBS. The cell culture medium was collected and purified using a 10 kd filter (MRCPRT010, Microcon-10 kDa Centrifugal Filter Unit). In addition, the medium was incubated with proteinase K (P6556, Sigma®) for 1 h at 37 degrees. Samples were sent for MS/MS Mass spec analysis at the Small Molecule Mass Spectrometry Facility, Harvard University.

### Quantification and Analysis of Calcium Imaging Data

To trace the calcium transient in real time, GCaMP5G (#31788, Addgene®) was transfected into wild-type or BCL9 knockout RKO and Colo320 cell lines. 2 mg/ml neomycin was added to the cell culture medium 48 hrs after transfection to generate cell lines with stable GCaMP5 expression and removed one week prior to the initiation of experiment. Calcium transients were captured by live-cell imaging using a confocal microscope. Time lapse imaging was setup so that an image was captured every 0.5 s for 30 minutes, with an exposure time of 0.5 s. Calcium measurements were normalized to background fluorescence and the relative change in calcium (F(t)) over time was calculated by the formula:

$$F ( t ) = \frac{F - {F}_{0}}{{F}_{0}} = \frac{Δ F}{{F}_{0}}$$

where F0 was defined as the average intensity of the 20% lowest grey values in a region of interest (ROI), F was defined as the normalized grey values of ROI in time point t. Global synchronicity was calculated by cross correlation method in FluoroSNNAP (seas.upenn.edu/˜molneuro/fluorosnnap.html). The frequency spectrum was calculated by fast Fourier transform (Uhlen, P. (2004). Sci. STKE 2004, p 115), the time difference between each sample is 0.5 s.

### Statistical Analysis

Statistical significance was evaluated in GraphPad Prism using the unpaired Student's t-test. A P-value≤0.05 was considered statistically significant.

### Example 2: BCL9 Expression is Negatively Correlated with Patient Survival in a CRC Subtype Characterized by Stromal Cell Infiltration and Expression of Neural-Associated Genes

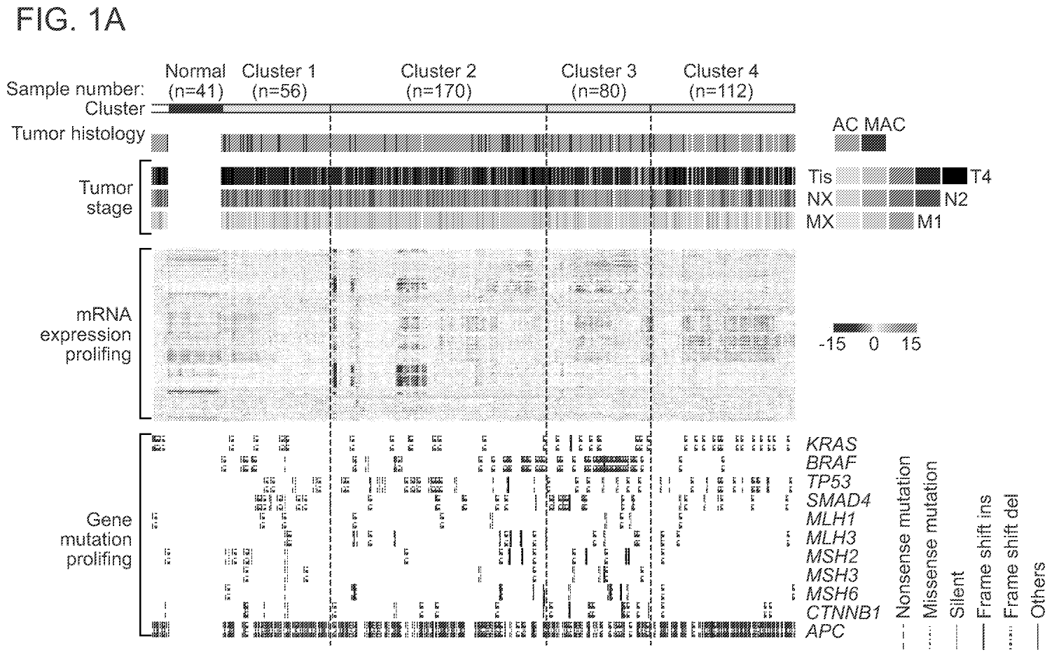

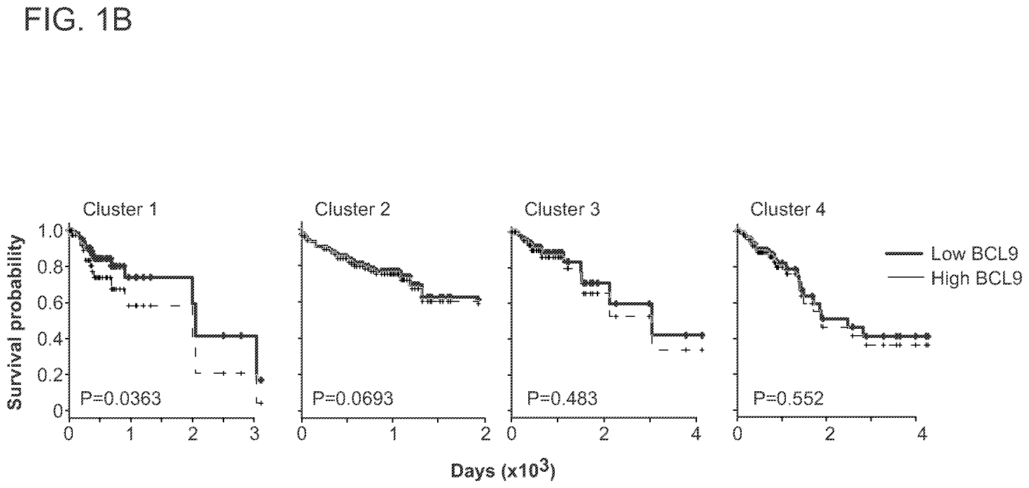

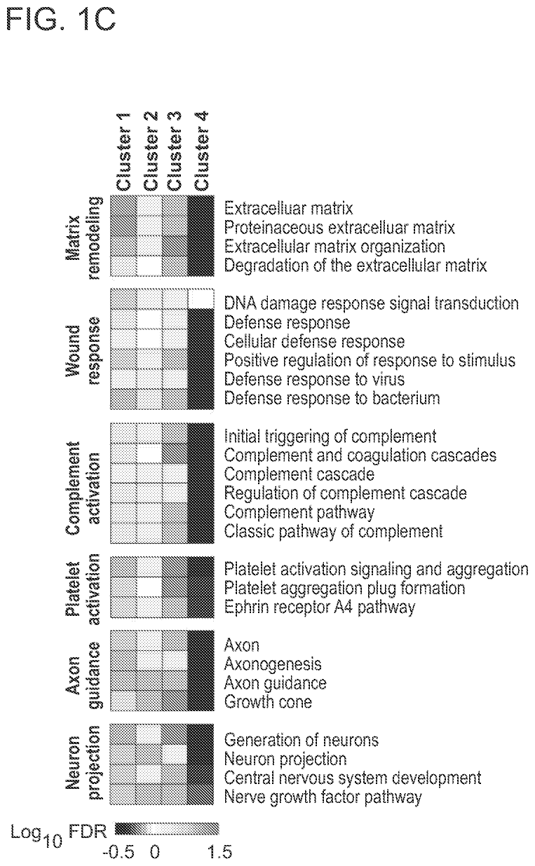

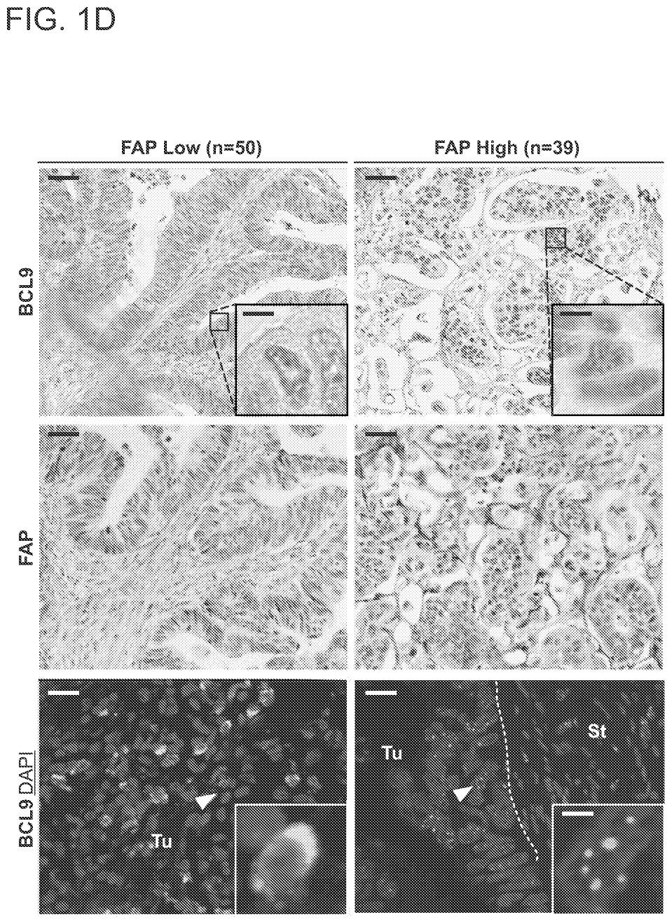

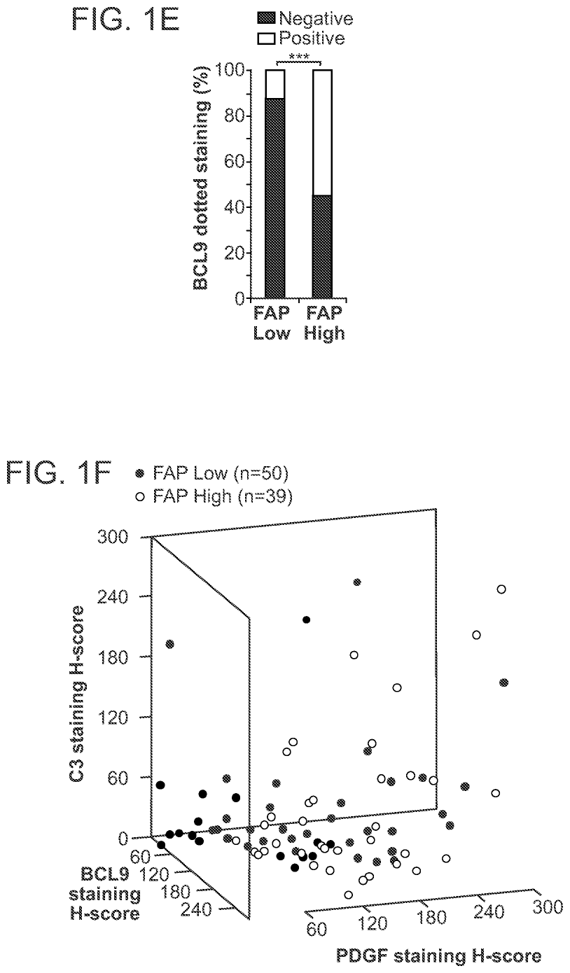

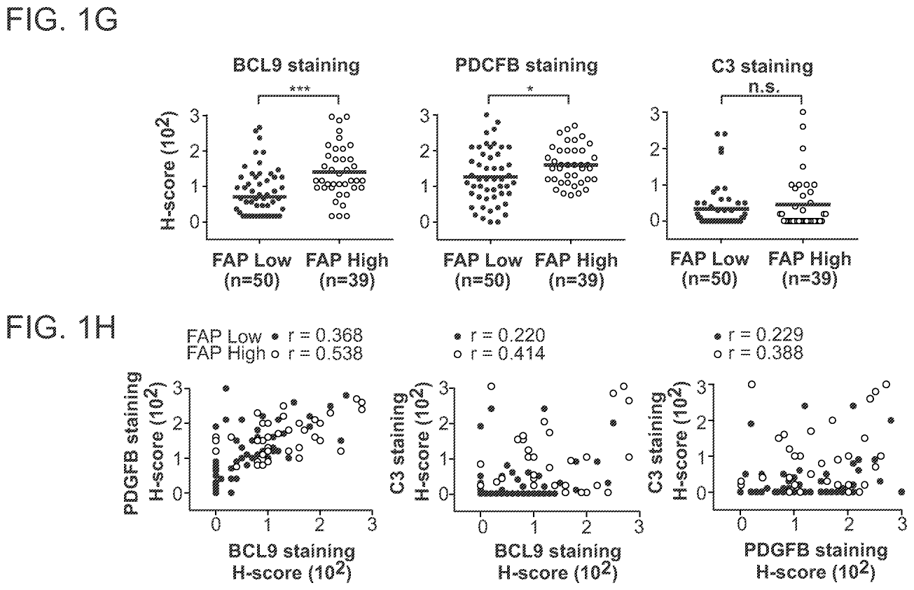

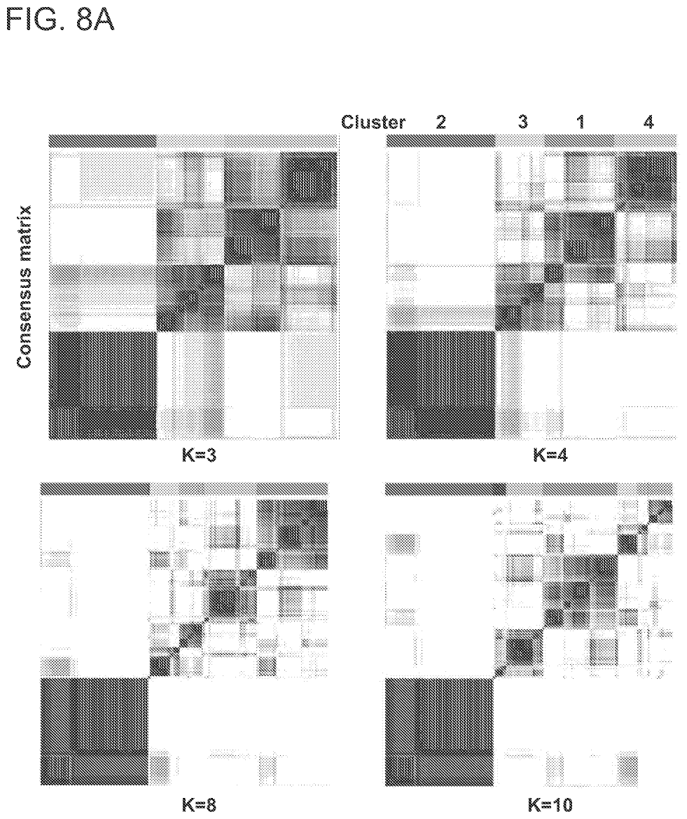

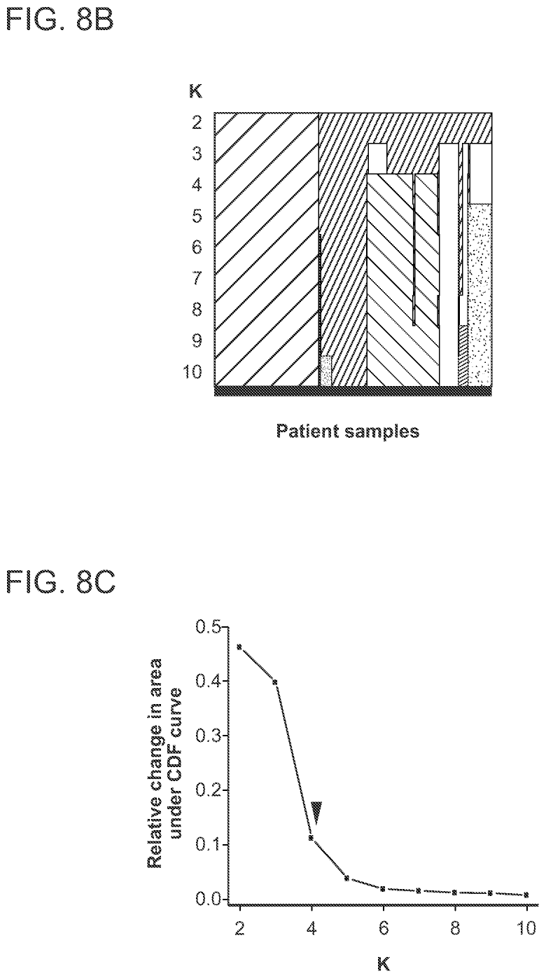

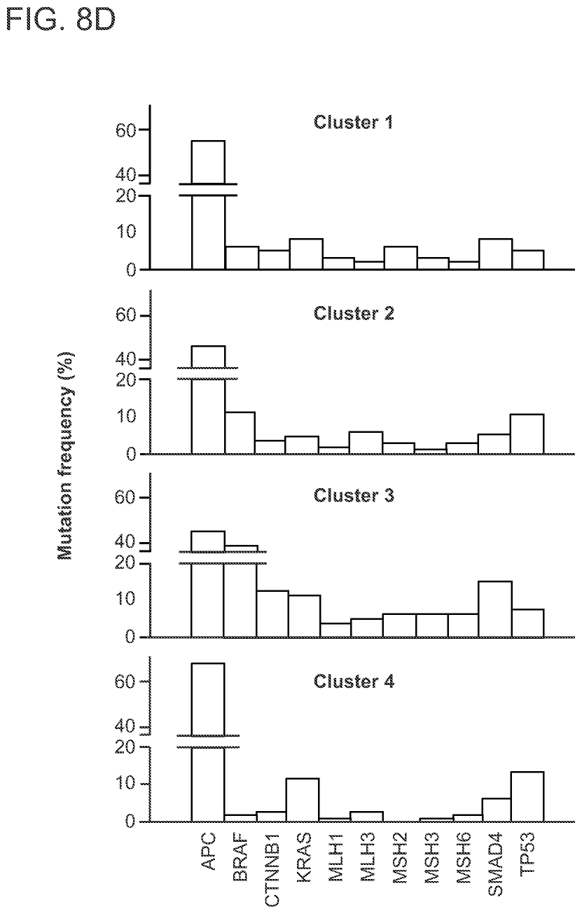

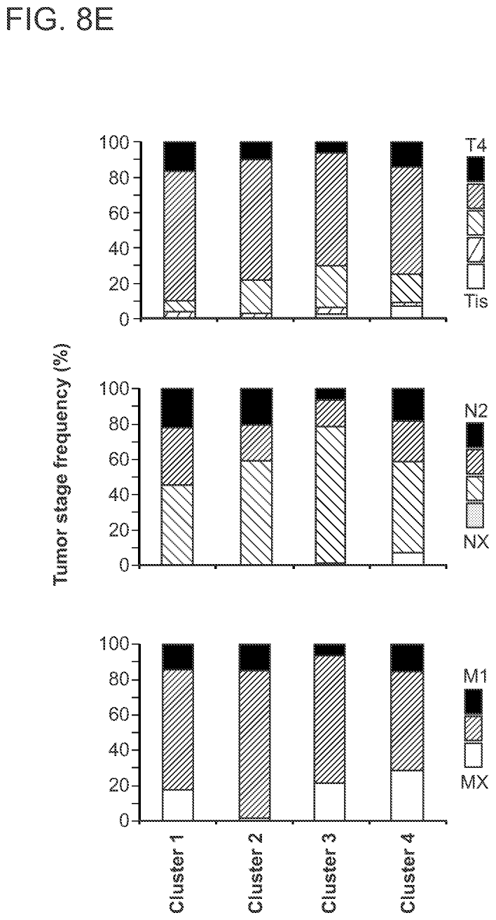

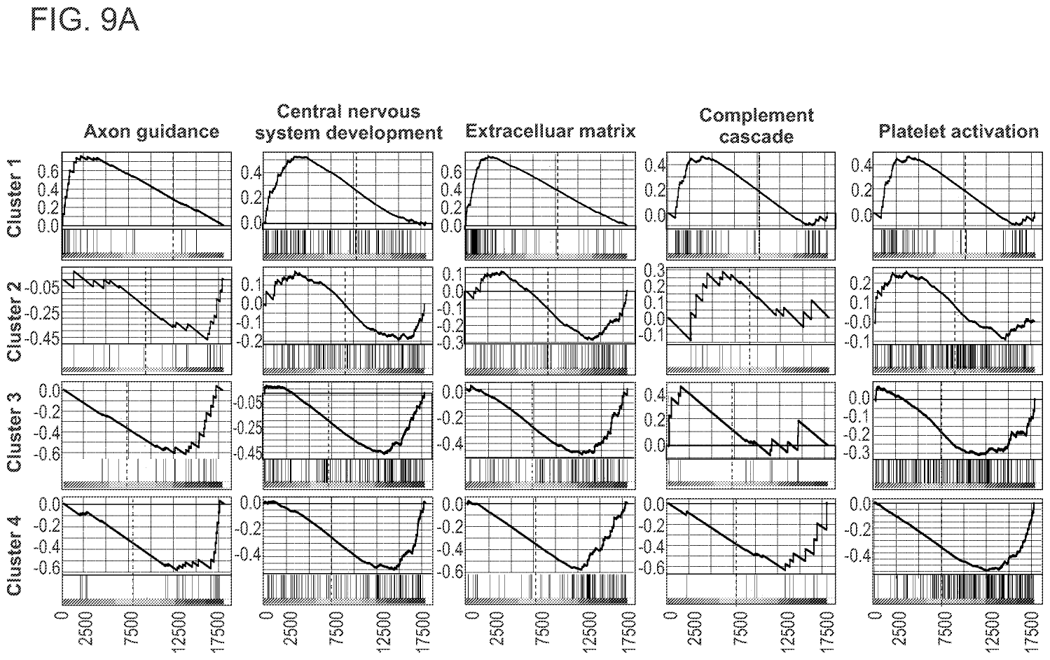

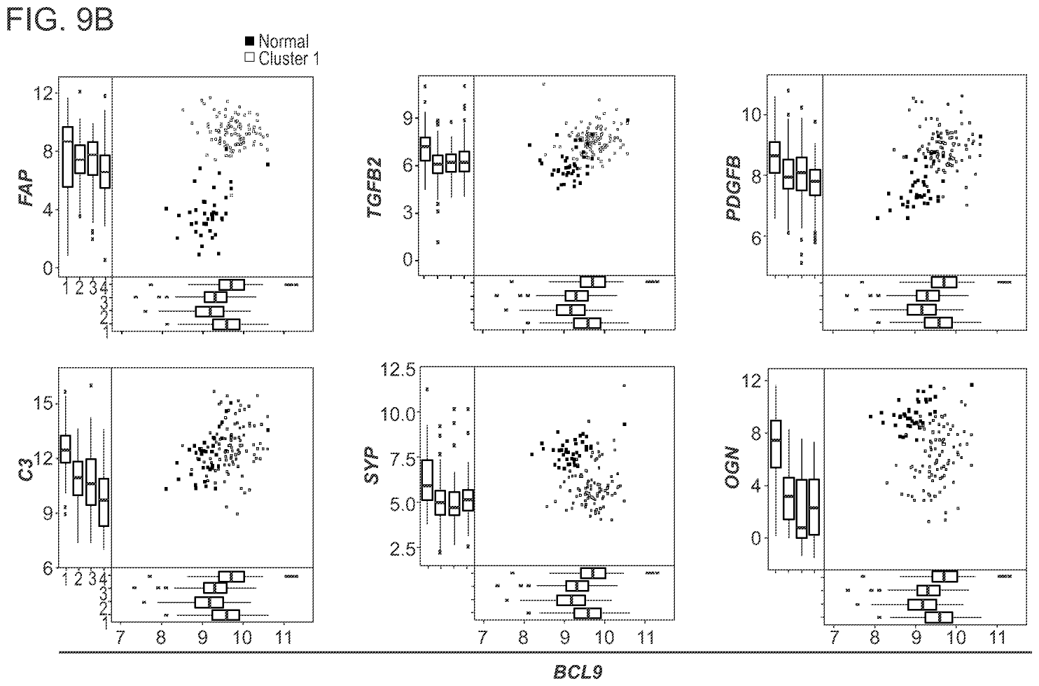

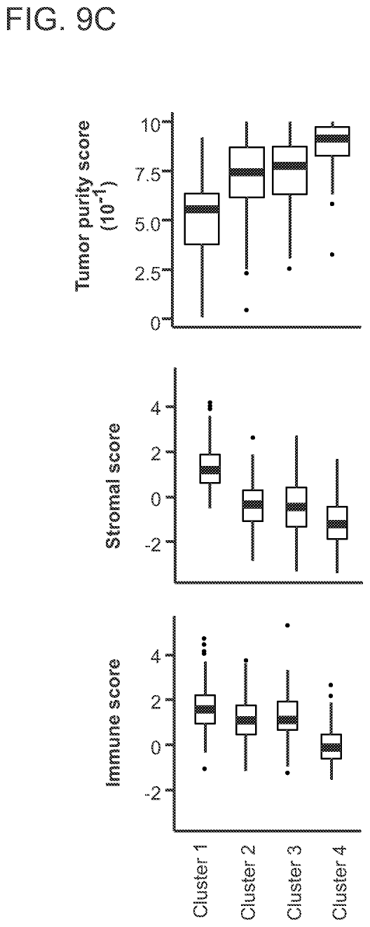

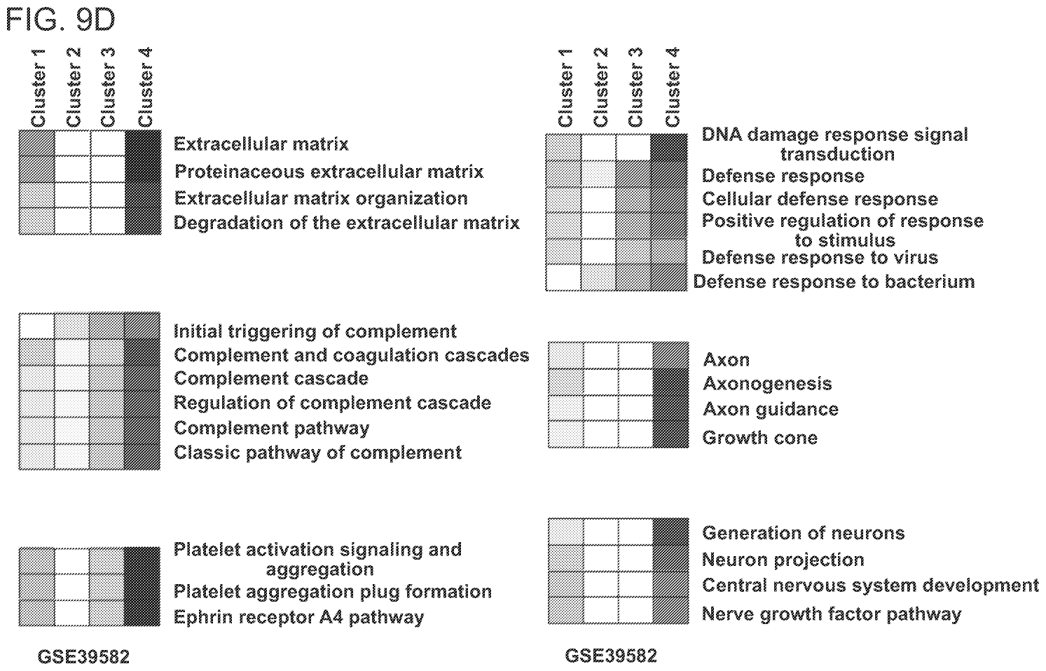

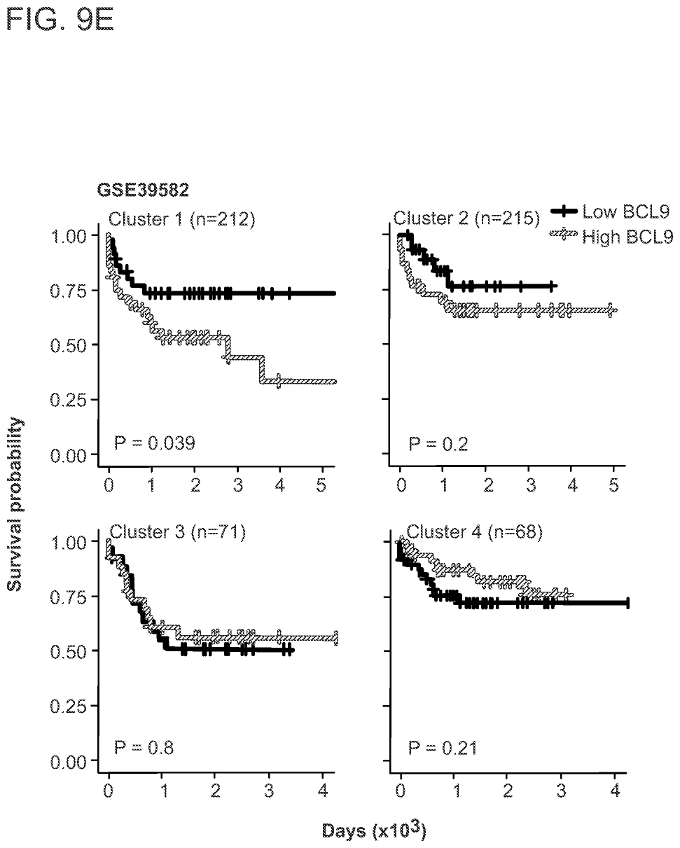

The gene expression profile of a cell reflects its type, state, and biological behavior, with similarities in expression profiles representing similarities in the biology of samples (Brown et al., (2000) Proc. Natl. Acad. Sci. USA, 97:262-267; Cheng et al., (2000) Proc. Int. Conf. Intell Syst. Mol. Biol. 8:93-103). Unsupervised consensus clustering was performed (De Sousa et al., (2013), Nat. Med. 19, 614-618; Dienstmann et al., (2017), Nat. Rev. Cancer 17, 79-92) on the RNA-Seq-based gene expression datasets from The Cancer Genome Atlas for CRC and normal colon epithelial cells to reduce the challenges of tumor cell heterogeneity and provide a relatively pure biological context to investigate oncogene function of BCL9. Among the 418 CRC samples analyzed, up to four distinct molecular clusters (C1-C4) were found (FIG. 8A-FIG. 8C), each characterized by unique gene expression but not histologic, clinic, or genetic profiles (FIG. 1A, FIG. 8D, and FIG. 8E). C1, which comprised 13% (56/418) of the cases, was the only cluster showing significantly lower clinical outcome in samples with high BCL9 expression (FIG. 1B). Detailed analysis of this cluster by gene set enrichment analysis (GSEA) revealed upregulation of gene sets associated with wound healing, tissue remodeling, and neuron projection such as FAP, PDGFB, C3, and SYP compared to other clusters (FIG. 1C, FIG. 9A, and FIG. 9B). Estimate analysis of tumor purity revealed that C1 is the most heterogeneous by cellular composition, and that tumors within this group were infiltrated by stromal but not by immune cells (FIG. 9C). This was validated through the observation of another microarray gene set (GSE39582). Similarly to the analysis results of TCGA, only one cluster showed significantly lower survival in samples with high BCL9 expression, and this cluster displayed enrichment of stromal cell and neural associated genes (FIG. 9D and FIG. 9E).

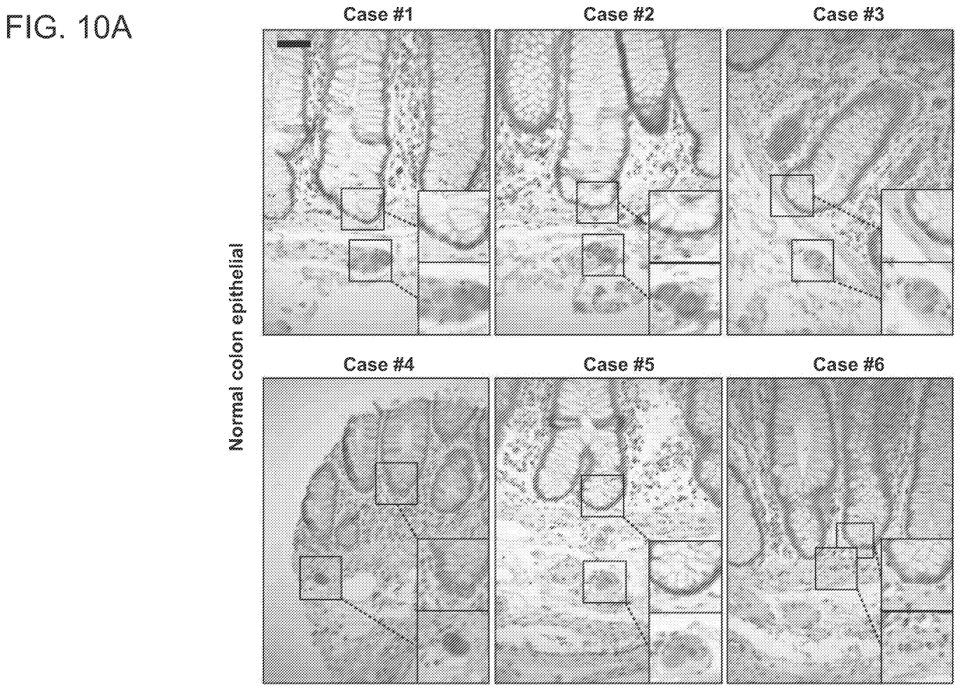

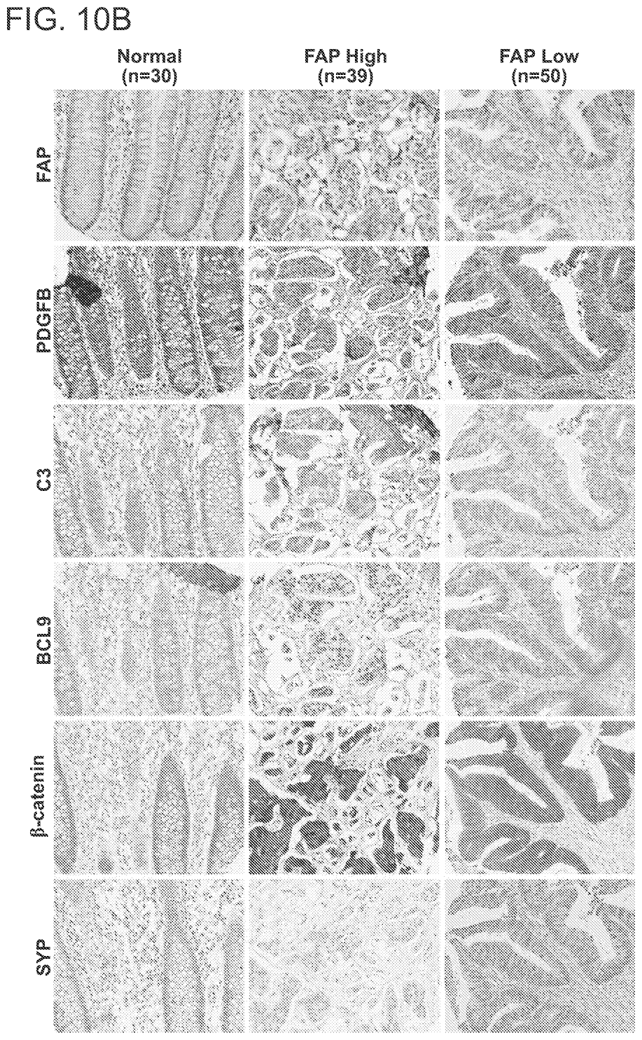

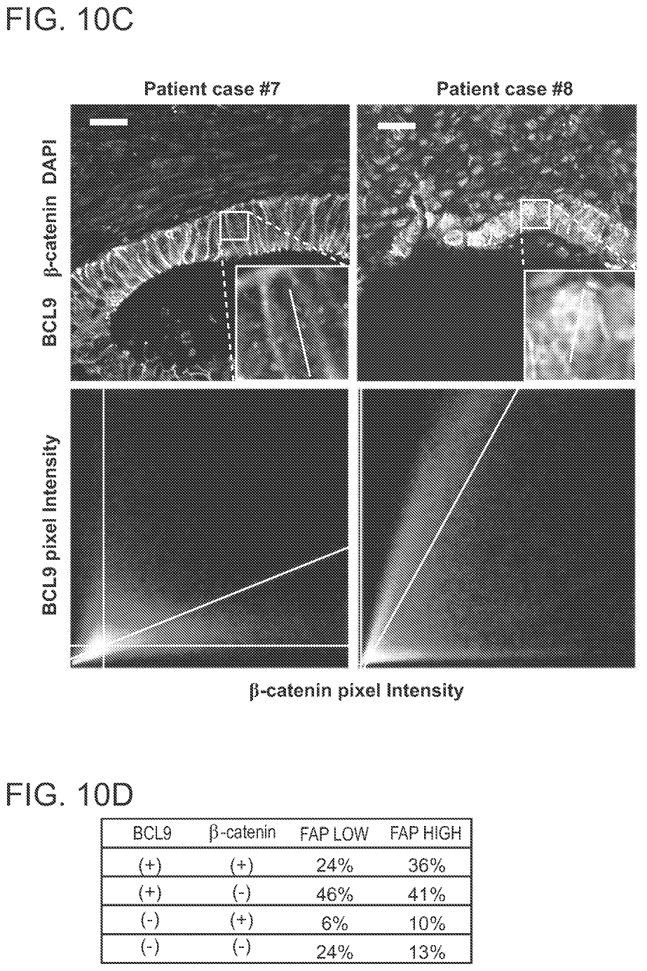

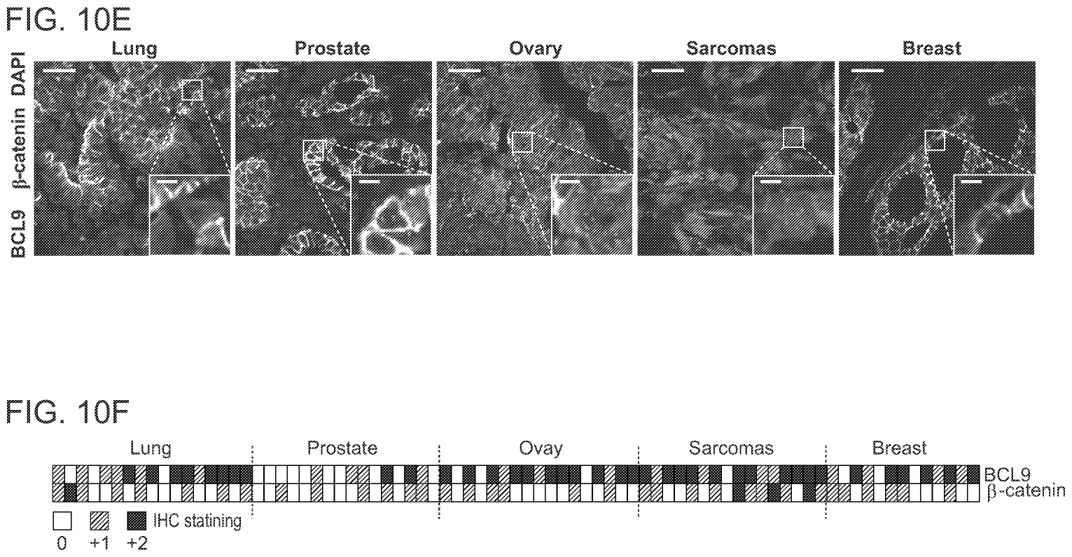

Immunohistochemistry (IHC) on tissue microarray (TMA) (n=89) was performed to investigate the histologic pattern of BCL9 expression and C1-featured probes using FAP as a marker of stromal cell infiltration (Tyulkina et al., (2016), Dokl Biochem Biophys 470, 319-321). In normal colon mucosa, the highest levels of BCL9 staining were detected in stromal and ganglion cells as compared with epithelial cells (FIG. 10A). In tumors however, BCL9 expression was detected in malignant epithelium, and higher correlation with PDGFB and C3 was observed in high FAP-expressing groups rather than low FAP groups (FIG. 1D-FIG. 1H and FIG. 10B). BCL9 frequently displayed a punctate pattern of nuclear staining in the former group (FIG. 1E), which was observed also in a subset of other cancer types and very remarkably was not correlated with β-catenin staining (FIG. 10C-FIG. 10F). These results suggest that BCL9 may play a role in the progression of C1, a CRC molecular subtype characterized by stromal cell infiltration and a punctate pattern of nuclear BCL9 staining, which occurs independently of β-catenin activation.

### Example 3: BCL9 Regulates Expression of Neural-Associated Genes and Plays Key Biological Roles in Patient's Survival of C1 Cluster Subtype

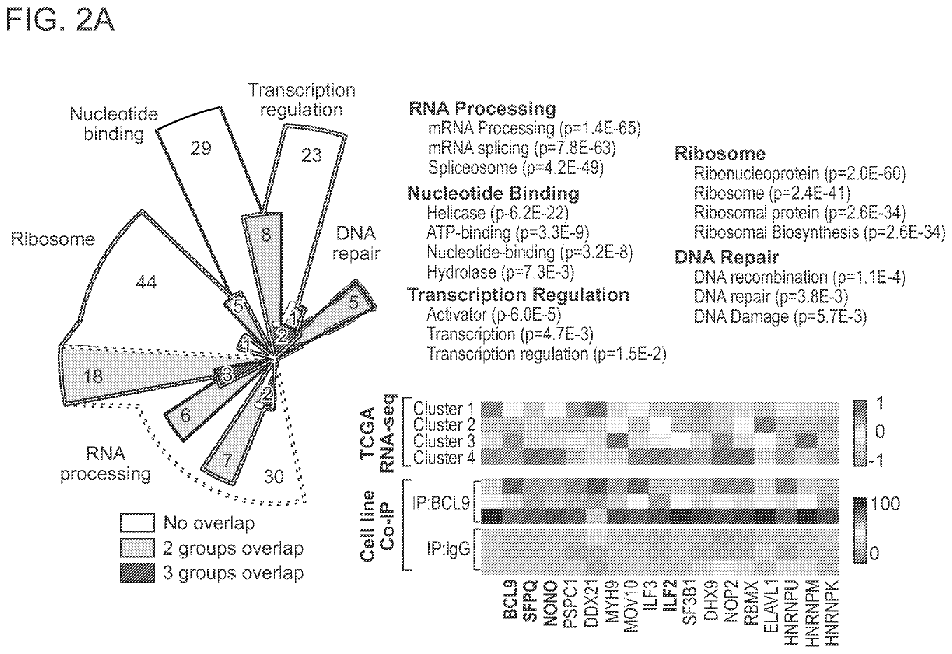

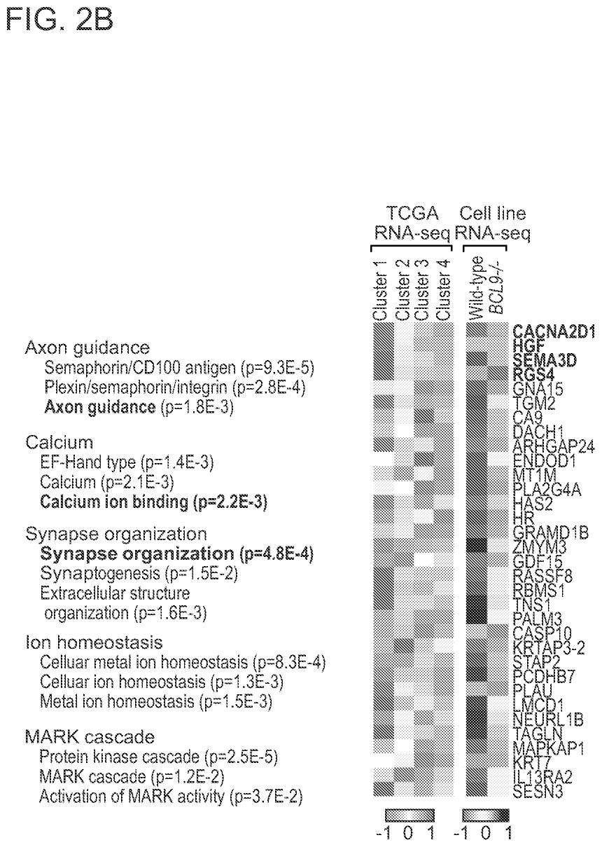

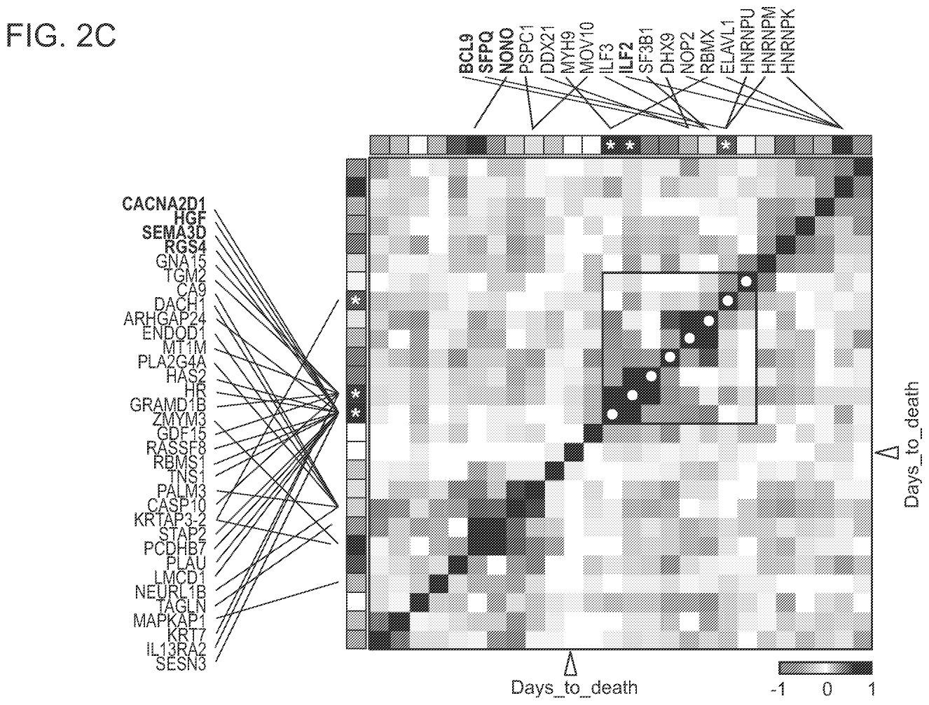

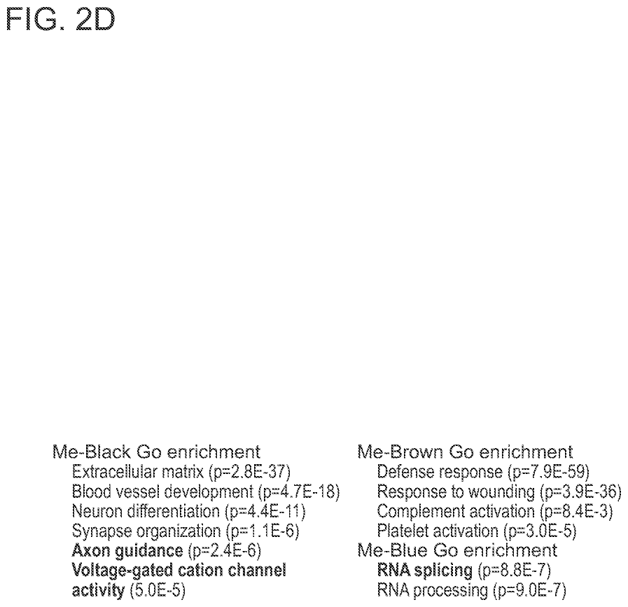

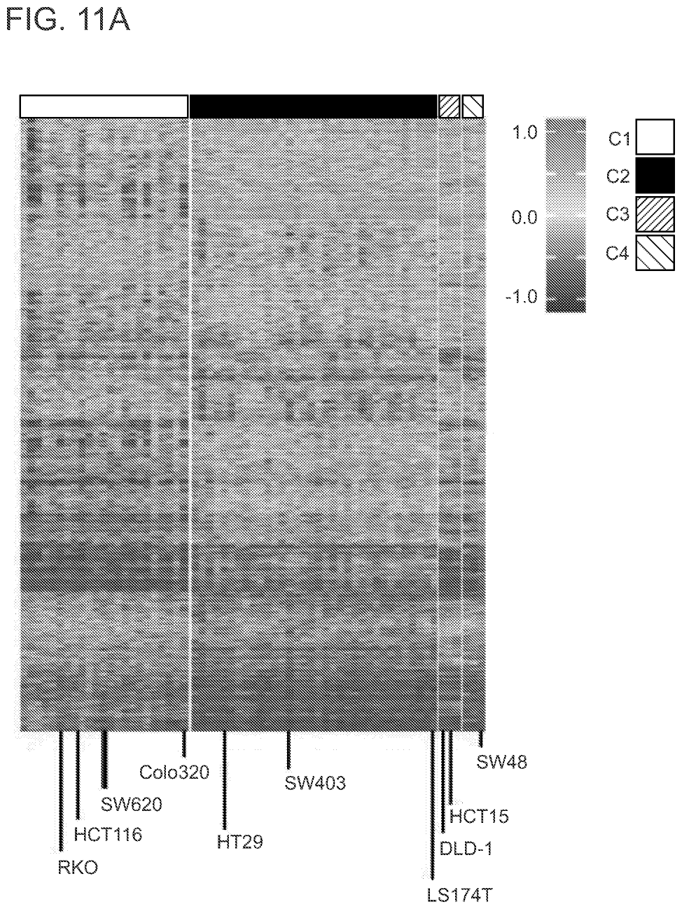

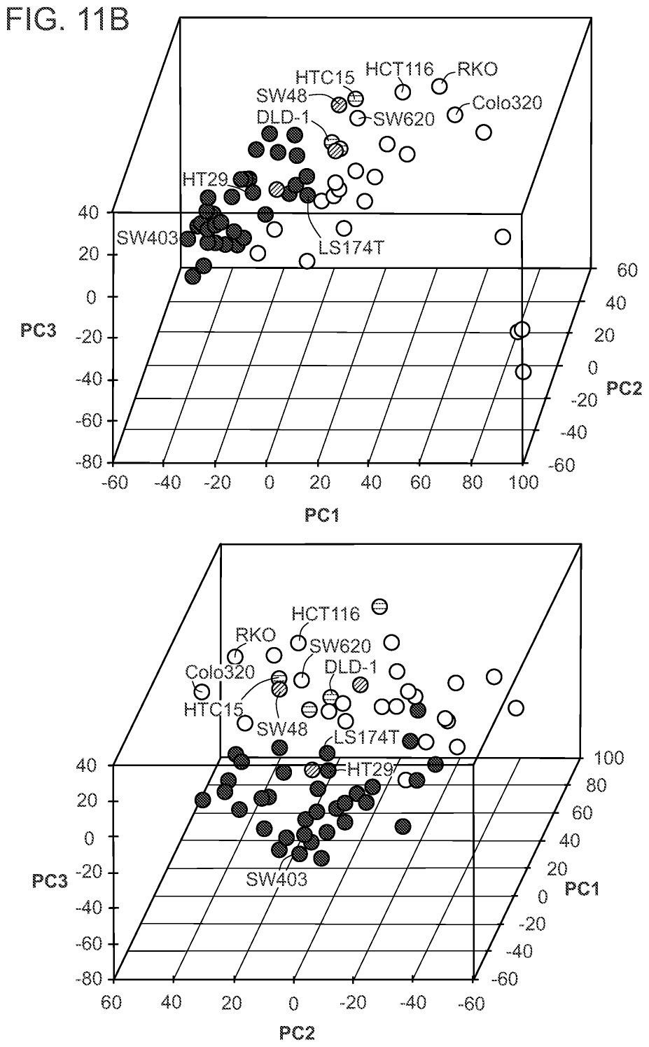

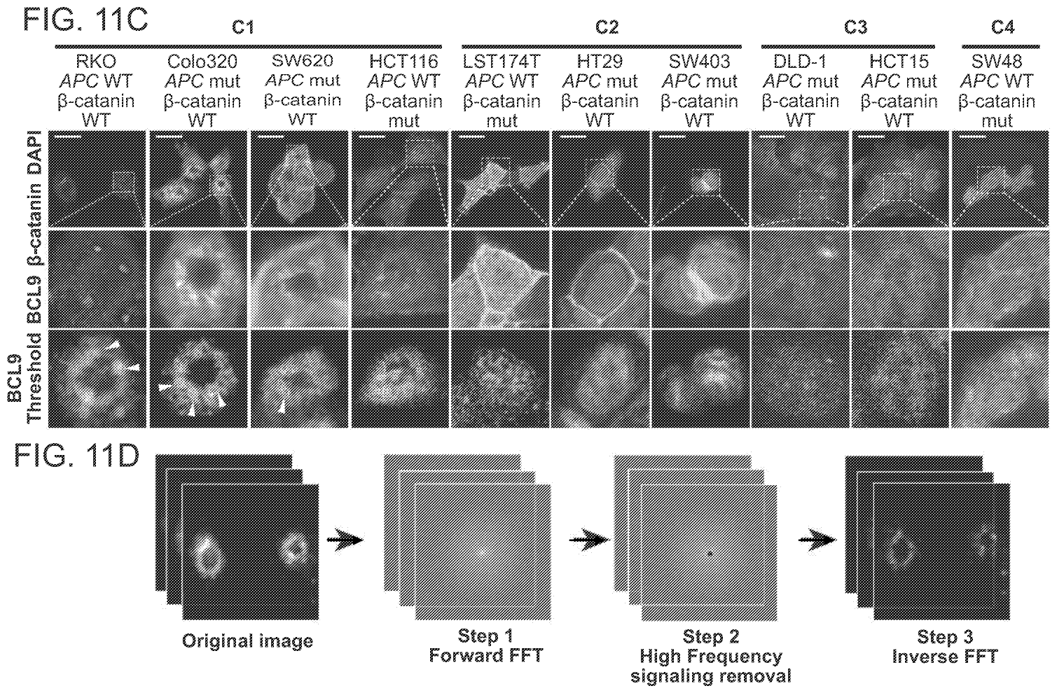

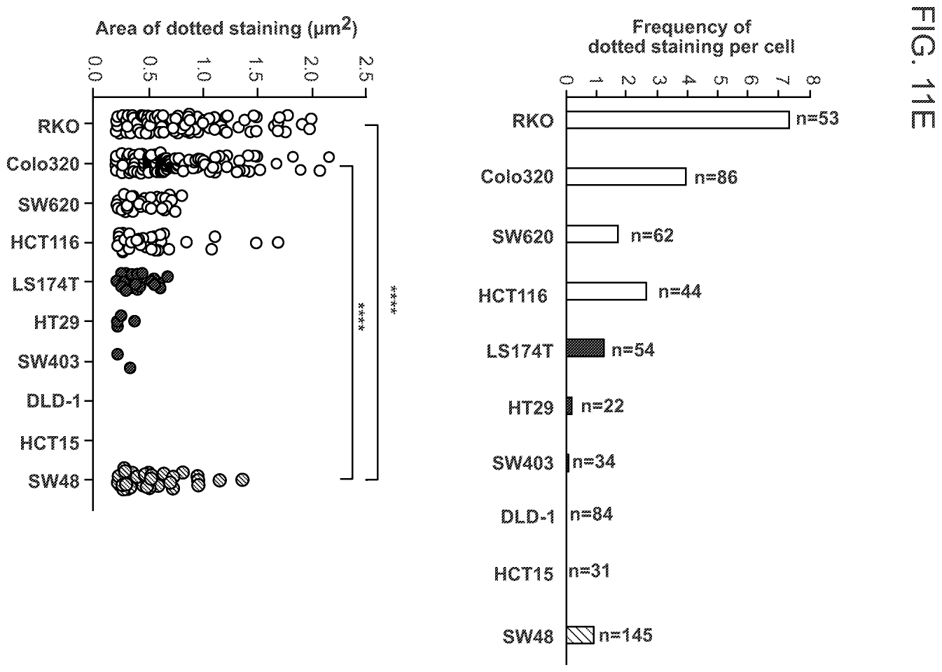

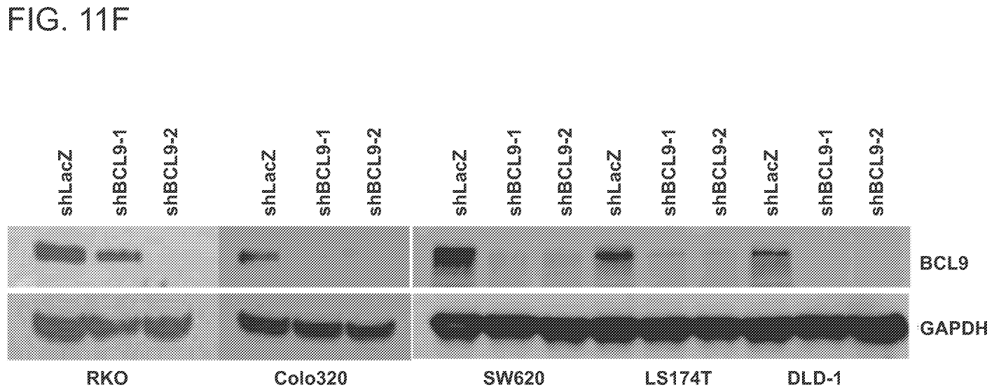

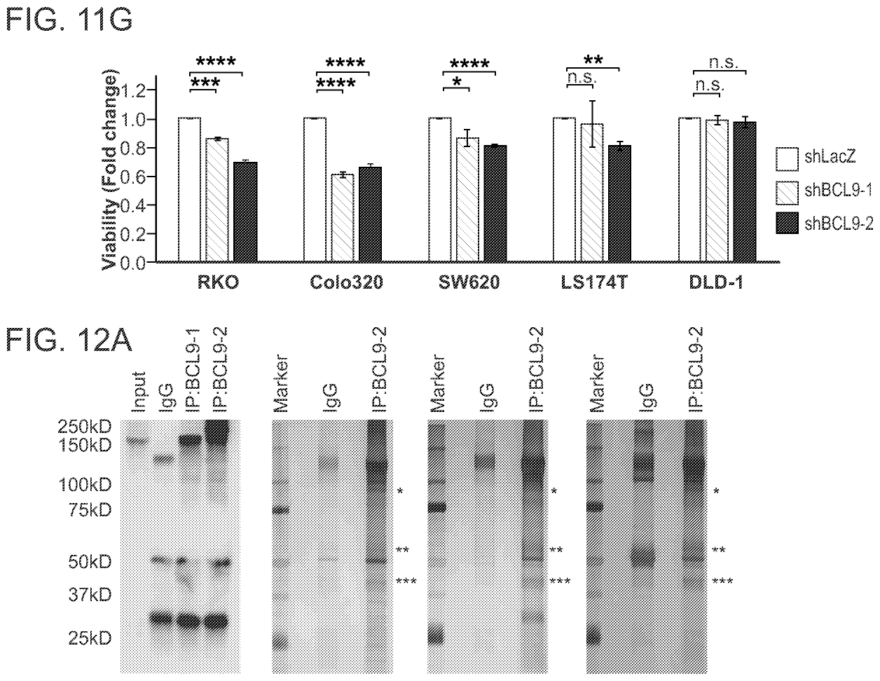

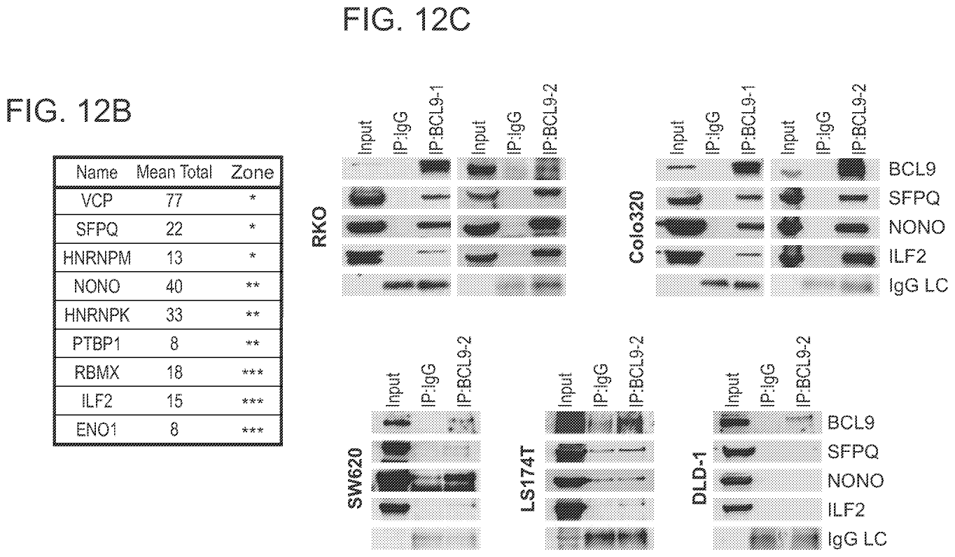

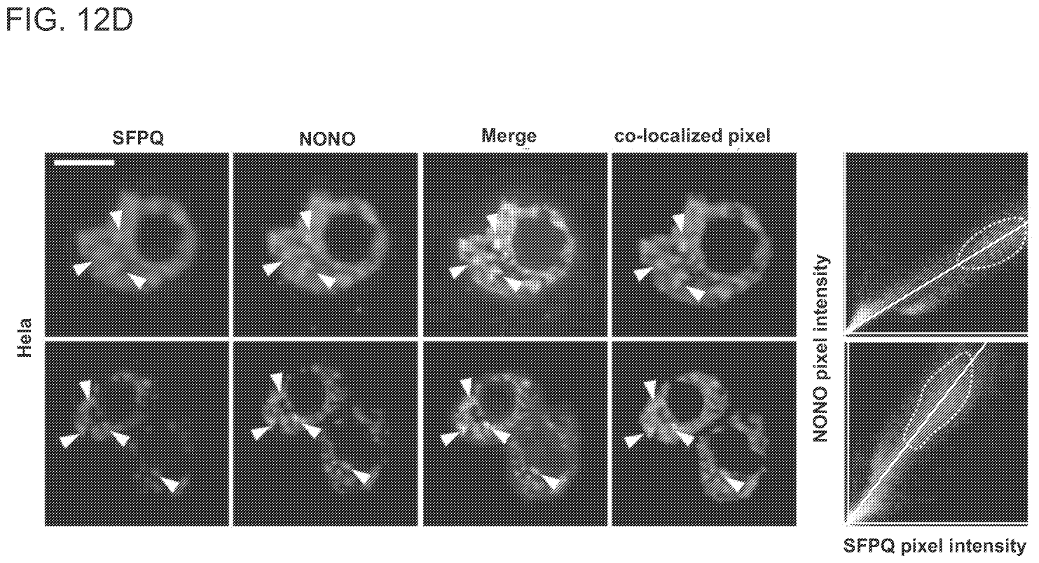

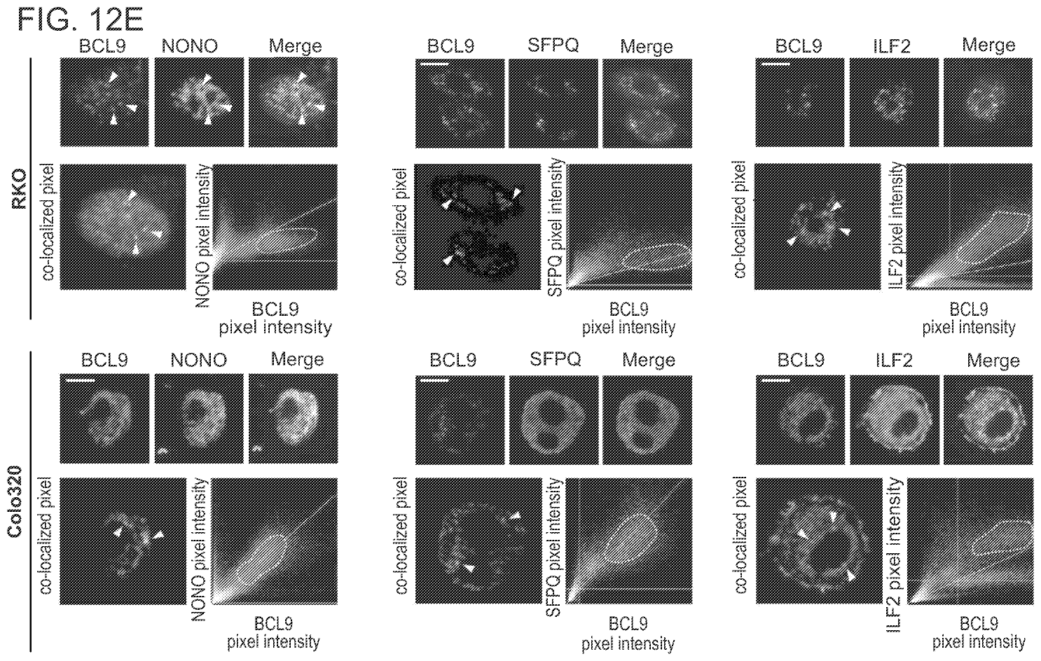

The gene expression data of 63 CRC cell lines were merged with the CRC patients' samples to identify representative cellular models of C1, and then a consensus clustering was performed to estimate the similarity of gene expression profiling between them (FIG. 11A). The results revealed that a well-validated BCL9 dependent cell line, Colo320, displayed a similar transcriptional profile to the C1 patient samples (FIG. 11B) (Mani et al., (2009) Cancer Res. 69:7577-7586). Additionally, three other cell lines (RKO, SW620, and HCT116) were identified, which also belong to C1. Other cell lines belonging to other clusters were also used to perform IF. To this end, Colo320 and RKO cell lines displayed the most obvious dotted staining of BCL9 (FIG. 11C-FIG. 11E). In addition, RKO and Colo320 cell lines showed the largest decrease in survival among all cell lines analyzed after shBCL9-induced knockdown (FIG. 11F and FIG. 11G). Therefore, these cell lines were chosen for immunoprecipitation (IP) experiments combined with total protein mass spectrometry (MS) to identify BCL9 interacting proteins related to C1, and to better understand the functional consequences of the dotted nuclear staining. Two hundred and fifty proteins were pulled down by anti-BCL9 specific antibodies but not by normal rabbit IgG. GSEA revealed these proteins were involved in RNA splicing/processing, transcription regulation, DNA repair, nucleotide binding, and ribosome function (FIG. 2A). The binding results were further validated by IP experiments using two different anti-BCL9 antibodies, along with MS of protein bands recovered from silver-stained gels (FIG. 12A and FIG. 12B), in which Valosin Containing Protein (VCP), Non-POU Domain Containing Octamer Binding (NONO), Splicing Factor Proline and Glutamine Rich (SFPQ), and Interleukin Enhancer Binding Factor 2 (ILF2) displayed the greatest number of peptides. In agreement with the size of BCL9 dotted staining in IF (FIG. 11C), interaction among these proteins was detected in RKO and Colo320 cells, barely so in SW620 and LS174T cells, but not at all in DLD-1 cells by conventional IP (FIG. 12C). IF and colocalization threshold analysis showed that BCL9 co-localizes with NONO, SFPQ and ILF2 in RKO and Colo320 cells. However, this degree of co-localization is lower compared to that of NONO and SFPQ in Hela cells, which are regarded as examples of full co-localization (Imamura et al., (2014) Mol. Cell 53:393-406), indicating that BCL9 partially co-localized with these proteins (FIG. 12D and FIG. 12E).

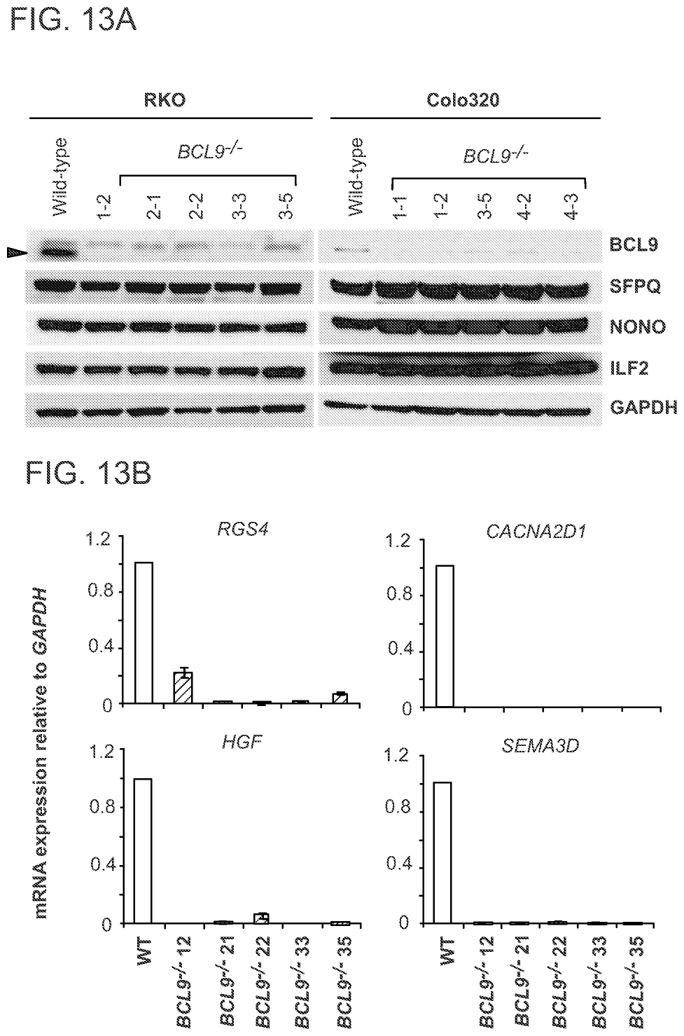

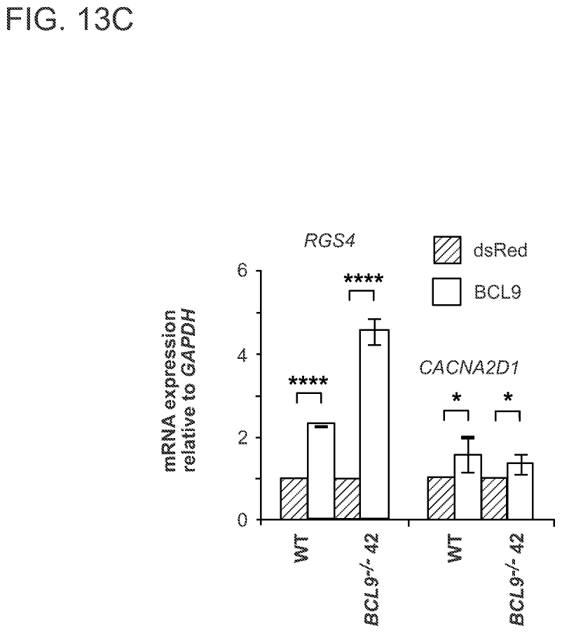

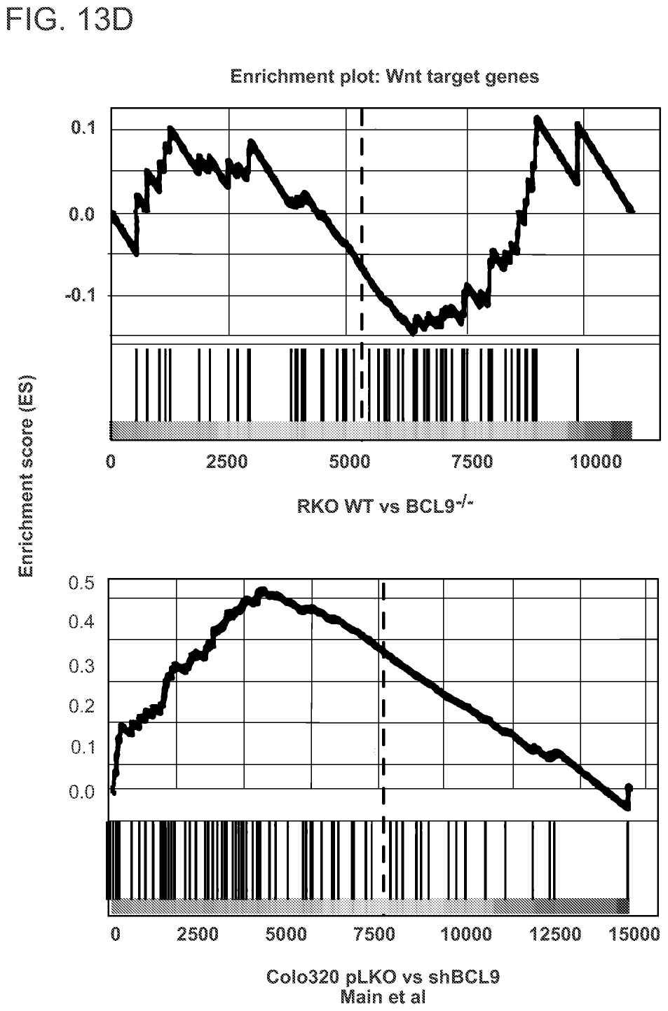

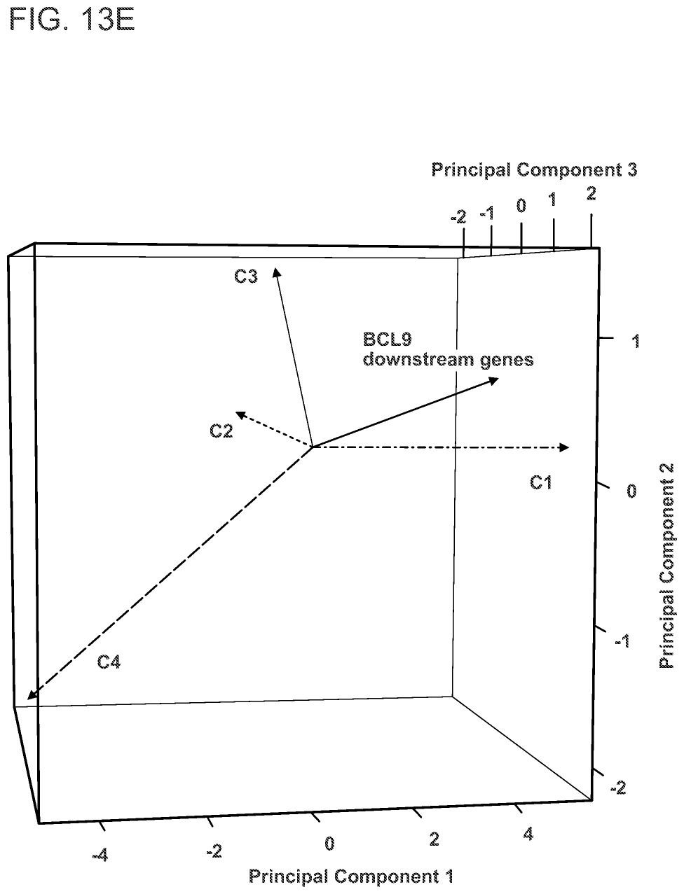

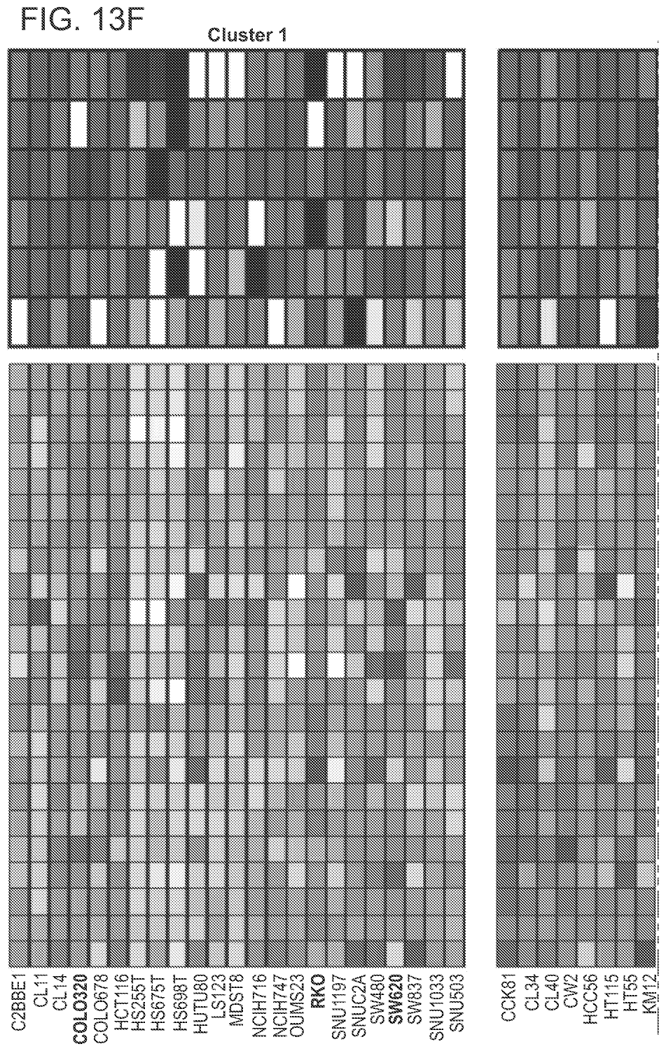

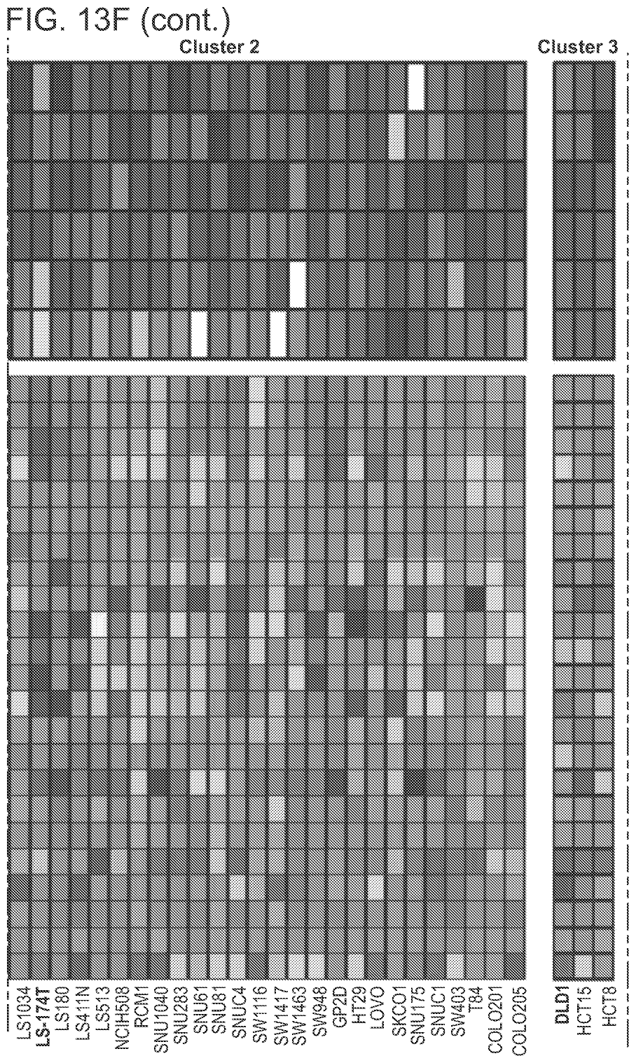

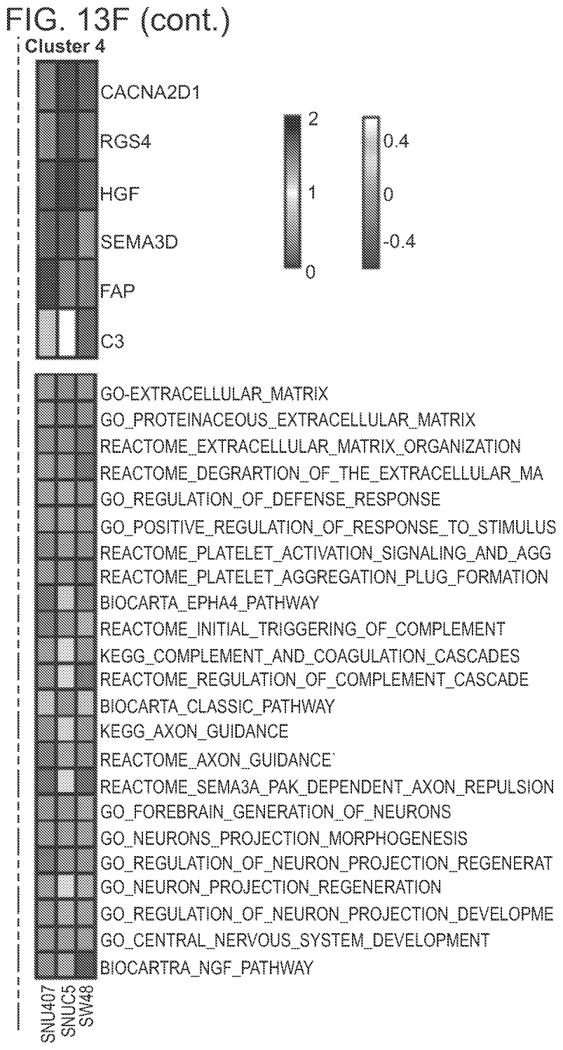

RNA-seq analysis was performed to identify genes whose expression could be regulated by BCL9 by comparing wild-type vs. BCL9 knockout RKO cells; a list of 975 downregulated genes was selected based on p-value (<0.05) and fold-change (>1). The levels of NONO, SFPQ, and ILF2 proteins did not change in BCL9 knockout cells (FIG. 13A), indicating that expression of these BCL9-interacting proteins is not regulated by BCL9. The top four genes, RGS4 (Regulator of G Protein Signaling 4), CACNA2D1 (Calcium Voltage Gated Channel Auxiliary Subunit Alpha2 delta 1), HGF (Hepatocyte Growth Factor) and SEMA3D (Semaphorin 3D) were verified by RT-qPCR in five different knockout clones and one rescued clone (FIG. 13B and FIG. 13C). The genes whose expression was decreased by BCL9 knockout were involved in axon guidance, calcium ion binding, and synapse organization (FIG. 2B, left), and they were not enriched as canonical Wnt target genes (FIG. 13D, top). Contrary to that seen in RKO cells, GSEA revealed that in Colo 320 cells, there was enrichment in canonical Wnt target genes (FIG. 13D, bottom). Importantly, in PCA analysis, the vector composed of differentially expressed genes between wild-type and BCL9 knockout RKO cells, points towards to C1 direction (FIG. 13E), and these genes were frequently overexpressed in C1 and its representative cell lines, but not in other CRC patient or cell subtypes (FIG. 2B right and FIG. 13F). These features enable the CRC cells that express high levels of BCL9 and its downstream genes to be reasonably classified as C1.

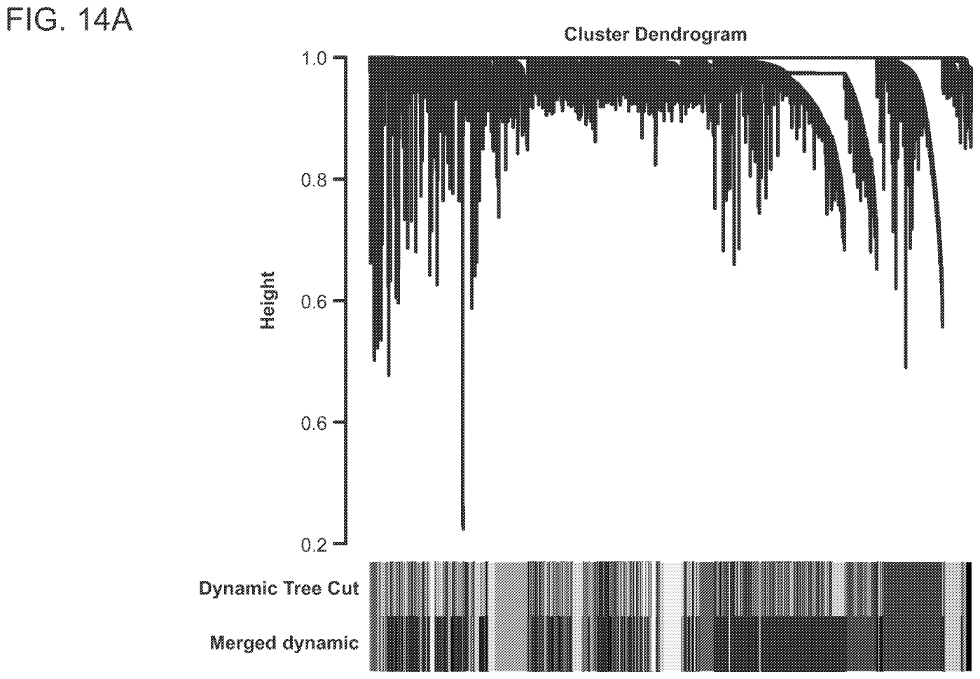

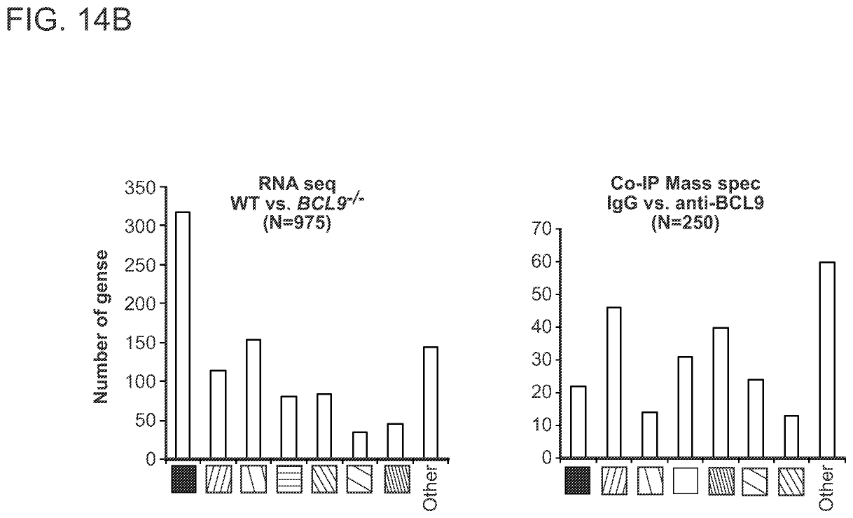

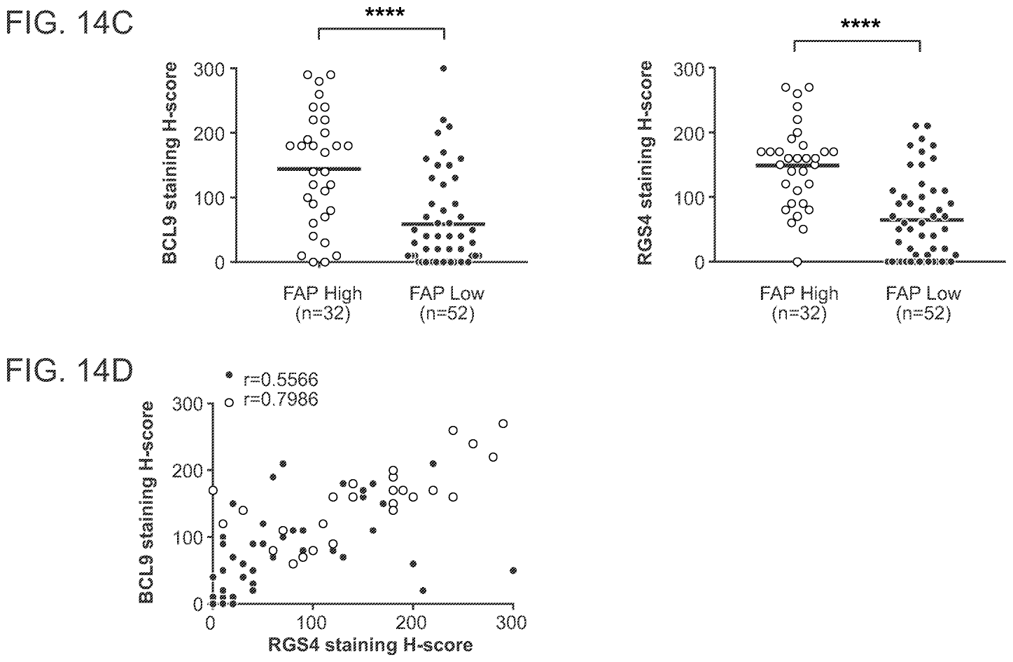

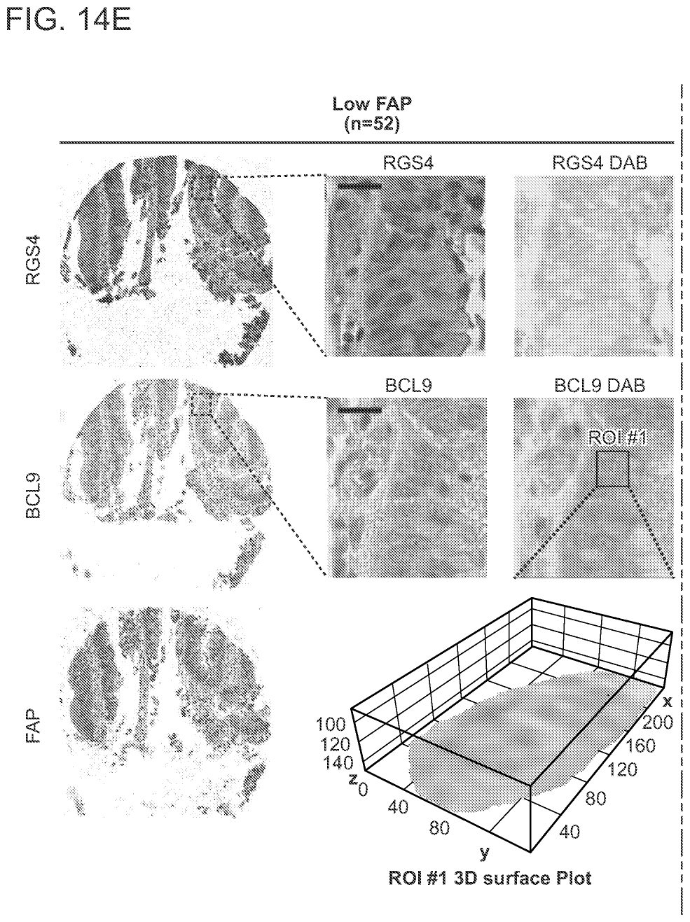

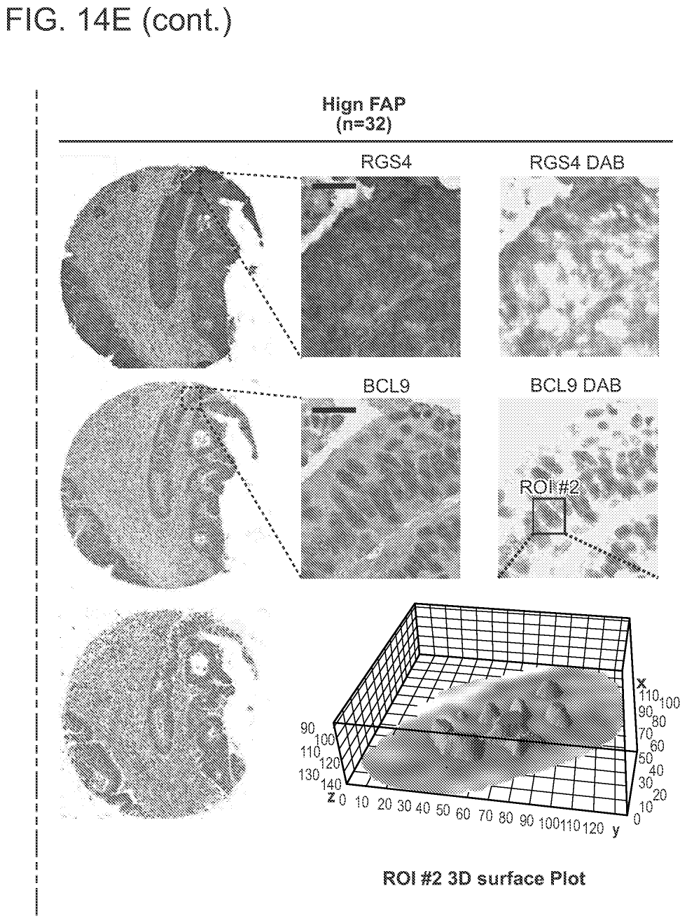

Gene expression profiling presents a highly ordered structure due to some genes being co-regulated within the same biological processes (Bergmann et al., (2003) Phys. Rev. E Stat. Nonlin. Soft Matter Phys. 67:031902). If BCL9 is associated with poor prognosis, then its downstream genes or partners should also be associated with poor prognosis and correlated with each other in the context of C1. Therefore, a global correlation coefficient matrix (Langfelder et al., (2008), BMC Bioinformatics 9, 559) was subsequently used to calculate the contribution of each cross-correlated gene set to patient survival (FIG. 14A) and to help identify key biological processes driving poor prognosis in C1. When all candidate BCL9-interacting proteins and downstream target genes were projected (colored lines) onto the matrix (FIG. 2C), most of the genes downstream of BCL9, but not the BCL9-interacting proteins, mapped into the Black, Brown and Blue groups (FIG. 14B), which were positively correlated to each other and negatively correlated with survival time (FIG. 2C). Additionally, GSEA revealed that genes in the Black and Brown groups were involved in processes such as extracellular matrix remodeling, neuron differentiation, and wound healing (FIG. 2D), which were previously regarded as “fingerprint” genes of C1 (FIG. 1C). This result was validated in a different TMA (n=84) from the one used in FIG. 1D-FIG. 1G, which showed high levels of BCL9 and RGS4 protein expression in FAP high compared with FAP low groups (FIG. 14C); RGS4 displayed a positive correlation with BCL9 expression (FIG. 14D and FIG. 14E). The above results suggest that BCL9 downstream genes form a co-regulation network that negatively correlates with prognosis. In addition to this finding, cells with high expression levels of genes downstream of BCL9 make them more like C1, which provides an explanation as to why BCL9 expression predicts a poor prognosis in C1 only.

### Example 4: BCL9 is Involved in Stabilizing the mRNA of Neural-Associated Genes by Interacting with Paraspeckle Proteins

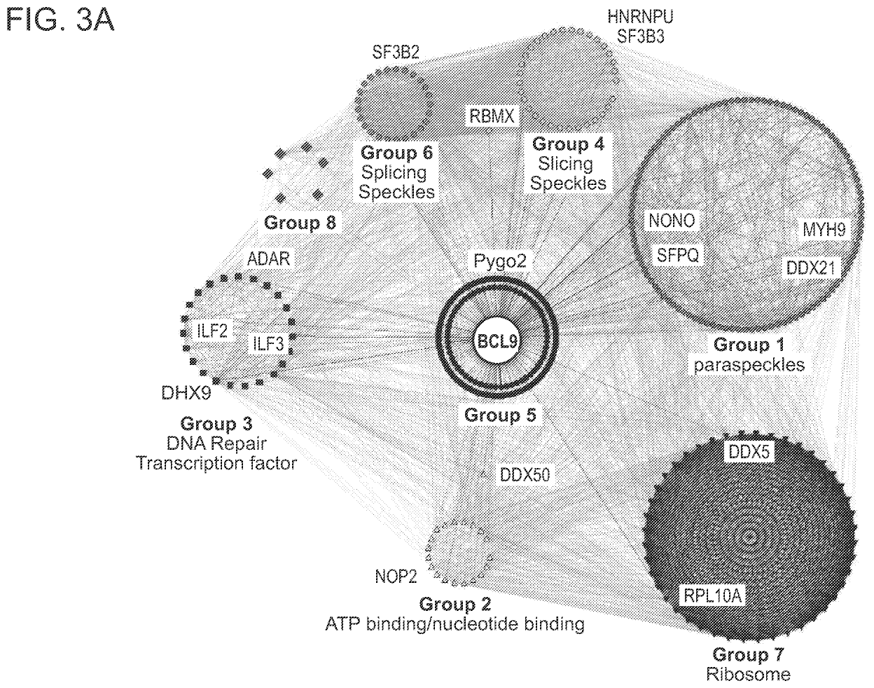

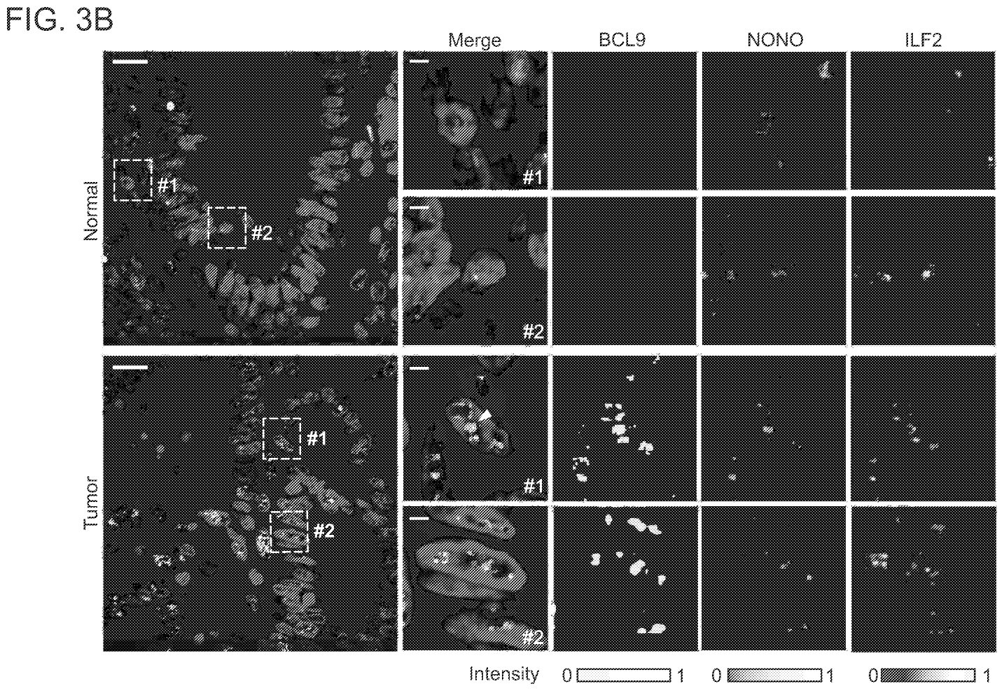

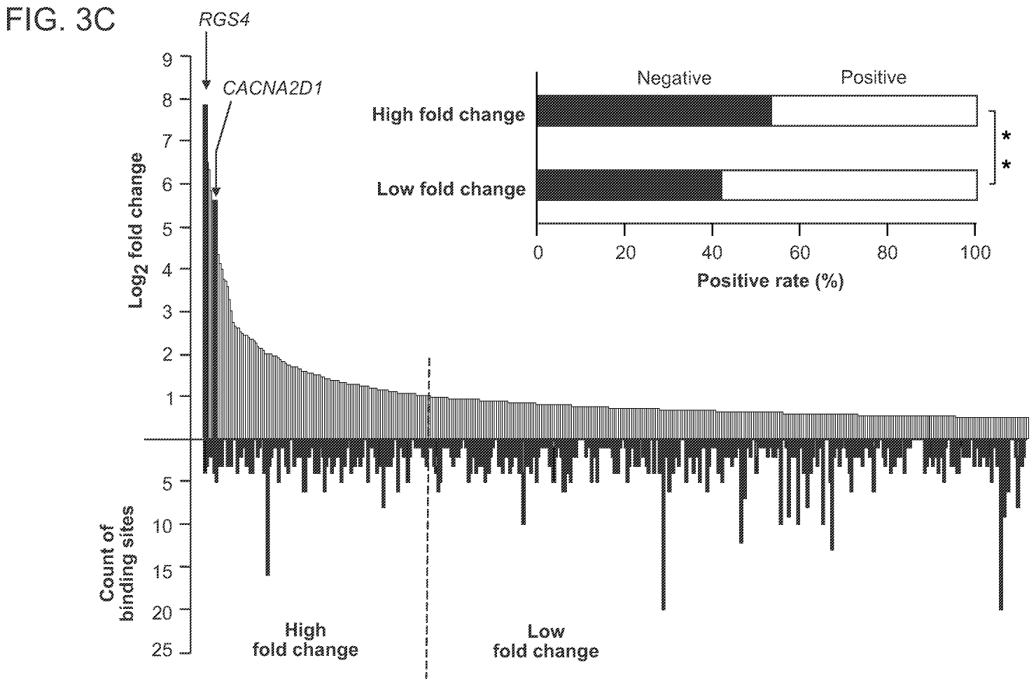

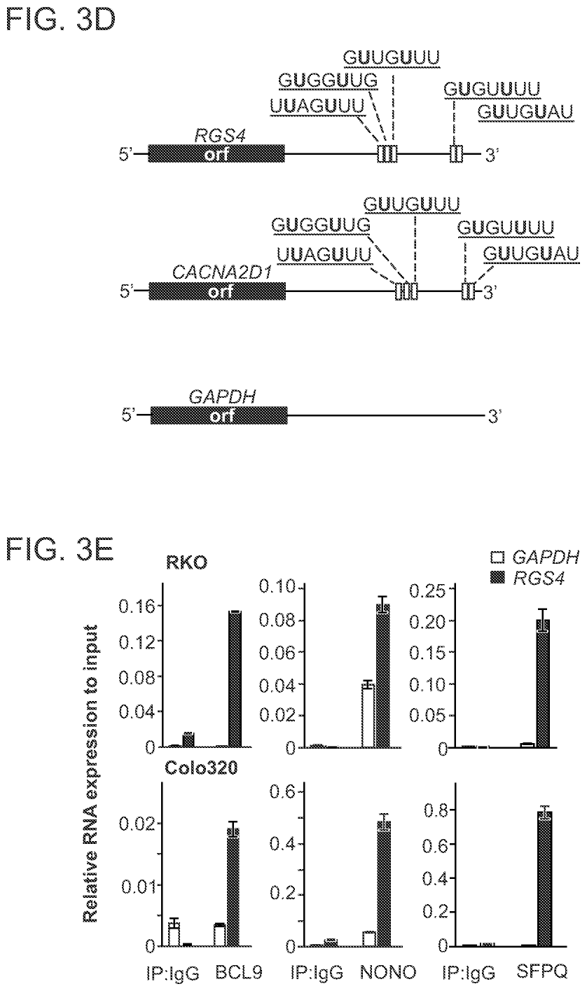

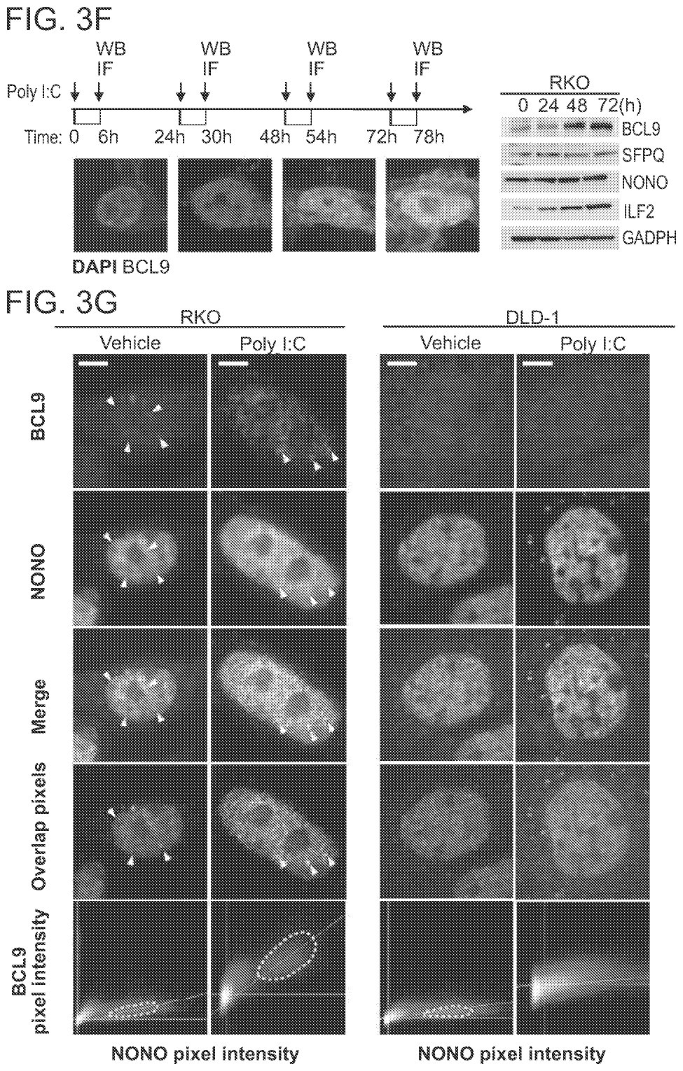

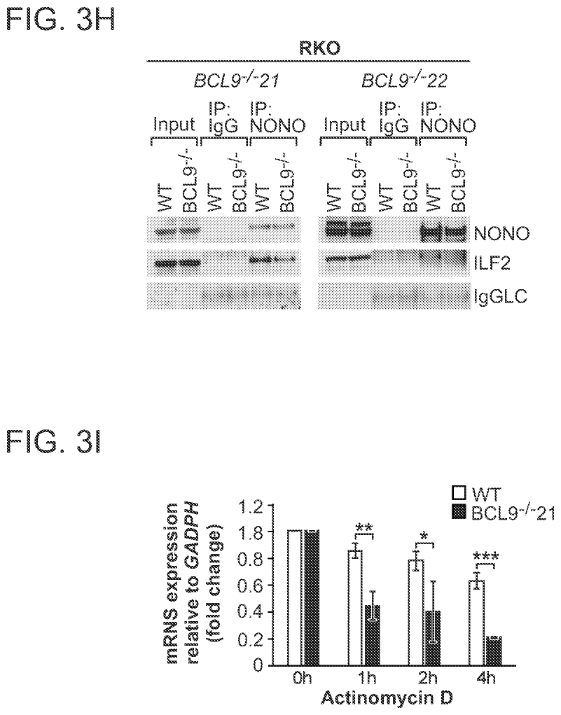

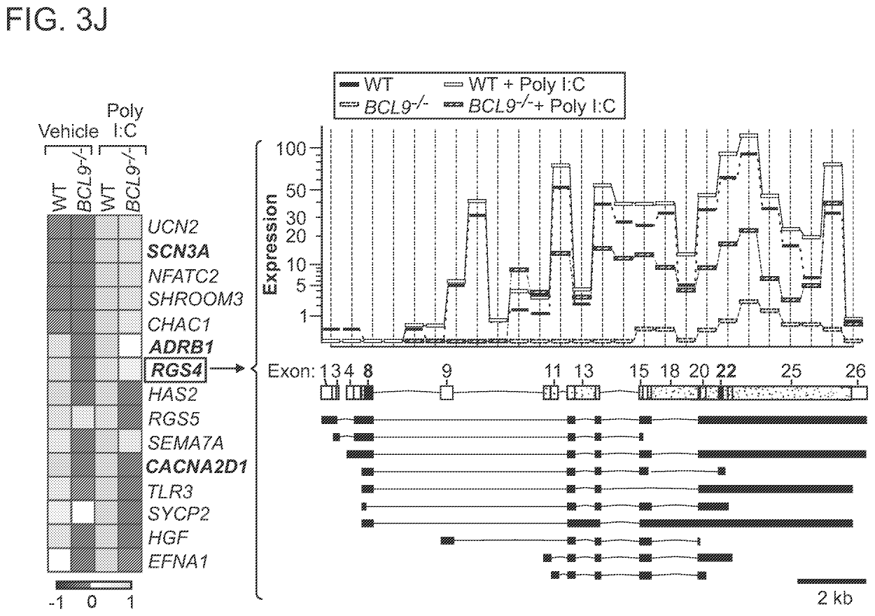

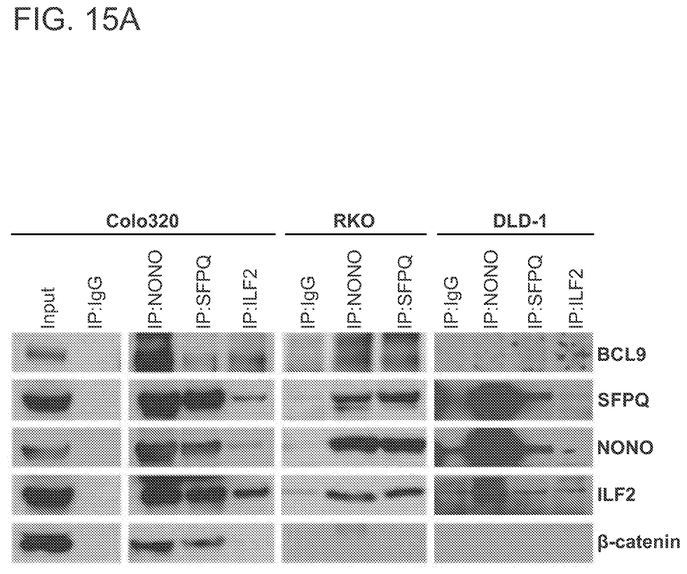

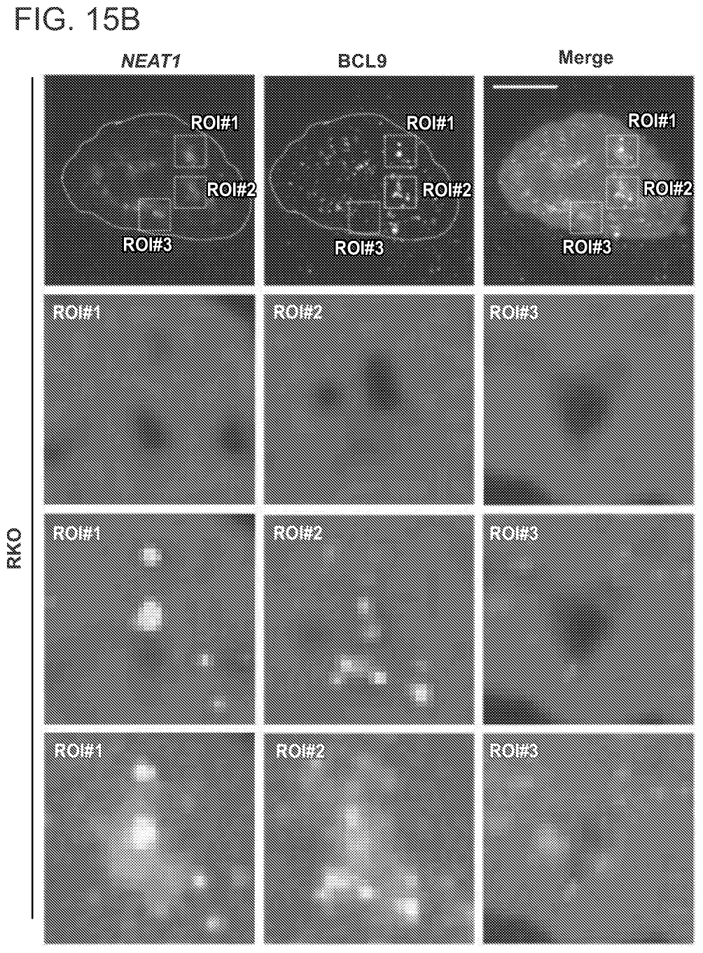

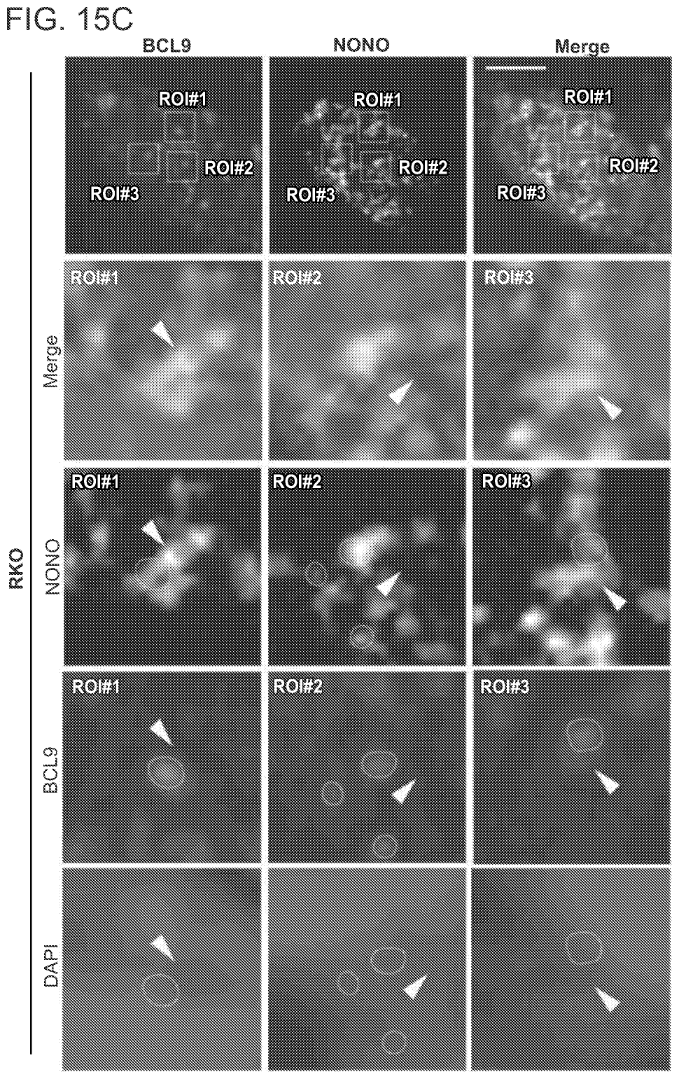

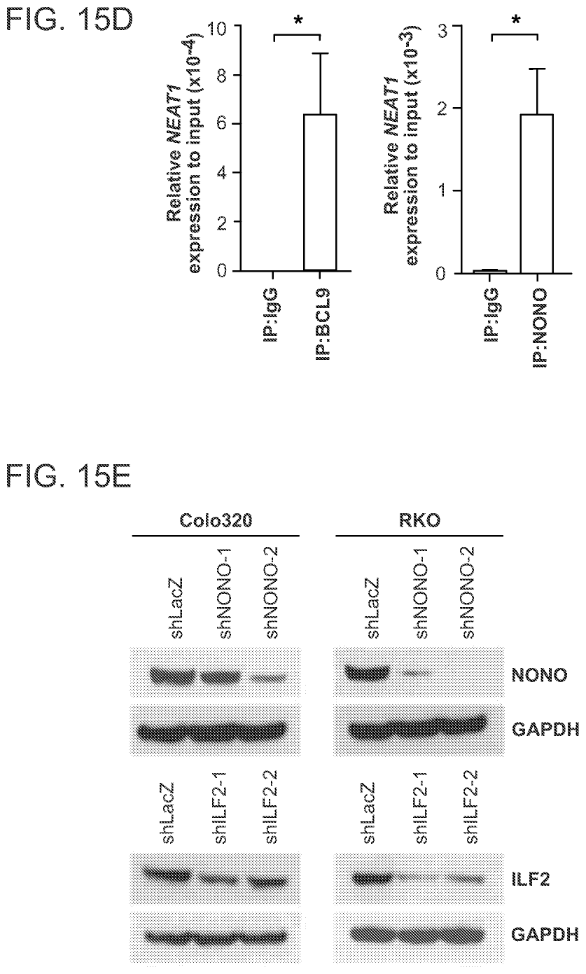

To further investigate the role of BCL9 in C1, a protein interaction network was generated to describe the relationship between all BCL9-interacting proteins identified by Co-IP MS. The proteins were clustered into eight different groups according to the “degree” of their interaction, which was assessed by the k-means unsupervised clustering algorithm (String database, string-db.org). Within each group there was not only binding, but also functional relationships. A high “degree” of interaction was displayed not only within proteins from the same group, but also among proteins from different groups (FIG. 3A). In support of this interpretation, IF studies revealed that BCL9, NONO, and ILF2, which all belong to different clusters, co-localized in punctate structures around the nucleolus in CRC but not in normal colon epithelial cells (FIG. 3B). Immunoprecipitation studies showed that Group 1 proteins (i.e., NONO and SFPQ), and Group 3 proteins (i.e., ILF2) co-immunoprecipitated with each other and with BCL9, in Colo320 and RKO cells but not in DLD1 cells (FIG. 15A). Notably, Group 1 is enriched in core protein components of paraspeckles, suggesting a functional link between BCL9 and this nuclear body. To further explore this possibility, a combination of immunofluorescence (IF) was performed with BCL9 antibody, and fluorescence in situ hybridization (FISH) with non-coding RNA NEAT1 probe used as a marker of paraspeckles. As shown in FIG. 15B and FIG. 15C, high intensity BCL9/IF dotted signal was enriched adjacent to and partly co-localized with the NEAT1/FISH and NONO IF signal around the interchromosomal region. RNA immunoprecipitation coupled with PCR (RIP-PCR) assay was carried out with anti-BCL9 antibodies and NEAT1 specific primers in whole cell lysates of RKO cells. As shown in FIG. 15D, NEAT1 was significantly enriched in the anti-BCL9 group but not in the IgG control. This result, in combination with the previous FISH/IF data, suggests that there is a physical connection and functional link between BCL9 and paraspeckles, but that BCL9 itself is not a core component of paraspeckles. Further supporting this functional link is the observation that overexpression of BCL9 in RKO cells increased viability of wild-type cells but did not rescue or affect the viability of cells with shRNA-induced knockout of NONO or ILF2 (FIG. 15E and FIG. 15F). In addition, the observation that overexpression of BCL9 did not induce expression of “bona-fide” Wnt downstream target genes (e.g., CD44 and Axin2) in RKO cells (FIG. 15G), indicates that in the C1 subtype the effect of BCL9 on cell survival/proliferation depends on its interaction with paraspeckle proteins (e.g., NONO and SFPQ), but not on the Wnt pathway.

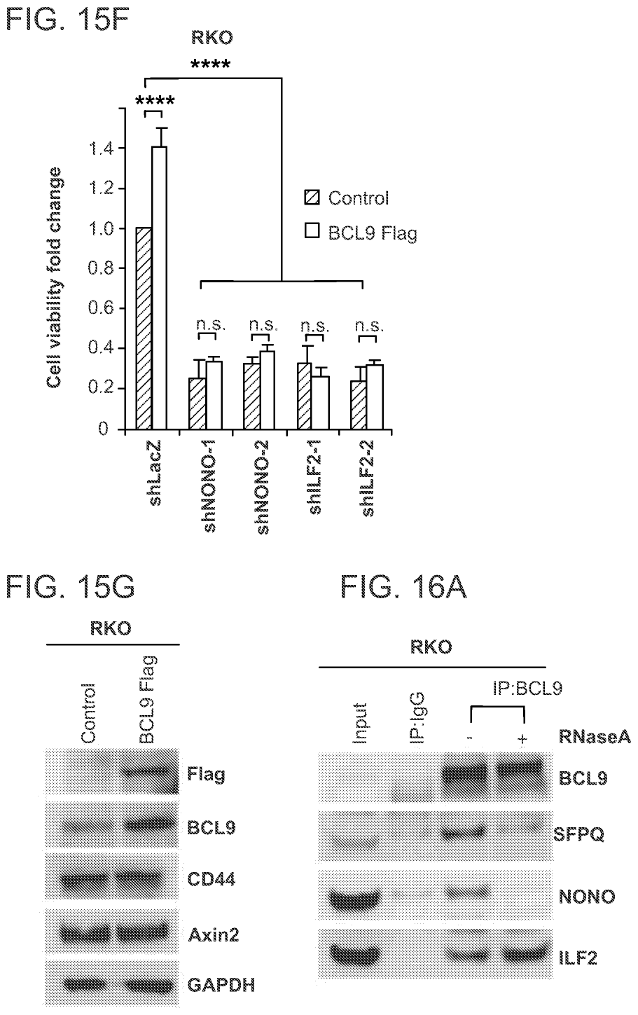

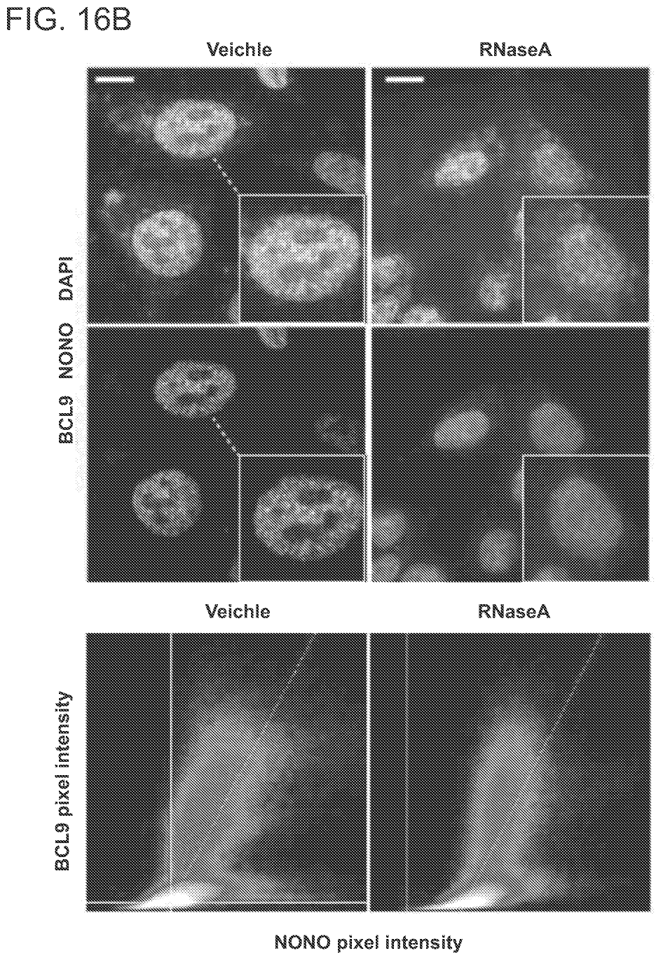

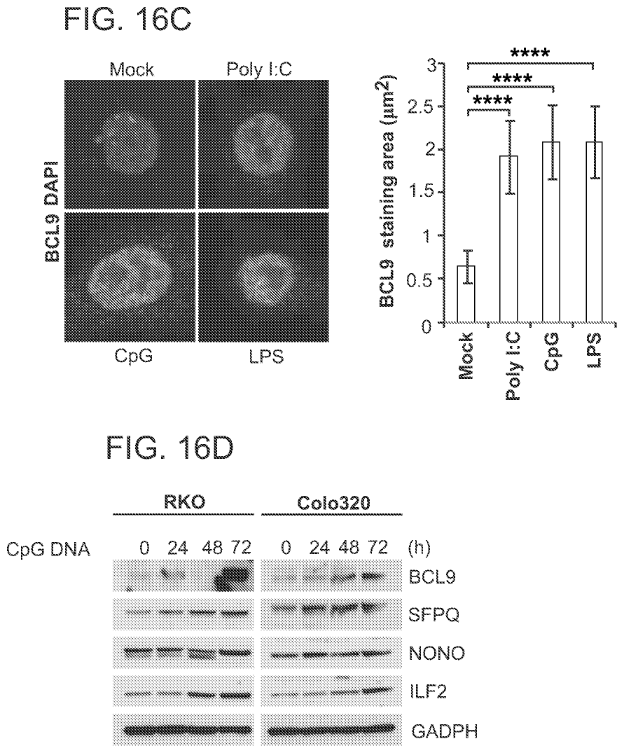

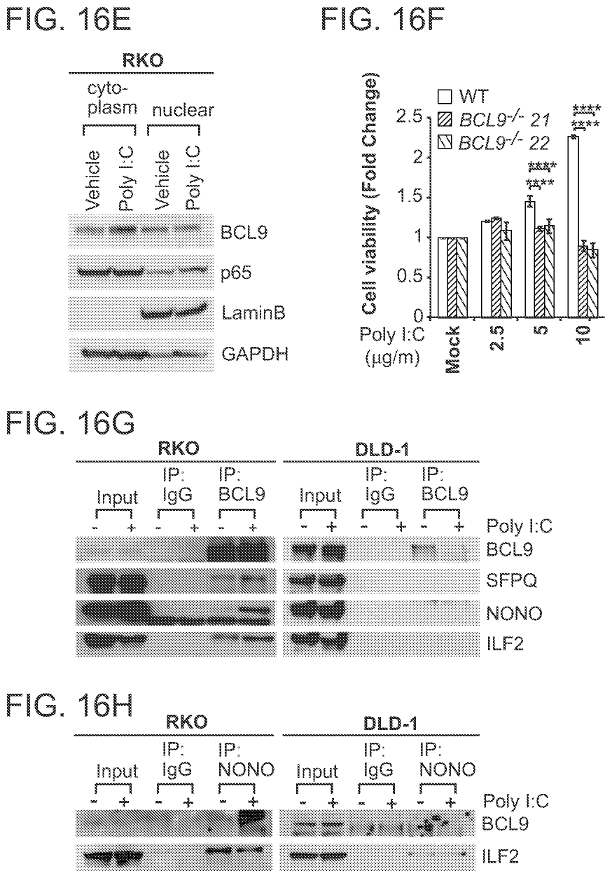

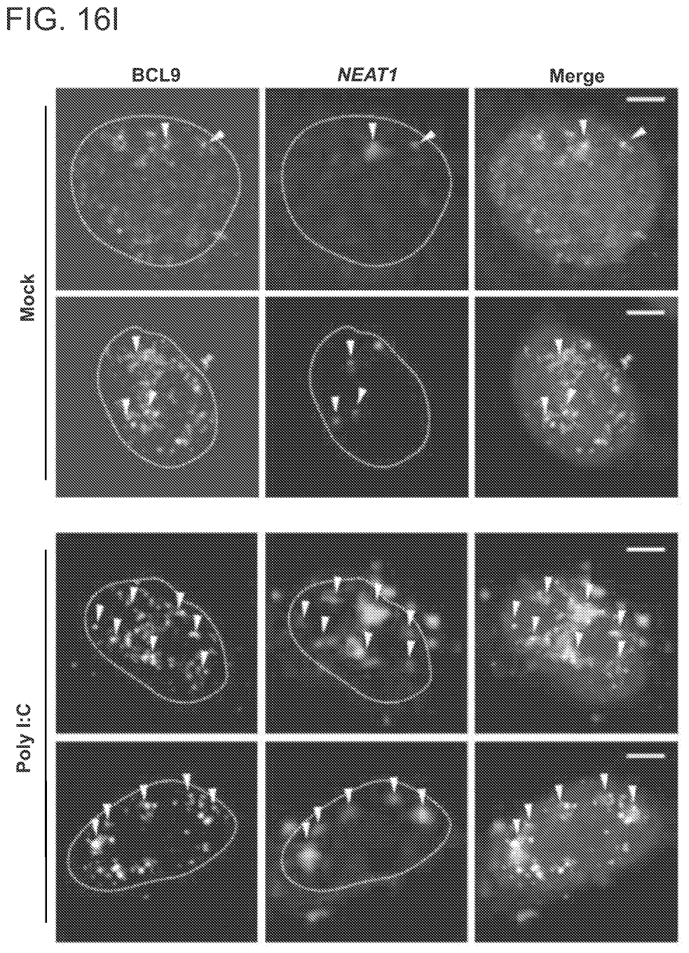

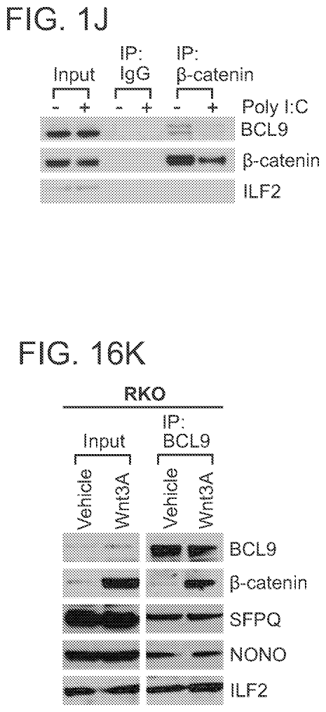

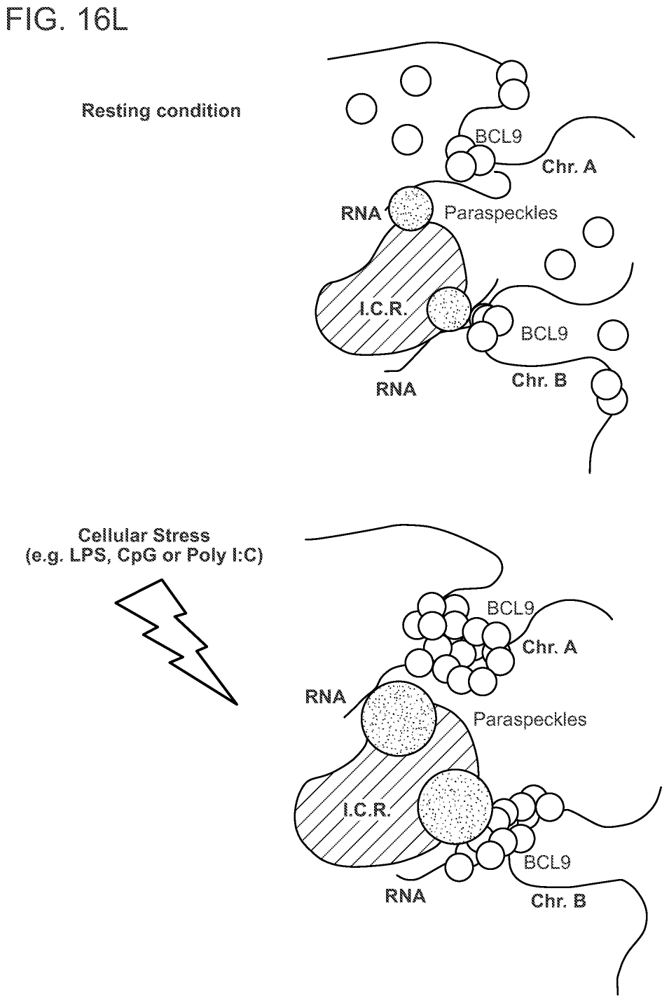

To investigate whether paraspeckle proteins could be involved in the mRNA processing of BCL9 downstream target genes, their 3′UTR regions were analyzed for the presence of the binding motif for the core paraspeckle protein SFPQ. In support of this possibility, the group of mRNAs showing a higher-fold decrease in expression in BCL9-deficient RKO cells were more likely to contain an SFPQ-binding motif than those that showed a low-fold change (FIG. 3C and FIG. 3D). In addition, when RNase A was added to the protein lysates before adding anti-BCL9 antibodies, the levels of immunoprecipitated SFPQ and NONO, but not of BCL9 and ILF2, were markedly decreased as compared with untreated control lysates (FIG. 16A). Disruption of intranuclear co-localization of BCL9 and NONO after RNaseA treatment was also detected by IF (FIG. 16B), indicating that BCL9/NONO/SFPQ but not BCL9/ILF2 interaction is dependent on an intact RNA, and that BCL9 itself should not be regarded as another core paraspeckle protein. Moreover, RIP-PCR assay of RKO and Colo320 whole cell lysates revealed the presence of RGS4 mRNA within the BCL9/NONO/ILF2 protein complex (FIG. 3E). Overall these results suggest a functional cooperation between BCL9 and paraspeckle proteins.

The BCL9-interacting protein complex regulates expression of target genes downstream of BCL9. When RKO cells were treated with poly I:C, CpG, or LPS to induce paraspeckle complexes formation (Imamura et al., (2014), Mol. Cell 53, 393-406), the accumulation of BCL9 around the nucleus was observed after 6 hrs (FIG. 16C). Longer exposure to poly I:C or CpG DNA induced further BCL9 accumulation around the nucleolus (FIG. 3F, left), which was also associated with increased BCL9, and ILF2 protein levels (FIG. 3F right and FIG. 16D). It was also noted that: i) increased levels of BCL9 protein were found in cytoplasmic but not nuclear fractions (FIG. 16E), ii) poly I:C increased the growth of wild-type RKO cells but not BCL9 knockout RKO cells (FIG. 16G), iii) consistent with previous results, the location and interaction of BCL9 with paraspeckle proteins in DLD-1 cells was unresponsive to poly I:C (FIG. 3G, FIG. 16G, and FIG. 16H), and iv) Poly I:C stimulation increased the partial co-localization between BCL9/IF and NEAT1/FISH signals (FIG. 16I), and poly I:C reduced the amount of BCL9/β-catenin protein complex (FIG. 16J). In addition, while Wnt3A increased the interaction between BCL9 and β-catenin, it did not affect BCL9 binding to paraspeckle proteins (FIG. 16K). These results indicate that the localization of BCL9 in the nucleoplasm is a dynamic process, and cellular stress in addition to promoting paraspeckle formation also increases the interaction of BCL9 with paraspeckle proteins (FIG. 16L).

In support of the involvement of BCL9 in RNA splicing/processing, BCL9 knockout decreased the interaction between NONO and ILF2 in comparison to wild-type cells was observed (FIG. 3H). In addition, when cells were treated with actinomycin D to block polymerase II-dependent transcription (Stellos et al., (2016), Nat Med 22, 1140-1150), the stability of BCL9 downstream target genes was decreased (FIG. 3I). Moreover, RNA-seq studies revealed that compared to untreated wild-type controls, genes that are downregulated in untreated BCL9 knockouts were increased in poly I:C-treated wild-type RKO cells (FIG. 3J), suggesting a role for BCL9 in processing mRNA of its downstream targets. In support of this view, expression of genes involved in calcium signaling and neural differentiation, such as RGS4, CACNA2D1 and ADRB1 (Adrenoreceptor Beta 1) (Ishizuka et al., (2012), Neurosci. Lett. 525, 60-65) also increased after poly I:C treatment (FIG. 3J). However, in BCL9 knockout cells, isoforms of RGS4 mRNA were shorter than in wild-type cells, even after poly I:C treatment, transcription of the full-length isoform was not rescued (FIG. 3J, right), further supporting a role of BCL9 in mRNA splicing/processing.

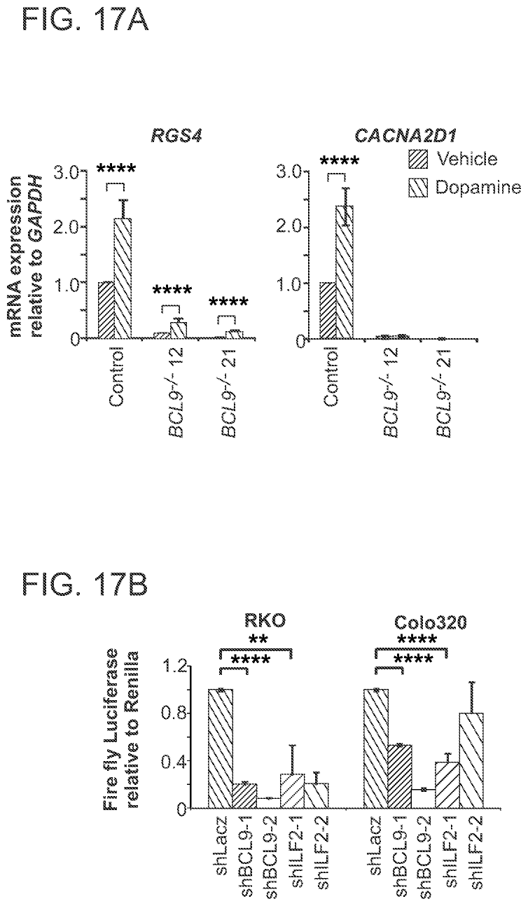

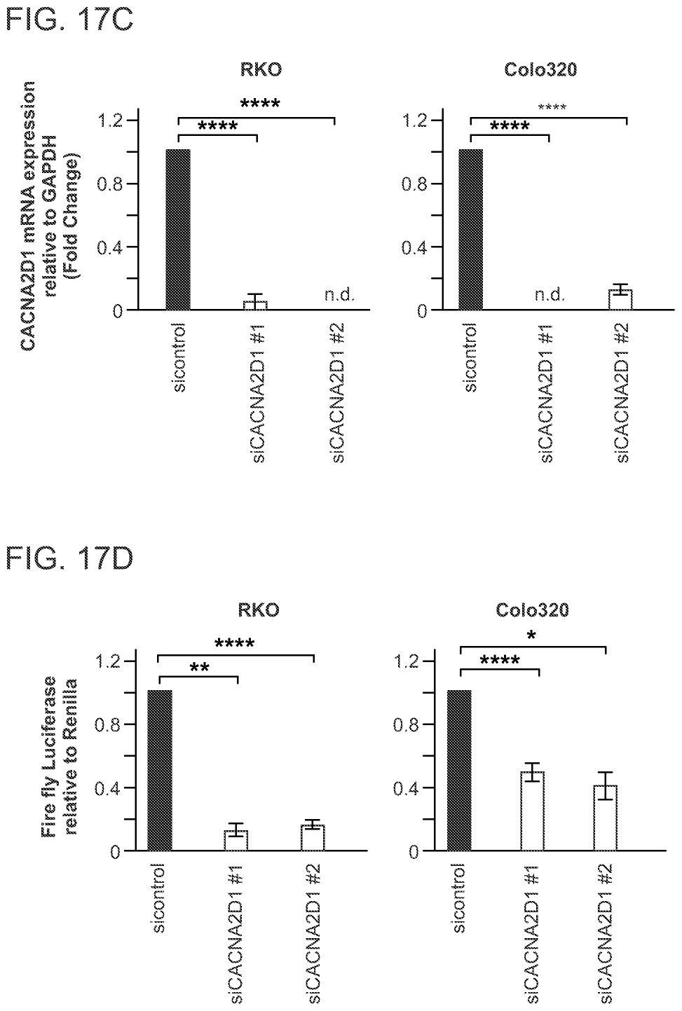

Given the role of BCL9 in regulating calcium signaling genes, the involvement of BCL9 in the activation of calcium signaling pathways was evaluated. Cells were treated with the adrenergic receptor beta agonist to activate the G protein coupled receptor-calcium axis (Taymans et al., (2004) Eur. J. Neurosci. 19:2249-2260). Expression of RGS4 and CACNA2D1 mRNAs were increased in both wild-type and BCL9 knockout cells after dopamine treatment; however, in BCL9 knockout cells mRNA expression was not completely rescued to wild-type levels (FIG. 17A). In addition, reporter activity dependent on NFATC2, a transcriptional factor whose activity is regulated by calcium waves (Hogan et al., (2003), Genes Dev. 17, 2205-2232), was reduced after knocking-down expression of BCL9, ILF2 or CACNA2D1 in RKO and Colo320 cells (FIG. 17B-FIG. 17D), suggesting a role for BCL9 in this process via mRNA stabilization of calcium-signaling genes. Overall, the above results indicate that through its interaction with paraspeckle proteins BCL9 regulates the expression of genes involved in calcium signaling.

### Example 5: BCL9 Regulates Synchronous Calcium Transient Dependent Communication Among CRC Cells

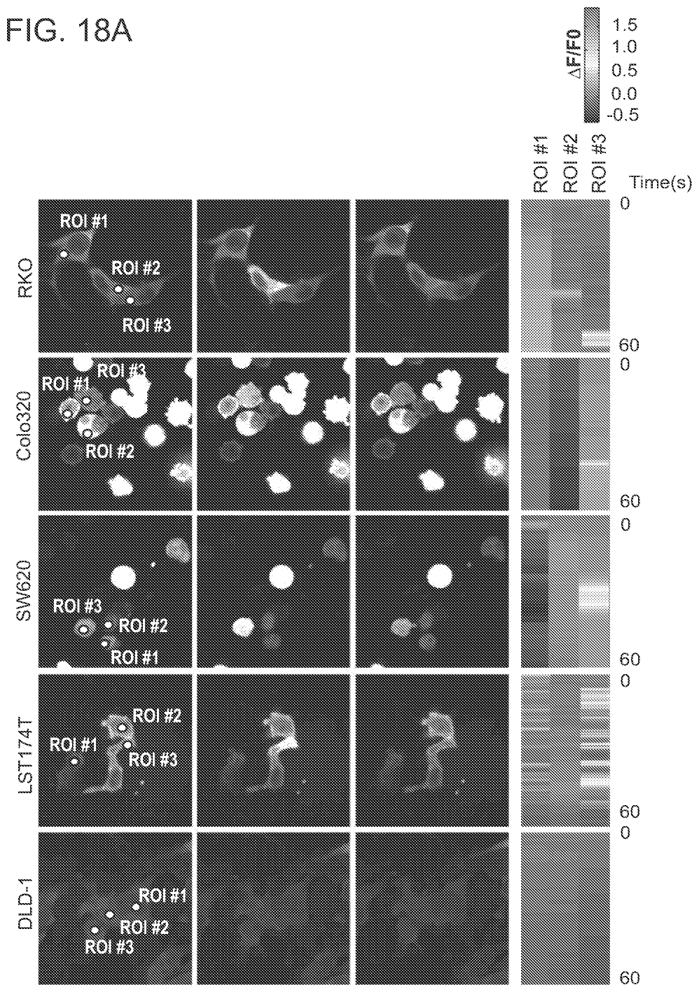

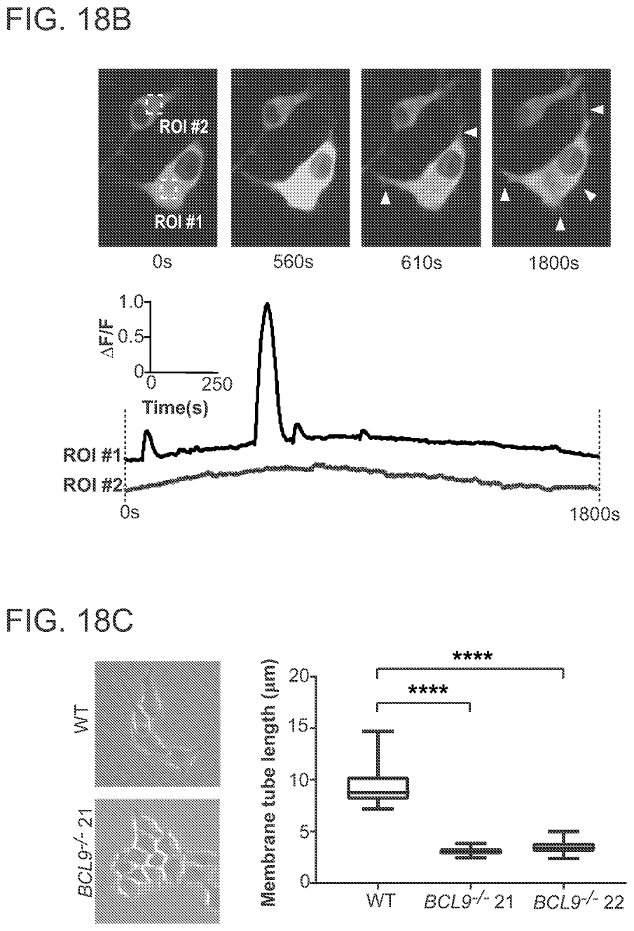

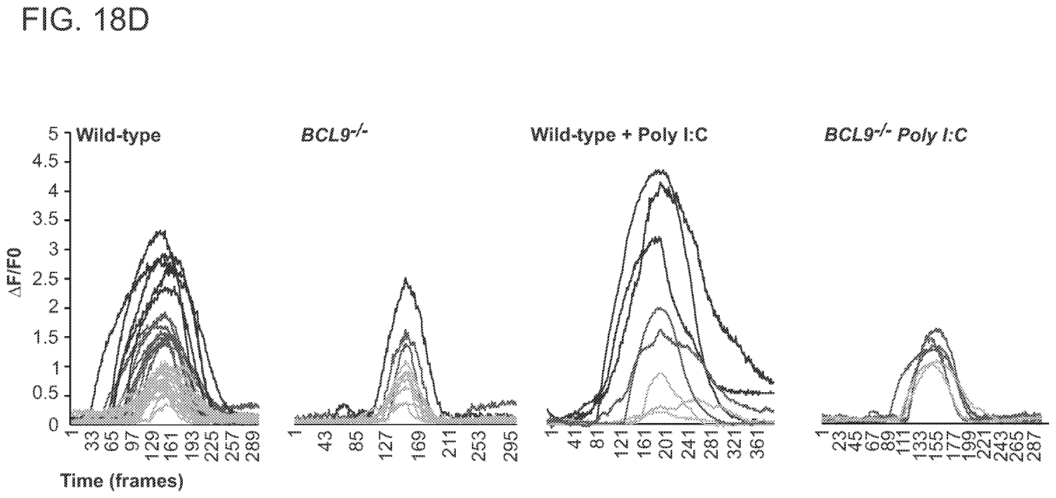

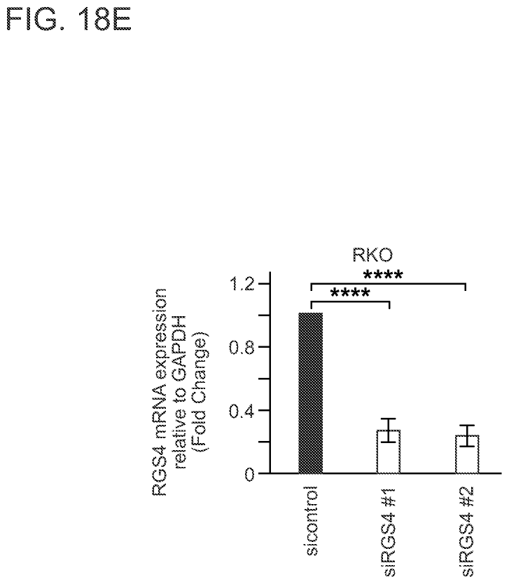

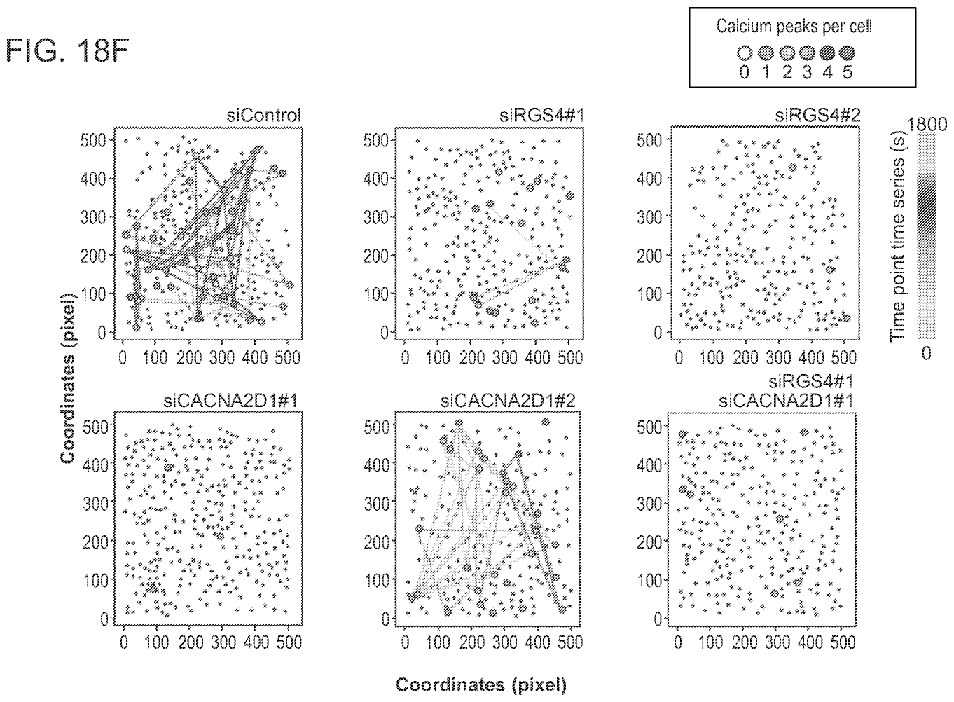

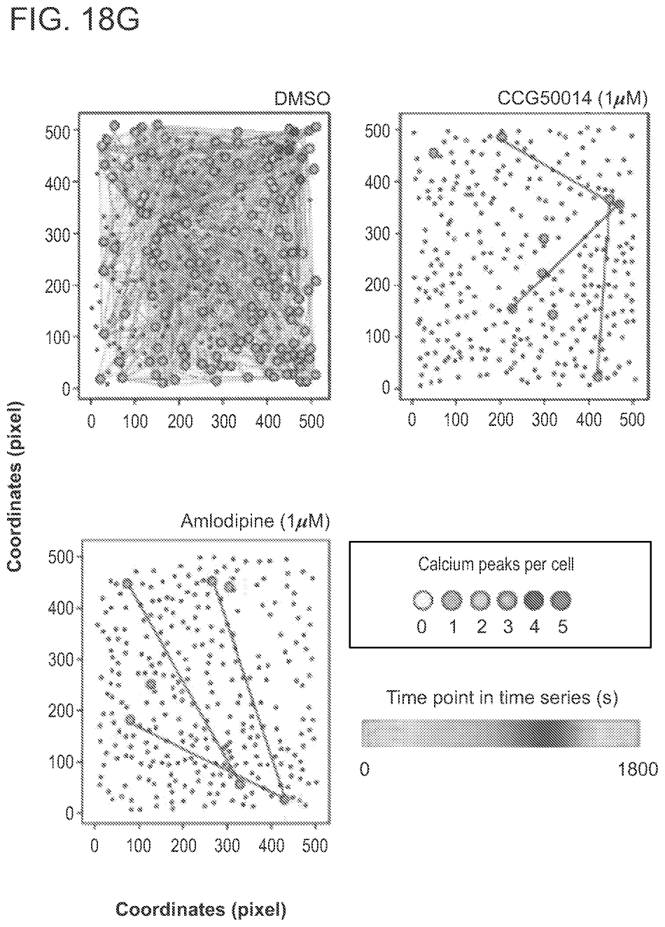

Global calcium changes were evaluated in CRC cells transduced with GFP-tagged GCAMP5 as a calcium indicator (Akerboom et al., (2012), J. Neurosci 32, 13819-13840). Consistent with previous Co-IP results, the occurrence of spontaneous calcium transients was detected in RKO, Colo320, SW620, and LS174T cells which displayed BCL9 dotted staining, but not in DLD-1 cells (FIG. 18A and FIG. 18B). As shown in a previous publication (Jacquemet et al., (2016) Nat. Commun. 7:13297), filopodia formation was observed after calcium transient (FIG. 18C). Notably, calcium transients were synchronous among individual tumor cells and independent of direct physical contact (FIG. 4A), but the lack of BCL9 reduced this synchronicity (FIG. 4B), as well as their amplitude and frequency (FIG. 4C and FIG. 4D). Calcium transients also disappeared after verapamil or EDTA treatments (FIG. 4C). Overall, these results indicate that: i) calcium transient synchronicity is dependent on BCL9, ii) calcium influx occurs through voltage-dependent calcium gates, and iii) the source of calcium influx is from the extracellular microenvironment. Because the cells that show synchronicity in calcium influx were not contiguous and didn't have direct physical contact, the spreading of calcium influx must be independent of gap junctions. This is responsible for synchronicity (Jorgensen et al., (2003) J. Biol. Chem. 278:4082-4086), which suggests the existence of a secreted factor that is involved in spreading of calcium influx among cells. In agreement with the RNA-seq results (FIG. 3J), a calcium wave spike library revealed that poly I:C enhanced the length and amplitude of calcium transients in wild-type but not in BCL9 knockout cells (FIG. 18C). Interestingly, knock down of RGS4 and CACNA2D1 mRNA levels and treating cells with RGS4 or L-type calcium channel inhibitors phenocopied the effect of BCL9 knockout in RKO cells (i.e. reducing the frequency and synchronization of calcium influx) (FIG. 4A and FIG. 4B vs. FIG. 18E-FIG. 18G), further linking BCL9 function with L-type calcium channel associated genes.

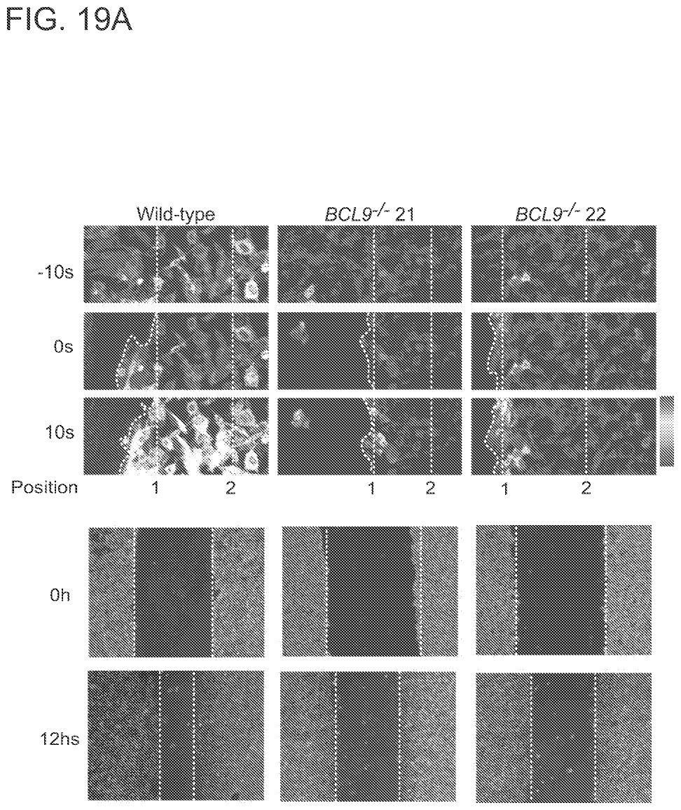

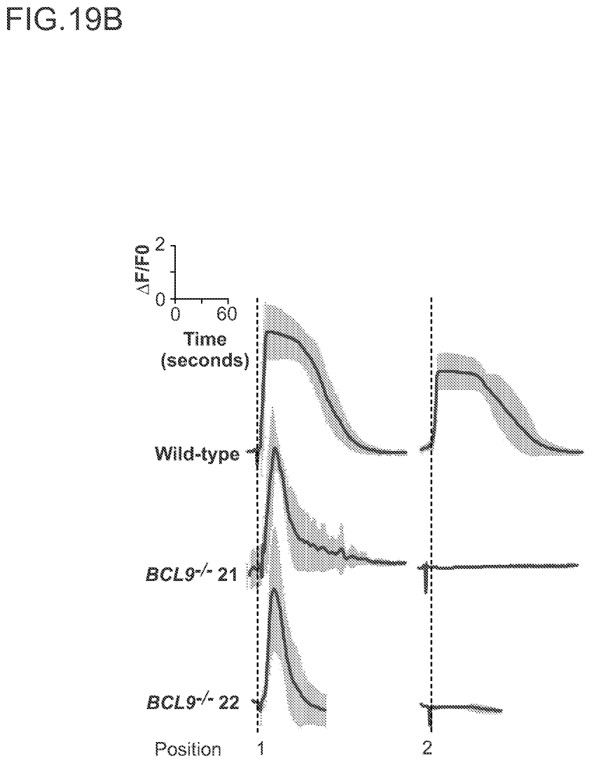

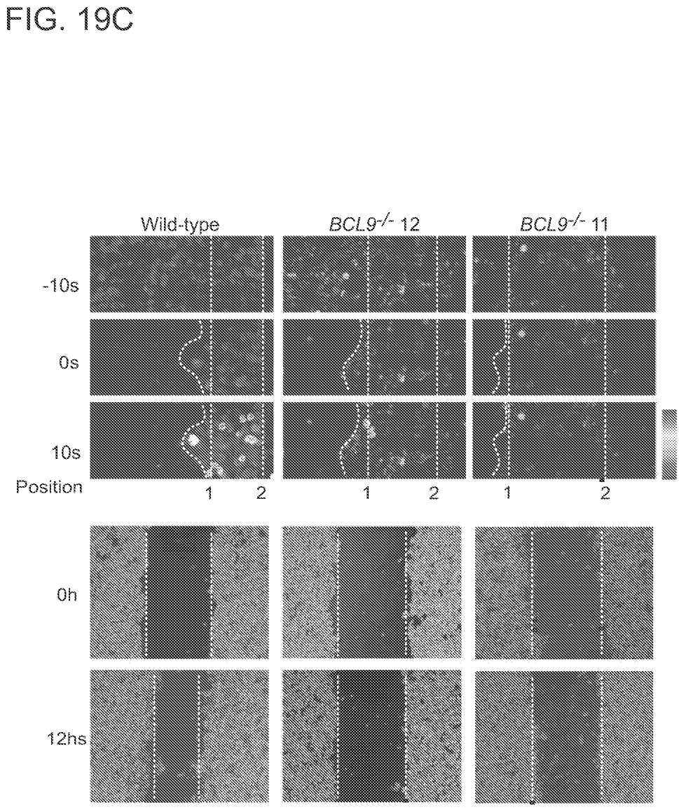

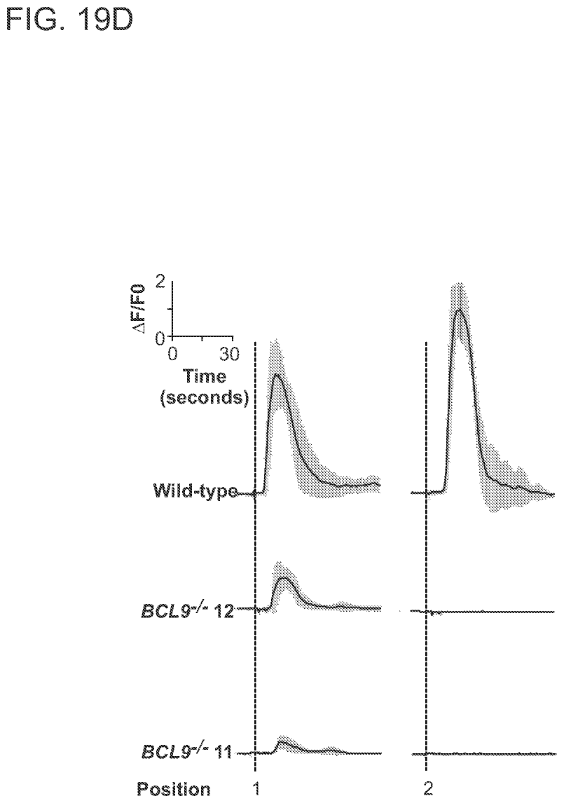

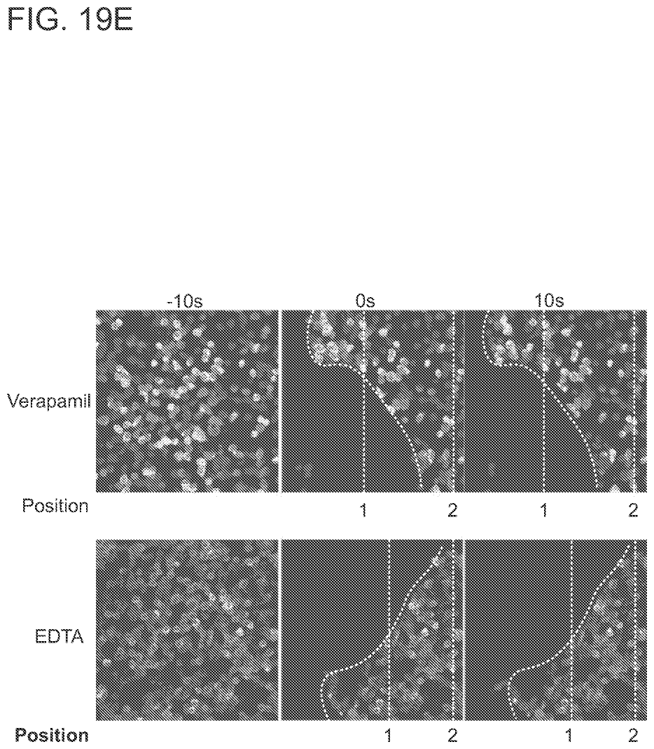

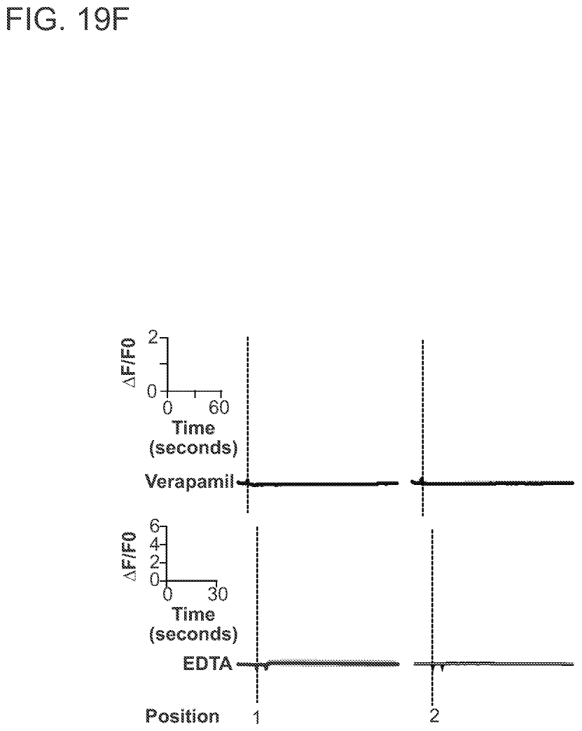

Using a model of epithelial wound-healing assays, it was shown that BCL9 is involved in the calcium transient spreading among tumor cells. It was previously shown that in this assay, one of the first reactions to injury of the cell monolayer is an intracellular calcium rise spreading as a wave from the injury site to the neighboring cells (Leiper et al., (2006), BMC Biol. 4, 27). By scratching the monolayer cell surface, calcium transients in the cells closest to the wound edge (hereafter referred to as the “primary” wave) were elicited and observed how the cells spread to distant cells in both wild-type and BCL9 knockout RKO and Colo320 cells (FIG. 19A-FIG. 19D). The amplitude and length of “primary” waves were significantly reduced and rapidly attenuated during propagation in BCL9 knockout compared to wild-type control cells (FIG. 19B and FIG. 19D), and only a few distant cells could receive calcium signaling from the scratched wound edge. Wound healing of the scratched monolayer was also delayed in BCL9 knockout cells (FIG. 19A and FIG. 19C). This was noteworthy in “secondary” calcium waves in the wound healing assay, which occurred later and were of shorter wave length than the “primary” wave (FIG. 4E and FIG. 4F). The timing of“secondary” wave spikes always lagged the “primary” wave. Although the onset time of the “primary” wave spike varied among different cells, “secondary” waves were synchronized, and independent of distance from the wound edge. Frequency spectrum analysis showed that “secondary” waves were also present in wild-type Colo320 but not in wild-type DLD-1 cells or in RKO and Colo320 cells lacking BCL9 (FIG. 4G). These results suggest that “secondary” waves are cell-type specific and must be triggered by calcium transients of “primary” waves. Since the cells presenting spontaneous “secondary” waves do not have direct physical contact with cells displaying “primary” waves, the occurrence of “secondary” waves may be induced by diffusible signaling molecules released by cells displaying “primary” waves.

In order to investigate whether BCL9 in paraspeckles is responsive to calcium transients, the functional correlation of BCL9 and calcium signaling was elucidated. The same was seen with spontaneous calcium transients, where the display of “primary” and “secondary” waves were blocked by treatment with verapamil (Palande et al., (2015), Comp. Biochem. Physiol. C Toxicol. Pharmacol. 176-177, 31-43) or EDTA (FIG. 19E and FIG. 19F). Co-IP experiments showed that the interaction of BCL9 with paraspeckle proteins was increased by verapamil; however, BCL9 protein levels were decreased (FIG. 4H). In addition, a time-course wound healing assay revealed that BCL9 complexation with paraspeckle proteins began approximately 10 minutes after the appearance of the “primary” wave, reaching a peak 20 minutes later in cells adjacent to the wound in vehicle-treated but not verapamil-treated cells (FIG. 4I). These data indicate that localization of BCL9 complexes with paraspeckle proteins is a response to cellular stress, and that BCL9 might mediate “secondary” waves via an unknown extracellular factor, whose release is dependent upon the opening of voltage-gated calcium channels. Moreover, these results indicate that the formation of BCL9/paraspeckles complex is induced by cell stress and that expression of BCL9 is regulated by cellular calcium signaling.

### Example 6: Neurotransmitters Mediate BCL9-Dependent Calcium Transients and Tumor Microenvironment Remodeling

The foregoing results prompted the investigation of what “extracellular factor(s)” might induce calcium wave propagation among CRC cells. As shown in FIG. 5A, RGS4 mRNA expression was increased in BCL9 knockout RKO cells after treatment with conditioned medium (CM) from wild-type RKO cells regardless of whether CM was pre-treated with proteinase K, suggesting that the “extracellular factor” is probably not a protein. Protein-free CM from wild-type and BCL9 knockouts RKO cells (FIG. 5B and FIG. 20A) revealed the presence of neurotransmitters with chemical structures and molecular weights resembling those of terbutaline, acetyltropine, and hygrine in wild-type but not in BCL9 knockout cells (FIG. 5C and FIG. 5D). This result, taken together with the finding that expression of adrenergic receptor β1 (ADRB1) is increased after poly I:C treatment of wild-type RKO cells (FIG. 3J), suggests that the “extracellular factor” most likely belongs to the phenethylamine family (i.e. terbutaline). In agreement with this, it was observed that the amplitude, frequency, and synchronicity of spontaneous calcium waves were reduced after treatment with propranolol, an inhibitor of ADRB (FIG. 20B).

Terbutaline induced propagation of calcium transients in wild-type but not in BCL9 knockout RKO cells (FIG. 5E, top). However, the occurring time of calcium transient after terbutaline treatment was variable among cells (FIG. 5E, middle) and displayed dissimilar frequency spectrum characteristics (FIG. 5E, bottom). In the wound healing assay, scratching activated calcium transient in most of cells, whilst very few of them were activated in the propranolol treated groups (FIG. 5F, top). In the heat map of response time distribution, cells located at the same distance from wound edge displayed different response times after scratching (FIG. 5F, bottom). These results indicated that terbutaline was indeed the trigger of calcium transient, and the lack of BCL9 reduced the cell sensitivity to terbutaline. The cells that received terbutaline treatment display the calcium transient and passed the signal to a specific subset but not to all the cells around them. Therefore, this type of signaling should be regarded as the result of neurotransmitter diffusion and specific predetermined intercellular functional connections.

This characteristic signaling process resembles neural cells and could allow CRC cells to establish complex communication networks on a multicellular scale and regulate the activity of cells from the tumor microenvironment. To test this possibility, RKO cells were cocultured with THP-1 cells, the latest being a representative model of human M2 macrophages, which promote tumor progression and tumor microenvironment remodeling (Grailer et al., (2014), J Innate Immun 6, 607-618; Genin et al., (2015), BMC Cancer 15, 577; Hardbower et al., (2017), Oncogene 36, 3807-3819). It was observed that wild-type but not BCL9 knockout RKO cells transmitted calcium transients to the THP-1 cells (FIG. 5G, left). Furthermore, synchronous calcium transient network (FIG. 5G, middle) and frequency spectrum (FIG. 5G, right) analysis displayed a complex communication network among these cells in wild-type but not BCL9 deficient RKO cells, indicating the influence of CRC cells in microenvironment remodeling. In support of this view, Phorbol 12-myristate 13-acetate (PMA) stimulation (which promote M2 differentiation) in combination with protein-free CM from wild-type RKO cells, but not from BCL9 knockouts, induced TGF-β and IL 10 expression in the THP-1 cells (FIG. 5H). Overall, these results highlight the role of BCL9 in cell to cell communication among CRC cells and with other cells in the tumor microenvironment, which could have important implications for CRC therapy. As supporting evidence of this possibility, propranolol and verapamil significantly reduced the viability of RKO, Colo320, and SW620 more than LS174T or DLD-1 cells, a finding which could have important implications for CRC therapy (FIG. 20C).

### Example 7: Interaction of BCL9 with Paraspeckle Proteins is Associated with Increased Proliferation of CRC Cells and Stromal Cell Infiltration In Vivo

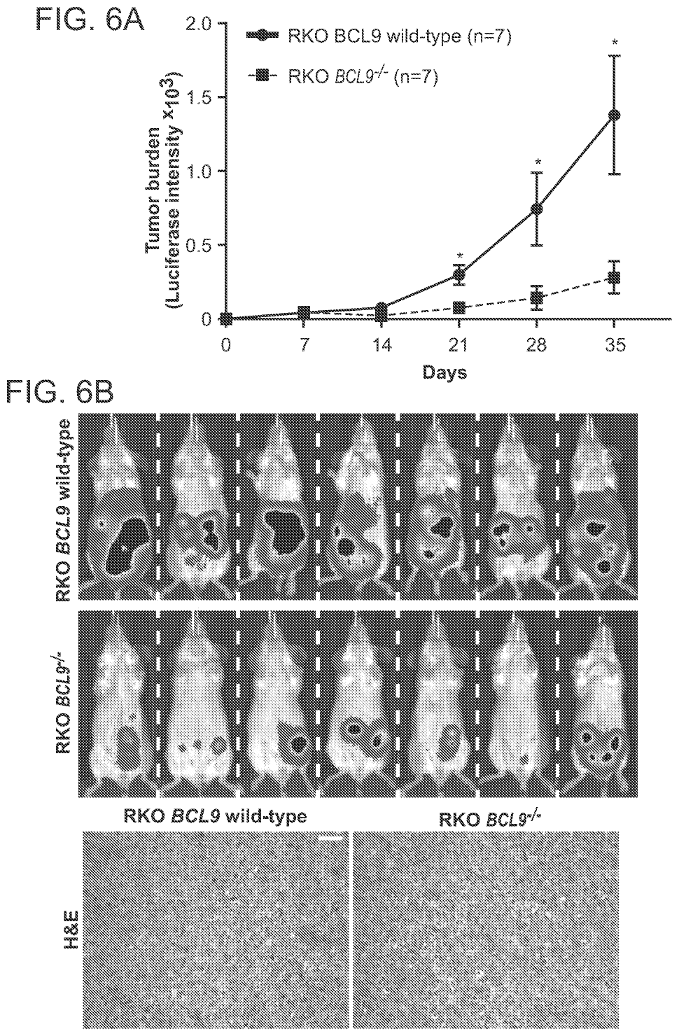

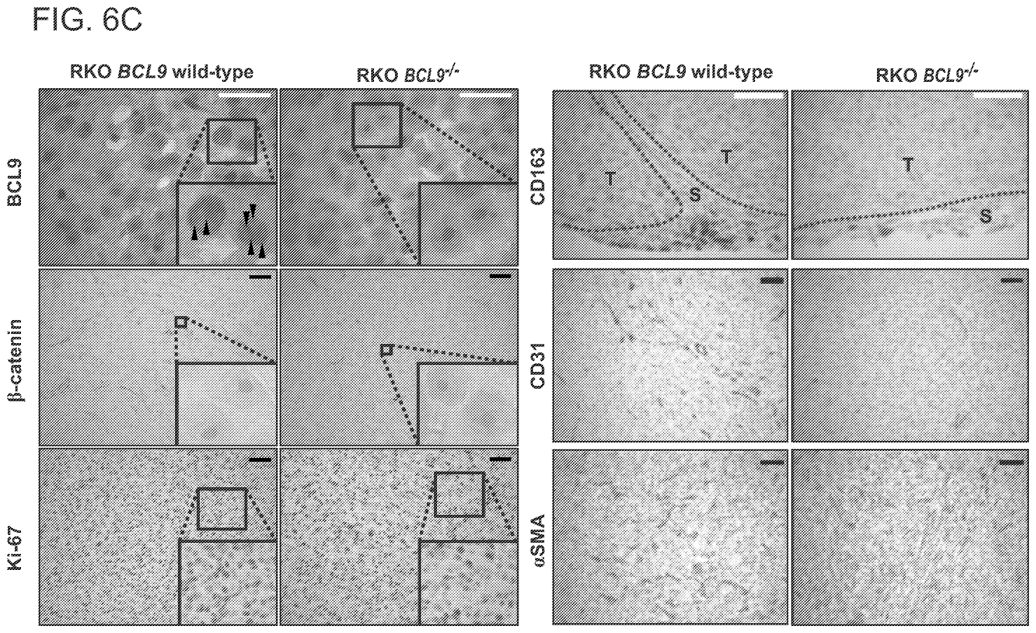

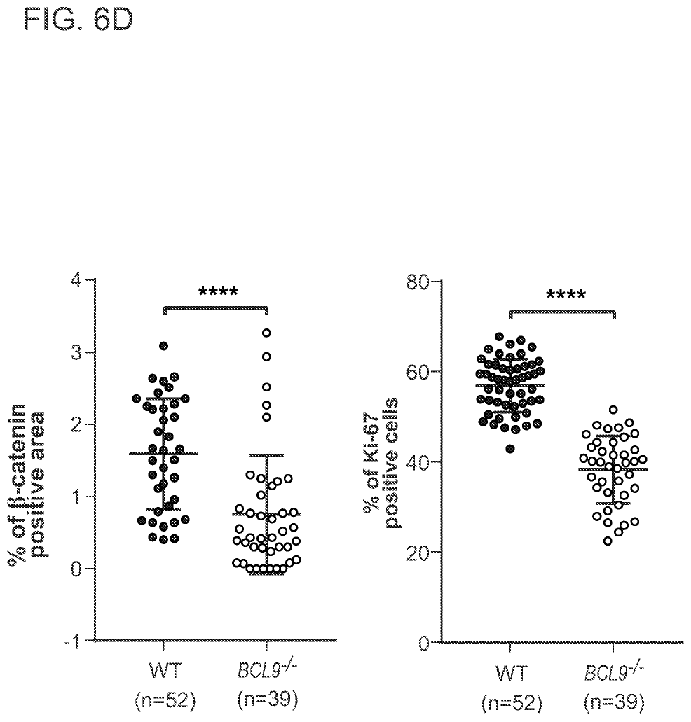

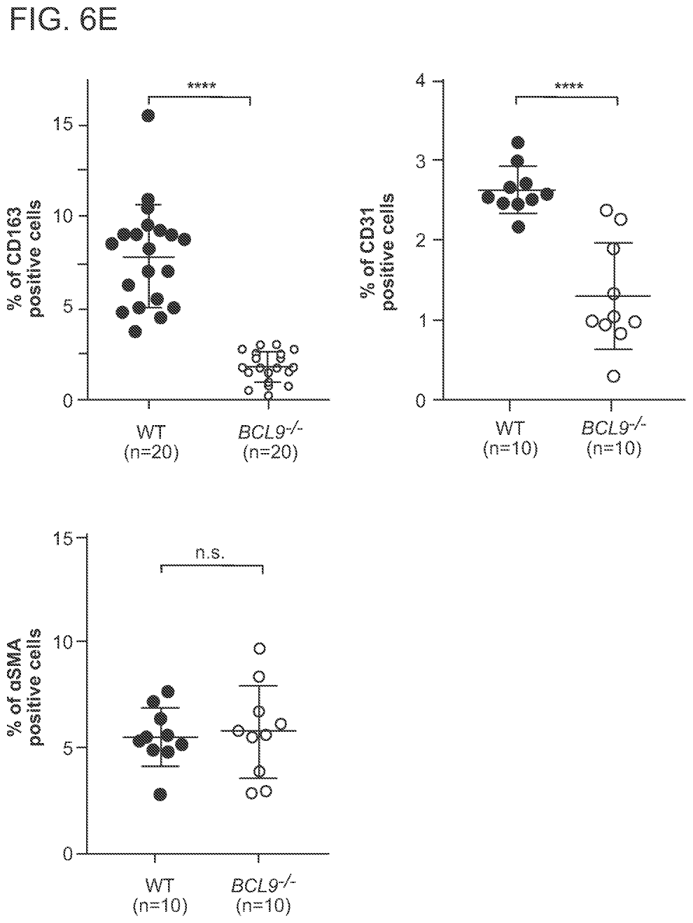

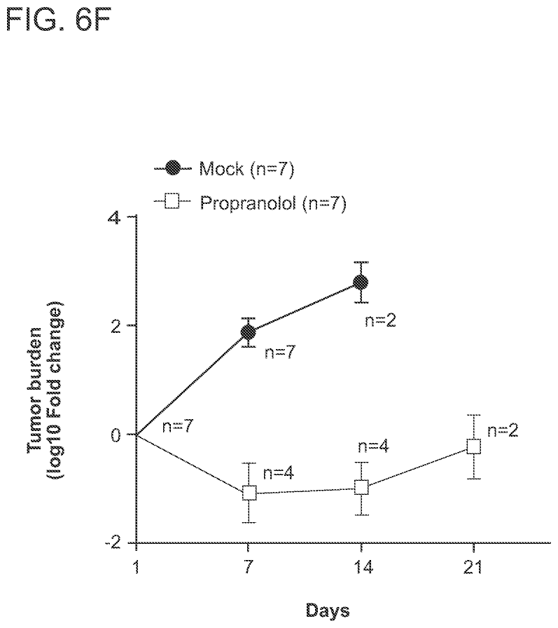

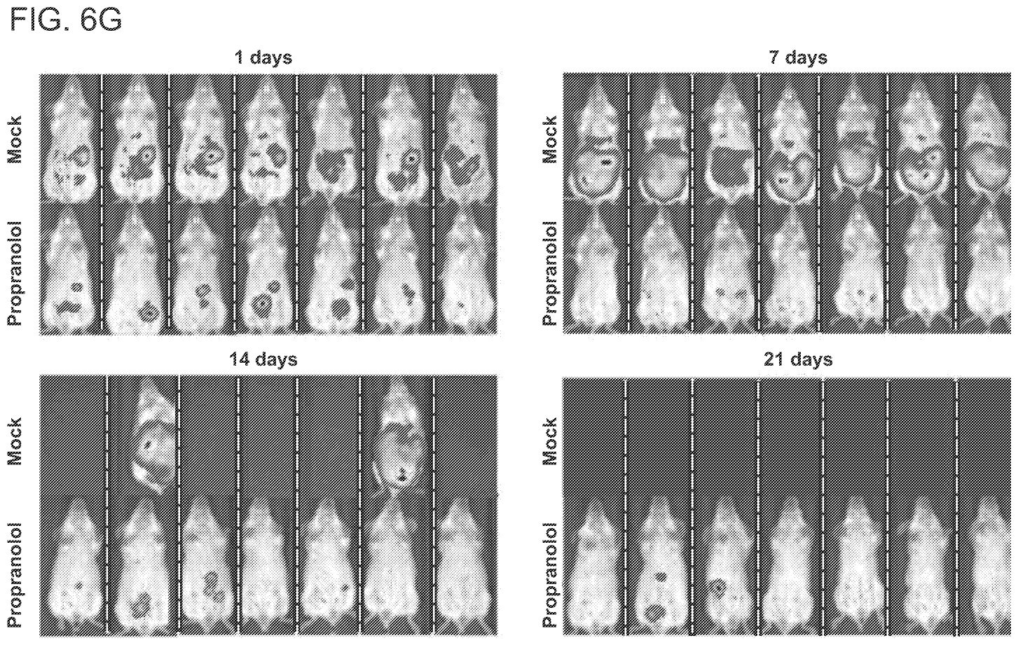

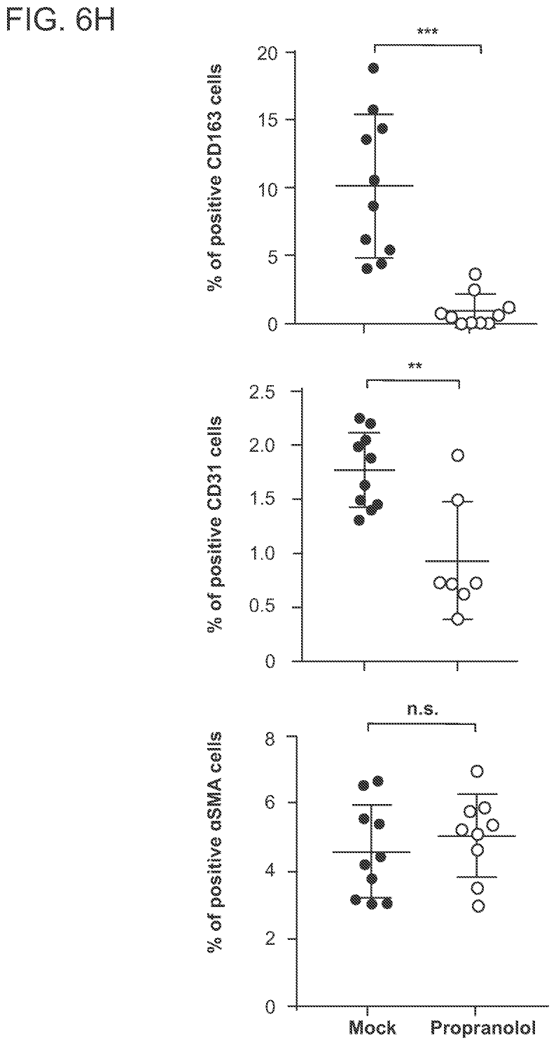

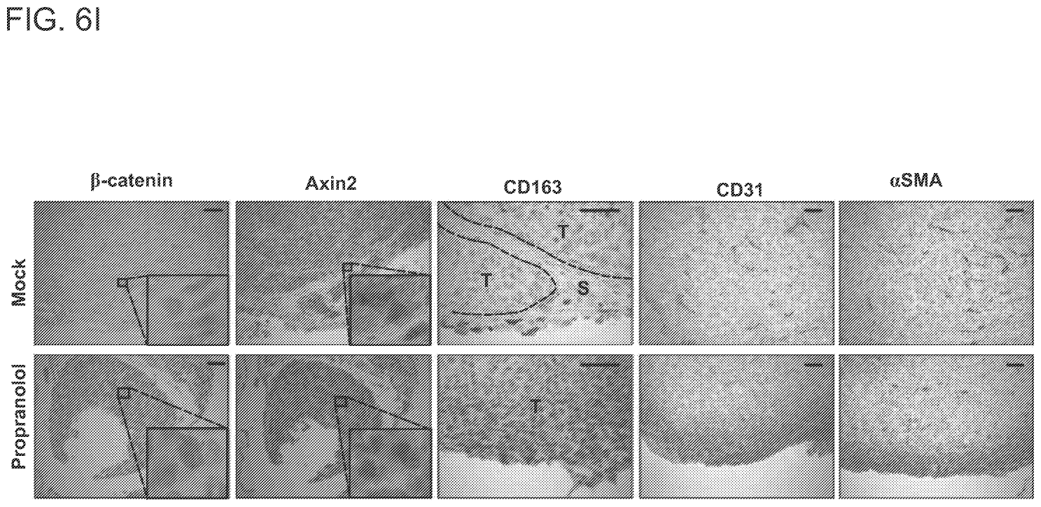

To investigate the biological consequences of BCL9 localization in paraspeckles in vivo, RKO cells, which do not display nuclear β-catenin activity (Rosenbluh et al., (2012), Cell 151, 1457-1473), were labelled with luciferase and implanted in immunodeficient mice (CB17.Cg-PrkdcscidLystbg-J/Crl, Beige) and tumor growth was evaluated by whole body imaging. As shown in FIG. 6A and FIG. 6B (top), tumor growth was significantly reduced in mice implanted with BCL9 knockout RKO cells as compared with control mice implanted with wild-type RKO cells. After 35 days of implantation, the mice were euthanized and all tumor nodules tumor nodules from the peritoneal cavity were harvested and processed for histological and immunohistochemical analysis. Although no major histomorphologic differences were observed between engrafted wild-type and BCL9 knockout RKO cells, the tumor cell component in RKO wild-type engrafted tumors appeared to be more heterogeneous than in RKO BCL9 knockout engrafted tumors (FIG. 6B, bottom). As expected, dotted BCL9 staining was detected in wild-type RKO cells but not in BCL9 knockout cells. β-catenin was not detected in any of the RKO cells and expression of the Wnt target gene Axin2 did not display any differences between the two groups (FIG. 6B and FIG. 6C). Furthermore, Ki-67 immunostains revealed increased number of proliferating cells in tumor nodules in mice implanted with wild-type compared with BCL9 knockout RKO cells (FIG. 6C and FIG. 6D). Immunostains were used to evaluate the expression of CD163, CD31 and αSMA, which were used as markers of infiltrating mouse derived M2 macrophages, as well as endothelial and myofibroblast cells, respectively. The number of M2 macrophages and endothelial cells, but not myofibroblasts, were significantly reduced within BCL9 knockout RKO engrafted tumors as compared with wild-type RKO engrafted tumors (FIG. 6C and FIG. 6E). The effect of inhibiting calcium influx on in vivo tumor growth was investigated by comparing the tumor burden and infiltration by cells of the tumor microenvironment in mice engrafted with RKO cells. The mice were subsequently treated with vehicle or propranolol in the drinking water. Three weeks after RKO cell injection, all seven vehicle treated mice but none of the propranolol treated mice had died with extensive tumor burden. Remarkably, three weeks post tumor injection, except for rare small tumor nodules, the tumor cells had disappeared in most of the propranolol treated mice (FIG. 6F and FIG. 6G). As seen in mice engrafted with BCL9 knockout cells, nuclear β-catenin was not detected in RKo cells and expression of Axin2 did not display differences between the control and treated groups. Consistently, propranolol treated mice demonstrated a marked reduction in the number of mouse derived M2 macrophages, and endothelial, but not myofibroblast cells within the tumor microenvironment (FIG. 6H and FIG. 6I). These in vivo results are consistent with previous in vitro results (FIG. 5G). The in vivo data support the role of BCL9 interaction with paraspeckle proteins in sustaining calcium influx-dependent, cell to cell communication, and promoting remodeling of the tumor microenvironment and also support a mechanistic role for BCL9 as target for CRC therapy.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A method of determining whether a subject has a C1 subtype of colorectal cancer (CRC), comprising:
obtaining a test sample from a subject having or at risk of having CRC;
determining the expression level of at least one C1 subtype-associated gene in the test sample;
comparing the expression level of the C1 subtype-associate gene in the test sample with the expression level of the C1 subtype-associated gene in a reference sample; and
identifying an elevated expression level of at least one C1 subtype-associated gene in the test sample as compared to the expression level of the C1 subtype-associated gene in a reference sample, wherein the C1 subtype-associated gene comprises a gene associated with wound healing, tissue remodeling, or neuron protection;
thereby determining that the subject has a C1 subtype of colorectal cancer (CRC).

2. The method of claim 1, wherein the method comprises identifying an elevated expression level of at least two C1 subtype-associated genes in the test sample as compared to the expression level of the C1 subtype-associated gene in a reference sample; or wherein the method comprises identifying an elevated expression level of at least three C1 subtype-associated genes in the test sample as compared to the expression level of the C1 subtype-associated gene in a reference sample.

3. (canceled)

4. The method of claim 1, wherein the C1 subtype-associated gene comprises fibroblast activating protein (FAP), platelet derived growth factor subunit B (PDGFB), complement C3 (C3), or synaptophysin (SYP).

5. The method of claim 1, further comprising identifying an elevated level of stromal cells in the test sample as compared to a reference sample.

6. The method of claim 5, wherein the stromal cell comprises a fibroblast, a pericyte, or a macrophage.

7. The method of claim 1, further comprising identifying an elevated level of stromal cells in the test sample as compared to the level of immune cells in the test sample; or further comprising identifying an elevated level of neural cells (ganglion cells) in the test sample as compared to a reference sample.

8. (canceled)

9. The method of claim 1, further comprising identifying an elevated level of nuclear B-Cell Lymphoma 9 Protein (BCL9) expression in tumor cells as compared to stromal cells from the test sample.

10. The method of claim 9, wherein the BCL9 expression is localized adjacent to one or more paraspeckles within the nucleus; or wherein the nuclear BCL9 expression in tumor cells exhibits a punctate pattern; or wherein the BCL9 expression or activity is independent of B-catenin expression or activity.

11. (canceled)

12. (canceled)

13. The method of claim 9, wherein the BCL9 expression is localized adjacent to one or more paraspeckles within the nucleus and wherein the BCL9 co-localizes adjacent to one or more paraspeckle proteins selected from the group consisting of valosin containing protein (VCP), non-POU domain octamer binding protein (NONO), splicing factor proline and glutamine rich protein (SFPQ), and interleukin enhancer binding factor 2 protein (ILF2).

14. The method of claim 1, wherein the test sample is obtained from a CRC tissue, a tumor microenvironment, a plasma sample, or a blood sample.

15. The method of claim 1, wherein the test sample comprises a deoxyribonucleic acid (DNA), a ribonucleic acid (RNA), or an amino acid.

16. The method of claim 1, wherein the reference sample is obtained from healthy normal tissue or CRC tissue.

17. The method of claim 1, wherein the reference sample is obtained from healthy normal tissue from the same individual as the test sample or one or more healthy normal tissues from different individuals.

18. The method of claim 1, wherein the expression level of the C1 subtype-associated gene is detected via an Affymetrix Gene Array hybridization, next generation sequencing, ribonucleic acid sequencing (RNA-seq), a real time reverse transcriptase polymerase chain reaction (real time RT-PCR) assay, immunohistochemistry (IHC), or immunofluorescence.

19. The method of claim 1, wherein the subject is human.

20. A method of treating a subject with a C1 subtype of CRC comprising:
determining whether a subject has a C1 subtype of CRC according to the method of claim 1; and
administering a therapeutically effective amount of a BCL9 inhibitor to the subject,
thereby treating a subject with a C1 subtype of CRC.

21. The method of claim 20, wherein the BCL9 inhibitor comprises a small molecule inhibitor, RNA interference (RNAi), microRNA (miRNA), an antibody, an antibody fragment, an antibody drug conjugate, an aptamer, a chimeric antigen receptor (CAR), a T cell receptor, or any combination thereof.

22. The method of claim 21, wherein the antibody or antibody fragment is partially humanized, fully humanized, or chimeric.

23. The method of claim 20, wherein the BCL9 inhibitor comprises a stabilized alpha helix (SAH), hydrocarbon-stapled, BCL9.

24. The method of claim 23, wherein the BCL9 inhibitor comprises

| (SEQ ID NO: 1) | |
|---|---|
| | LSQEQLEHRERSLXTLRXIQRBLF, |
| (SEQ ID NO: 2) | |
| | LSQEQLEHRERSLXTLRXIQRMLF, |
| (SEQ ID NO: 3) | |
| | LSQEQLEHRERSLQTLRXIQRXLF, |
| | or |
| (SEQ ID NO: 4) | |
| | LSQEQLEHREXSLQXLRDIQRBLF. |

25. The method of claim 20, wherein the BCL9 inhibitor comprises a miR-30 polynucleotide.

26. The method of claim 21, wherein the miR-30 polynucleotide comprises a polynucleotide comprising one or more sequences selected from the group consisting of SEQ ID NOs: 9-13.

27. The method of claim 20, wherein the BCL9 inhibitor comprises a nanoparticle.

28. The method of claim 27, wherein the nanoparticle comprises a BCL9 siRNA comprising SEQ ID NO: 5 or SEQ ID NO: 6.

29. The method of claim 20, wherein the BCL9 inhibitor reduces the interaction between BCL9 and one or more paraspeckles; or wherein BCL9 inhibition reduces tumor cell proliferation, tumor metastases, stromal cell infiltration, and response to cellular stress; or wherein BCL9 inhibition reduces expression or activity of one or more genes associated with calcium signaling or neural differentiation including regulator of G protein signaling 4 (RGS4), calcium voltage-gated channel auxiliary subunit alpha 2 delta 1 (CACNA2D1), calcium channel, voltage-dependent, L type, alpha 1D subunit (CACNAID), and adrenoceptor beta 1 (ADRB1).

30. (canceled)

31. (canceled)

32. The method of claim 20, further comprising administering a calcium channel receptor inhibitor or a beta-adrenergic antagonist.

33. The method of claim 32, wherein the calcium channel receptor inhibitor comprises verapamil, fendiline, gallopamil, amlodipine, aranidipine, azelnidipine, barnidipine, benidipine, cilidipine, clevidipine, efonidipine, felodipine, isradipine, lacidipine, lercanidipine, manidipine, nicardipine, nifedipine, nilvadipine, nimodipine, nisoldipine, nitrendipine, or pranidipine; or wherein the beta-adrenergic antagonist comprises propranolol, bucindolol, carteolol, carvedilol, labetalol, nadolol, oxprenolol, penbutolol, pindolol, sotalol, timolol, acebutolol, atenolol, betaxolol, bisoprolol, celiprolol, metoprolol, nebivolol, esmolol, butaxamine, or nebivolol.

34. (canceled)

35. The method of claim 20, further comprising treating the subject with a chemotherapeutic agent, radiation therapy, cryotherapy, hormone therapy, or immunotherapy.

36. The method of claim 35, wherein the chemotherapeutic agent comprises fluorouracil, capecitabine, oxaliplatin, irinotecan, or tegafur/uracil.

37. The method of claim 20, wherein the CRC comprises adenocarcinoma, gastrointestinal stromal tumors (GIST), lymphoma, a carcinoid tumor, familial colorectal cancer (FCC), or juvenile polyposis coli.


SEQUENCE LISTING (50 sequences)

SEQ ID NO: 1 - protein, 24 aa, Homo sapiens
  misc_feature (14)..(14): Xaa can be any naturally occurring amino acid
  misc_feature (18)..(18): Xaa can be any naturally occurring amino acid
LSQEQLEHRERSLXTLRXIQRBLF

SEQ ID NO: 2 - protein, 24 aa, Homo sapiens
  misc_feature (14)..(14): Xaa can be any naturally occurring amino acid
  misc_feature (18)..(18): Xaa can be any naturally occurring amino acid
LSQEQLEHRERSLXTLRXIQRMLF

SEQ ID NO: 3 - protein, 24 aa, Homo sapiens
  misc_feature (18)..(18): Xaa can be any naturally occurring amino acid
  misc_feature (22)..(22): Xaa can be any naturally occurring amino acid
LSQEQLEHRERSLQTLRXIQRXLF

SEQ ID NO: 4 - protein, 24 aa, Homo sapiens
  misc_feature (11)..(11): Xaa can be any naturally occurring amino acid
  misc_feature (15)..(15): Xaa can be any naturally occurring amino acid
LSQEQLEHREXSLQXLRDIQRBLF

SEQ ID NO: 5 - RNA, 21 nt, Homo sapiens
aauaacacuguguauugcagc

SEQ ID NO: 6 - RNA, 21 nt, Homo sapiens
uuccagggauauucacagagg

SEQ ID NO: 7 - protein, 1426 aa, Homo sapiens
MHSSNPKVRSSPSGNTQSSPKSKQEVMVRPPTVMSPSGNPQLDSKFSNQGKQGGSASQSQPSPCDSKSGGHTPKALPGPGGSMGLKNGAGNGAKGKGKRERSISADSFDQRDPGTPNDDSDIKECNSADHIKSQDSQHTPHSMTPSNATAPRSSTPSHGQTTATEPTPAQKTPAKVVYVFSTEMANKAAEAVLKGQVETIVSFHIQNISNNKTERSTAPLNTQISALRNDPKPLPQQPPAPANQDQNSSQNTRLQPTPPIPAPAPKPAAPPRPLDRESPGVENKLIPSVGSPASSTPLPPDGTGPNSTPNNRAVTPVSQGSNSSSADPKAPPPPPVSSGEPPTLGENPDGLSQEQLEHRERSLQTLRDIQRMLFPDEKEFTGAQSGGPQQNPGVLDGPQKKPEGPIQAMMAQSQSLGKGPGPRTDVGAPFGPQGHRDVPFSPDEMVPPSMNSQSGTIGPDHLDHMTPEQIAWLKLQQEFYEEKRRKQEQVVVQQCSLQDMMVHQHGPRGVVRGPPPPYQMTPSEGWAPGGTEPFSDGINMPHSLPPRGMAPHPNMPGSQMRLPGFAGMINSEMEGPNVPNPASRPGLSGVSWPDDVPKIPDGRNFPPGQGIFSGPGRGERFPNPQGLSEEMFQQQLAEKQLGLPPGMAMEGIRPSMEMNRMIPGSQRHMEPGNNPIFPRIPVEGPLSPSRGDFPKGIPPQMGPGRELEFGMVPSGMKGDVNLNVNMGSNSQMIPQKMREAGAGPEEMLKLRPGGSDMLPAQQKMVPLPFGEHPQQEYGMGPRPFLPMSQGPGSNSGLRNLREPIGPDQRTNSRLSHMPPLPLNPSSNPTSLNTAPPVQRGLGRKPLDISVAGSQVHSPGINPLKSPTMHQVQSPMLGSPSGNLKSPQTPSQLAGMLAGPAAAASIKSPPVLGSAAASPVHLKSPSLPAPSPGWTSSPKPPLQSPGIPPNHKAPLTMASPAMLGNVESGGPPPPTASQPASVNIPGSLPSSTPYTMPPEPTLSQNPLSIMMSRMSKFAMPSSTPLYHDAIKTVASSDDDSPPARSPNLPSMNNMPGMGINTQNPRISGPNPVVPMPTLSPMGMTQPLSHSNQMPSPNAVGPNIPPHGVPMGPGLMSHNPIMGHGSQEPPMVPQGRMGFPQGFPPVQSPPQQVPFPHNGPSGGQGSFPGGMGFPGEGPLGRPSNLPQSSADAALCKPGGPGGPDSFTVLGNSMPSVFTDPDLQEVIRPGATGIPEFDLSRIIPSEKPSQTLQYFPRGEVPGRKQPQGPGPGFSHMQGMMGEQAPRMGLALPGMGGPGPVGTPDIPLGTAPSMPGHNPMRPPAFLQQGMMGPHHRMMSPAQSTMPGQPTLMSNPAAAVGMIPGKDRGPAGLYTHPGPVGSPGMMMSMQGMMGPQQNIMIPPQMRPRGMAADVGMGGFSQGPGNPGNMMF

SEQ ID NO: 8 - DNA, 6192 bp, Homo sapiens
agaccagagcagacaataggcccctaaagtgttccccctaagttgctttgatgttgtcctggtgtcttgataccaggaggccagggattgcgggaaaagggtcttttttgtcttcattcactttcccccctcagtttctgaaatgattctccagaatttctcctcataaaaaaggactgaatgtgggcccagttggcgtcattctgctttgacctaaacattcccatctgattgggtggcagagatcatttttggaaagttcttccgtgtcccgatgtagaagaaatagcaaattggacatattgaaagacaagggtcatctttgagaagggggttcctggactcctcacctccaggatgagcactgcagtgtcgtgacccttggggttttgtatgccctggagatgcgagattttcctctggcagcaggaggcacgcacccagagaatgctggagctgcaaggggaaaggacccacttccacagcagagaaaaacaaagaggaaaaaggcatacaggcagcgagcgctaagggacgcacccagcaagcagtgggccagtgccactgcccccagcagctgtttctgctgcaacccgagaggaactcggtgagcctgtcccgtttgtgactgcaagctcaggatttcaatcaatgcattccagtaaccctaaagtgaggagctctccatcaggaaacacacagagtagccctaagtcaaagcaggaggtgatggtccgtccccctacagtgatgtccccatctggaaacccccagctggattccaaattctccaatcagggtaaacaggggggctcagccagccaatcccagccatccccctgtgactccaagagtgggggccatacccctaaagcactccctggcccaggtgggagcatggggctgaagaatggggctggaaatggtgccaagggcaaggggaaaagggagcgaagtatttccgccgactcctttgatcagagagatcctgggactccaaacgatgactctgacattaaagaatgtaattctgctgaccacataaagtcccaggattcccagcacacaccacactcgatgaccccatcaaatgctacagcccccaggtcttctaccccctcccatggccaaactactgccacagagcccacacctgctcagaagactccagccaaagtggtgtacgtgttttctactgagatggccaataaagctgcagaagctgttttgaagggccaggttgaaactatcgtctctttccacatccagaacatttctaacaacaagacagagagaagcacagcgcctctgaacacacagatatctgcccttcggaatgatccgaaacctctcccacaacagcccccagctccggccaaccaggaccagaattcttcccagaataccagactgcagccaactccacccattccggcaccagcacccaagcctgccgcacccccacgtcccctggaccgggagagtcctggggtagaaaacaaactgattccttctgtaggaagtcctgccagctccactccactgcccccagatggtactgggcccaactcaactcccaacaatagggcagtgacccctgtctcccaggggagcaatagctcttcagcagatcccaaagcccctccgcctccaccagtgtccagtggcgagccccccacactgggagagaatcccgatggcctatctcaggagcagctggagcaccgggagcgctccttacaaactctcagagatatccagcgcatgctttttcctgatgagaaagaattcacaggagcacaaagtgggggaccgcagcagaatcctggggtattagatgggcctcagaaaaaaccagaagggccaatacaggccatgatggcccaatcccaaagcctaggtaagggacctgggccccggacagacgtgggagctccatttggccctcaaggacatagagatgtacccttttctccagatgaaatggttccaccttctatgaactcccagtctgggaccataggacccgaccaccttgaccatatgactcccgagcagatagcgtggctgaaactgcagcaggagttttatgaagagaagaggaggaagcaggaacaagtggttgtccagcagtgttccctccaggacatgatggtccatcagcacgggcctcggggagtggtccgaggacccccccctccataccagatgacccctagtgaaggctgggcacctgggggtacagagccattttctgatggtatcaacatgccacattctctgcccccgaggggcatggctccccaccccaacatgccagggagccagatgcgcctccctggatttgcaggcatgataaactctgaaatggaagggccgaatgtccccaaccctgcatctagaccaggtctttctggagtcagttggccagatgatgtgccaaaaatcccagatggtcgaaattttcctcctggccagggcattttcagcggtcctggccgaggggaacgcttcccaaacccccaaggattgtctgaagagatgtttcagcagcagctggcagagaaacagctgggtctccccccagggatggccatggaaggcatcaggcccagcatggagatgaacaggatgattccaggctcccagcgccacatggagcctgggaataaccccattttccctcgaataccagttgagggccctctgagtccttctaggggtgactttccaaaaggaattcccccacagatgggccctggtcgggaacttgagtttgggatggttcctagtgggatgaagggagatgtcaatctaaatgtcaacatgggatccaactctcagatgatacctcagaagatgagagaggctggggcgggccctgaggagatgctgaaattacgcccaggtggctcagacatgctgcctgctcagcagaagatggtgccactgccatttggtgagcacccccagcaggagtatggcatgggccccagaccattccttcccatgtctcagggtccaggcagcaacagtggcttgcggaatctcagagaaccaattgggcccgaccagaggactaacagccggctcagtcatatgccaccactacctctcaacccttccagtaaccccaccagcctcaacacagctcctccagttcagcgcggcctggggcggaagcccttggatatatctgtggcaggcagccaggtgcattccccaggcattaaccctctgaagtctcccacgatgcaccaagtccagtcaccaatgctgggctcgccctcggggaacctcaagtccccccagactccatcgcagctggcaggcatgctggcgggcccagctgctgctgcttccattaagtccccccctgttttggggtctgctgctgcttcacctgtccacctcaagtctccatcacttcctgccccgtcacctggatggacctcttctccaaaacctccccttcagagtcctgggatccctccaaaccataaagcacccctcaccatggcctccccagccatgctgggaaatgtagagtcaggtggccccccacctcctacagccagccagcctgcctctgtgaatatccctggaagtcttccctctagtacaccttataccatgcctccagagccaaccctttcccagaacccactctctattatgatgtctcgaatgtccaagtttgcaatgcccagttccaccccgttataccatgatgctatcaagactgtggccagctcagatgacgactcccctccagctcgttctcccaacttgccatcaatgaataatatgccaggaatgggcattaatacacagaatcctcgaatttcaggtccaaaccccgtggttccgatgccaaccctcagcccaatgggaatgacccagccactttctcactccaatcagatgccctctccaaatgccgtgggacccaacatacctcctcatggggtcccaatggggcctggcttgatgtcacacaatcctatcatggggcatgggtcccaggagccaccgatggtacctcaaggacggatgggcttcccccagggcttccctccagtacagtctcccccacagcaggttccattccctcacaatggccccagtggggggcagggcagcttcccaggagggatgggtttcccaggagaaggcccccttggccgccccagcaacctgccccaaagttcagcagatgcagcactttgcaagcctggaggccccgggggtcctgactccttcactgtcctggggaacagcatgccttcggtgtttacagacccagatctgcaggaggtcatccgacctggagccaccggaatacctgagtttgatctatcccgcattattccatctgagaagcccagccagacgctgcaatatttccctcgaggggaagttccaggccgtaaacagccccagggtcctggacctgggttttcacacatgcaggggatgatgggcgaacaagcccccagaatgggactagcattacctggcatgggaggtccagggccagtgggaactccggacatccctcttggtacagctccatccatgccaggccacaaccccatgagaccaccagcctttctccaacaaggcatgatgggacctcaccatcggatgatgtcaccagcacaatctacaatgcccggccagcccaccctgatgagcaatccagctgctgccgtgggcatgattcctggcaaggatcgggggcctgccgggctctacacccaccctgggcctgtgggctctccaggcatgatgatgtccatgcagggcatgatgggaccccaacagaacatcatgatccccccacagatgaggccccggggcatggctgctgacgtgggcatgggtggatttagccaaggacctggcaacccaggaaacatgatgttttaagctgctaagatgggatgtgccgatccttgtcaagatgagattccaggtcctgagagctgctttgagggagttccaggagtacttactattggtcatgcaataggagaacagagacccgagggctgctttgggggaggggggaactcgagaatgtatggatttacctgaaaacaaattattcatttaatcaacaggtgtgttttttttaagatttattttttaaaaattatttttgtggacttgggtatcaatgatggcacctacttttgggaatctgtagctgtgctttgagaattgccatcggtcatgtgttgcaccgttctctgtatgtttacgtcctttggactggcttctcccaggattcttttctgtttttgtttttttgatttgggctttatttttttctgtgtactgtactatattgtaaaagggattttagcagagactttagtctttggggcaagaggagaacaggaatgctgggctgtttactttaggtggagaatccatcttcagacctttggactattttctttcaactgcagtgtatagaaaaaccaaactacgacctcagagcagagtattaatgaaaagcacaaaaaaaggaactaagttcagcgaggggtggggggaggggggagatttttcttttgaaaaataatgactcttaggacatttgtttttcagttcaagtgctcttcagcactgtcttgtctcccaatataccaacccactggcacatttttctcttgttttctctctccgattttgctctgtctcctcagttaagtgtttccttcctttgtgccccccgctggtgaccctctgcttccctctctctttccctttggcagctgcaatacacagtgttattttggggaaataaatctagcaaagcctcgccttccatgccgagcgtcctcttggctctgagagggaaaggtctgtccttgggatgctctctggtcttttttccccctaagtctttctctttcccatcatacccttccctgcccaccttgttttctgttctccttttattaggaattcccaagtgaattttattaatgtgggagtggaacagatgctaaaagctatccaggattttgtttctgtttgttttaaattttgtggttccttccctttcctccccctcccatgcgtaagacgttctgtgtaacctccattaaatttggtacaaaaccactcgccagagctgtggtgtcagaaaaataaaatatattgtttcttacaaaattg

SEQ ID NO: 9 - RNA, 22 nt, Homo sapiens
uguaaacauccucgacuggaag

SEQ ID NO: 10 - RNA, 22 nt, Homo sapiens
uguaaacauccuacacucagcu

SEQ ID NO: 11 - RNA, 23 nt, Homo sapiens
uguaaacauccuacacucucagc

SEQ ID NO: 12 - RNA, 22 nt, Homo sapiens
uguaaacauccccgacuggaag

SEQ ID NO: 13 - RNA, 21 nt, Homo sapiens
uguaaacauccuugacugaag

SEQ ID NO: 14 - DNA, 1416 bp, Homo sapiens
atgcagagtaataaaacttttaacttggagaagcaaaaccatactccaagaaagcatcatcaacatcaccaccagcagcagcaccaccagcagcaacagcagcagccgccaccaccgccaatacctgcaaatgggcaacaggccagcagccaaaatgaaggcttgactattgacctgaagaattttagaaaaccaggagagaagaccttcacccaacgaagccgtctttttgtgggaaatcttcctcccgacatcactgaggaagaaatgaggaaactatttgagaaatatggaaaggcaggcgaagtcttcattcataaggataaaggatttggctttatccgcttggaaacccgaaccctagcggagattgccaaagtggagctggacaatatgccactccgtggaaagcagctgcgtgtgcgctttgcctgccatagtgcatcccttacagttcgaaaccttcctcagtatgtgtccaacgaactgctggaagaagccttttctgtgtttggccaggtagagagggctgtagtcattgtggatgatcgaggaaggccctcaggaaaaggcattgttgagttctcagggaagccagctgctcggaaagctctggacagatgcagtgaaggctccttcctgctaaccacatttcctcgtcctgtgactgtggagcccatggaccagttagatgatgaagagggacttccagagaagctggttataaaaaaccagcaatttcacaaggaacgagagcagccacccagatttgcacagcctggctcctttgagtatgaatatgccatgcgctggaaggcactcattgagatggagaagcagcagcaggaccaagtggaccgcaacatcaaggaggctcgtgagaagctggagatggagatggaagctgcacgccatgagcaccaggtcatgctaatgagacaggatttgatgaggcgccaagaagaacttcggaggatggaagagctgcacaaccaagaggtgcaaaaacgaaagcaactggagctcaggcaggaggaagagcgcaggcgccgtgaagaagagatgcggcggcagcaagaagaaatgatgcggcgacagcaggaaggattcaagggaaccttccctgatgcgagagagcaggagattcggatgggtcagatggctatgggaggtgctatgggcataaacaacagaggtgccatgccccctgctcctgtgccagctggtaccccagctcctccaggacccgccactatgatgccggatggaactttgggattgaccccaccaacaactgaacgctttggtcaggctgctacaatggaaggaattggggcaattggtggaactcctcctgcattcaaccgtgcagctcctggagctgaatttgccccaaacaaacgtcgccgatattaa

SEQ ID NO: 15 - protein, 390 aa, Homo sapiens
MRGDRGRGRGGRFGSRGGPGGGFRPFVPHIPFDFYLCEMAFPRVKPAPDETSFSEALLKRNQDLAPNSAEQASILSLVTKINNVIDNLIVAPGTFEVQIEEVRQVGSYKKGTMTTGHNVADLVVILKILPTLEAVAALGNKVVESLRAQDPSEVLTMLTNETGFEISSSDATVKILITTVPPNLRKLDPELHLDIKVLQSALAAIRHARWFEENASQSTVKVLIRLLKDLRIRFPGFEPLTPWILDLLGHYAVMNNPTRQPLALNVAYRRCLQILAAGLFLPGSVGITDPCESGNFRVHTVMTLEQQDMVCYTAQTLVRILSHGGFRKILGQEGDASYLASEISTWDGVIVTPSEKAYEKPPEKKEGEEEEENTEEPPQGEEEESMETQE

SEQ ID NO: 16 - DNA, 1852 bp, Homo sapiens
ctcttcagttgtctgctactcagaggaaggggcggttggtgcggcctccattgttcgtgttttaaggcgccatgaggggtgacagaggccgtggtcgtggtgggcgctttggttccagaggaggcccaggaggagggttcaggccctttgtaccacatatcccatttgacttctatttgtgtgaaatggcctttccccgggtcaagccagcacctgatgaaacttccttcagtgaggccttgctgaagaggaatcaggacctggctcccaattctgctgaacaggcatctatcctttctctggtgacaaaaataaacaatgtgattgataatctgattgtggctccagggacatttgaagtgcaaattgaagaagttcgacaggtgggatcctataaaaaggggacaatgactacaggacacaatgtggctgacctggtggtgatactcaagattctgccaacgttggaagctgttgctgccctggggaacaaagtcgtggaaagcctaagagcacaggatccttctgaagttttaaccatgctgaccaacgaaactggctttgaaatcagttcttctgatgctacagtgaagattctcattacaacagtgccacccaatcttcgaaaactggatccagaactccatttggatatcaaagtattgcagagtgccttagcagccatccgacatgcccgctggttcgaggaaaatgcttctcagtccacagttaaagttctcatcagactactgaaggacttgaggattcgttttcctggctttgagcccctcacaccctggatccttgacctactaggccattatgctgtgatgaacaaccccaccagacagcctttggccctaaacgttgcatacaggcgctgcttgcagattctggctgcaggactgttcctgccaggttcagtgggtatcactgacccctgtgagagtggcaactttagagtacacacagtcatgaccctagaacagcaggacatggtctgctatacagctcagactctcgtccgaatcctctcacatggtggctttaggaagatccttggccaggagggtgatgccagctatcttgcttctgaaatatctacctgggatggagtgatagtaacaccttcagaaaaggcttatgagaagccaccagagaagaaggaaggagaggaagaagaggagaatacagaagaaccacctcaaggagaggaagaagaaagcatggaaactcaggagtgacattcccttcactccttttcctacccaagggggaagactggagcctaagctgcctgctactgggctttacatggtgacagacatttccgtgggatagggaagatagcaggaagaaaagtaaactccatagaagtgtcattccactgggttttgatattggcttagctgccagtctcccatttgtgacctatgccatccatctataatggaggataccaacatttcttcctaatattctataatctccaactcctgaaaacccctctctcaactaatactttgctgttgaaatgttgtgaaatgttaagtgtctggaaatttttttttctaagaaaaactattaaagtacttcctagtagggctcttggtctttctggttggtgttttttgtttgtttgtttttttgcatgtggcaaccttttttgcttttggctttgtgtactcttttgttgaaaatgataattgagtcatttaattataaatactcagatcacaatttgaagctgtctttgaattaaaggacaaactcctggaacctttccatataccagagtaaactagaatatcattttaagtatttattgaataaaatgattctatcaacactg

SEQ ID NO: 17 - protein, 707 aa, Homo sapiens
MSRDRFRSRGGGGGGFHRRGGGGGRGGLHDFRSPPPGMGLNQNRGPMGPGPGQSGPKPPIPPPPPHQQQQQPPPQQPPPQQPPPHQPPPHPQPHQQQQPPPPPQDSSKPVVAQGPGPAPGVGSAPPASSSAPPATPPTSGAPPGSGPGPTPTPPPAVTSAPPGAPPPTPPSSGVPTTPPQAGGPPPPPAAVPGPGPGPKQGPGPGGPKGGKMPGGPKPGGGPGLSTPGGHPKPPHRGGGEPRGGRQHHPPYHQQHHQGPPPGGPGGRSEEKISDSEGFKANLSLLRRPGEKTYTQRCRLFVGNLPADITEDEFKRLFAKYGEPGEVFINKGKGFGFIKLESRALAEIAKAELDDTPMRGRQLRVRFATHAAALSVRNLSPYVSNELLEEAFSQFGPIERAVVIVDDRGRSTGKGIVEFASKPAARKAFERCSEGVFLLTTTPRPVIVEPLEQLDDEDGLPEKLAQKNPMYQKERETPPRFAQHGTFEYEYSQRWKSLDEMEKQQREQVEKNMKDAKDKLESEMEDAYHEHQANLLRQDLMRRQEELRRMEELHNQEMQKRKEMQLRQEEERRRREEEMMIRQREMEEQMRRQREESYSRMGYMDPRERDMRMGGGGAMNMGDPYGSGGQKFPPLGGGGGIGYEANPGVPPATMSGSMMGSDMRTERFGQGGAGPVGGQGPRGMGPGTPAGYGRGREEYEGPNKKPRF

SEQ ID NO: 18 - DNA, 3740 bp, Homo sapiens
gcctgtgtcatccgccattttgtgagaagcaaggtggcctccacgtttcctgagcgtcttcttcgcttttgcctcgaccgccccttgaccacagacatgtctcgggatcggttccggagtcgtggcggtggcggtggtggcttccacaggcgtggaggaggcggcggccgcggcggcctccacgacttccgttctccgccgcccggcatgggcctcaatcagaatcgcggccccatgggtcctggcccgggccagagcggccctaagcctccgatcccgccaccgcctccacaccaacagcagcaacagccaccaccgcagcagccaccgccgcagcagccgccaccgcatcagccgccgccgcatccacagccgcatcagcagcagcagccgccgccaccgccgcaggactcttccaagcccgtcgttgctcagggacccggccccgctcccggagtaggcagcgcaccaccagcctccagctcggccccgcccgccactccaccaacctcgggggccccgccagggtccgggccaggcccgactccgaccccgccgcctgcagtcacctcggcccctcccggggcgccgccacccaccccgccaagcagcggggtccctaccacacctcctcaggccggaggcccgccgcctccgcccgcggcagtcccgggcccgggtccagggcctaagcagggcccaggtccgggtggtcccaaaggcggcaaaatgcctggcgggccgaagccaggtggcggcccgggcctaagtacgcctggcggccaccccaagccgccgcatcgaggcggcggggagccccgcgggggccgccagcaccacccgccctaccaccagcagcatcaccaggggcccccgcccggcgggcccggcggccgcagcgaggagaagatctcggactcggaggggtttaaagccaatttgtctctcttgaggaggcctggagagaaaacttacacacagcgatgtcggttgtttgttgggaatctacctgctgatatcacggaggatgaattcaaaagactatttgctaaatatggagaaccaggagaagtttttatcaacaaaggcaaaggattcggatttattaagcttgaatctagagctttggctgaaattgccaaagccgaactggatgatacacccatgagaggtagacagcttcgagttcgctttgccacacatgctgctgccctttctgttcgtaatctttcaccttatgtttccaatgaactgttggaagaagcctttagccaatttggtcctattgaaagggctgttgtaatagtggatgatcgtggaagatctacagggaaaggcattgttgaatttgcttctaagccagcagcaagaaaggcatttgaacgatgcagtgaaggtgttttcttactgacgacaactcctcgtccagtcattgtggaaccacttgaacaactagatgatgaagatggtcttcctgaaaaacttgcccagaagaatccaatgtatcaaaaggagagagaaacccctcctcgttttgcccagcatggcacgtttgagtacgaatattctcagcgatggaagtctttggatgaaatggaaaaacagcaaagggaacaagttgaaaaaaacatgaaagatgcaaaagacaaattggaaagtgaaatggaagatgcctatcatgaacatcaggcaaatcttttgcgccaagatctgatgagacgacaggaagaattaagacgcatggaagaacttcacaatcaagaaatgcagaaacgtaaagaaatgcaattgaggcaagaggaggaacgacgtagaagagaggaagagatgatgattcgtcaacgtgagatggaagaacaaatgaggcgccaaagagaggaaagttacagccgaatgggctacatggatccacgggaaagagacatgcgaatgggtggcggaggagcaatgaacatgggagatccctatggttcaggaggccagaaatttccacctctaggaggtggtggtggcataggttatgaagctaatcctggcgttccaccagcaaccatgagtggttccatgatgggaagtgacatgcgtactgagcgctttgggcagggaggtgcggggcctgtgggtggacagggtcctagaggaatggggcctggaactccagcaggatatggtagagggagagaagagtacgaaggcccaaacaaaaaaccccgattttagatgtgatatttaggctttcattccagtttgttttgtttttttgtttagataccaatcttttaaattcttgcattttagtaagaaagctatctttttatggatgttagcagtttattgacctaatatttgtaaatggtctgtttgggcaggtaaaattatgtaatgcagtgtttggaacaggagaatttttttttcctttttatttctttattttttcttttttactgtataatgtccctcaagtttatggcagtgtaccttgtgccactgaatttccaaagtgtaccaatttttttttttttactgtgcttcaaataaatagaaaaatagttataatattgatcttcaactttgccattcatgcttctatgcatattaggctacgtattccacattgaaagcatgagagtgtctaggcctttgaatggcatatgccatttctgggaaatgcatctggaggctaagtattgctttctacaaataattgccccctttgttttaaaaagaagaaatgcatattgaagtagtttgatgatttgtttggcatataggaagcacgctggtgctaagtattttttaaatggttatgtaagcaaagctgaactgtaaatcttcaggaatatgtattaagattgtggaatgggtgtaagacaattggtagggggtgaaagtgggtttgattaaatggatcttttatggccctatgatctatcctttacttgaaagcttttgaaaagtggaaaggtcattttgttgcatttccccatttcttgtttttaaaagaccaacaaatctcaagccctataaatggcttgtattgaacttttacatttgaattaaagatgttaaacatgaagtcttttcatgtattcaagtgatttataaagatggggtgtagtctaaaaaacaaaacttgaaagtaagaaggtttagtaactgggccgggcgcggtggctcacgcctgtaatcccagcactttgggaggctgaggtgggcaaatcatgagatcaggagttcaagaccagcctggccaacatggtgaaaccccgtctctactaaaaatacaaaaaattagctgggcgtggtggcaggcgcctgtaatcccagctactcgggaggctgagccaggagaattgcttgaacctggagatggaggttgcagtgagctgagactgccactgcactccagcctggacaacagagcgagactctgtctcaaaaaaaaaaaaaggtttagtactgaagtaggtttagttatatatacatttgtactctgtaacttttacatctccttgtgctacttctgttgggcaaaatctataggactataaaaatctattggttctcttctaagagctagtggtgattgttaacaaatagggttgcagataatcattcttattgtaaatttaacttgttttcacatagtttcgttttcatgtagcactaaatctggagaaagttgctcatgctacacagtacaaatatcccttcctcccaccataaaggcttggaatgtt

SEQ ID NO: 19 - protein, 806 aa, Homo sapiens
MASGADSKGDDLSTAILKQKNRPNRLIVDEAINEDNSVVSLSQPKMDELQLFRGDTVLLKGKKRREAVCIVLSDDTCSDEKIRMNRVVRNNLRVRLGDVISIQPCPDVKYGKRIHVLPIDDTVEGITGNLFEVYLKPYFLEAYRPIRKGDIFLVRGGMRAVEFKVVETDPSPYCIVAPDTVIHCEGEPIKREDEEESLNEVGYDDIGGCRKQLAQIKEMVELPLRHPALFKAIGVKPPRGILLYGPPGTGKTLIARAVANETGAFFFLINGPEIMSKLAGESESNLRKAFEEAEKNAPAIIFIDELDAIAPKREKTHGEVERRIVSQLLTLMDGLKQRAHVIVMAATNRPNSIDPALRRFGRFDREVDIGIPDATGRLEILQIHTKNMKLADDVDLEQVANETHGHVGADLAALCSEAALQAIRKKMDLIDLEDETIDAEVMNSLAVTMDDFRWALSQSNPSALRETVVEVPQVTWEDIGGLEDVKRELQELVQYPVEHPDKFLKFGMTPSKGVLFYGPPGCGKTLLAKAIANECQANFISIKGPELLTMWFGESEANVREIFDKARQAAPCVLFFDELDSIAKARGGNIGDGGGAADRVINQILTEMDGMSTKKNVFIIGATNRPDIIDPAILRPGRLDQLIYIPLPDEKSRVAILKANLRKSPVAKDVDLEFLAKMTNGFSGADLTEICQRACKLAIRESIESEIRRERERQTNPSAMEVEEDDPVPEIRRDHFEEAMRFARRSVSDNDIRKYEMFAQTLQQSRGFGSFRFPSGNQGGAGPSQGSGGGTGGSVYTEDNDDDLYG

SEQ ID NO: 20 - DNA, 3743 bp, Homo sapiens
agtctcagcgaagcgtctgcgaccgtcgtttgagtcgtcgctgccgctgccgctgccactgccactgccacctcgcggatcaggagccagcgttgttcgcccgacgcctcgctgccggtgggaggaagcgagagggaagccgcttgcgggtttgtcgccgctgctcgcccaccgcctggaagagccgagccccggcccagtcggtcgcttgccaccgctcgtagccgttacccgcgggccgccacagccgccggccgggagaggcgcgcgccatggcttctggagccgattcaaaaggtgatgacctatcaacagccattctcaaacagaagaaccgtcccaatcggttaattgttgatgaagccatcaatgaggacaacagtgtggtgtccttgtcccagcccaagatggatgaattgcagttgttccgaggtgacacagtgttgctgaaaggaaagaagagacgagaagctgtttgcatcgtcctttctgatgatacttgttctgatgagaagattcggatgaatagagttgttcggaataaccttcgtgtacgcctaggggatgtcatcagcatccagccatgccctgatgtgaagtacggcaaacgtatccatgtgctgcccattgatgacacagtggaaggcattactggtaatctcttcgaggtataccttaagccgtacttcctggaagcgtatcgacccatccggaaaggagacatttttcttgtccgtggtgggatgcgtgctgtggagttcaaagtggtggaaacagatcctagcccttattgcattgttgctccagacacagtgatccactgcgaaggggagcctatcaaacgagaggatgaggaagagtccttgaatgaagtagggtatgatgacattggtggctgcaggaagcagctagctcagataaaggagatggtggaactgcccctgagacatcctgccctctttaaggcaattggtgtgaagcctcctagaggaatcctgctttacggacctcctggaacaggaaagaccctgattgctcgagctgtagcaaatgagactggagccttcttcttcttgatcaatggtcctgagatcatgagcaaattggctggtgagtctgagagcaaccttcgtaaagcctttgaggaggctgagaagaatgctcctgccatcatcttcattgatgagctagatgccatcgctcccaaaagagagaaaactcatggcgaggtggagcggcgcattgtatcacagttgttgaccctcatggatggcctaaagcagagggcacatgtgattgttatggcagcaaccaacagacccaacagcattgacccagctctacggcgatttggtcgctttgacagggaggtagatattggaattcctgatgctacaggacgcttagagattcttcagatccataccaagaacatgaagctggcagatgatgtggacctggaacaggtagccaatgagactcacgggcatgtgggtgctgacttagcagccctgtgctcagaggctgctctgcaagccatccgcaagaagatggatctcattgacctagaggatgagaccattgatgccgaggtcatgaactctctagcagttactatggatgacttccggtgggccttgagccagagtaacccatcagcactgcgggaaaccgtggtagaggtgccacaggtaacctgggaagacatcgggggcctagaggatgtcaaacgtgagctacaggagctggtccagtatcctgtggagcacccagacaaattcctgaagtttggcatgacaccttccaagggagttctgttctatggacctcctggctgtgggaaaactttgttggccaaagccattgctaatgaatgccaggccaacttcatctccatcaagggtcctgagctgctcaccatgtggtttggggagtctgaggccaatgtcagagaaatctttgacaaggcccgccaagctgccccctgtgtgctattctttgatgagctggattcgattgccaaggctcgtggaggtaacattggagatggtggtggggctgctgaccgagtcatcaaccagatcctgacagaaatggatggcatgtccacaaaaaaaaatgtgttcatcattggcgctaccaaccggcctgacatcattgatcctgccatcctcagacctggccgtcttgatcagctcatctacatcccacttcctgatgagaagtcccgtgttgccatcctcaaggctaacctgcgcaagtccccagttgccaaggatgtggacttggagttcctggctaaaatgactaatggcttctctggagctgacctgacagagatttgccagcgtgcttgcaagctggccatccgtgaatccatcgagagtgagattaggcgagaacgagagaggcagacaaacccatcagccatggaggtagaagaggatgatccagtgcctgagatccgtcgagatcactttgaagaagccatgcgctttgcgcgccgttctgtcagtgacaatgacattcggaagtatgagatgtttgcccagacccttcagcagagtcggggctttggcagcttcagattcccttcagggaaccagggtggagctggccccagtcagggcagtggaggcggcacaggtggcagtgtatacacagaagacaatgatgatgacctgtatggctaagtggtggtggccagcgtgcagtgagctggcctgcctggaccttgttccctgggggtgggggcgcttgcccaggagagggaccaggggtgcgcccacagcctgctccattctccagtctgaacagttcagctacagtctgactctggacagggggtttctgttgcaaaaatacaaaacaaaagcgataaaataaaagcgattttcatttggtaggcggagagtgaattaccaacagggaattgggccttgggcctatgccatttctgttgtagtttggggcagtgcaggggacctgtgtggggtgtgaaccaaggcactactgccacctgccacagtaaagcatctgcacttgactcaatgctgcccgagccctcccttccccctatccaacctgggtaggtgggtaggggccacagttgctggatgtttatatagagagtaggttgatttattttacatgcttttgagttaatgttggaaaactaatcacaagcagtttctaaaccaaaaaatgacatgttgtaaaaggacaataaacgttgggtcaaaatggagcctgagtcctgggccctgtgcctgcttcttttcctgggaacagccttgggctacccaccactcccaaggcattcttccaaatgtgaaatcctggaagtaagattgcaccttcttcctctcctgatcaacatcggtatgatgtctcctgttgcctcaccctttgtctgcagtatcactggataggactggtggaaagggagcagcctgacagagctccaaatgtggagaatatggcatccctccacctatatttgatgtggacggtaaggctaggcctgcaggatcccttatcctgaccaaagactgtgttggggtgccatttgaaaatcgcagggttgcaaaagaatacaatcttacttgcaggtggatattctctatactctcttttaatgcatctaaaaatcccaaacatcccctggttggtgatcacttacagttgtgtccacctttattttatgtactttgattaaaaaaaaactttttgttaatataaaa

SEQ ID NO: 21 - protein, 760 aa, Homo sapiens
MKTWVKIVFGVATSAVLALLVMCIVLRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 22 - DNA, 2696 bp, Homo sapiens
gtcctggcttcagcttccaactacaaagacagacttggtccttttcaacggttttcacagatccagtgacccacgctctgaagacagaattagctaactttcaaaaacatctggaaaaatgaagacttgggtaaaaatcgtatttggagttgccacctctgctgtgcttgccttattggtgatgtgcattgtcttacgcccttcaagagttcataactctgaagaaaatacaatgagagcactcacactgaaggatattttaaatggaacattttcttataaaacattttttccaaactggatttcaggacaagaatatcttcatcaatctgcagataacaatatagtactttataatattgaaacaggacaatcatataccattttgagtaatagaaccatgaaaagtgtgaatgcttcaaattacggcttatcacctgatcggcaatttgtatatctagaaagtgattattcaaagctttggagatactcttacacagcaacatattacatctatgaccttagcaatggagaatttgtaagaggaaatgagcttcctcgtccaattcagtatttatgctggtcgcctgttgggagtaaattagcatatgtctatcaaaacaatatctatttgaaacaaagaccaggagatccaccttttcaaataacatttaatggaagagaaaataaaatatttaatggaatcccagactgggtttatgaagaggaaatgcttgctacaaaatatgctctctggtggtctcctaatggaaaatttttggcatatgcggaatttaatgatacggatataccagttattgcctattcctattatggcgatgaacaatatcctagaacaataaatattccatacccaaaggctggagctaagaatcccgttgttcggatatttattatcgataccacttaccctgcgtatgtaggtccccaggaagtgcctgttccagcaatgatagcctcaagtgattattatttcagttggctcacgtgggttactgatgaacgagtatgtttgcagtggctaaaaagagtccagaatgtttcggtcctgtctatatgtgacttcagggaagactggcagacatgggattgtccaaagacccaggagcatatagaagaaagcagaactggatgggctggtggattctttgtttcaacaccagttttcagctatgatgccatttcgtactacaaaatatttagtgacaaggatggctacaaacatattcactatatcaaagacactgtggaaaatgctattcaaattacaagtggcaagtgggaggccataaatatattcagagtaacacaggattcactgttttattctagcaatgaatttgaagaataccctggaagaagaaacatctacagaattagcattggaagctatcctccaagcaagaagtgtgttacttgccatctaaggaaagaaaggtgccaatattacacagcaagtttcagcgactacgccaagtactatgcacttgtctgctacggcccaggcatccccatttccacccttcatgatggacgcactgatcaagaaattaaaatcctggaagaaaacaaggaattggaaaatgctttgaaaaatatccagctgcctaaagaggaaattaagaaacttgaagtagatgaaattactttatggtacaagatgattcttcctcctcaatttgacagatcaaagaagtatcccttgctaattcaagtgtatggtggtccctgcagtcagagtgtaaggtctgtatttgctgttaattggatatcttatcttgcaagtaaggaagggatggtcattgccttggtggatggtcgaggaacagctttccaaggtgacaaactcctctatgcagtgtatcgaaagctgggtgtttatgaagttgaagaccagattacagctgtcagaaaattcatagaaatgggtttcattgatgaaaaaagaatagccatatggggctggtcctatggaggatacgtttcatcactggcccttgcatctggaactggtcttttcaaatgtggtatagcagtggctccagtctccagctgggaatattacgcgtctgtctacacagagagattcatgggtctcccaacaaaggatgataatcttgagcactataagaattcaactgtgatggcaagagcagaatatttcagaaatgtagactatcttctcatccacggaacagcagatgataatgtgcactttcaaaactcagcacagattgctaaagctctggttaatgcacaagtggatttccaggcaatgtggtactctgaccagaaccacggcttatccggcctgtccacgaaccacttatacacccacatgacccacttcctaaagcagtgtttctctttgtcagactaaaaacgatgcagatgcaagcctgtatcagaatctgaaaaccttatataaacccctcagacagtttgcttattttattttttatgttgtaaaatgctagtataaacaaacaaattaatgttgttctaaaggctgttaaaaaaaagatgaggactcagaagttcaagctaaatattgtttacattttctggtactctgtgaaagaagagaaaagggagtcatgcattttgctttggacacagtgttttatcacctgttcatttgaagaaaaataataaagtcagaagttcaagtgcta

SEQ ID NO: 23 - protein, 241 aa, Homo sapiens
MNRCWALFLSLCCYLRLVSAEGDPIPEELYEMLSDHSIRSFDDLQRLLHGDPGEEDGAELDLNMTRSHSGGELESLARGRRSLGSLTIAEPAMIAECKTRTEVFEISRRLIDRTNANFLVWPPCVEVQRCSGCCNNRNVQCRPTQVQLRPVQVRKIEIVRKKPIFKKATVTLEDHLACKCETVAAARPVTRSPGGSQEQRAKTPQTRVTIRTVRVRRPPKGKHRKFKHTHDKTALKETLGA

SEQ ID NO: 24 - DNA, 3801 bp, Homo sapiens
gaggaaaggctgtctccacccacctctcgcactctcccttctcctttataaaggccggaacagctgaaagggtggcaacttctcctcctgcagccgggagcggcctgcctgcctccctgcgcacccgcagcctcccccgctgcctccctagggctcccctccggccgccagcgcccatttttcattccctagatagagatactttgcgcgcacacacatacatacgcgcgcaaaaaggaaaaaaaaaaaaaaaagcccaccctccagcctcgctgcaaagagaaaaccggagcagccgcagctcgcagctcgcagctcgcagcccgcagcccgcagaggacgcccagagcggcgagcgggcgggcagacggaccgacggactcgcgccgcgtccacctgtcggccgggcccagccgagcgcgcagcgggcacgccgcgcgcgcggagcagccgtgcccgccgcccgggccccgcgccagggcgcacacgctcccgcccccctacccggcccgggcgggagtttgcacctctccctgcccgggtgctcgagctgccgttgcaaagccaactttggaaaaagttttttgggggagacttgggccttgaggtgcccagctccgcgctttccgattttgggggcctttccagaaaatgttgcaaaaaagctaagccggcgggcagaggaaaacgcctgtagccggcgagtgaagacgaaccatcgactgccgtgttccttttcctcttggaggttggagtcccctgggcgcccccacacggctagacgcctcggctggttcgcgacgcagccccccggccgtggatgctcactcgggctcgggatccgcccaggtagcggcctcggacccaggtcctgcgcccaggtcctcccctgccccccagcgacggagccggggccgggggcggcggcgcccgggggccatgcgggtgagccgcggctgcagaggcctgagcgcctgatcgccgcggacccgagccgagcccacccccctccccagccccccaccctggccgcgggggcggcgcgctcgatctacgcgtccggggccccgcggggccgggcccggagtcggcatgaatcgctgctgggcgctcttcctgtctctctgctgctacctgcgtctggtcagcgccgagggggaccccattcccgaggagctttatgagatgctgagtgaccactcgatccgctcctttgatgatctccaacgcctgctgcacggagaccccggagaggaagatggggccgagttggacctgaacatgacccgctcccactctggaggcgagctggagagcttggctcgtggaagaaggagcctgggttccctgaccattgctgagccggccatgatcgccgagtgcaagacgcgcaccgaggtgttcgagatctcccggcgcctcatagaccgcaccaacgccaacttcctggtgtggccgccctgtgtggaggtgcagcgctgctccggctgctgcaacaaccgcaacgtgcagtgccgccccacccaggtgcagctgcgacctgtccaggtgagaaagatcgagattgtgcggaagaagccaatctttaagaaggccacggtgacgctggaagaccacctggcatgcaagtgtgagacagtggcagctgcacggcctgtgacccgaagcccggggggttcccaggagcagcgagccaaaacgccccaaactcgggtgaccattcggacggtgcgagtccgccggccccccaagggcaagcaccggaaattcaagcacacgcatgacaagacggcactgaaggagacccttggagcctaggggcatcggcaggagagtgtgtgggcagggttatttaatatggtatttgctgtattgcccccatggggtccttggagtgataatattgtttccctcgtccgtctgtctcgatgcctgattcggacggccaatggtgcttcccccacccctccacgtgtccgtccacccttccatcagcgggtctcctcccagcggcctccggcgtcttgcccagcagctcaagaagaaaaagaaggactgaactccatcgccatcttcttcccttaactccaagaacttgggataagagtgtgagagagactgatggggtcgctctttgggggaaacgggctccttcccctgcacctggcctgggccacacctgagcgctgtggactgtcctgaggagccctgaggacctctcagcatagcctgcctgatccctgaacccctggccagctctgaggggaggcacctccaggcaggccaggctgcctcggactccatggctaagaccacagacgggcacacagactggagaaaacccctcccacggtgcccaaacaccagtcacctcgtctccctggtgcctctgtgcacagtggcttcttttcgttttcgttttgaagacgtggactcctcttggtgggtgtggccagcacaccaagtggctgggtgccctctcaggtgggttagagatggagtttgctgttgaggtggctgtagatggtgacctgggtatcccctgcctcctgccaccccttcctccccacactccactctgattcacctcttcctctggttcctttcatctctctacctccaccctgcattttcctcttgtcctggcccttcagtctgctccaccaaggggctcttgaaccccttattaaggccccagatgatcccagtcactcctctctagggcagaagactagaggccagggcagcaagggacctgctcatcatattccaacccagccacgactgccatgtaaggttgtgcagggtgtgtactgcacaaggacattgtatgcagggagcactgttcacatcatagataaagctgatttgtatatttattatgacaatttctggcagatgtaggtaaagaggaaaaggatccttttcctaattcacacaaagactccttgtggactggctgtgcccctgatgcagcctgtggcttggagtggccaaataggagggagactgtggtaggggcagggaggcaacactgctgtccacatgacctccatttcccaaagtcctctgctccagcaactgcccttccaggtgggtgtgggacacctgggagaaggtctccaagggagggtgcagccctcttgcccgcacccctccctgcttgcacacttccccatctttgatccttctgagctccacctctggtggctcctcctaggaaaccagctcgtgggctgggaatgggggagagaagggaaaagatccccaagaccccctggggtgggatctgagctcccacctcccttcccacctactgcactttcccccttcccgccttccaaaacctgcttccttcagtttgtaaagtcggtgattatatttttgggggctttccttttattttttaaatgtaaaatttatttatattccgtatttaaagttgtaaaaaaaaataaccacaaaacaaaaccaaatgaatccgccggaggtctgtctgttggcatcgtgcgtgacaattaacctttctgccttggcaggatgtgccgacagcttgcggcgtgttcctctcactctgggagcctcaggcgtgatctcacacactggcgtgcacatacacacacacacacatacatgctcacacatgcgtgcacatacacgcaggcctgcaacttgggggaggcctctgtctggcgggaagaagagacacacaggctactctgttggtcttggtcctggcacagctcctgacacgtggacttgtgcgtgtctctggcagtgacgagagatgggtttctgcag

SEQ ID NO: 25 - protein, 1663 aa, Homo sapiens
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 26 - DNA, 5148 bp, Homo sapiens
agataaaaagccagctccagcaggcgctgctcactcctccccatcctctccctctgtccctctgtccctctgaccctgcactgtcccagcaccatgggacccacctcaggtcccagcctgctgctcctgctactaacccacctccccctggctctggggagtcccatgtactctatcatcacccccaacatcttgcggctggagagcgaggagaccatggtgctggaggcccacgacgcgcaaggggatgttccagtcactgttactgtccacgacttcccaggcaaaaaactagtgctgtccagtgagaagactgtgctgacccctgccaccaaccacatgggcaacgtcaccttcacgatcccagccaacagggagttcaagtcagaaaaggggcgcaacaagttcgtgaccgtgcaggccaccttcgggacccaagtggtggagaaggtggtgctggtcagcctgcagagcgggtacctcttcatccagacagacaagaccatctacacccctggctccacagttctctatcggatcttcaccgtcaaccacaagctgctacccgtgggccggacggtcatggtcaacattgagaacccggaaggcatcccggtcaagcaggactccttgtcttctcagaaccagcttggcgtcttgcccttgtcttgggacattccggaactcgtcaacatgggccagtggaagatccgagcctactatgaaaactcaccacagcaggtcttctccactgagtttgaggtgaaggagtacgtgctgcccagtttcgaggtcatagtggagcctacagagaaattctactacatctataacgagaagggcctggaggtcaccatcaccgccaggttcctctacgggaagaaagtggagggaactgcctttgtcatcttcgggatccaggatggcgaacagaggatttccctgcctgaatccctcaagcgcattccgattgaggatggctcgggggaggttgtgctgagccggaaggtactgctggacggggtgcagaacccccgagcagaagacctggtggggaagtctttgtacgtgtctgccaccgtcatcttgcactcaggcagtgacatggtgcaggcagagcgcagcgggatccccatcgtgacctctccctaccagatccacttcaccaagacacccaagtacttcaaaccaggaatgccctttgacctcatggtgttcgtgacgaaccctgatggctctccagcctaccgagtccccgtggcagtccagggcgaggacactgtgcagtctctaacccagggagatggcgtggccaaactcagcatcaacacacaccccagccagaagcccttgagcatcacggtgcgcacgaagaagcaggagctctcggaggcagagcaggctaccaggaccatgcaggctctgccctacagcaccgtgggcaactccaacaattacctgcatctctcagtgctacgtacagagctcagacccggggagaccctcaacgtcaacttcctcctgcgaatggaccgcgcccacgaggccaagatccgctactacacctacctgatcatgaacaagggcaggctgttgaaggcgggacgccaggtgcgagagcccggccaggacctggtggtgctgcccctgtccatcaccaccgacttcatcccttccttccgcctggtggcgtactacacgctgatcggtgccagcggccagagggaggtggtggccgactccgtgtgggtggacgtcaaggactcctgcgtgggctcgctggtggtaaaaagcggccagtcagaagaccggcagcctgtacctgggcagcagatgaccctgaagatagagggtgaccacggggcccgggtggtactggtggccgtggacaagggcgtgttcgtgctgaataagaagaacaaactgacgcagagtaagatctgggacgtggtggagaaggcagacatcggctgcaccccgggcagtgggaaggattacgccggtgtcttctccgacgcagggctgaccttcacgagcagcagtggccagcagaccgcccagagggcagaacttcagtgcccgcagccagccgcccgccgacgccgttccgtgcagctcacggagaagcgaatggacaaagtcggcaagtaccccaaggagctgcgcaagtgctgcgaggacggcatgcgggagaaccccatgaggttctcgtgccagcgccggacccgtttcatctccctgggcgaggcgtgcaagaaggtcttcctggactgctgcaactacatcacagagctgcggcggcagcacgcgcgggccagccacctgggcctggccaggagtaacctggatgaggacatcattgcagaagagaacatcgtttcccgaagtgagttcccagagagctggctgtggaacgttgaggacttgaaagagccaccgaaaaatggaatctctacgaagctcatgaatatatttttgaaagactccatcaccacgtgggagattctggctgtgagcatgtcggacaagaaagggatctgtgtggcagaccccttcgaggtcacagtaatgcaggacttcttcatcgacctgcggctaccctactctgttgttcgaaacgagcaggtggaaatccgagccgttctctacaattaccggcagaaccaagagctcaaggtgagggtggaactactccacaatccagccttctgcagcctggccaccaccaagaggcgtcaccagcagaccgtaaccatcccccccaagtcctcgttgtccgttccatatgtcatcgtgccgctaaagaccggcctgcaggaagtggaagtcaaggctgctgtctaccatcatttcatcagtgacggtgtcaggaagtccctgaaggtcgtgccggaaggaatcagaatgaacaaaactgtggctgttcgcaccctggatccagaacgcctgggccgtgaaggagtgcagaaagaggacatcccacctgcagacctcagtgaccaagtcccggacaccgagtctgagaccagaattctcctgcaagggaccccagtggcccagatgacagaggatgccgtcgacgcggaacggctgaagcacctcattgtgaccccctcgggctgcggggaacagaacatgatcggcatgacgcccacggtcatcgctgtgcattacctggatgaaacggagcagtgggagaagttcggcctagagaagcggcagggggccttggagctcatcaagaaggggtacacccagcagctggccttcagacaacccagctctgcctttgcggccttcgtgaaacgggcacccagcacctggctgaccgcctacgtggtcaaggtcttctctctggctgtcaacctcatcgccatcgactcccaagtcctctgcggggctgttaaatggctgatcctggagaagcagaagcccgacggggtcttccaggaggatgcgcccgtgatacaccaagaaatgattggtggattacggaacaacaacgagaaagacatggccctcacggcctttgttctcatctcgctgcaggaggctaaagatatttgcgaggagcaggtcaacagcctgccaggcagcatcactaaagcaggagacttccttgaagccaactacatgaacctacagagatcctacactgtggccattgctggctatgctctggcccagatgggcaggctgaaggggcctcttcttaacaaatttctgaccacagccaaagataagaaccgctgggaggaccctggtaagcagctctacaacgtggaggccacatcctatgccctcttggccctactgcagctaaaagactttgactttgtgcctcccgtcgtgcgttggctcaatgaacagagatactacggtggtggctatggctctacccaggccaccttcatggtgttccaagccttggctcaataccaaaaggacgcccctgaccaccaggaactgaaccttgatgtgtccctccaactgcccagccgcagctccaagatcacccaccgtatccactgggaatctgccagcctcctgcgatcagaagagaccaaggaaaatgagggtttcacagtcacagctgaaggaaaaggccaaggcaccttgtcggtggtgacaatgtaccatgctaaggccaaagatcaactcacctgtaataaattcgacctcaaggtcaccataaaaccagcaccggaaacagaaaagaggcctcaggatgccaagaacactatgatccttgagatctgtaccaggtaccggggagaccaggatgccactatgtctatattggacatatccatgatgactggctttgctccagacacagatgacctgaagcagctggccaatggtgttgacagatacatctccaagtatgagctggacaaagccttctccgataggaacaccctcatcatctacctggacaaggtctcacactctgaggatgactgtctagctttcaaagttcaccaatactttaatgtagagcttatccagcctggagcagtcaaggtctacgcctattacaacctggaggaaagctgtacccggttctaccatccggaaaaggaggatggaaagctgaacaagctctgccgtgatgaactgtgccgctgtgctgaggagaattgcttcatacaaaagtcggatgacaaggtcaccctggaagaacggctggacaaggcctgtgagccaggagtggactatgtgtacaagacccgactggtcaaggttcagctgtccaatgactttgacgagtacatcatggccattgagcagaccatcaagtcaggctcggatgaggtgcaggttggacagcagcgcacgttcatcagccccatcaagtgcagagaagccctgaagctggaggagaagaaacactacctcatgtggggtctctcctccgatttctggggagagaagcccaacctcagctacatcatcgggaaggacacttgggtggagcactggcccgaggaggacgaatgccaagacgaagagaaccagaaacaatgccaggacctcggcgccttcaccgagagcatggttgtctttgggtgccccaactgaccacacccccattcccccactccagataaagcttcagttatatctcaaaaaaaaaaaaaaaaa

SEQ ID NO: 27 - protein, 1091 aa, Homo sapiens
MAAGCLLALTLTLFQSLLIGPSSEEPFPSAVTIKSWVDKMQEDLVTLAKTASGVNQLVDIYEKYQDLYTVEPNNARQLVEIAARDIEKLLSNRSKALVRLALEAEKVQAAHQWREDFASNEVVYYNAKDDLDPEKNDSEPGSQRIKPVFIEDANFGRQISYQHAAVHIPTDIYEGSTIVLNELNWTSALDEVFKKNREEDPSLLWQVFGSATGLARYYPASPWVDNSRTPNKIDLYDVRRRPWYIQGAASPKDMLILVDVSGSVSGLTLKLIRTSVSEMLETLSDDDFVNVASFNSNAQDVSCFQHLVQANVRNKKVLKDAVNNITAKGITDYKKGFSFAFEQLLNYNVSRANCNKIIMLFTDGGEERAQEIFNKYNKDKKVRVFTFSVGQHNYDRGPIQWMACENKGYYYEIPSIGAIRINTQEYLDVLGRPMVLAGDKAKQVQWTNVYLDALELGLVITGTLPVFNITGQFENKTNLKNQLILGVMGVDVSLEDIKRLTPRFTLCPNGYYFAIDPNGYVLLHPNLQPKNPKSQEPVTLDFLDAELENDIKVEIRNKMIDGESGEKTFRTLVKSQDERYIDKGNRTYTWTPVNGTDYSLALVLPTYSFYYIKAKLEETITQARSKKGKMKDSETLKPDNFEESGYTFIAPRDYCNDLKISDNNTEFLLNFNEFIDRKTPNNPSCNADLINRVLLDAGFTNELVQNYWSKQKNIKGVKARFVVTDGGITRVYPKEAGENWQENPETYEDSFYKRSLDNDNYVFTAPYFNKSGPGAYESGIMVSKAVEIYIQGKLLKPAVVGIKIDVNSWIENFTKTSIRDPCAGPVCDCKRNSDVMDCVILDDGGFLLMANHDDYTNQIGRFFGEIDPSLMRHLVNISVYAFNKSYDYQSVCEPGAAPKQGAGHRSAYVPSVADILQIGWWATAAAWSILQQFLLSLTFPRLLEAVEMEDDDFTASLSKQSCITEQTQYFFDNDSKSFSGVLDCGNCSRIFHGEKLMNTNLIFIMVESKGTCPCDTRLLIQAEQTSDGPNPCDMVKQPRYRKGPDVCFDNNVLEDYTDCGGVSGLNPSLWYIIGIQFLLLWLVSGSTHRLL

SEQ ID NO: 28 - DNA, 7542 bp, Homo sapiens
agcgagagcgcgcgagcgccggcgggctcgccgaggtctgtttccaaagtcgcccttgatggcggcggaggcaaggcggccgcggcgcggagcagccgacgcacgctagtgggtccgcccgccaccgccccttcctcggcgtccgctcccgcccttgccgtcccccgcgcggctccgcgcctcgggccccgggcgcagccagccctccagacgcccgcggtcccggcggcgtgtgctgctcttcctccgcccgcggtttccagcgccgctccttcccccgcttgggcagggagggggcattgatcttcgatcgcgaagatggctgctggctgcctgctggccttgactctgacacttttccaatctttgctcatcggcccctcgtcggaggagccgttcccttcggccgtcactatcaaatcatgggtggataagatgcaagaagaccttgtcacactggcaaaaacagcaagtggagtcaatcagcttgttgatatttatgagaaatatcaagatttgtatactgtggaaccaaataatgcacgccagctggtagaaattgcagccagggatattgagaaacttctgagcaacagatctaaagccctggtgcgcctggcattggaagcggagaaagttcaagcagctcaccagtggagagaagattttgcaagcaatgaagttgtctactacaatgcaaaggatgatctcgatcctgagaaaaatgacagtgagccaggcagccagaggataaaacctgttttcattgaagatgctaattttggacgacaaatatcttatcagcacgcagcagtccatattcctactgacatctatgagggctcaacaattgtgttaaatgaactcaactggacaagtgccttagatgaagttttcaaaaagaatcgcgaggaagacccttcattattgtggcaggtttttggcagtgccactggcctagctcgatattatccagcttcaccatgggttgataatagtagaactccaaataagattgacctttatgatgtacgcagaagaccatggtacatccaaggagctgcatctcctaaagacatgcttattctggtggatgtgagtggaagtgttagtggattgacacttaaactgatccgaacatctgtctccgaaatgttagaaaccctctcagatgatgatttcgtgaatgtagcttcatttaacagcaatgctcaggatgtaagctgttttcagcaccttgtccaagcaaatgtaagaaataaaaaagtgttgaaagacgcggtgaataatatcacagccaaaggaattacagattataagaagggctttagttttgcttttgaacagctgcttaattataatgtttccagagcaaactgcaataagattattatgctattcacggatggaggagaagagagagcccaggagatatttaacaaatacaataaagataaaaaagtacgtgtattcacgttttcagttggtcaacacaattatgacagaggacctattcagtggatggcctgtgaaaacaaaggttattattatgaaattccttccattggtgcaataagaatcaatactcaggaatatttggatgttttgggaagaccaatggttttagcaggagacaaagctaagcaagtccaatggacaaatgtgtacctggatgcattggaactgggacttgtcattactggaactcttccggtcttcaacataaccggccaatttgaaaataagacaaacttaaagaaccagctgattcttggtgtgatgggagtagatgtgtctttggaagatattaaaagactgacaccacgttttacactgtgccccaatgggtattactttgcaatcgatcctaatggttatgttttattacatccaaatcttcagccaaagaaccccaaatctcaggagccagtaacattggatttccttgatgcagagttagagaatgatattaaagtggagattcgaaataagatgattgatggggaaagtggagaaaaaacattcagaactctggttaaatctcaagatgagagatatattgacaaaggaaacaggacatacacatggacacctgtcaatggcacagattacagtttggccttggtattaccaacctacagtttttactatataaaagccaaactagaagagacaataactcaggccagatcaaaaaagggcaaaatgaaggattcggaaaccctgaagccagataattttgaagaatctggctatacattcatagcaccaagagattactgcaatgacctgaaaatatcggataataacactgaatttcttttaaatttcaacgagtttattgatagaaaaactccaaacaacccatcatgtaacgcggatttgattaatagagtcttgcttgatgcaggctttacaaatgaacttgtccaaaattactggagtaagcagaaaaatatcaagggagtgaaagcacgatttgttgtgactgatggtgggattaccagagtttatcccaaagaggctggagaaaattggcaagaaaacccagagacatatgaggacagcttctataaaaggagcctagataatgataactatgttttcactgctccctactttaacaaaagtggacctggtgcctatgaatcgggcattatggtaagcaaagctgtagaaatatatattcaagggaaacttcttaaacctgcagttgttggaattaaaattgatgtaaattcctggatagagaatttcaccaaaacctcaatcagagatccgtgtgctggtccagtttgtgactgcaaaagaaacagtgacgtaatggattgtgtgattctggatgatggtgggtttcttctgatggcaaatcatgatgattatactaatcagattggaagattttttggagagattgatcccagcttgatgagacacctggttaatatatcagtttatgcttttaacaaatcttatgattatcagtcagtatgtgagcccggtgctgcaccaaaacaaggagcaggacatcgctcagcatatgtgccatcagtagcagacatattacaaattggctggtgggccactgctgctgcctggtctattctacagcagtttctcttgagtttgacctttccacgactccttgaggcagttgagatggaggatgatgacttcacggcctccctgtccaagcagagctgcattactgaacaaacccagtatttcttcgataacgacagtaaatcattcagtggtgtattagactgtggaaactgttccagaatctttcatggagaaaagcttatgaacaccaacttaatattcataatggttgagagcaaagggacatgtccatgtgacacacgactgctcatacaagcggagcagacttctgacggtccaaatccttgtgacatggttaagcaacccagataccgaaaagggcctgatgtctgctttgataacaatgtcttggaggattatactgactgtggtggtgtttctggattaaatccctccctgtggtatatcattggaatccagtttctactactttggctggtatctggcagcacacaccgcctgttatgaccttctaaaaaccaaatctgcatagttaaactccagaccctgccaaaacatgagccctgccctcaattacagtaacgtagggtcagctataaaatcagacaaacattagctgggcctgttccatggcataacactaaggcgcagactcctaaggcacccactggctgcatgtcagggtgtcagatccttaaacgtgtgtgaatgctgcatcatctatgtgtaacatcaaagcaaaatcctatacgtgtcctctattggaaaatttgggagtttgttgttgcattgttggtgattacatgtgaaagggttccccatacaattgttaatgaaccataagaaatgtcttgatattgacctggaattttgacttgctgcaattttactaagaaaatctctaaggggaaagaaattatttttgccttcactttttcttctgtgaaaagttaattcctgctttaagtagcaattattgaaatatatagagaaagagagaattaacattggtctaaattgttgaaatataaataatggctaattttctatgaaaaatttgccatgaataaaatgccttatgaagaacggcttctttgccaaaaataaaacacaattgatgaccaattcaattttgatggggtaatgaattgatggtttgaaaatagtaactaagaaattattaaactaaaggtgcttgagggagaaattagaatatgtgacatatttcctcataaatgtaagcaagcccttaataaatgctttgatgtaatgttccttttgttcacagtacagttatgtagtgctagccagatatttaaaaatgctctaagaaaagcttgtcaggggggagggggaaatgtttttcttgtgataaatgaactttggtttaccaagggtattttgcatgatgctgctgctattttaacttttcagttgttttttctcttactgtagagtgtagtcattgaaaagttaactgagtgatattgttatgtaagaatctgaaccttgcagtgtaaatgcacttaagtcatctttaaaatgttgttaaactactctgaatatactgtgcaatgtaaatgctgaatgattggggttagataaatggtgaaatgagaattaatgaattttatacagattcatgctttcgcctgataacctatgtacagatattttaagtacataaatcagatttgacacatttattttttgaaaagtacatatatgttctcaatgaagattcatcctaggtttcctctttgttcgtttcaatagtagcacatacaatccaaaagttcctgctactgctgcttttgcccttccattttaaatggtatgatcgtttggccaaaatttccatgcaagttgataagggttttacaataatctgaacaaataggaaaccttgaatctctatgtatgcacatgaatacttgattgtgaataataaaagttgttttttatacagcctcattggtgaaaatggttagtgtagtaaatcagatatttcattatagactaatatggtagcagattatttatgcttttaaaaattattttctggagcaaacagtatttgaagaaaaggtaaaaactatgaatgtgtaattctaagaatagttaataattttactgaaggctgaacgtatgtagatattttttatgataaaatgcagtttttctttttttggaacatgccaattactggtatgtgtatatgctggaaattttcttacatatcctttttaagtcaaagttattaacattatccagcatgctttaatatgctccacatatcaaagatataaccaataatttaacgtagcttaaaagataacatttgttttgtttttagctttgcatacatttgtaattccctcccaccaccaatataccttaggtaccatttgtgtacatttcaagattttttttttcaataaaaaggtggggggaaatggaatcactttgccaactattgttctaagagttgacatcagttaccatttctaatgcattgttgtgattttgtagtgtcattcataactggtattgacattttagaaaacaccaaagtgatttaaaatatatgtggggaaaaaaacttctcactctagtaacaatgttttaatcatgttgtttgttaaatatttaaaagttattgttcaatttttgctagactatcttaaatcatgatagtgttgggaagggggtatcaaaatataaaattgttagtttgtgtatataaacaatgcacattaaaacaattgaattgcttccagcatacaacaaactaccttaaaattatgttcaaaatctattaaatatattttcatggctaatgtcgagtatatgttttcatcaattacattgttattgtattaatcgcggttaaacttggcatctcttggcaggatcctatttaatttctgttgccatcaaaatatatttatatgcatgagttttgctataaacctttattttctgctataaagttatatattatgacaaccataaatgcaattacaaattgggtaagaacaagctgaataaaagttgttcccatgactaatattagtcttcatgcttttagcatattttttaagacagtaacactcatcccatcaaattattacattacatttagtatttttctgagttacattgacaagaacttaaattatagcctgttattaatattgatttccaacgactgaacagtacaacagatcaataaaaaagtttatacactgctttaactaatttttgttatactcatgtggcttagtgtagttagtcactaaaacagagttcaagtgagcttggcaatggtgttgagtaacaagaaagaattttgcattaatgatttctttttattttcctcatttaaacaaccagcattactgccaaacaccaaccgtggtccatatttgtaacaggtttttaacatgacatagttagcaagtttgattgaagagatttttaggtcctgggcctataagattttagtatgatcttttttcatgtttctaatgcttgaggtgttactgcttaacttgggaaaaaaaaaaacaaaaaacagtgttcctgccatttgtaagttttcctataatattccttttattaactttaaaactggacttatcgggttcaaataggcagtatcccttttaagtgacctttgcaacccaagtgttacttgcttatttgatgctctcttgtattttaaatctcatttaaatctttgtctgtgaccaacaaagcttaagattttgtcttcatttaaaactctgaatctattccattgatcatgttagacgttcaaatgtttaatggtatagtctgacctagtgaaacaaaatggatgttgttactttttttaaagaatagaatgaaataactatttcacttgcccaaaattttaaatattttttcatctaaacttgtattgtttcccatagcagaatgtcaatattcacagtacatttctgtaaagagcaaaccaatataatgttttgagtgttgaaaaaaattccagatttttgaagaattagacaactcttcatctaccttatttctagttcacacagttatctcaaattccactgaaactaatgggatactgtcttgtgtagatgccagttgagtttataatgtgacctagtaaagctgtcttttttgttgtgttgtatgagtgtcggatcatgcttttaggaatacttttattaaaatggtgtgcattcatgcaaaaggccaactggcttttgtgaacaatagatcttttctcccctttattttgttctcttgacacttttgtgaaaattacctagcctgataaaaacattttgtaaaaatttgtataatactgttataaaatatttataatcccagaaaatgttgtgttaaatcttgtgcaaaaacagtatattcagaataaaagtttatcatttaaagtca

SEQ ID NO: 29 - protein, 2181 aa, Homo sapiens
MMMMMMMKKMQHQRQQQADHANEANYARGTRLPLSGEGPTSQPNSSKQTVLSWQAAIDAARQAKAAQTMSTSAPPPVGSLSQRKRQQYAKSKKQGNSSNSRPARALFCLSLNNPIRRACISIVEWKPFDIFILLAIFANCVALAIYIPFPEDDSNSTNHNLEKVEYAFLIIFTVETFLKIIAYGLLLHPNAYVRNGWNLLDFVIVIVGLFSVILEQLTKETEGGNHSSGKSGGFDVKALRAFRVLRPLRLVSGVPSLQVVLNSIIKAMVPLLHIALLVLFVIIIYAIIGLELFIGKMHKTCFFADSDIVAEEDPAPCAFSGNGRQCTANGTECRSGWVGPNGGITNFDNFAFAMLTVFQCITMEGWTDVLYWVNDAIGWEWPWVYFVSLIILGSFFVLNLVLGVLSGEFSKEREKAKARGDFQKLREKQQLEEDLKGYLDWITQAEDIDPENEEEGGEEGKRNTSMPTSETESVNTENVSGEGENRGCCGSLWCWWRRRGAAKAGPSGCRRWGQAISKSKLSRRWRRWNRFNRRRCRAAVKSVTFYWLVIVLVFLNTLTISSEHYNQPDWLTQIQDIANKVLLALFTCEMLVKMYSLGLQAYFVSLFNRFDCFVVCGGITETILVELEIMSPLGISVFRCVRLLRIFKVTRHWTSLSNLVASLLNSMKSIASLLLLLFLFIIIFSLLGMQLFGGKFNFDETQTKRSTFDNFPQALLTVFQILTGEDWNAVMYDGIMAYGGPSSSGMIVCIYFIILFICGNYILLNVFLAIAVDNLADAESLNTAQKEEAEEKERKKIARKESLENKKNNKPEVNQIANSDNKVTIDDYREEDEDKDPYPPCDVPVGEEEEEEEEDEPEVPAGPRPRRISELNMKEKIAPIPEGSAFFILSKTNPIRVGCHKLINHHIFTNLILVFIMLSSAALAAEDPIRSHSFRNTILGYFDYAFTAIFTVEILLKMTTFGAFLHKGAFCRNYFNLLDMLVVGVSLVSFGIQSSAISVVKILRVLRVLRPLRAINRAKGLKHVVQCVFVAIRTIGNIMIVTTLLQFMFACIGVQLFKGKFYRCTDEAKSNPEECRGLFILYKDGDVDSPVVRERIWQNSDFNFDNVLSAMMALFTVSTFEGWPALLYKAIDSNGENIGPIYNHRVEISIFFIIYIIIVAFFMMNIFVGFVIVTFQEQGEKEYKNCELDKNQRQCVEYALKARPLRRYIPKNPYQYKFWYVVNSSPFEYMMFVLIMLNTLCLAMQHYEQSKMFNDAMDILNMVFTGVFTVEMVLKVIAFKPKGYFSDAWNTFDSLIVIGSIIDVALSEADPTESENVPVPTATPGNSEESNRISITFFRLFRVMRLVKLLSRGEGIRTLLWTFIKSFQALPYVALLIAMLFFIYAVIGMQMFGKVAMRDNNQINRNNNFQTFPQAVLLLFRCATGEAWQEIMLACLPGKLCDPESDYNPGEEYTCGSNFAIVYFISFYMLCAFLIINLFVAVIMDNFDYLTRDWSILGPHHLDEFKRIWSEYDPEAKGRIKHLDVVTLLRRIQPPLGFGKLCPHRVACKRLVAMNMPLNSDGTVMFNATLFALVRTALKIKTEGNLEQANEELRAVIKKIWKKTSMKLLDQVVPPAGDDEVTVGKFYATFLIQDYFRKFKKRKEQGLVGKYPAKNTTIALQAGLRTLHDIGPEIRRAISCDLQDDEPEETKREEEDDVFKRNGALLGNHVNHVNSDRRDSLQQTNTTHRPLHVQRPSIPPASDTEKPLFPPAGNSVCHNHHNHNSIGKQVPTSTNANLNNANMSKAAHGKRPSIGNLEHVSENGHHSSHKHDREPQRRSSVKRTRYYETYIRSDSGDEQLPTICREDPEIHGYFRDPHCLGEQEYFSSEECYEDDSSPTWSRQNYGYYSRYPGRNIDSERPRGYHHPQGFLEDDDSPVCYDSRRSPRRRLLPPTPASHRRSSFNFECLRRQSSQEEVPSSPIFPHRTALPLHLMQQQIMAVAGLDSSKAQKYSPSHSTRSWATPPATPPYRDWTPCYTPLIQVEQSEALDQVNGSLPSLHRSSWYTDEPDISYRTFTPASLTVPSSFRNKNSDKQRSADSLVEAVLISEGLGRYARDPKFVSATKHEIADACDLTIDEMESAASTLLNGNVRPRANGDVGPLSHRQDYELQDFGPGYSDEEPDPGRDEEDLADEMICITTL

SEQ ID NO: 30 - DNA, 7726 bp, Homo sapiens
agaataagggcagggaccgcggctcctacctcttggtgatccccttccccattccgcccccgcctcaacgcccagcacagtgccctgcacacagtagtcgctcaataaatgttcgtggatgatgatgatgatgatgatgaaaaaaatgcagcatcaacggcagcagcaagcggaccacgcgaacgaggcaaactatgcaagaggcaccagacttcctctttctggtgaaggaccaacttctcagccgaatagctccaagcaaactgtcctgtcttggcaagctgcaatcgatgctgctagacaggccaaggctgcccaaactatgagcacctctgcacccccacctgtaggatctctctcccaaagaaaacgtcagcaatacgccaagagcaaaaaacagggtaactcgtccaacagccgacctgcccgcgcccttttctgtttatcactcaataaccccatccgaagagcctgcattagtatagtggaatggaaaccatttgacatatttatattattggctatttttgccaattgtgtggccttagctatttacatcccattccctgaagatgattctaattcaacaaatcataacttggaaaaagtagaatatgccttcctgattatttttacagtcgagacatttttgaagattatagcgtatggattattgctacatcctaatgcttatgttaggaatggatggaatttactggattttgttatagtaatagtaggattgtttagtgtaattttggaacaattaaccaaagaaacagaaggcgggaaccactcaagcggcaaatctggaggctttgatgtcaaagccctccgtgcctttcgagtgttgcgaccacttcgactagtgtcaggagtgcccagtttacaagttgtcctgaactccattataaaagccatggttcccctccttcacatagcccttttggtattatttgtaatcataatctatgctattataggattggaactttttattggaaaaatgcacaaaacatgtttttttgctgactcagatatcgtagctgaagaggacccagctccatgtgcgttctcagggaatggacgccagtgtactgccaatggcacggaatgtaggagtggctgggttggcccgaacggaggcatcaccaactttgataactttgcctttgccatgcttactgtgtttcagtgcatcaccatggagggctggacagatgtgctctactgggtaaatgatgcgataggatgggaatggccatgggtgtattttgttagtctgatcatccttggctcatttttcgtccttaacctggttcttggtgtccttagtggagaattctcaaaggaaagagagaaggcaaaagcacggggagatttccagaagctccgggagaagcagcagctggaggaggatctaaagggctacttggattggatcacccaagctgaggacatcgatccggagaatgaggaagaaggaggagaggaaggcaaacgaaatactagcatgcccaccagcgagactgagtctgtgaacacagagaacgtcagcggtgaaggcgagaaccgaggctgctgtggaagtctctggtgctggtggagacggagaggcgcggccaaggcggggccctctgggtgtcggcggtggggtcaagccatctcaaaatccaaactcagccgacgctggcgtcgctggaaccgattcaatcgcagaagatgtagggccgccgtgaagtctgtcacgttttactggctggttatcgtcctggtgtttctgaacaccttaaccatttcctctgagcactacaatcagccagattggttgacacagattcaagatattgccaacaaagtcctcttggctctgttcacctgcgagatgctggtaaaaatgtacagcttgggcctccaagcatatttcgtctctcttttcaaccggtttgattgcttcgtggtgtgtggtggaatcactgagacgatcttggtggaactggaaatcatgtctcccctggggatctctgtgtttcggtgtgtgcgcctcttaagaatcttcaaagtgaccaggcactggacttccctgagcaacttagtggcatccttattaaactccatgaagtccatcgcttcgctgttgcttctgctttttctcttcattatcatcttttccttgcttgggatgcagctgtttggcggcaagtttaattttgatgaaacgcaaaccaagcggagcacctttgacaatttccctcaagcacttctcacagtgttccagatcctgacaggcgaagactggaatgctgtgatgtacgatggcatcatggcttacgggggcccatcctcttcaggaatgatcgtctgcatctacttcatcatcctcttcatttgtggtaactatattctactgaatgtcttcttggccatcgctgtagacaatttggctgatgctgaaagtctgaacactgctcagaaagaagaagcggaagaaaaggagaggaaaaagattgccagaaaagagagcctagaaaataaaaagaacaacaaaccagaagtcaaccagatagccaacagtgacaacaaggttacaattgatgactatagagaagaggatgaagacaaggacccctatccgccttgcgatgtgccagtaggggaagaggaagaggaagaggaggaggatgaacctgaggttcctgccggaccccgtcctcgaaggatctcggagttgaacatgaaggaaaaaattgcccccatccctgaagggagcgctttcttcattcttagcaagaccaacccgatccgcgtaggctgccacaagctcatcaaccaccacatcttcaccaacctcatccttgtcttcatcatgctgagcagcgctgccctggccgcagaggaccccatccgcagccactccttccggaacacgatactgggttactttgactatgccttcacagccatctttactgttgagatcctgttgaagatgacaacttttggagctttcctccacaaaggggccttctgcaggaactacttcaatttgctggatatgctggtggttggggtgtctctggtgtcatttgggattcaatccagtgccatctccgttgtgaagattctgagggtcttaagggtcctgcgtcccctcagggccatcaacagagcaaaaggacttaagcacgtggtccagtgcgtcttcgtggccatccggaccatcggcaacatcatgatcgtcaccaccctcctgcagttcatgtttgcctgtatcggggtccagttgttcaaggggaagttctatcgctgtacggatgaagccaaaagtaaccctgaagaatgcaggggacttttcatcctctacaaggatggggatgttgacagtcctgtggtccgtgaacggatctggcaaaacagtgatttcaacttcgacaacgtcctctctgctatgatggcgctcttcacagtctccacgtttgagggctggcctgcgttgctgtataaagccatcgactcgaatggagagaacatcggcccaatctacaaccaccgcgtggagatctccatcttcttcatcatctacatcatcattgtagctttcttcatgatgaacatctttgtgggctttgtcatcgttacatttcaggaacaaggagaaaaagagtataagaactgtgagctggacaaaaatcagcgtcagtgtgttgaatacgccttgaaagcacgtcccttgcggagatacatccccaaaaacccctaccagtacaagttctggtacgtggtgaactcttcgcctttcgaatacatgatgtttgtcctcatcatgctcaacacactctgcttggccatgcagcactacgagcagtccaagatgttcaatgatgccatggacattctgaacatggtcttcaccggggtgttcaccgtcgagatggttttgaaagtcatcgcatttaagcctaaggggtattttagtgacgcctggaacacgtttgactccctcatcgtaatcggcagcattatagacgtggccctcagcgaagcagacccaactgaaagtgaaaatgtccctgtcccaactgctacacctgggaactctgaagagagcaatagaatctccatcacctttttccgtcttttccgagtgatgcgattggtgaagcttctcagcaggggggaaggcatccggacattgctgtggacttttattaagtcctttcaggcgctcccgtatgtggccctcctcatagccatgctgttcttcatctatgcggtcattggcatgcagatgtttgggaaagttgccatgagagataacaaccagatcaataggaacaataacttccagacgtttccccaggcggtgctgctgctcttcaggtgtgcaacaggtgaggcctggcaggagatcatgctggcctgtctcccagggaagctctgtgaccctgagtcagattacaaccccggggaggagtatacatgtgggagcaactttgccattgtctatttcatcagtttttacatgctctgtgcatttctgatcatcaatctgtttgtggctgtcatcatggataatttcgactatctgacccgggactggtctattttggggcctcaccatttagatgaattcaaaagaatatggtcagaatatgaccctgaggcaaagggaaggataaaacaccttgatgtggtcactctgcttcgacgcatccagcctcccctggggtttgggaagttatgtccacacagggtagcgtgcaagagattagttgccatgaacatgcctctcaacagtgacgggacagtcatgtttaatgcaaccctgtttgctttggttcgaacggctcttaagatcaagaccgaagggaacctggagcaagctaatgaagaacttcgggctgtgataaagaaaatttggaagaaaaccagcatgaaattacttgaccaagttgtccctccagctggtgatgatgaggtaaccgtggggaagttctatgccactttcctgatacaggactactttaggaaattcaagaaacggaaagaacaaggactggtgggaaagtaccctgcgaagaacaccacaattgccctacaggcgggattaaggacactgcatgacattgggccagaaatccggcgtgctatatcgtgtgatttgcaagatgacgagcctgaggaaacaaaacgagaagaagaagatgatgtgttcaaaagaaatggtgccctgcttggaaaccatgtcaatcatgttaatagtgataggagagattcccttcagcagaccaataccacccaccgtcccctgcatgtccaaaggccttcaattccacctgcaagtgatactgagaaaccgctgtttcctccagcaggaaattcggtgtgtcataaccatcataaccataattccataggaaagcaagttcccacctcaacaaatgccaatctcaataatgccaatatgtccaaagctgcccatggaaagcggcccagcattgggaaccttgagcatgtgtctgaaaatgggcatcattcttcccacaagcatgaccgggagcctcagagaaggtccagtgtgaaaagaacccgctattatgaaacttacattaggtccgactcaggagatgaacagctcccaactatttgccgggaagacccagagatacatggctatttcagggacccccactgcttgggggagcaggagtatttcagtagtgaggaatgctacgaggatgacagctcgcccacctggagcaggcaaaactatggctactacagcagatacccaggcagaaacatcgactctgagaggccccgaggctaccatcatccccaaggattcttggaggacgatgactcgcccgtttgctatgattcacggagatctccaaggagacgcctactacctcccaccccagcatcccaccggagatcctccttcaactttgagtgcctgcgccggcagagcagccaggaagaggtcccgtcgtctcccatcttcccccatcgcacggccctgcctctgcatctaatgcagcaacagatcatggcagttgccggcctagattcaagtaaagcccagaagtactcaccgagtcactcgacccggtcgtgggccacccctccagcaacccctccctaccgggactggacaccgtgctacacccccctgatccaagtggagcagtcagaggccctggaccaggtgaacggcagcctgccgtccctgcaccgcagctcctggtacacagacgagcccgacatctcctaccggactttcacaccagccagcctgactgtccccagcagcttccggaacaaaaacagcgacaagcagaggagtgcggacagcttggtggaggcagtcctgatatccgaaggcttgggacgctatgcaagggacccaaaatttgtgtcagcaacaaaacacgaaatcgctgatgcctgtgacctcaccatcgacgagatggagagtgcagccagcaccctgcttaatgggaacgtgcgtccccgagccaacggggatgtgggccccctctcacaccggcaggactatgagctacaggactttggtcctggctacagcgacgaagagccagaccctgggagggatgaggaggacctggcggatgaaatgatatgcatcaccaccttgtagcccccagcgaggggcagactggctctggcctcaggtggggcgcaggagagccaggggaaaagtgcctcatagttaggaaagtttaggcactagttgggagtaatattcaattaattagacttttgtataagagatgtcatgcctcaagaaagccataaacctggtaggaacaggtcccaagcggttgagcctggcagagtaccatgcgctcggccccagctgcaggaaacagcaggccccgccctctcacagaggatgggtgaggaggccagacctgccctgccccattgtccagatgggcactgctgtggagtctgcttctcccatgtaccagggcaccaggcccacccaactgaaggcatggcggcggggtgcaggggaaagttaaaggtgatgacgatcatcacacctgtgtcgttacctcagccatcggtctagcatatcagtcactgggcccaacatatccatttttaaaccctttcccccaaatacactgcgtcctggttcctgtttagctgttctgaaatacggtgtgtaagtaagtcagaacccagctaccagtgattattgcgagggcaatgggacctcataaataaggttttctgtgatgtgacgccagtttacataagagaatatcactccgatggtcggtttctgactgtcacgctaagggcaactgtaaactggaataataatgcactcgcaaccaggtaaacttagatacactagtttgtttaaaattatagatttactgtacatgacttgtaatatactataatttgtatttgtaaagagatggtctatattttgtaattactgtattgtatttgaactgcagcaatatccatgggtcctaataattgtagttccccactaaaatctagaaattattagtatttttactcgggctatccagaagtagaagaaatagagccaattctcatttattcagcgaaaatcctctggggttaaaattttaagtttgaaagaacttgacactacagaaatttttctaaaatattttgagtcactataaacctatcatctttccacaagataaaa

SEQ ID NO: 31 - protein, 205 aa, Homo sapiens
MCKGLAGLPASCLRSAKDMKHRLGFLLQKSDSCEHNSSHNKKDKVVICQRVSQEEVKKWAESLENLISHECGLAAFKAFLKSEYSEENIDFWISCEEYKKIKSPSKLSPKAKKIYNEFISVQATKEVNLDSCTREETSRNMLEPTITCFDEAQKKIFNLMEKDSYRRFLKSRFYLDLVNPSSCGAEKQKGAKSSADCASLVPQCA

SEQ ID NO: 32 - DNA, 3055 bp, Homo sapiens
actttcccgaggtgcttctacagttccctctgccagcaggggaacagatggaaatagcaatcacctgccagaaggtggcgtgcagcaaggatgtgcatcttttgccgctactgctttctgattcctaaaaattactcagagatcactcatgtgttcagtgattcaggttctgttgaagataccaaagatattcggttggtcaaaatgacgggcatataaaggcttctcaggtttctgagtgcaaaagatatgaaacatcggctaggtttcctgctgcaaaaatctgattcctgtgaacacaattcttcccacaacaagaaggacaaagtggttatttgccagagagtgagccaagaggaagtcaagaaatgggctgaatcactggaaaacctgattagtcatgaatgtgggctggcagctttcaaagctttcttgaagtctgaatatagtgaggagaatattgacttctggatcagctgtgaagagtacaagaaaatcaaatcaccatctaaactaagtcccaaggccaaaaagatctataatgaattcatctcagtccaggcaaccaaagaggtgaacctggattcttgcaccagggaagagacaagccggaacatgctagagcctacaataacctgctttgatgaggcccagaagaagattttcaacctgatggagaaggattcctaccgccgcttcctcaagtctcgattctatcttgatttggtcaacccgtccagctgtggggcagaaaagcagaaaggagccaagagttcagcagactgtgcttccctggtccctcagtgtgcctaattctcacctgaaggcagagggatgaaatgccaagactctatgctctggaaaacctgaggccaaatattgatctgtattaagctccagtgctttatccacattgtagcctaatattcatgctgcctgccatgtgtgagtcacttctacgcataaactagatatagcttttggtgtttgagtgttcatcagggtgggaccccattccagtccaattttcctaagtttctttgagggttccatgggagcaaatatctaaataatggcctggtaggtctggattttcaaagattgttggcagtttcctcctcccaacagttttacctcgggatggttggttagtgcatgtcacatgacatccacatgcacatgtattctgttggccagcacgttctccagactctagatgtttagatgaggttgagctatgatatgtgcttgtgtgtatgtctatgtgtatatattatatatacattagacacacatatacattatttctgtatatagatgtctgtgtatacatatgtatgtgtgagtgtatgtatacacacacacacacacacacacacacacttttgcaagagtgatgggaaagaccctaggtgctcataactagagtatgtgtatgtacttacatgggtgttttgatctctgttctttcatactacatttgaacagggcaaaatgaactaactgccatgtaggctaagaaagaaatgctaacctgtggaaagttggttttgtaaaattccatggatcttgctggagaagcatccaaggaacttcatgcttgatttgaccactgacagcctccaccttgagcactattctaaggagcaaataccttagctcccttgagctggttttctctgatggcacttttgagctcctaagctgccagccttcccttcttttcctgggtgctcagggcatgcttattagcagctgggttggtatggagttggcagacaggatgttcaacttaatgaagaaatacagctaaggccttgccagcaacacctgccgtaagttactggctgagtgagggcatagaagttaaaggttactgtttttatcctctatccttttttcctttcctgatcaaggtgctcttctcattttttcctgagaaccttagccatcagatgaggctccttagtttattgtggttggttgttttttctttataatggctctgggctatatgcctatatttataaaccagcagcaggggaaagattatattttataagagggaacaaattttcacaatttgaaaagcccacataagttttctcttttaaggtagaatcttgttaatttcattccaaacatcggggctaacagagactggaggcatttctttttaggctctgagactaaatgagaggaaaagaaaagaaaaaaaaaatgattgtctaaccaattgtgagaattactgtttgaaacttttcaaggcacattgaaatacttgaaaacttctcatttatgttatttatgatgttattttgtacgtgttattattattatattgttttataaatggaggtacaggatatcacctgaattattaatgaatgcccaggaagtaattttcttctcattcttctaaaactactgcctttcaaagtgcacacacacgcgtccacatacactgcattcgttgctccagtataaattacatgcatgagcacctttctggcttttaagccaatataatgggctgcaaaatgaagacaccagagtgtatgcatacaaatctcactgtattaaagatgcaggttttctaattgtacccttcttgtctctctggcaatcttgcccttaatatccctggagttcctcatcagtgtcattttctgttatacacagttccacaattttgtctctagttgacttcaaatgtgtaactttattggtcttgccctattataattgtcatgactttcagattgtatctgaactcacagactgctgtcttactaataggtctggaaggtcacgctgaatgagaagtaaattattttatgtaatacatttttgagtgtgtttttcagttgtatttccctgttatttcatcactatttccaatggtgagcttgcctgctcatgctccctggacagaatactccttccttttgcatgcctgtttctatcatgtgcttgataggcctcaaagctaatgcttccagtgaaacacacgcatcttaataataagggtaaataaacgctccatatgaaacta

SEQ ID NO: 33 - protein, 313 aa, Homo sapiens
MLLLADMDVVNQLVAGGQFRVVKEPLGFVKVLQWVFAIFAFATCGSYSGELQLSVDCANKTESDLSIEVEFEYPFRLHQVYFDAPTCRGGTTKVFLVGDYSSSAEFFVTVAVFAFLYSMGALATYIFLQNKYRENNKGPMLDFLATAVFAFMWLVSSSAWAKGLSDVKMATDPENIIKEMPVCRQTGNTCKELRDPVTSGLNTSVVFGFLNLVLWVGNLWFVFKETGWAAPFLRAPPGAPEKQPAPGDAYGDAGYGQGPGGYGPQDSYGPQGGYQPDYGQPAGSGGSGYGPQGDYGQQGYGPQGAPTSFSNQM

SEQ ID NO: 34 - DNA, 2449 bp, Homo sapiens
gccccctgcattgctgatgctgctgctggcggacatggacgtggtgaatcagctggtggctgggggtcagttccgggtggtcaaggagcccctcggctttgtgaaggtgctgcaatgggtcttcgccatcttcgcctttgccacatgcggcagctacagtggggagctccagctgagcgtggattgtgccaacaagaccgagagtgacctcagcatcgaggtcgagttcgagtaccccttcaggctgcaccaagtgtactttgatgcacccacctgccgagggggcaccaccaaggtcttcttagttggggactactcctcgtcagccgaattctttgtcaccgtggccgtgtttgccttcctctactccatgggggctctggccacctacatcttcctgcagaacaagtaccgagagaataacaaagggcccatgctggactttctggccacggctgtgttcgccttcatgtggctagttagctcatcggcatgggccaaggggctgtcagatgtgaagatggccacagacccagagaacattatcaaggagatgcctgtctgccgccagacagggaacacatgcaaggagctgagagaccctgtgacctcgggactcaacacctcggtggtgttcggcttcctgaacctggtgctctgggtcggcaacctgtggttcgtgtttaaggagacaggctgggccgccccgttcctgcgcgcgcctcccggcgcccccgagaaacaaccggcacccggggacgcctacggcgatgcaggctacgggcagggccccggcgggtacgggccccaggattcctacgggcctcagggcggctaccagcctgactatggtcaaccagccggcagcggtggcagtggctacgggcctcagggcgactatgggcagcaaggctacggcccgcagggtgcacccacctccttctccaatcagatgtagtctggtcagtgaagcccaggaggacctggggggggcaagagctcaggagaaggcctgccccccttcccacccctataccctaggtctccacccctcaagccaggagaccctgtctttgctgtttatatatatatatattatatataaatatctatttatctgtctgagccctgccctcactccactcccctcatccactaggtgcccagtcttgagtgggccccctctcttaccccgtccctttccctgcatcccttggcccctctctgtttaccctccctgtcccctgaggttaaggggatctaaaaggaggacagggagggaacagacctcggctgtgtggggagggtgggcgtgacttcagactctctcctctctctccctccactcctcccaactctggccttggttcctccagcaatgcctgcctgaacaaaggccgttagggaaatccaactccagggttaaagaaaggcagagattgggggggcttggggtagagaggacagtttaggacccaaggtggtcttggagaggaggtgtggagtggaggggtcagcaggggggttgggttccagacagagtggatctggagtctgaaggagaggagtgcgctagagcattctggggtggggcttggaagggcgctgagggcagggttctagaaggggcgaggctttaagcgaggcagaatggtgggctccagagtaggtgggtcttggattggtaccagagcctatggaaagggtgtggcttggaacatttgggagactgagcttgattctaaaggggacagatcttgagcaaggcaagaagtgggattcaggaatgggccaagccagggttccagacagggtggggcttagaatggggcttccatggtggtttcagaaagggcagcccctccccatggtgcagtgaagaaaatgttttacaatggctgggtttgggcagtggagaggggacttggataggagcttccagatgggttttgttaggggtgggggagaatggctctggctacgacttgggacggaagtggcctgagaagagtcgagtgatatggcttgtagggtgaggcgtgggatccagagagaagcaccccaccacacacacccttccccactcccgtgatgaaacagctaggttaataggaggacagaaccaacgggtctgtgggactggcccacccctcttcccccttcccctgcgccctccctccctccacacctccacccgtcctggggtggttggaggcctggtctggagcccctatcctgcaccctctgctatgtctgtgatgtcagtagtgcctgtgatcgtgtgttgccattttgtctggctgtggcccctccttctcccctccagacccctaccctttcccaaacccttcggtattgttcaaagaacccccctccccaaggaagaacaaatatgattctcctctcccaaataaactccttaaccacctagtcaaaaaaaaaaaaaaaa

SEQ ID NO: 35 - DNA, 19 bp, Homo sapiens
cagtagtttggccatggga

SEQ ID NO: 36 - DNA, 20 bp, Artificial Sequence
  Chemically Sythesized Primer
ctgacggtccaaatccttgt

SEQ ID NO: 37 - DNA, 20 bp, Artificial Sequence
  Chemically Synthesized Primer
tgccagataccagccaaagt

SEQ ID NO: 38 - DNA, 20 bp, Artificial Sequence
  Chemically Synthesized Primer
ccagagagtgagccaagagg

SEQ ID NO: 39 - DNA, 20 bp, Artificial Sequence
  Chemically Synthesized Sequence
atctttttggccttgggact

SEQ ID NO: 40 - DNA, 20 bp, Artificial Sequence
  Chemically Synthesized Primer
catgtcctcctgcatctcct

SEQ ID NO: 41 - DNA, 20 bp, Artificial Sequence
  Chemically Synthesized Primer
agccttgcaagtgaatggaa

SEQ ID NO: 42 - DNA, 20 bp, Artificial Sequence
  Chemically Synthesized Primer
gcatcgagaggagttgaagc

SEQ ID NO: 43 - DNA, 20 bp, Artificial Sequence
  Chemically Synthesized Primer
gccctctgggtattttccat

SEQ ID NO: 44 - DNA, 20 bp, Artificial Sequence
  Chemically Synthesized Primer
attgctgcctacgtccactt

SEQ ID NO: 45 - DNA, 20 bp, Artificial Sequence
  Chemically Synthesized Primer
ttgggtgttttgccaatgta

SEQ ID NO: 46 - DNA, 20 bp, Artificial Sequence
  Chemically Synthesized Primer
tccctgtgaaaacaagagca

SEQ ID NO: 47 - DNA, 20 bp, Artificial Sequence
  Chemically Synthesized Primer
atagagtcgccaccctgatg

SEQ ID NO: 48 - DNA, 27 bp, Artificial Sequence
  Chemically Synthesized Primer
gatcttttccaccccaagagtacataa

SEQ ID NO: 49 - DNA, 25 bp, Artificial Sequence
  Chemically Synthesized Primer
ctcacacaaacacagattccacaac

SEQ ID NO: 50 - protein, 471 aa, Homo sapiens
MQSNKTFNLEKQNHTPRKHHQHHHQQQHHQQQQQQPPPPPIPANGQQASSQNEGLTIDLKNFRKPGEKTFTQRSRLFVGNLPPDITEEEMRKLFEKYGKAGEVFIHKDKGFGFIRLETRTLAEIAKVELDNMPLRGKQLRVRFACHSASLTVRNLPQYVSNELLEEAFSVFGQVERAVVIVDDRGRPSGKGIVEFSGKPAARKALDRCSEGSFLLTTFPRPVTVEPMDQLDDEEGLPEKLVIKNQQFHKEREQPPRFAQPGSFEYEYAMRWKALIEMEKQQQDQVDRNIKEAREKLEMEMEAARHEHQVMLMRQDLMRRQEELRRMEELHNQEVQKRKQLELRQEEERRRREEEMRRQQEEMMRRQQEGFKGTFPDAREQEIRMGQMAMGGAMGINNRGAMPPAPVPAGTPAPPGPATMMPDGTLGLTPPTTERFGQAATMEGIGAIGGTPPAFNRAAPGAEFAPNKRRRY